(19) United States
(12) Patent Application Publication
(10) Pub. No.: US 2024/0409975 A1
(43) Pub. Date: Dec. 12, 2024

(54) SYNTHETIC PRODUCTION OF CIRCULAR DNA VECTORS

(71) Applicant: Intergalactic Therapeutics, Inc., Cambridge, MA (US)

(72) Inventors: Jin HUH, Cambridge, MA (US); Eileen HIGHAM, Cambridge, MA (US); Jose LORA, Cambridge, MA (US); Jodi KENNEDY, Cambridge, MA (US); Anne MAGUIRE, Cambridge, MA (US); Meisam BAKHSHAYESH, Cambridge, MA (US); Eliza DORNBUSH, Cambridge, MA (US)

(21) Appl. No.: 18/695,514

(22) PCT Filed: Sep. 27, 2022
(86) PCT No.: PCT/US2022/077108

§ 371 Date: Mar. 26, 2024

Related U.S. Application Data
(60) Provisional application No. 63/248,801, filed on Sep. 27, 2021.

(51) Int. Cl. C12P 19/34; C12N 15/85

(52) U.S. Cl. CPC C12P 19/34; C12N 15/85; C12N 2800/10

(57) ABSTRACT

Provided herein are improved methods of producing therapeutic circular DNA vectors, pharmaceutical compositions produced by such methods, and methods of using pharmaceutical compositions. The invention is based, at least in part, on cell-free manufacturing processes involving restriction digest and ligation schemes, such as restriction digest processes involving type IIs restriction enzymes. Methods provided herein are amenable to large scale production of high-purity compositions of therapeutic circular DNA vectors.

### CROSS REFERENCE

This international application claims priority to U.S. Provisional Patent Application No. 63/248,801.

### FIELD OF THE INVENTION

In general, the invention involves synthetic circular DNA vectors.

### BACKGROUND

Gene therapy is emerging as a promising approach to treat a wide variety of diseases and disorders in human patients. Recombinant adeno-associated viral (rAAV) vectors have an established record of high-efficiency gene transfer in human patients and a variety of model systems. Genomes of rAAV vectors are advantageous for their ability to persist in vivo as circular episomes for the life of the target cell. On the other hand, rAAV-based vectors suffer substantial drawbacks, such as limited maximum payload, immunogenicity, and manufacturing inefficiencies.

To address some of these challenges in rAAV technology, non-viral alternatives have gained traction in recent years. However, development of a scalable non-viral gene therapy platform that enjoys the efficiency and persistence of rAAV has proven elusive. For example, traditional bacterial plasmid DNA vectors represent a versatile tool in gene delivery but are limited by their bacterial origin. Bacterial components of plasmid DNA vectors, such as antibiotic resistance genes, origins of replication, and impurities from the bacterial host, such as endotoxins, bacterial genomic DNA and RNA, and host cell protein, can lead to immunogenicity and loss of gene expression by transcriptional silencing.

While improvements to plasmid DNA vectors have been achieved on a small scale by removing bacterial components through site-specific recombination, such processes nevertheless rely on production in bacterial host cells, which inherently carries risks of unacceptable impurity profiles in resulting pharmaceutical compositions. Synthetic DNA vectors are made in cell-free conditions and obviate these risks; however, their scalability, to date, is constrained by inefficient manufacturing processes, often requiring gel purification steps and multiple restriction enzymes.

Thus, there is a need in the field for controllable, scalable methods of producing non-viral DNA vectors with high purity and efficiency.

### SUMMARY OF THE INVENTION

Provided herein are improved, cell-free methods of producing therapeutic circular DNA vectors, pharmaceutical compositions produced by such methods, and methods of using pharmaceutical compositions. The invention is based, at least in part, on the development of cell-free manufacturing processes involving restriction digest and ligation schemes, such as restriction digest processes involving type IIs restriction enzymes. Moreover, Applicant has identified conditions (e.g., DNA and ligase concentrations), process sequences, and high-efficiency overhang compositions that confer dramatic improvements in synthetic DNA vector manufacturing efficiency. Methods and compositions provided herein are amenable to large-scale production of high-purity compositions of therapeutic circular DNA vectors.

In one aspect, provided herein is a method of producing a therapeutic circular DNA vector involving the following steps: (a) providing a sample comprising a template DNA vector (e.g., a plasmid DNA vector) comprising a therapeutic sequence and a backbone sequence; (b) amplifying the template DNA vector using a polymerase-mediated rolling-circle amplification (e.g., Phi29-mediated rolling-circle amplification) to generate a linear concatemer; (c) digesting the linear concatemer with a type IIs restriction enzyme that cuts a first site and a second site per unit of the linear concatemer, wherein the first and second sites flank the therapeutic sequence and form self-complementary overhangs, thereby producing a linear therapeutic fragment and a linear backbone fragment, wherein the linear therapeutic fragment comprises the therapeutic sequence and the linear backbone fragment comprises the backbone sequence, or a portion thereof, and (d) contacting the linear backbone fragment and the linear therapeutic fragment with a ligase to produce a circular backbone and a therapeutic circular DNA vector lacking a type IIs restriction site. In some embodiments, the linear backbone fragment of (c) comprises a type IIs restriction site, the circular backbone of (d) comprises the type IIs restriction site, and the type IIs restriction enzyme cuts the circular backbone and does not cut the therapeutic circular DNA vector.

In another aspect, provided herein is a method of producing a therapeutic circular DNA vector involving the following steps: (a) providing a sample comprising a template DNA vector (e.g., plasmid DNA vector) comprising a therapeutic sequence and a backbone sequence; (b) amplifying the template DNA vector using a polymerase-mediated rolling-circle amplification to generate a linear concatemer; (c) digesting the linear concatemer with one or more restriction enzymes that cut at least a first site, a second site, and a third site per unit of the linear concatemer, wherein: (i) the first and second sites flank the therapeutic sequence and form self-complementary overhangs, and (ii) the third site is within the backbone sequence and forms an overhang that is non-complementary to the first or second site, thereby producing a linear therapeutic fragment comprising the therapeutic sequence and at least two linear backbone fragments each comprising a portion of the backbone sequence; and (d) contacting the linear therapeutic fragment with a ligase to produce a therapeutic circular DNA vector in solution.

In some embodiments of either of the preceding aspects, the method further comprises diluting the DNA between steps (c) and step (d). In some embodiments, the DNA concentration at the beginning of step (d) is greater than or equal to 20 μg/mL but less than 160 μg/mL. In some embodiments, the DNA concentration at the beginning of step (d) is about 40 μg/mL. In some embodiments, the DNA concentration at the beginning of step (d) is about 80 μg/mL. In some embodiments, the ligase concentration in step (d) is from about 10 to about 20 U ligase per μg DNA. In some embodiments, the ligase is a T4 ligase. In some embodiments, no temperature increase is performed immediately after step (d).

In some embodiments, the linear concatemer is digested with a single restriction enzyme that cuts the first site, the second site, and the third site (e.g., step (b) involves a single (i.e., one and only one) restriction enzyme (e.g., a type IIs restriction enzyme, e.g., BsaI). In some embodiments, the one or more restriction enzymes cut a fourth site of the linear concatemer per unit, wherein the fourth site is within the backbone sequence and forms an overhang that is non-complementary to the first or second site, and wherein the digestion produces at least three linear backbone fragments each comprising a portion of the backbone sequence. In some embodiments, step (b) involves a single restriction enzyme (e.g., a type IIs restriction enzyme, e.g., BsaI) that cuts a fourth site of the linear concatemer per unit, wherein the fourth site is within the backbone sequence and forms an overhang that is non-complementary to the first or second site, and wherein the digestion produces at least three linear backbone fragments each comprising a portion of the backbone sequence.

In some embodiments, no restriction enzyme inactivation step precedes step (d) (e.g., no heat inactivation of the restriction enzyme precedes step (d)). In some embodiments, no temperature increase is performed between steps (c) and (d). In some embodiments, the temperature is reduced between steps (c) and (d). In some embodiments, no temperature increase is performed immediately after step (d). In some embodiments in which no heat inactivation is performed, the reaction is carried out in a single-use vessel not suitable for high temperatures. In some embodiments, steps (c) and (d) occur simultaneously.

In some embodiments, the method further involves raising the temperature of the solution containing the therapeutic circular DNA vector to about 65° C.

In some embodiments, the method further involves (e) contacting the therapeutic circular DNA vector with a topoisomerase or a helicase. In some embodiments, step (e) is performed at about 37° C.

In some embodiments, the method further involves (f) contacting the linear backbone fragments with an exonuclease (e.g., a terminal exonuclease, e.g., T5 exonuclease). In some embodiments, step (f) is performed at about 37° C.

In some embodiments, the method further includes (e) contacting the therapeutic circular DNA vector with a topoisomerase or a helicase; and (f) contacting the linear backbone fragments with an exonuclease (e.g., a terminal exonuclease, e.g., T5 exonuclease), wherein no enzyme inactivation step is performed between steps (e) and (f). In some embodiments, contacting the therapeutic circular DNA vector with a topoisomerase or a helicase occurs before contacting the linear backbone fragments with an exonuclease (e.g., a terminal exonuclease). In other embodiments, contacting the therapeutic circular DNA vector with a topoisomerase or a helicase occurs after contacting the linear backbone fragments with an exonuclease (e.g., a terminal exonuclease).

In some embodiments of any of the preceding methods, the restriction enzyme is provided at a concentration from about 0.5 U/μg to about 20 U/μg, e.g., from about 1 U/μg DNA to about 10 U/μg DNA, e.g., from about 2 U/μg DNA to about 5 U/μg DNA, e.g., about 2.5 U/μg DNA. For example, the restriction enzyme may be provided at a concentration of about 0.5 U/μg DNA, 1.0 U/μg DNA, 1.5 U/μg DNA, 2.0 U/μg DNA, 2.5 U/μg DNA, 3.0 U/μg DNA, 3.5 U/μg DNA, 4.0 U/μg DNA, 4.5 U/μg DNA, 5.0 U/μg DNA, 5.5 U/μg DNA, 6.0 U/μg DNA, 6.5 U/μg DNA, 7.0 U/μg DNA, 7.5 U/μg DNA, 8.0 U/μg DNA, 8.5 U/μg DNA, 9.0 U/μg DNA, 9.5 U/μg DNA, 10.0 U/μg DNA, 11 U/μg DNA, 12 U/μg DNA, 13 U/μg DNA, 14 U/μg DNA, 15 U/μg DNA, 16 U/μg DNA, 17 U/μg DNA, 18 U/μg DNA, 19 U/μg DNA, or 20 U/μg DNA. In some embodiments, the restriction enzyme is provided at a concentration from about 0.5 U/μg to about 2.5 U/μg.

In some embodiments, the restriction enzyme is provided at a concentration of about 2.5 U/μg.

In some embodiments, digestion (e.g., step (c)) involves incubation from one to 12 hours, e.g., for about one hour. In some embodiments, digestion (e.g., step (c)) involves incubation for one hour or less.

In some embodiments, the ligase is provided at a concentration no greater than 50 U ligase per μg DNA (U/μg) (e.g., no greater than 40 U/μg DNA, no greater than 30 U/μg DNA, no greater than 25 U/μg DNA, no greater than 20 U/μg DNA, no greater than 15 U/μg DNA, no greater than 10 U/μg DNA, no greater than 5 U/μg DNA, no greater than 4 U/μg DNA, no greater than 3 U/μg DNA, no greater than 2.5 U/μg DNA, no greater than 2.0 U/μg DNA, no greater than 1.5 U/μg DNA, or no greater than 1.0 U/μg DNA; e.g., from 0.1 U/μg DNA to 20 U/μg DNA; e.g., from 0.1 U/μg DNA to 30 U/μg DNA, from 0.1 U/μg DNA to 20 U/μg DNA, from 0.2 U/μg DNA to 15 U/μg DNA, from 0.5 U/μg DNA to 12 U/μg DNA, or from 1 U/μg DNA to 10 U/μg DNA; e.g., from 0.1 U/μg DNA to 0.5 U/μg DNA, from 0.5 U/μg DNA to 1.0 U/μg DNA, from 1.0 U/μg DNA to 2.0 U/μg DNA, from 2.0 U/μg DNA to 3.0 U/μg DNA, from 3.0 U/μg DNA to 4.0 U/μg DNA, from 4.0 U/μg DNA to 5.0 U/μg DNA, from 5.0 to 6.0 U/μg DNA, from 6.0 U/μg DNA to 7.0 U/μg DNA, from 7.0 U/μg DNA to 8.0 U/μg DNA, from 8.0 U/μg DNA to 9.0 U/μg DNA, from 9.0 U/μg DNA to 11 U/μg DNA, from 11 U/μg DNA to 12 U/μg DNA, from 12 U/μg DNA to 15 U/μg DNA, from 15 U/μg DNA to 20 U/μg DNA, from 20 U/μg DNA to 25 U/μg DNA, from 25 U/μg DNA to 30 U/μg DNA, from 30 U/μg DNA to 35 U/μg DNA, from 35 U/μg DNA to 40 U/μg DNA, or from 40 U/μg DNA to 50 U/μg DNA). In some embodiments, the ligase is provided at a concentration no greater than 20 U/μg DNA. In some embodiments, the ligase is provided at a concentration of about 10 U/μg. In some embodiments, the ligase is T4 ligase.

In some embodiments, the topoisomerase is provided at a concentration no greater than 10 U topoisomerase per μg DNA (U/μg) (e.g., no greater than 5 U/μg DNA, no greater than 4 U/μg DNA, no greater than 3 U/μg DNA, no greater than 2.5 U/μg DNA, no greater than 2.0 U/μg DNA, no greater than 1.5 U/μg DNA, or no greater than 1.0 U/μg DNA; e.g., from 0.1 U/μg DNA to 10 U/μg DNA; e.g., from 0.5 U/μg DNA to 8 U/μg DNA, or from 1 U/μg DNA to 5 U/μg DNA; e.g., from 0.1 U/μg DNA to 0.5 U/μg DNA, from 0.5 U/μg DNA to 1.0 U/μg DNA, from 1.0 U/μg DNA to 2.0 U/μg DNA, from 2.0 U/μg DNA to 3.0 U/μg DNA, from 3.0 U/μg DNA to 4.0 U/μg DNA, from 4.0 U/μg DNA to 5.0 U/μg DNA, from 5.0 to 6.0 U/μg DNA, from 6.0 U/μg DNA to 7.0 U/μg DNA, from 7.0 U/μg DNA to 8.0 U/μg DNA, from 8.0 U/μg DNA to 9.0 U/μg DNA, or from 9.0 U/μg DNA to 10 U/μg DNA).

In some embodiments, the topoisomerase is a type II topoisomerase. In some embodiments, the topoisomerase is gyrase. In some embodiments, the topoisomerase is topoisomerase IV.

In some embodiments, the exonuclease (e.g., terminal exonuclease, e.g., T5 exonuclease) is provided at a concentration from about 0.5 U/μg to about 20 U/μg, e.g., from about 0.5 U/μg to about 10 U/μg, e.g., from about 1 U/μg to about 10 U/μg, e.g., from about 2 U/μg to about 5 U/μg, e.g., about 2.5 U/μg. For example, the exonuclease (e.g., terminal exonuclease) may be provided at a concentration of about 0.5 U/μg, 1.0 U/μg, 1.5 U/μg, 2.0 U/μg, 2.5 U/μg, 3.0 U/μg, 3.5 U/μg, 4.0 U/μg, 4.5 U/μg, 5.0 U/μg, 5.5 U/μg, 6.0 U/μg, 6.5 U/μg, 7.0 U/μg, 7.5 U/μg, 8.0 U/μg, 8.5 U/μg, 9.0 U/μg, 9.5 U/μg, 10.0 U/μg, 11 U/μg, 12 U/μg, 13 U/μg, 14 U/μg, 15 U/μg, 16 U/μg, 17 U/μg, 18 U/μg, 19 U/μg, or 20 U/μg.

In some embodiments, step (f) is performed two or more times (e.g., two times, three times, or four times). In some embodiments, step (f) comprises incubation from one hour to 12 hours. In some embodiments, step (f) comprises incubation from one hour to 18 hours. In some embodiments, step (f) comprises incubation from three hours to 18 hours. In some embodiments, the exonuclease is a terminal exonuclease, e.g., T5 exonuclease.

In some embodiments of any of the preceding methods, the method further includes: (g) running the therapeutic circular DNA vector through a column (e.g., a capture column); and/or (h) precipitating the therapeutic circular DNA vector with isopropyl alcohol.

In some embodiments, step (b) is performed using site-specific primers. In other embodiments, step (b) is performed using random primers.

In some embodiments, the quantity of therapeutic circular DNA vector produced is at least five-fold the quantity of template DNA vector (e.g., plasmid DNA vector) in the sample of step (a).

In some embodiments, no DNA purification or gel extraction step is performed before step (d).

In some embodiments, the amount of the therapeutic circular DNA in the solution of step (d) is at least 2.0% of the amount of the linear concatemer in step (b) by weight (e.g., at least 3.0%, at least 4.0%, at least 5.0%, at least 6.0%, at least 7.0%, at least 8.0%, at least 9.0%, at least 10%, at least 20%, at least 30%, at least 40%, or at least 50% of the amount of the linear concatemer in step (b) by weight).

In some embodiments, the amount of the therapeutic circular DNA produced in step (d) is at least 1.0 mg (e.g., from 1.0 mg to 10 mg, from 2.0 mg to 10 mg, from 3.0 mg to 10 mg, from 4.0 mg to 10 mg, or from 5.0 mg to 10 mg; e.g., from 1.0 mg to 2.5 mg, from 2.5 mg to 5.0 mg, from 5.0 mg to 7.5 mg, or from 7.5 mg to 10 mg). In some embodiments, the amount of the therapeutic circular DNA produced in step (d) is at least 2.0 mg. For example, in some embodiments, the amount of the therapeutic circular DNA produced in step (d) is at least 5.0 mg.

In some embodiments, the concentration of the therapeutic circular DNA in the solution after step (d) is from 1.0 μg/mL to 1.0 mg/mL without any purification or concentration being performed (e.g., from 5.0 μg/mL to 100 μg/mL, or from 10 μg/mL to 50 μg/mL without any purification or concentration being performed, e.g., from 1.0 μg/mL to 10 μg/mL, from 5.0 g/mL to 10 μg/mL, from 10 μg/mL to 50 μg/mL, from 50 μg/mL to 100 μg/mL, or more, without any purification or concentration being performed).

In some embodiments, the volume of the solution of step (d) is at least 5 liters (e.g., from 5 liters to 200 liters, e.g., from 7 liters to 100 liters, from 10 liters to 80 liters, from 15 liters to 75 liters, or from 20 liters to 70 liters, e.g., at least 1.0 liters, at least 2.0 liters, at least 5.0 liters, at least 10 liters, at least 20 liters, at least 50 liters, or at least 100 liters).

In some embodiments, step (b) is performed in a reaction vessel having a volume of at least 0.5 liters (e.g., at least 1.0 liters, at least 2.0 liters, at least 5.0 liters, at least 10 liters, at least 20 liters, at least 50 liters, at least 100 liters, at least 150 liters, or at least 200 liters). In some embodiments, step (b) is performed in a reaction vessel having a volume of at least 5 liters.

Additionally, or alternatively, steps (c) and (d) are performed in a reaction vessel having a volume of at least 0.5 liters (e.g., at least 1.0 liters, at least 2.0 liters, at least 5.0 liters, at least 10 liters, at least 20 liters, at least 50 liters, at least 100 liters, at least 150 liters, or at least 200 liters). For example, in some embodiments, steps (c) and (d) are performed in a reaction vessel having a volume of at least 5 liters. In some embodiments, steps (b)-(d) are each performed in a reaction vessel having a volume of at least 0.5 liters (e.g., at least 1.0 liters, at least 2.0 liters, at least 5.0 liters, at least 10 liters, at least 20 liters, at least 50 liters, at least 100 liters, at least 150 liters, or at least 200 liters). In some embodiments, steps (b)-(d) are each performed in a reaction vessel having a volume of at least 5 liters.

In some embodiments, the amount of the therapeutic circular DNA produced in step (d) is at least 20% of the amount of the template DNA vector (e.g., plasmid DNA vector) provided in step (a) (e.g., at least 50%, at least 75%, at least 100%, at least 150%, at least twice, at least three-fold, at least four-fold, at least five-fold, or at least ten-fold the amount of template DNA vector (e.g., plasmid DNA vector) provided in step (a); e.g., at least twice the amount, at least three-fold the amount, at least five-fold the amount, at least 10-fold the amount, at least 20-fold the amount, at least 30-fold the amount, at least 40-fold the amount, at least 50-fold the amount, or at least 100-fold the amount of template DNA vector (e.g., plasmid DNA vector) provided in step (a)). In particular embodiments, the amount of the therapeutic circular DNA produced in step (d) is at least five-fold the amount of the template DNA vector (e.g., plasmid DNA vector) provided in step (a). In some embodiments, the amount of the therapeutic circular DNA produced in step (d) is at least ten-fold the amount of the template DNA vector (e.g., plasmid DNA vector) provided in step (a).

In another aspect, provided herein is a method of removing a backbone sequence from a DNA molecule to produce a therapeutic circular DNA vector, wherein the DNA molecule comprises the backbone sequence and a therapeutic sequence, the method comprising: (a) digesting the DNA molecule with a type ITs restriction enzyme that cuts a first site and a second site per unit of the linear concatemer, wherein the first and second sites flank the therapeutic sequence and form self-complementary overhangs, thereby producing a linear therapeutic fragment and a linear backbone fragment, wherein the linear therapeutic fragment comprises the therapeutic sequence and the linear backbone fragment comprises at least a portion of the backbone sequence and a type IIs restriction site; and (b) contacting the linear backbone fragment and the linear therapeutic fragment with a ligase to produce a circular backbone comprising the type ITs restriction site and a therapeutic circular DNA vector lacking a type IIs restriction site.

In another aspect, the method includes providing a sample that includes a template DNA vector (e.g., plasmid DNA vector) including a therapeutic sequence and amplifying the template DNA vector using a polymerase-mediated rolling-circle amplification to generate a linear concatemer. The linear concatemer is digested with a restriction enzyme that cuts at least two sites of the linear concatemer per unit of the template DNA vector to generate linearized fragments of the DNA vector. The method further includes self-ligating the linearized fragment of the DNA vector that includes the therapeutic sequence to produce a therapeutic circular DNA vector. In some embodiments, the digesting and self-ligating are performed simultaneously. The sample can then be treated with a topoisomerase or a helicase. In some embodiments, the method further includes digesting the sample with an exonuclease (e.g., a terminal exonuclease, e.g., T5 exonuclease).

In another aspect, the method includes providing a sample that includes a template DNA vector (e.g., plasmid DNA vector) including a therapeutic sequence and amplifying the template DNA vector using a polymerase-mediated rolling-circle amplification to generate a linear concatemer. The method further includes digesting the linear concatemer with a restriction enzyme to generate a linearized fragment of the DNA vector. The linear concatemer contains multiple copies of the template DNA vector, each copy having a unit length and the linear concatemer having multiple unit lengths of the vector. The restriction enzyme cuts at least two sites of the linear concatemer per unit of the template DNA vector. The method further includes self-ligating the linearized fragment of the DNA vector to produce a therapeutic circular DNA vector. The method also includes digesting the sample with an exonuclease (e.g., a terminal exonuclease, e.g., T5 exonuclease). In some embodiments, the method further includes treating the sample with a topoisomerase or a helicase. In some embodiments, the digesting and self-ligating are performed simultaneously.

In another aspect, the method includes providing a sample having a template DNA vector (e.g., plasmid DNA vector) including a therapeutic sequence and amplifying the template DNA vector using a polymerase-mediated rolling-circle amplification to generate a linear concatemer. The method further includes digesting the linear concatemer with a restriction enzyme to generate a linearized fragment of the DNA vector. The restriction enzyme cuts at least two sites of the linear concatemer per unit of the template DNA vector. The method further includes self-ligating the linearized fragment of the DNA vector to produce a therapeutic circular DNA vector. The method may further include treating the sample with a topoisomerase or a helicase and digesting the sample with an exonuclease (e.g., a terminal exonuclease). In some embodiments, the digesting and self-ligating are performed simultaneously (in the same reaction conditions).

In another aspect, the invention provides a method of removing a backbone sequence from a DNA molecule to produce a therapeutic circular DNA vector. The DNA molecule comprises the backbone sequence and a therapeutic sequence. The method involves the steps of (a) digesting the DNA molecule with one or more restriction enzymes that cut at least a first site, a second site, and a third site per unit of the DNA molecule, wherein: (i) the first and second sites flank the therapeutic sequence and form self-complementary overhangs, and (ii) the third site is within the backbone sequence and forms an overhang that is non-complementary to the first or second site, thereby producing a linear therapeutic fragment comprising the therapeutic sequence and at least two linear backbone fragments each comprising a portion of the backbone sequence; and (b) contacting the linear therapeutic fragment with a ligase to produce a therapeutic circular DNA vector in solution.

In some embodiments, the linear concatemer is digested with a single restriction enzyme that cuts the first site, the second site, and the third site. In some embodiments, the one or more restriction enzymes cut a fourth site of the DNA molecule, wherein the fourth site is within the backbone sequence and forms an overhang that is non-complementary to the first or second site, and wherein the digestion produces at least three linear backbone fragments each comprising a portion of the backbone sequence. In some embodiments, the single restriction enzyme cuts a fourth site of the DNA molecule, wherein the fourth site is within the backbone sequence and forms an overhang that is non-complementary to the first or second site, and wherein the digestion produces at least three linear backbone fragments each comprising a portion of the backbone sequence.

In some embodiments, the DNA molecule is a concatemer produced by amplification of a template DNA vector. In some embodiments, the DNA molecule is a template DNA vector.

In some embodiments, the template DNA vector is a plasmid DNA vector.

In some embodiments, the single restriction enzyme is a type IIs restriction enzyme, e.g., BsaI.

In some embodiments, no restriction enzyme inactivation step precedes step (b).

In some embodiments, no temperature increase is performed between steps (a) and (b).

In some embodiments, steps (a) and (b) occur simultaneously.

In some embodiments, the method further includes raising the temperature of the solution containing the therapeutic circular DNA vector to about 65° C.

In some embodiments, the method further includes (c) contacting the therapeutic circular DNA vector with a topoisomerase or a helicase. In some embodiments, step (c) is performed at about 37° C. In some embodiments, the method further includes: (d) contacting the linear backbone fragments with an exonuclease (e.g., a terminal exonuclease). In some embodiments, step (d) is performed at about 37° C.

In some embodiments, the method further includes: (c) contacting the therapeutic circular DNA vector with a topoisomerase or a helicase; and (d) contacting the linear backbone fragments with an exonuclease (e.g., a terminal exonuclease), wherein no enzyme inactivation step is performed between steps (c) and (d). In some embodiments, step (c) occurs before step (d).

In some embodiments, the restriction enzyme is provided at a concentration of from about 0.5 U/μg to about 20 U/μg, e.g., from about 1 U/μg DNA to about 10 U/μg DNA, e.g., from about 2 U/Ig DNA to about 5 U/μg DNA, e.g., about 2.5 U/μg DNA. For example, the restriction enzyme may be provided at a concentration of about 0.5 U/μg DNA, 1.0 U/μg DNA, 1.5 U/μg DNA, 2.0 U/μg DNA, 2.5 U/μg DNA, 3.0 U/μg DNA, 3.5 U/μg DNA, 4.0 U/μg DNA, 4.5 U/μg DNA, 5.0 U/μg DNA, 5.5 U/μg DNA, 6.0 U/μg DNA, 6.5 U/μg DNA, 7.0 U/μg DNA, 7.5 U/μg DNA, 8.0 U/μg DNA, 8.5 U/μg DNA, 9.0 U/μg DNA, 9.5 U/μg DNA, 10.0 U/μg DNA, 11 U/μg DNA, 12 U/μg DNA, 13 U/μg DNA, 14 U/μg DNA, 15 U/μg DNA, 16 U/μg DNA, 17 U/μg DNA, 18 U/μg DNA, 19 U/μg DNA, or 20 U/μg DNA. In some embodiments, the restriction enzyme is provided at a concentration of about 2.5 U/μg.

In some embodiments, step (a) comprises incubation from one to 12 hours (e.g., about one hour).

In some embodiments, the ligase is provided at a concentration no greater than 20 U ligase per μg DNA (U/μg) (e.g., no greater than 15 U/μg DNA, no greater than 10 U/μg DNA, no greater than 5 U/μg DNA, no greater than 4 U/μg DNA, no greater than 3 U/μg DNA, no greater than 2.5 U/μg DNA, no greater than 2.0 U/μg DNA, no greater than 1.5 U/μg DNA, or no greater than 1.0 U/μg DNA; e.g., from 0.1 U/μg DNA to 20 U/μg DNA; e.g., from 0.2 U/μg DNA to 15 U/μg DNA, from 0.5 U/μg DNA to 12 U/μg DNA, or from 1 U/μg DNA to 10 U/μg DNA; e.g., from 0.1 U/μg DNA to 0.5 U/μg DNA, from 0.5 U/μg DNA to 1.0 U/μg DNA, from 1.0 U/μg DNA to 2.0 U/μg DNA, from 2.0 U/μg DNA to 3.0 U/μg DNA, from 3.0 U/μg DNA to 4.0 U/μg DNA, from 4.0 U/μg DNA to 5.0 U/μg DNA, from 5.0 to 6.0 U/μg DNA, from 6.0 U/μg DNA to 7.0 U/μg DNA, from 7.0 U/μg DNA to 8.0 U/μg DNA, from 8.0 U/μg DNA to 9.0 U/μg DNA, from 9.0 U/μg DNA to 11 U/μg DNA, from 11 U/μg DNA to 12 U/μg DNA, from 12 U/μg DNA to 15 U/μg DNA, or from 15 U/μg DNA to 20 U/μg DNA). In some embodiments, the ligase is at a concentration of about 10 U/μg DNA. In some embodiments, the ligase is T4 ligase.

In some embodiments, the topoisomerase is provided at a concentration no greater than 10 U topoisomerase per μg DNA (U/μg) (e.g., no greater than 5 U/μg DNA, no greater than 4 U/μg DNA, no greater than 3 U/μg DNA, no greater than 2.5 U/μg DNA, no greater than 2.0 U/μg DNA, no greater than 1.5 U/μg DNA, or no greater than 1.0 U/μg DNA; e.g., from 0.1 U/μg DNA to 10 U/μg DNA; e.g., from 0.5 U/μg DNA to 8 U/μg DNA, or from 1 U/μg DNA to 5 U/μg DNA; e.g., from 0.1 U/μg DNA to 0.5 U/μg DNA, from 0.5 U/μg DNA to 1.0 U/μg DNA, from 1.0 U/μg DNA to 2.0 U/μg DNA, from 2.0 U/μg DNA to 3.0 U/μg DNA, from 3.0 U/μg DNA to 4.0 U/μg DNA, from 4.0 U/μg DNA to 5.0 U/μg DNA, from 5.0 to 6.0 U/μg DNA, from 6.0 U/μg DNA to 7.0 U/μg DNA, from 7.0 U/μg DNA to 8.0 U/μg DNA, from 8.0 U/μg DNA to 9.0 U/μg DNA, or from 9.0 U/μg DNA to 10 U/μg DNA).

In some embodiments, the topoisomerase is a type II topoisomerase. In some embodiments, the topoisomerase is gyrase or topoisomerase IV. In some embodiments, the exonuclease (e.g., the terminal exonuclease, e.g., T5 exonuclease) is provided at a concentration from about 0.5 U/μg to about 20 U/μg, e.g., from about 0.5 U/μg to about 10 U/μg, e.g., from about 1 U/μg to about 10 U/μg, e.g., from about 2 U/μg to about 5 U/μg, e.g., about 2.5 U/μg.

For example, the exonuclease (e.g., the terminal exonuclease) may be provided at a concentration of about 0.5 U/μg, 1.0 U/μg, 1.5 U/μg, 2.0 U/μg, 2.5 U/μg, 3.0 U/μg, 3.5 U/μg, 4.0 U/μg, 4.5 U/μg, 5.0 U/μg, 5.5 U/μg, 6.0 U/μg, 6.5 U/μg, 7.0 U/μg, 7.5 U/μg, 8.0 U/μg, 8.5 U/μg, 9.0 U/μg, 9.5 U/μg, 10.0 U/μg, 11 U/μg, 12 U/μg, 13 U/μg, 14 U/μg, 15 U/μg, 16 U/μg, 17 U/μg, 18 U/μg, 19 U/μg, or 20 U/μg.

In some embodiments, step (d) is performed two or more times. In some embodiments, step (d) comprises incubation from one hour to 12 hours. In some embodiments, the exonuclease is a terminal exonuclease. In some embodiments, the terminal exonuclease is T5 exonuclease. In some embodiments, the method further includes: (e) running the therapeutic circular DNA vector through a column (e.g., capture column); and/or (f) precipitating the therapeutic circular DNA vector with isopropyl alcohol.

In some of any of the preceding embodiments, the therapeutic circular DNA vector is produced in the absence of a gel extraction step (e.g., an in-process gel extraction step).

In another aspect, provided is a method of producing a supercoiled therapeutic circular DNA vector, the method comprising: (a) providing a sample comprising a template DNA vector comprising a therapeutic sequence and a backbone sequence; (b) amplifying the template DNA vector using a polymerase-mediated rolling-circle amplification to generate a linear concatemer; (c) digesting the linear concatemer with a type IIs restriction enzyme that cuts a first site and a second site per unit of the linear concatemer, wherein the first and second sites flank the therapeutic sequence and form self-complementary overhangs, thereby producing a linear therapeutic fragment and a linear backbone fragment, wherein the linear therapeutic fragment comprises the therapeutic sequence and the linear backbone fragment comprises at least a portion of the backbone sequence; (d) diluting the linear therapeutic fragment and the linear backbone fragment to a cumulative DNA concentration from 20 μg/mL to 160 μg/mL; (e) contacting the diluted linear backbone fragment and the linear therapeutic fragment with a ligase to produce a circular backbone and a therapeutic circular DNA vector lacking a type Us restriction site; (f) contacting the therapeutic circular DNA vector with gyrase at a concentration of about 1.5 U per g DNA to produce a mixture of supercoiled therapeutic circular DNA vectors and linear backbone fragments; and (g) after step (f), digesting the linear backbone fragments with an exonuclease.

In another aspect, provided is a method of producing a supercoiled therapeutic circular DNA vector, the method comprising: (a) providing a sample comprising a template DNA vector comprising a therapeutic sequence and a backbone sequence; (b) amplifying the template DNA vector using a polymerase-mediated rolling-circle amplification to generate a linear concatemer; (c) digesting the linear concatemer with a type IIs restriction enzyme that cuts a first site and a second site per unit of the linear concatemer, wherein the first and second sites flank the therapeutic sequence and form self-complementary overhangs, thereby producing a linear therapeutic fragment and a linear backbone fragment, wherein the linear therapeutic fragment comprises the therapeutic sequence and the linear backbone fragment comprises at least a portion of the backbone sequence; (d) diluting the linear therapeutic fragment and the linear backbone fragment to a cumulative DNA concentration from 20 μg/mL to 160 μg/mL; (e) contacting the diluted linear backbone fragment and the linear therapeutic fragment with a ligase to produce a circular backbone and a therapeutic circular DNA vector lacking a type IIs restriction site; (f) digesting the linear backbone fragment with an exonuclease; and (g) after step (f), supercoiling the therapeutic circular DNA vector with gyrase at a concentration of less than 1.5 U per μg DNA. In some embodiments, the ligase of step (e) is at a concentration from 10 to 20 U ligase per μg DNA. In some embodiments, the diluted cumulative DNA concentration of step (d) is about 10% to about 80% of cumulative DNA concentration immediately after step (c). In some embodiments, the cumulative DNA concentration immediately after step (c) is between 100 μg/mL and 300 μg/mL. In some embodiments, the first or second cut sites flanking the therapeutic sequence comprises AAAA or AACC.

In another aspect, provided is a method for large-scale production of a therapeutic circular DNA vector, the method comprising: (a) providing a sample of a template DNA vector (e.g., plasmid DNA vector) comprising a therapeutic sequence and a backbone sequence; (b) amplifying the template DNA vector in a reaction volume of at least 1.0 liter (e.g., at least 2.0 liters, at least 5.0 liters, at least 10 liters, at least 20 liters, at least 50 liters, at least 100 liters, at least 150 liters, or at least 200 liters) using a polymerase-mediated rolling-circle amplification to generate a linear concatemer; (c) digesting the linear concatemer with one or more restriction enzymes that cut at least a first site, a second site, and a third site per unit of the linear concatemer, wherein: (i) the first and second sites flank the therapeutic sequence and form self-complementary overhangs, and (ii) the third site is within the backbone sequence and forms an overhang that is non-complementary to the first or second site, thereby producing a linear therapeutic fragment comprising the therapeutic sequence and at least two linear backbone fragments each comprising a portion of the backbone sequence; and (d) contacting the linear therapeutic fragment with a ligase to produce a therapeutic circular DNA vector in solution.

In some embodiments, the amount of the template DNA vector provided in step (a) is at least 0.5 mg, at least 0.75 mg, or at least 1.0 mg (e.g., from 1.0 mg to 10 mg, from 2.0 mg to 10 mg, from 3.0 mg to 10 mg, from 4.0 mg to 10 mg, or from 5.0 mg to 10 mg; e.g., from 1.0 mg to 2.5 mg, from 2.5 mg to 5.0 mg, from 5.0 mg to 7.5 mg, or from 7.5 mg to 10 mg). In some embodiments, the amount of the template DNA vector provided in step (a) is at least 5.0 mg. For example, in some embodiments, the amount of the template DNA vector provided in step (a) is at least 10.0 mg.

In some embodiments, step (b) produces at least 100 mg of the linear concatemer (e.g., from 100 mg to 10 g, from 500 mg to 5 g, or from 1 g to 3 g; e.g., at least 200 mg, at least 300 mg, at least 400 mg, at least 500 mg, at least 1 g, at least 2 g, or at least 3 g; e.g., from 200 mg to 10 g, from 300 mg to 10 g, from 400 mg to 10 g, from 500 mg to 10 g, or from 1 g to 10 g). In some embodiments, step (b) produces a solution containing from 0.5 g to 2 g DNA per liter reaction volume (e.g., about 1 g DNA per liter reaction volume).

In some embodiments, step (d) produces at least 1.0 mg of the therapeutic circular DNA vector (e.g., from 1.0 mg to 10 mg, from 2.0 mg to 10 mg, from 3.0 mg to 10 mg, from 4.0 mg to 10 mg, or from 5.0 mg to 10 mg; e.g., from 1.0 mg to 2.5 mg, from 2.5 mg to 5.0 mg, from 5.0 mg to 7.5 mg, or from 7.5 mg to 10 mg). In some embodiments, the amount of the therapeutic circular DNA produced in step (d) is at least 2.0 mg (e.g., as in large-scale production). For example, in some embodiments, the amount of the therapeutic circular DNA produced in step (d) is at least 5.0 mg.

In some embodiments, steps (c) and (d) occur simultaneously. In some embodiments, no DNA purification is performed during or between steps (b), (c), and (d).

In some embodiments, the amount of the therapeutic circular DNA in the solution of step (d) is at least 2.0% of the amount of the linear concatemer in step (b) by weight (e.g., at least 3.0%, at least 4.0%, at least 5.0%, at least 6.0%, at least 7.0%, at least 8.0%, at least 9.0%, at least 10%, at least 20%, at least 30%, at least 40%, or at least 50% of the amount of the linear concatemer in step (b) by weight).

In some embodiments, the amount of the therapeutic circular DNA produced in step (d) is at least 20% of the amount of the template DNA vector (e.g., plasmid DNA vector) provided in step (a) (e.g., at least 50%, at least 75%, at least 100%, or at least 150% of the amount of template DNA vector (e.g., plasmid DNA vector) provided in step (a); e.g., at least twice the amount, at least three-fold the amount, at least five-fold the amount, at least 10-fold the amount, at least 20-fold the amount, at least 30-fold the amount, at least 40-fold the amount, at least 50-fold the amount, or at least 100-fold the amount of template DNA vector (e.g., plasmid DNA vector) provided in step (a)). In particular embodiments, the amount of the therapeutic circular DNA produced in step (d) is at least five-fold the amount of the template DNA vector (e.g., plasmid DNA vector) provided in step (a). In some embodiments, the amount of the therapeutic circular DNA produced in step (d) is at least ten-fold the amount of the template DNA vector (e.g., plasmid DNA vector) provided in step (a).

In some embodiments, the DNA concentration at the beginning of step (d) is greater than or equal to 20 μg/mL but less than 160 μg/mL. In some embodiments, the DNA concentration at the beginning of step (d) is from about 40 μg/mL to about 80 μg/mL. In some embodiments, the DNA concentration at the beginning of step (d) is about 40 μg/mL. In some embodiments, the DNA concentration at the beginning of step (d) is about 80 μg/mL. In some embodiments, the ligase concentration (e.g., T4 ligase concentration) in step (d) is from about 10 to about 20 U ligase per g DNA. In some embodiments, no temperature increase is performed immediately after step (d).

In another aspect, provided is a method producing a therapeutic circular DNA vector, the method comprising: (a) providing a solution comprising DNA molecules, wherein each DNA molecule comprises a backbone sequence and a therapeutic sequence; (b) adding a type IIs restriction enzyme to the solution to digest the DNA molecules, thereby separating the backbone sequences from the therapeutic sequences; (c) adding a ligase to the solution to produce a reaction in a mixture comprising: (i) the ligase; (ii) the type IIs restriction enzyme; (iii) therapeutic circular DNA vectors each comprising a single therapeutic sequence, wherein the therapeutic circular DNA vectors each lack a type Us recognition site; and (iv) byproducts, wherein each byproduct comprises one or more type IIs restriction sites, wherein the ratio of the therapeutic circular DNA vectors to the byproducts comprising one or more type IIs restriction sites increases as the reaction proceeds. In some embodiments, some or all of the byproducts comprise one, two, three, four, or more backbone sequences (e.g., circularized DNA containing two or more backbone sequences connected through type IIs restriction sites, and/or linear DNA containing two or more backbone sequences connected through type IIs restriction sites). In some embodiments, some or all of the byproducts further comprise two, three, four, or more therapeutic sequences (e.g., circularized DNA containing two or more copies of the therapeutic sequence connected through type IIs restriction sites, and/or linear DNA containing two or more copies of the therapeutic sequence connected through type Us restriction sites). In some embodiments, some or all of the byproducts are circularized. In some embodiments, the DNA molecules of (a) are concatemers.

In some embodiments, the method further comprises, prior to step (a), amplifying a template DNA vector (e.g., a plasmid DNA vector) using rolling circle amplification to generate concatemers.

In some embodiments, the type IIs restriction enzyme is BsaI.

In some embodiments, no restriction enzyme inactivation step precedes step (c). In some embodiments, no temperature increase is performed between steps (b) and (c). In some embodiments, the method further includes raising the temperature of the solution containing the therapeutic circular DNA vector to about 65° C. In some embodiments, the method further includes: (d) contacting the therapeutic circular DNA vector with a topoisomerase or a helicase. In some embodiments, step (d) is performed at about 37° C. In some embodiments, the method additionally or alternatively includes: (e) contacting linear byproducts with an exonuclease. In some embodiments, step (e) is performed at about 37° C.

In some embodiments, the method further includes: (d) contacting the therapeutic circular DNA vector with a topoisomerase or a helicase; and (e) contacting linear byproducts with an exonuclease, wherein no enzyme inactivation step is performed between steps (d) and (e). In some embodiments, step (d) occurs before step (e).

In some embodiments, the restriction enzyme is provided at a concentration from about 0.5 U/μg to about 20 U/μg, e.g., from about 1 U/μg DNA to about 10 U/μg DNA, e.g., from about 2 U/μg DNA to about 5 U/μg DNA, e.g., about 2.5 U/μg DNA. For example, the restriction enzyme may be provided at a concentration of about 0.5 U/μg DNA, 1.0 U/μg DNA, 1.5 U/μg DNA, 2.0 U/μg DNA, 2.5 U/μg DNA, 3.0 U/μg DNA, 3.5 U/μg DNA, 4.0 U/μg DNA, 4.5 U/μg DNA, 5.0 U/μg DNA, 5.5 U/μg DNA, 6.0 U/μg DNA, 6.5 U/μg DNA, 7.0 U/μg DNA, 7.5 U/μg DNA, 8.0 U/μg DNA, 8.5 U/μg DNA, 9.0 U/μg DNA, 9.5 U/μg DNA, 10.0 U/μg DNA, 11 U/μg DNA, 12 U/μg DNA, 13 U/μg DNA, 14 U/μg DNA, 15 U/μg DNA, 16 U/μg DNA, 17 U/μg DNA, 18 U/μg DNA, 19 U/μg DNA, or 20 U/μg DNA. In some embodiments, the restriction enzyme is provided at a concentration of about 2.5 U/μg. In some embodiments, the restriction enzyme is provided at a concentration from about 0.5 U/μg to about 2.5 U/μg.

In some embodiments, digestion (e.g., step (b)) involves incubation from one to 12 hours, e.g., for about one hour.

In some embodiments, the ligase is provided at a concentration no greater than 50 U ligase per g DNA (U/μg) (e.g., no greater than 40 U/μg DNA, no greater than 30 U/μg DNA, no greater than 25 U/μg DNA, no greater than 20 U/μg DNA, no greater than 15 U/μg DNA, no greater than 10 U/μg DNA, no greater than 5 U/μg DNA, no greater than 4 U/μg DNA, no greater than 3 U/μg DNA, no greater than 2.5 U/μg DNA, no greater than 2.0 U/μg DNA, no greater than 1.5 U/μg DNA, or no greater than 1.0 U/μg DNA; e.g., from 0.1 U/μg DNA to 20 U/μg DNA; e.g., from 0.1 U/μg DNA to 30 U/μg DNA, from 0.1 U/μg DNA to 20 U/μg DNA, from 0.2 U/μg DNA to 15 U/μg DNA, from 0.5 U/μg DNA to 12 U/μg DNA, or from 1 U/μg DNA to 10 U/μg DNA; e.g., from 0.1 U/μg DNA to 0.5 U/μg DNA, from 0.5 U/μg DNA to 1.0 U/μg DNA, from 1.0 U/μg DNA to 2.0 U/μg DNA, from 2.0 U/μg DNA to 3.0 U/μg DNA, from 3.0 U/μg DNA to 4.0 U/μg DNA, from 4.0 U/μg DNA to 5.0 U/μg DNA, from 5.0 to 6.0 U/μg DNA, from 6.0 U/μg DNA to 7.0 U/μg DNA, from 7.0 U/μg DNA to 8.0 U/μg DNA, from 8.0 U/μg DNA to 9.0 U/μg DNA, from 9.0 U/μg DNA to 11 U/μg DNA, from 11 U/μg DNA to 12 U/μg DNA, from 12 U/μg DNA to 15 U/μg DNA, from 15 U/μg DNA to 20 U/μg DNA, from 20 U/μg DNA to 25 U/μg DNA, from 25 U/μg DNA to 30 U/μg DNA, from 30 U/μg DNA to 35 U/μg DNA, from 35 U/μg DNA to 40 U/μg DNA, or from 40 U/μg DNA to 50 U/μg DNA). In some embodiments, the ligase is provided at a concentration no greater than 20 U/μg DNA, e.g., about 10 U/μg DNA. In some embodiments, the ligase is T4 ligase.

In some embodiments, the topoisomerase is provided at a concentration no greater than 10 U topoisomerase per μg DNA (U/μg) (e.g., no greater than 5 U/μg DNA, no greater than 4 U/μg DNA, no greater than 3 U/μg DNA, no greater than 2.5 U/μg DNA, no greater than 2.0 U/μg DNA, no greater than 1.5 U/μg DNA, or no greater than 1.0 U/μg DNA; e.g., from 0.1 U/μg DNA to 10 U/μg DNA; e.g., from 0.5 U/μg DNA to 8 U/μg DNA, or from 1 U/μg DNA to 5 U/μg DNA; e.g., from 0.1 U/μg DNA to 0.5 U/μg DNA, from 0.5 U/μg DNA to 1.0 U/μg DNA, from 1.0 U/μg DNA to 2.0 U/μg DNA, from 2.0 U/μg DNA to 3.0 U/μg DNA, from 3.0 U/μg DNA to 4.0 U/μg DNA, from 4.0 U/μg DNA to 5.0 U/μg DNA, from 5.0 to 6.0 U/μg DNA, from 6.0 U/μg DNA to 7.0 U/μg DNA, from 7.0 U/μg DNA to 8.0 U/μg DNA, from 8.0 U/μg DNA to 9.0 U/μg DNA, or from 9.0 U/μg DNA to 10 U/μg DNA).

In some embodiments, the topoisomerase is a type II topoisomerase. In some embodiments, the topoisomerase is gyrase. In some embodiments, the topoisomerase is topoisomerase IV.

In some embodiments, the exonuclease (e.g., terminal exonuclease, e.g., T5 exonuclease) is provided at a concentration from about 0.5 U/μg to about 20 U/μg, e.g., from about 0.5 U/μg to about 10 U/μg, e.g., from about 1 U/μg to about 10 U/μg, e.g., from about 2 U/μg to about 5 U/μg, e.g., about 2.5 U/μg. For example, the exonuclease (e.g., terminal exonuclease) may be provided at a concentration of about 0.5 U/μg, 1.0 U/μg, 1.5 U/μg, 2.0 U/μg, 2.5 U/μg, 3.0 U/μg, 3.5 U/μg, 4.0 U/μg, 4.5 U/μg, 5.0 U/μg, 5.5 U/μg, 6.0 U/μg, 6.5 U/μg, 7.0 U/μg, 7.5 U/μg, 8.0 U/μg, 8.5 U/μg, 9.0 U/μg, 9.5 U/μg, 10.0 U/μg, 11 U/μg, 12 U/μg, 13 U/μg, 14 U/μg, 15 U/μg, 16 U/μg, 17 U/μg, 18 U/μg, 19 U/μg, or 20 U/μg.

In some embodiments, step (e) is performed two or more times (e.g., two times, three times, or four times). In some embodiments, step (e) comprises incubation from one hour to 12 hours. In some embodiments, the exonuclease is a terminal exonuclease, e.g., T5 exonuclease.

In some embodiments of any of the preceding methods, the method further includes: (f) running the therapeutic circular DNA vector through a column (e.g., a capture column or an anion exchange column); and/or (g) precipitating the therapeutic circular DNA vector with isopropyl alcohol.

In some embodiments, the amplification is performed using site-specific primers. In other embodiments, the amplification is performed using random primers.

In some embodiments, no in-process gel extraction step is performed before step (c). In some embodiments, no in-process DNA purification is performed before step (c).

In some embodiments, the amount of the therapeutic circular DNA in the solution of step (c) is at least 2.0% of the amount of the DNA molecule in step (a) by weight (e.g., at least 3.0%, at least 4.0%, at least 5.0%, at least 6.0%, at least 7.0%, at least 8.0%, at least 9.0%, at least 10%, at least 20%, at least 30%, at least 40%, or at least 50% of the DNA molecule in step (a) by weight).

In some embodiments, the amount of the therapeutic circular DNA produced in step (c) is at least 1.0 mg (e.g., from 1.0 mg to 10 mg, from 2.0 mg to 10 mg, from 3.0 mg to 10 mg, from 4.0 mg to 10 mg, or from 5.0 mg to 10 mg; e.g., from 1.0 mg to 2.5 mg, from 2.5 mg to 5.0 mg, from 5.0 mg to 7.5 mg, or from 7.5 mg to 10 mg). In some embodiments, the amount of the therapeutic circular DNA produced in step (c) is at least 2.0 mg (e.g., as in large-scale production). For example, in some embodiments, the amount of the therapeutic circular DNA produced in step (c) is at least 5.0 mg.

In some embodiments, the concentration of the therapeutic circular DNA in the solution after step (c) is from 1.0 μg/mL to 1.0 mg/mL without any purification or concentration being performed (e.g., from 5.0 μg/mL to 100 μg/mL, or from 10 μg/mL to 50 μg/mL without any purification or concentration being performed, e.g., from 1.0 μg/mL to 10 μg/mL, from 5.0 μg/mL to 10 μg/mL, from 10 μg/mL to 50 μg/mL, from 50 μg/mL to 100 μg/mL, or more, without any purification or concentration being performed). In some embodiments, the volume of the solution of step (c) is at least 5 liters (e.g., from 5 liters to 200 liters, e.g., from 7 liters to 100 liters, from 10 liters to 80 liters, from 15 liters to 75 liters, or from 20 liters to 70 liters, e.g., at least 1.0 liters, at least 2.0 liters, at least 5.0 liters, at least 10 liters, at least 20 liters, at least 50 liters, or at least 100 liters).

In some embodiments, steps (b) and (c) are performed in a reaction vessel having a volume of at least 0.5 liters (e.g., at least 1.0 liter, at least 2.0 liters, at least 5.0 liters, at least 10 liters, at least 20 liters, at least 50 liters, at least 100 liters, at least 150 liters, or at least 200 liters). In some embodiments, steps (b) and (c) are performed in a reaction vessel having a volume of at least 5 liters (e.g., from 5 liters to 200 liters, e.g., from 7 liters to 100 liters, from 10 liters to 80 liters, from 15 liters to 75 liters, or from 20 liters to 70 liters, e.g., at least 1.0 liters, at least 2.0 liters, at least 5.0 liters, at least 10 liters, at least 20 liters, at least 50 liters, or at least 100 liters).

In another aspect, provided is a method of producing a therapeutic circular DNA vector, the method comprising: (a) providing a mixture of DNA comprising a plurality of linear therapeutic DNA fragments and a plurality of linear backbone DNA fragments, wherein each linear therapeutic DNA fragment comprises a therapeutic sequence and self-complementary ends, wherein the plurality of linear therapeutic DNA fragments and linear backbone DNA fragments are at a cumulative DNA concentration from 20 μg/mL to 160 μg/mL; and (b) performing a ligation reaction by contacting the mixture of DNA with a ligase at a concentration from 10 to 20 U ligase per μg DNA to produce a therapeutic circular DNA vector. In some embodiments, the mixture of DNA was produced by a type IIs restriction digest reaction, wherein a type IIs restriction enzyme cleaves the linear therapeutic DNA fragments from the linear backbone DNA fragments, wherein the self-complementary ends are type IIs overhangs.

In another aspect, provided is a method of producing a therapeutic circular DNA vector, the method comprising: (a) producing a mixture of DNA comprising a plurality of linear therapeutic DNA fragments and a plurality of linear backbone DNA fragments by a type IIs restriction digest reaction, wherein a type IIs restriction enzyme cleaves the linear therapeutic DNA fragments from the linear backbone DNA fragments, wherein each linear therapeutic DNA fragment comprises a therapeutic sequence and self-complementary type IIs overhangs, wherein the plurality of linear therapeutic DNA fragments and linear backbone DNA fragments are at a cumulative DNA concentration from 20 μg/mL to 160 μg/mL; and (b) performing a ligation reaction by contacting the mixture of DNA with a ligase at a concentration from 10 to 20 U ligase per g DNA to produce a therapeutic circular DNA vector.

In some embodiments of either of the previous two aspects, the cumulative DNA concentration of step (a) is achieved by adjusting (e.g., diluting) the cumulative DNA concentration immediately after the type IIs restriction digest. In some embodiments, the cumulative DNA concentration immediately after the type IIs restriction digest is diluted to achieve the cumulative DNA concentration of step (a). In some embodiments, the cumulative DNA concentration immediately after the type Us restriction digest is from 100 μg/mL to 300 g/mL. In some embodiments, the cumulative DNA concentration of step (a) is diluted to about 10% to about 80% of the cumulative DNA concentration immediately after the type IIs restriction digest. In some embodiments, the cumulative DNA concentration of step (a) is from about 40 μg/mL to about 80 μg/mL. In some embodiments, the type IIs restriction enzyme in the type IIs restriction digest reaction is at a concentration from about 0.5 to about 2.5 U per μg DNA. In some embodiments, the ligase (e.g., T4 ligase) is at a concentration of about 10 U/ug. In some embodiments, the ligation reaction is carried out for at least five hours, e.g., 18-24 hours.

In some embodiments, the type IIs restriction enzyme in the type IIs restriction digest reaction (e.g., BsaI) is at a concentration from about 0.5 to about 2.5 U per g DNA. In some embodiments, the type IIs restriction digest reaction is carried out for no more than two hours, e.g., 10 minutes to one hour.

In some embodiments, the type IIs overhangs each comprise four bases. In some embodiments, two and only two of the four bases are A or T. In some embodiments, the type IIs overhangs comprise AAAA or AACC.

In some embodiments, the method further includes (c) contacting the therapeutic circular DNA vector with a topoisomerase or a helicase and/or (d) contacting the linear backbone fragments with an exonuclease.

In some embodiments, the method further includes (c) contacting the therapeutic circular DNA vector with a topoisomerase or a helicase and (d) contacting the linear backbone fragments with an exonuclease. In some embodiments, no enzyme inactivation step is performed between steps (c) and (d). In some embodiments, step (c) occurs before step (d). In other embodiments, step (d) occurs before step (c).

In some embodiments, the topoisomerase (e.g., gyrase) is provided at a concentration no greater than 10 U topoisomerase per μg DNA (U/μg). In some embodiments, the topoisomerase is a type II topoisomerase. In some embodiments, the topoisomerase is gyrase or topoisomerase IV.

In some embodiments, the exonuclease (e.g., T5 exonuclease) is provided at a concentration from about 0.5 U/μg to about 20 U/μg. In some embodiments, step (d) is performed two or more times. In some embodiments, step (d) comprises incubation from one hour to 18 hours. In some embodiments, step (d) comprises incubation from 3-18 hours.

In some embodiments, the method further includes (e) running the therapeutic circular DNA vector through a column and/or (f) precipitating the therapeutic circular DNA vector with isopropyl alcohol.

In some embodiments, the amount of the therapeutic circular DNA produced in step (b) is at least 1.0 mg. In some embodiments, the concentration of the therapeutic circular DNA in the solution after step (b) is at least 5 μg/mL without any purification or concentration being performed. In some embodiments, the volume of the solution of step (d) is at least five liters. In some embodiments, step (b) is performed in a reaction vessel having a volume of at least one liter.

In some embodiments, the mixture of DNA is a product of in vitro amplification.

In some embodiments, the in vitro amplification is a polymerase-mediated rolling-circle amplification.

In some embodiments, the method does not comprise a gel-extraction step.

In some embodiments, the DNA mixture comprises only one species of linear backbone DNA fragment (e.g., the restriction digest produces a single fragment containing the backbone of the plasmid).

In another aspect, provided is a method of producing a supercoiled therapeutic circular DNA vector, the method comprising: (a) providing a sample comprising a therapeutic circular DNA vector in relaxed circular form, wherein the therapeutic circular DNA vector comprises a therapeutic sequence; (b) contacting the sample with a gyrase, wherein the concentration of the gyrase is about 1.5 U per mg of therapeutic circular DNA vector, thereby producing a composition of supercoiled therapeutic circular DNA vector. In some embodiments, the sample of (a) further comprises linear DNA byproducts, and wherein the method further comprises, after (b), contacting the composition of supercoiled therapeutic circular DNA vector with an exonuclease under conditions suitable to digest linear DNA byproducts.

In another aspect, the invention includes a method of producing a supercoiled therapeutic circular DNA vector, the method comprising: (a) providing a sample comprising a therapeutic circular DNA vector in relaxed circular form and linear DNA byproducts, wherein the therapeutic circular DNA vector comprises a therapeutic sequence; (b) contacting the sample with an exonuclease under conditions suitable to digest the linear DNA byproducts to form a digested sample; and (c) contacting the digested sample with a gyrase, wherein the concentration of the gyrase is greater than 0.1 U per mg of therapeutic circular DNA vector and less than 1.5 U per mg of therapeutic circular DNA vector, thereby producing a supercoiled therapeutic circular DNA vector.

In some embodiments of either of the preceding aspects, the exonuclease is a T5 exonuclease and/or the ligase is a T4 ligase. In some embodiments, the method includes, before step (a), contacting a linear therapeutic fragment with a ligase to produce the therapeutic circular DNA vector. In some embodiments, the method includes, before contacting the linear therapeutic fragment with the ligase, digesting a linear concatemer comprising a therapeutic sequence with a restriction enzyme to cut a first site and a second site per unit of the linear concatemer, wherein the first and second sites flank the therapeutic sequence and form self-complementary overhangs, thereby producing the linear therapeutic fragment and the linear DNA byproducts. In some embodiments, the supercoiled therapeutic circular DNA vector is within a composition of therapeutic circular DNA vectors, wherein at least 70% of the therapeutic circular DNA vectors are supercoiled (e.g., at least 80% of the therapeutic circular DNA vectors are supercoiled).

In some embodiments of any of the preceding aspects, the therapeutic circular DNA vector is formulated as a pharmaceutical composition. In some embodiments, the pharmaceutical composition comprises no more than 1.0% (e.g., no more than 0.5%) of residual protein or backbone sequence by weight in comparison to the amount of the therapeutic circular DNA vector. In some embodiments, the therapeutic sequence is greater than 5 kb. In some embodiments, the therapeutic sequence is from 5 kb to 15 kb. In some embodiments, the therapeutic sequence is from 5 kb to 10 kb. In some embodiments, the therapeutic sequence is from 10 kb to 15 kb. In some embodiments, the therapeutic sequence comprises two or more transcription units. In some embodiments, the therapeutic sequence encodes one or more therapeutic proteins. In some embodiments, the one or more therapeutic proteins is a multimeric protein. In some embodiments, the therapeutic sequence encodes a therapeutic nucleic acid. In some embodiments, the therapeutic nucleic acid is an RNA molecule. In some embodiments, the RNA molecule is a self-replicating RNA molecule, a short hairpin RNA, or a microRNA.

In some embodiments of any of the preceding aspects, the method further includes formulating the therapeutic circular DNA vector in a pharmaceutically acceptable carrier to produce a pharmaceutical composition. In some embodiments, the pharmaceutical composition comprises at least 1.0 mg therapeutic circular DNA vector in a pharmaceutically acceptable carrier. In some embodiments, the therapeutic circular DNA vector in the pharmaceutical composition is at least 70% supercoiled monomer (e.g., by densitometry analysis of gel electrophoresis). In some embodiments, the therapeutic circular DNA vector in the pharmaceutical composition is at least 80% supercoiled monomer (e.g., by densitometry analysis of gel electrophoresis). In some embodiments, the pharmaceutical composition comprises <1.0% protein content by mass, less than <1.0% RNA content by mass, and less than <0.5 EU/mg endotoxin.

In another aspect, provided herein is a composition (e.g., a pharmaceutical composition) produced by the method of any of the preceding embodiments of any of the preceding aspects.

In another aspect, provided herein is a method of expressing a therapeutic sequence in an individual, wherein the method comprises administering to the individual the pharmaceutical composition produced by the method of any of the preceding embodiments of any of the preceding aspects. Therapeutic sequences of any of the therapeutic circular DNA vectors described herein, or pharmaceutical compositions thereof, can be expressed in skin, skeletal muscle, tumors (including, e.g., melanomas), eye, or lung via in vivo electrotransfer.

In another aspect, provided herein is a method of treating a disease or disorder in an individual in need thereof, the method comprising administering to the individual the pharmaceutical composition produced by the method of any of the preceding embodiments of any of the preceding aspects. In some embodiments, the method includes in vivo electrotransfer of the therapeutic circular DNA vector to the skin, skeletal muscle, tumor (including, e.g., melanomas), eye, or lung of the individual.

In another aspect, provided is a therapeutic circular DNA vector comprising a therapeutic sequence having a 3′ end and 5′ end, wherein the 3′ end of the therapeutic sequence is connected to the 5′ end of the therapeutic sequence by a four-base pair sequence comprising at least two consecutive adenines (A's). In some embodiments, the four-base pair sequence consists of AAAA. In some embodiments, the therapeutic circular DNA vector comprises (e.g., consists of) a nucleic acid sequence having 85% sequence identity to SEQ ID NO: 1. In some embodiments, the therapeutic circular DNA vector comprises SEQ ID NO: 1. In some embodiments, two and only two consecutive bases of the four-base pair sequence are AA. In some embodiments, the four-base pair sequence consists of AACC. In some embodiments, the therapeutic circular DNA vector comprises a nucleic acid sequence having at least 85% sequence identity to (e.g., at least 90%, at least 91%, at least 92%, at least 93%, at least 94%, at least 95%, at least 96%, at least 97%, at least 98%, at least 99%, or 100% sequence identity to) SEQ ID NO: 3. In some embodiments, the therapeutic circular DNA vector comprises, or consists of, SEQ ID NO: 3.

In another aspect, the invention provides a pharmaceutical composition comprising the therapeutic circular DNA vector of the previous aspect. In some embodiments, the pharmaceutical composition comprises at least 1.0 mg of the therapeutic circular DNA vector in a pharmaceutically acceptable carrier. In some embodiments, the therapeutic circular DNA vector is at least 70% supercoiled monomer. In some embodiments, the pharmaceutical composition comprises no more than 1.0% of residual protein or backbone sequence. In some embodiments, the pharmaceutical composition comprises <1.0% protein content by mass, less than <1.0% RNA content by mass, and less than <5 EU/mg endotoxin.

In another aspect, the invention involves a method of expressing a therapeutic sequence in an individual (e.g. human), wherein the method comprises administering to the individual the pharmaceutical composition of any embodiment of the previous aspect. In some embodiments, the method comprises delivering the therapeutic circular DNA vector to an eye of the individual by in vivo electrotransfer.

In another aspect, the invention involves a method of treating an ocular disease or disorder in an individual (e.g. human) in need thereof, wherein the method comprises administering to the individual the pharmaceutical composition of any embodiment of the previous aspect. In some embodiments, the method comprises delivering the therapeutic circular DNA vector to an eye of the individual by in vivo electrotransfer. In another aspect, provided herein is a kit comprising any of the therapeutic circular DNA vectors or compositions thereof (e.g., pharmaceutical compositions) described herein (or produced by the methods described herein) and instructions for expressing the therapeutic circular DNA vector in a cell, or a culture of cells, using electroporation (e.g., in vitro or ex vivo electroporation) or electrotransfer (e.g., in vivo electrotransfer).

In another aspect, provided herein is a cell (e.g., a mammalian cell) that expresses any one of the therapeutic circular DNA vectors described herein (or produced by the methods described herein). In some embodiments, the cell has been electrotransfected with the vector by electroporation (e.g., in vitro or ex vivo electroporation).

### BRIEF DESCRIPTION OF THE DRAWINGS

FIGS. 1A-IE are a schematic diagram showing a series of reaction steps as described herein, in which two restriction enzymes are used in a cell-free process of producing c3DNA.

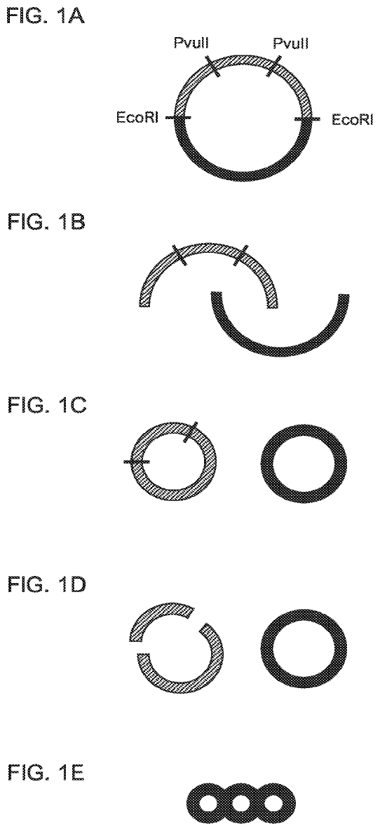

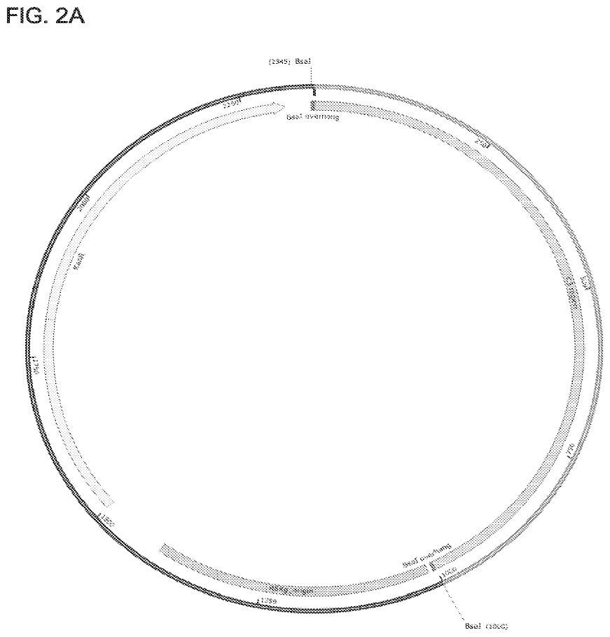

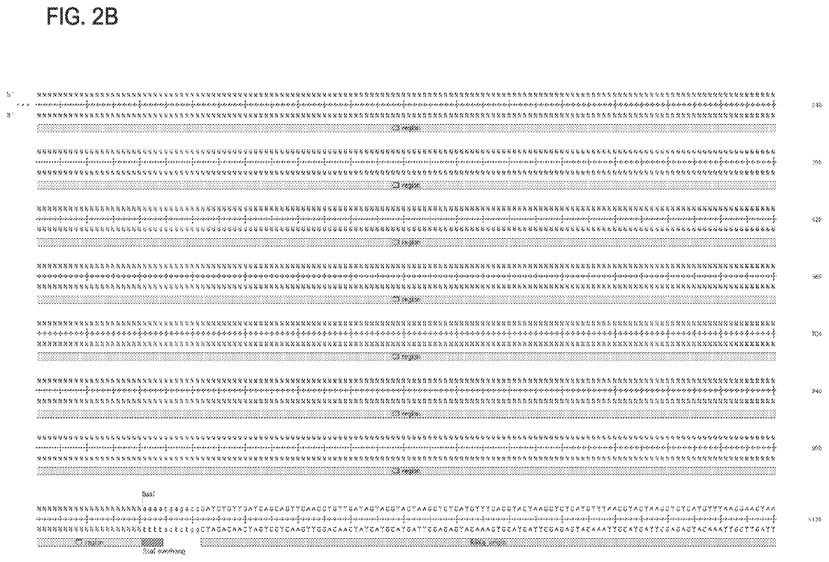

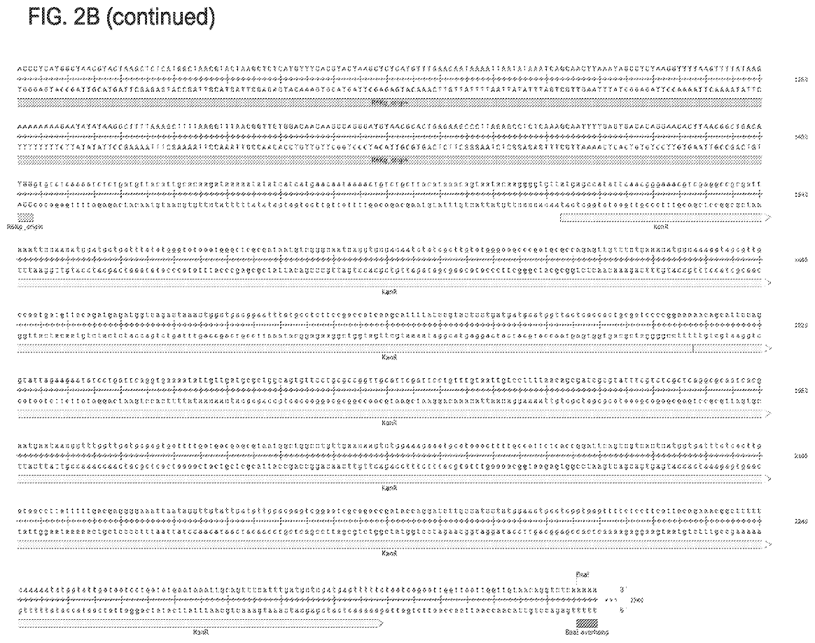

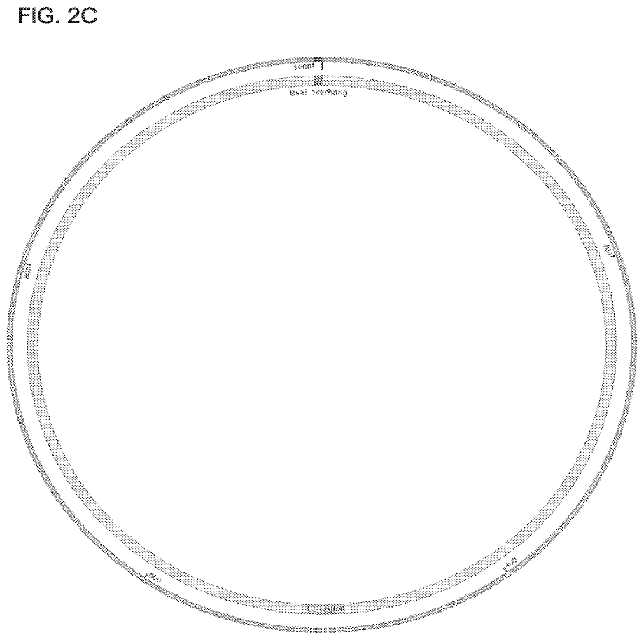

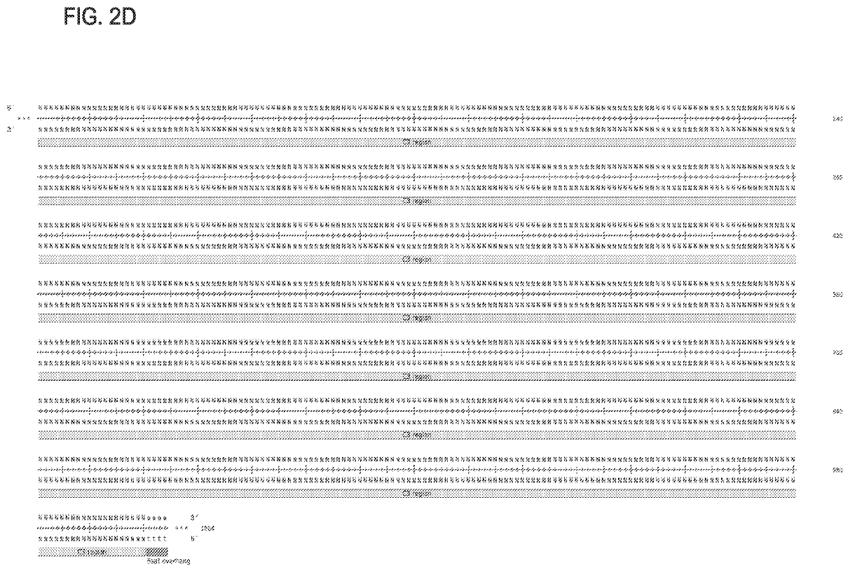

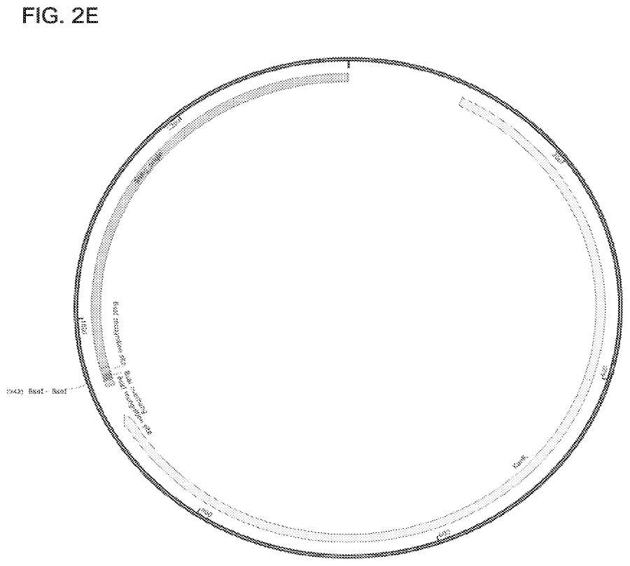

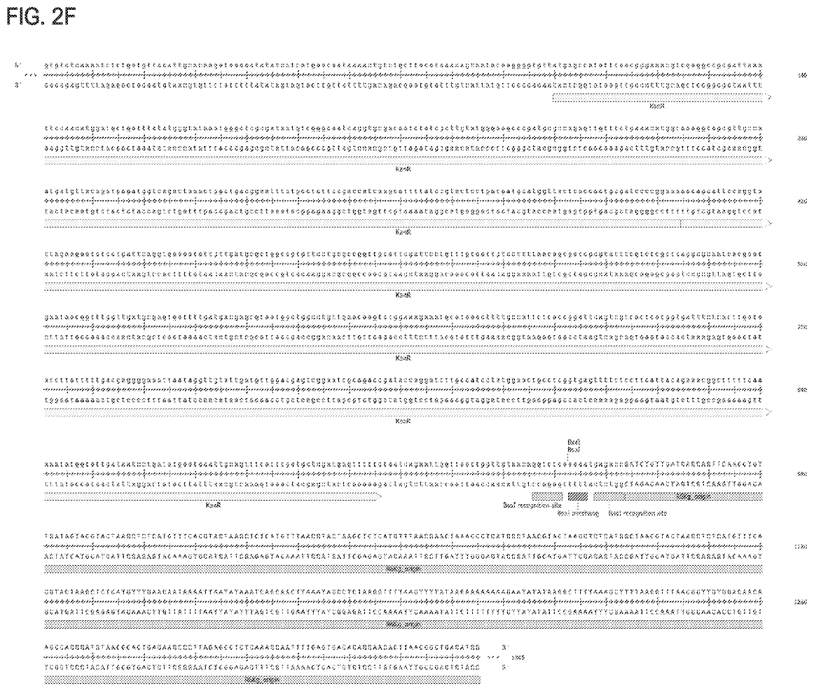

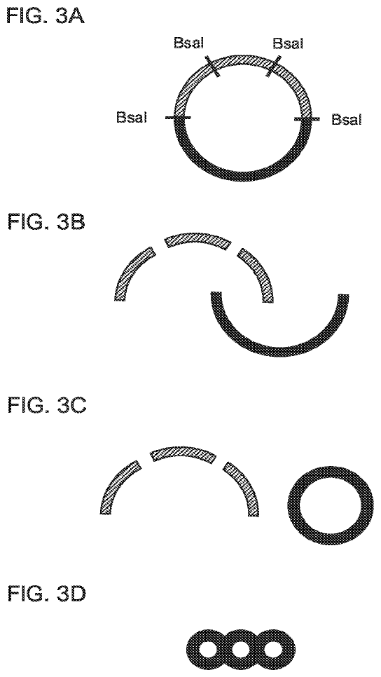

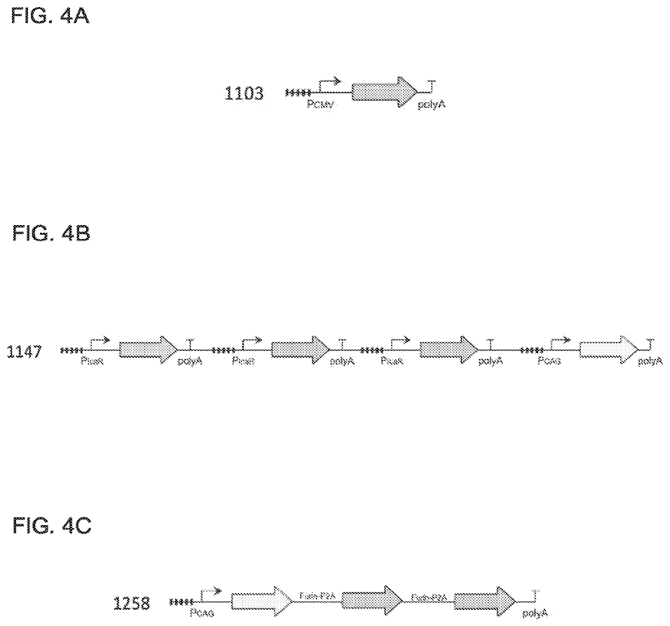

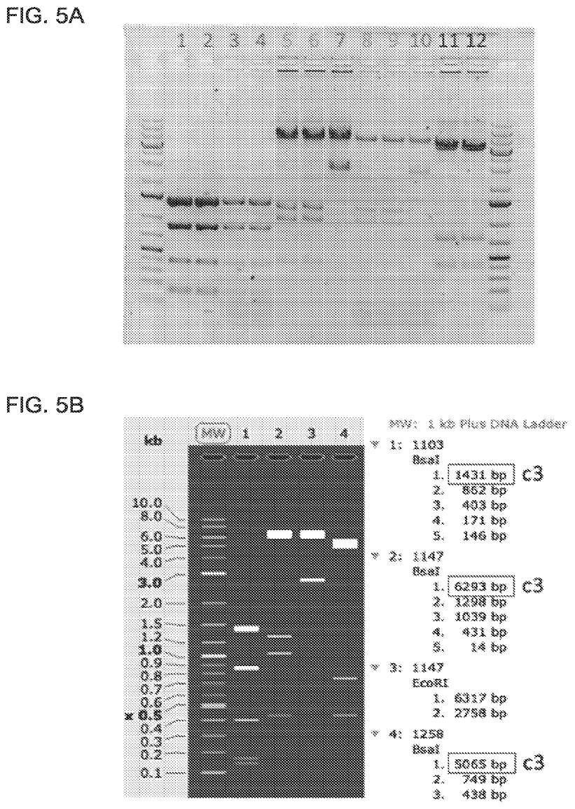

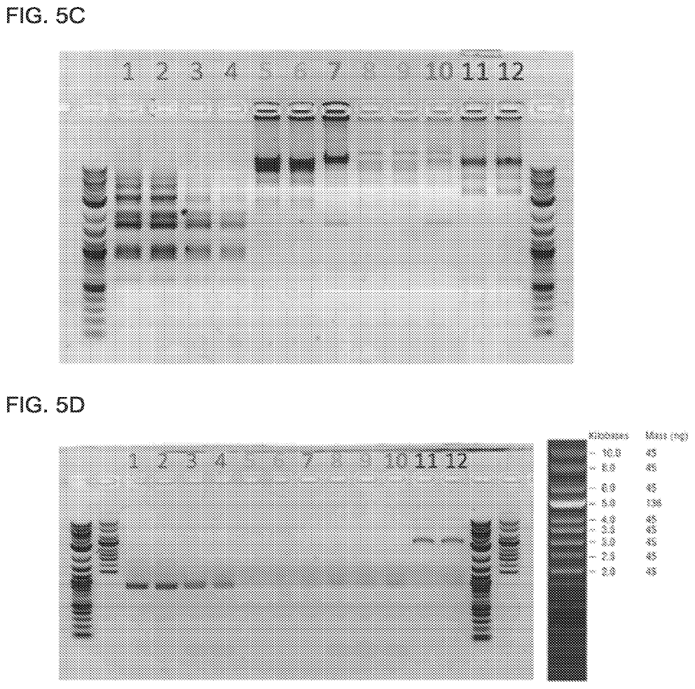

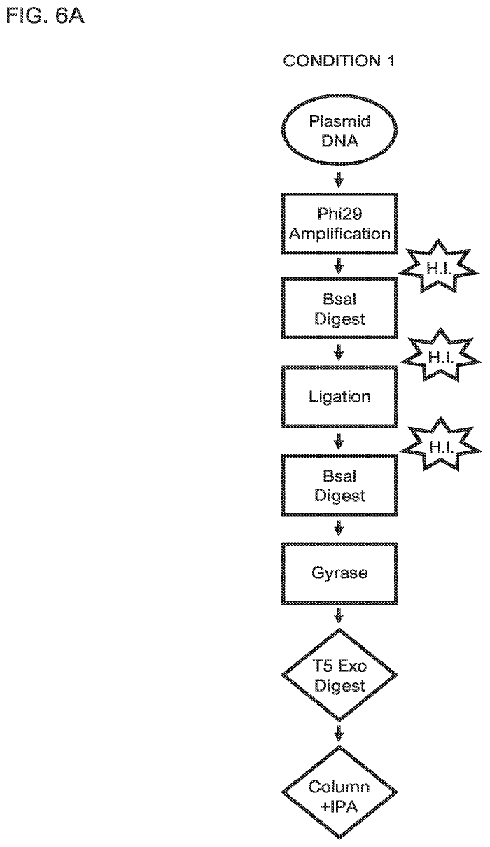

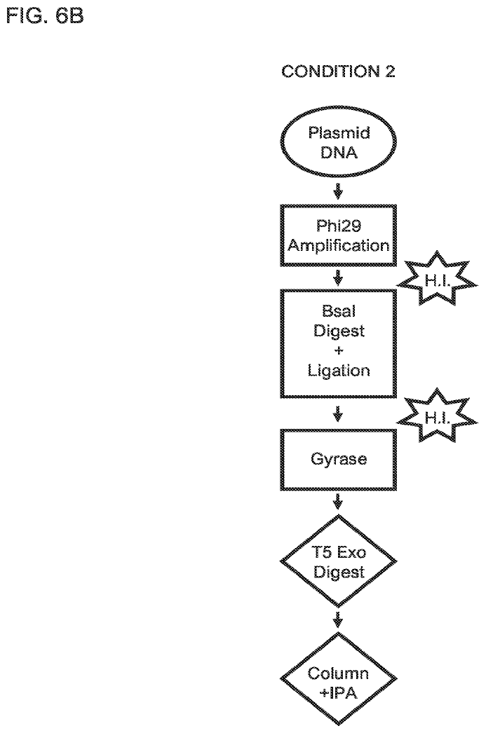

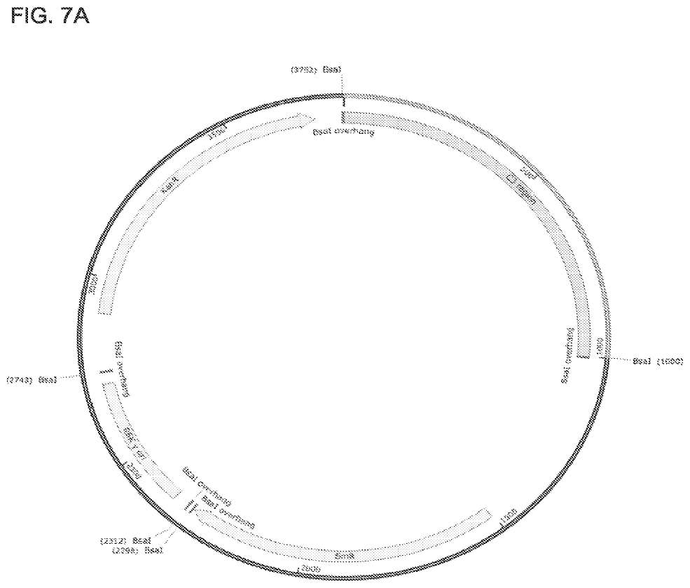

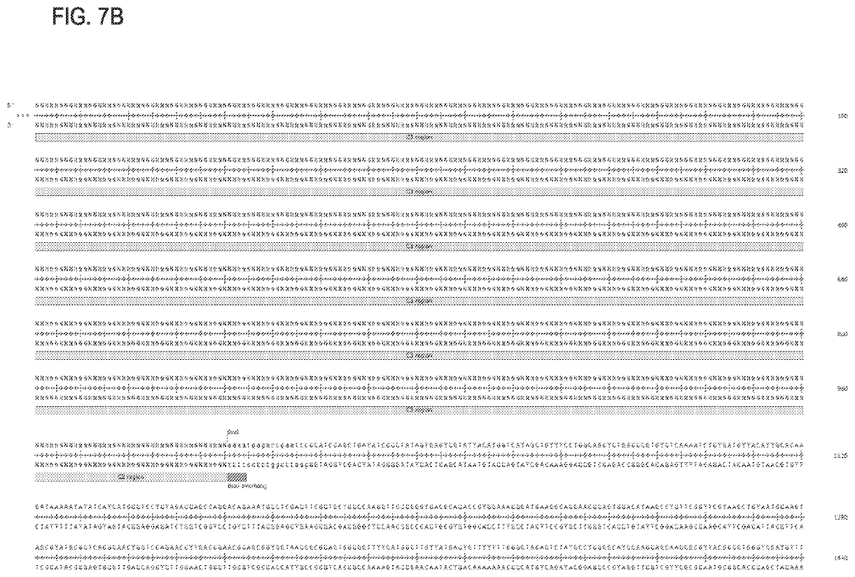

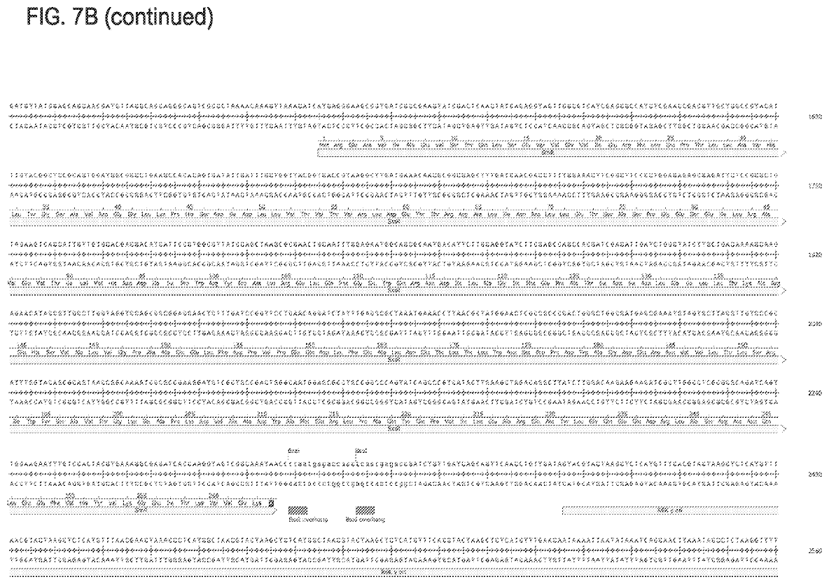

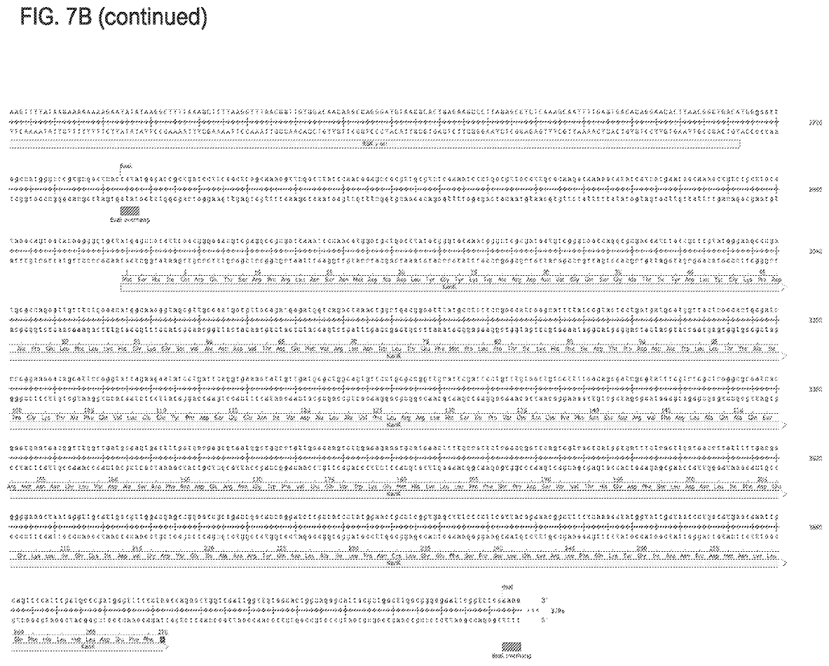

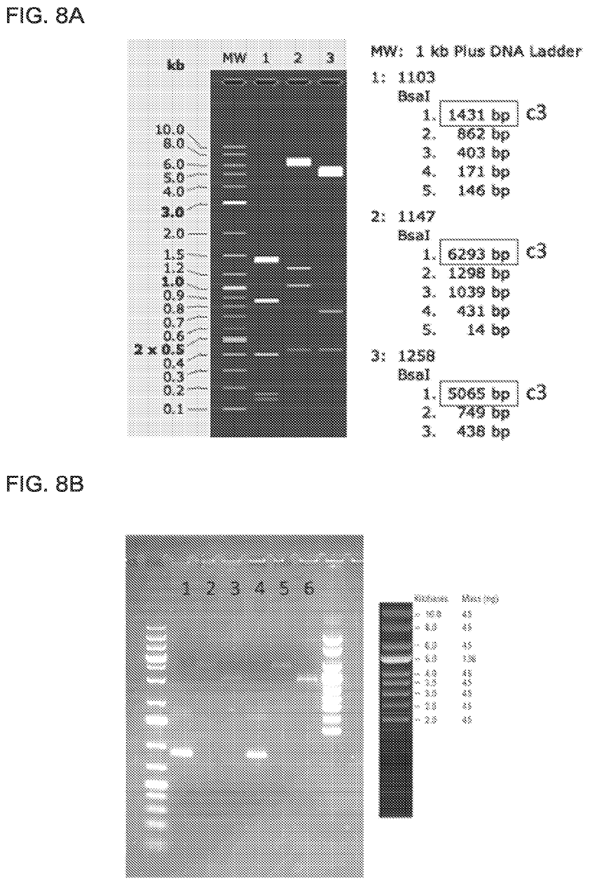

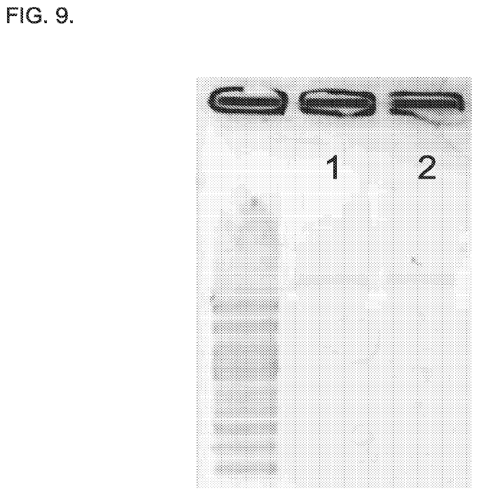

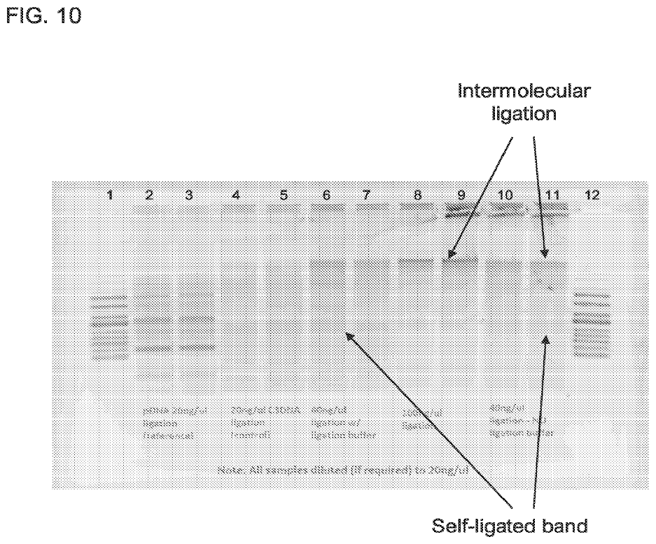

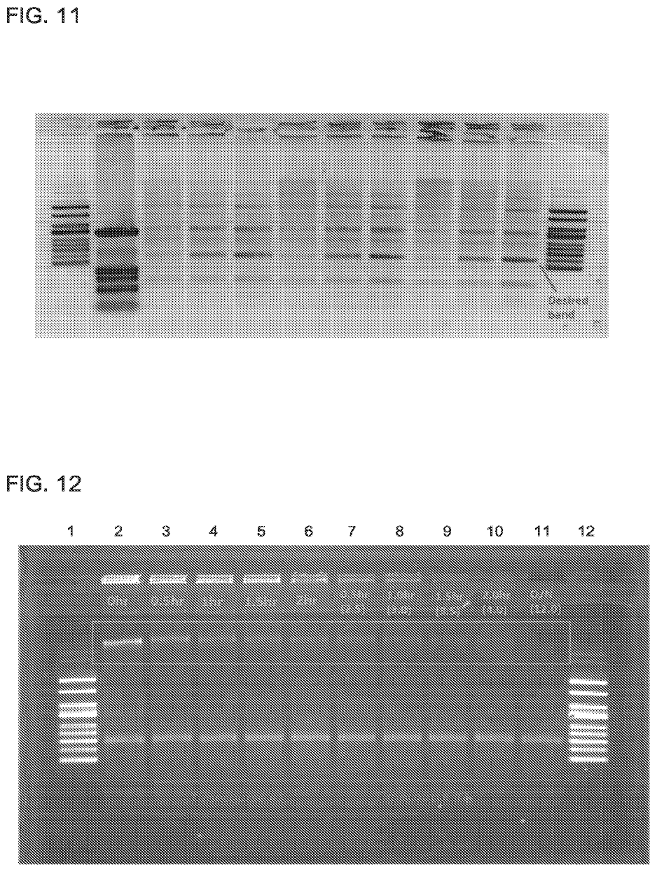

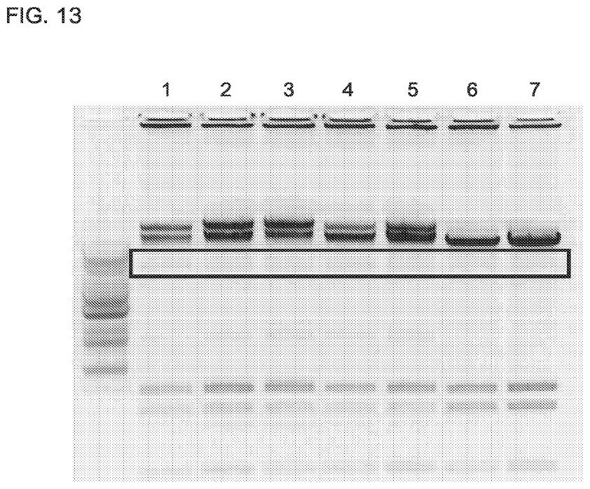

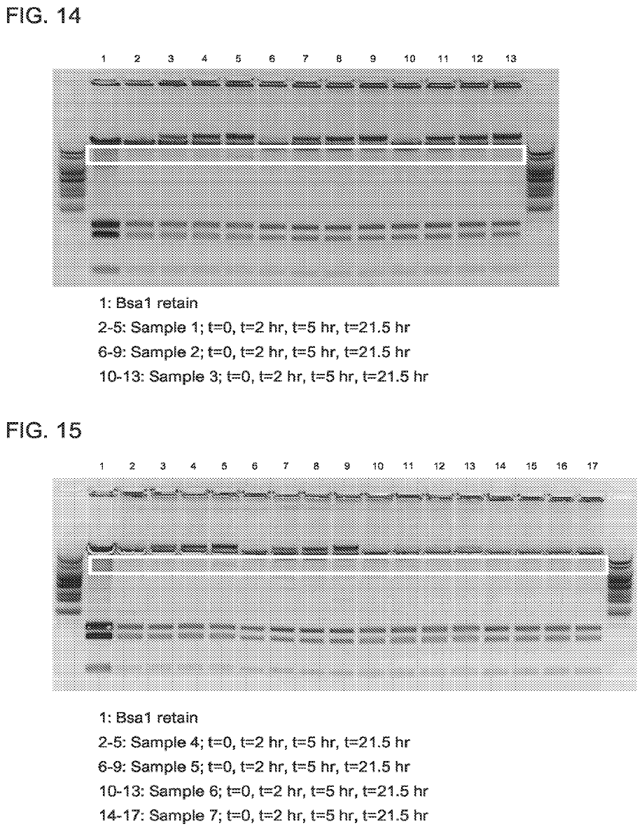

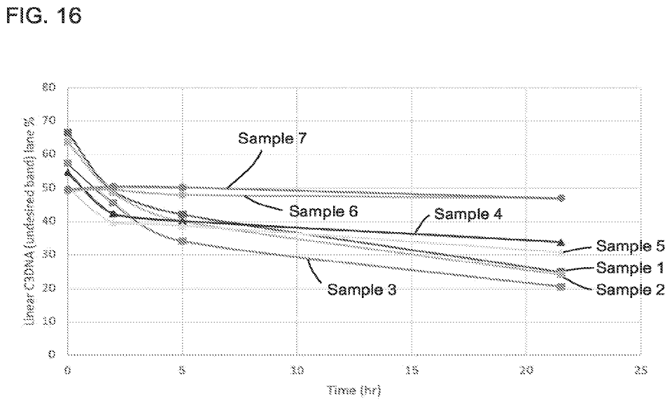

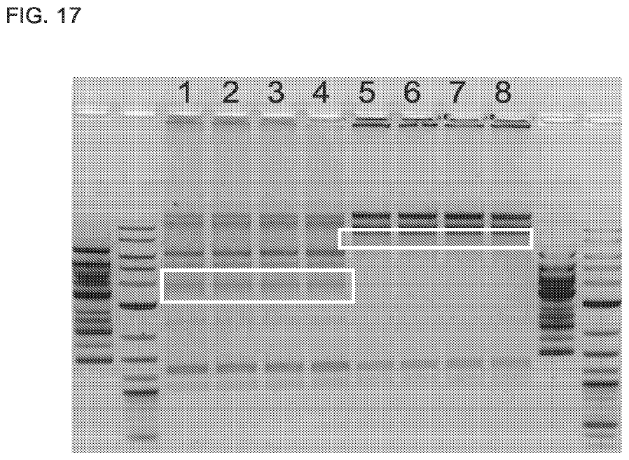

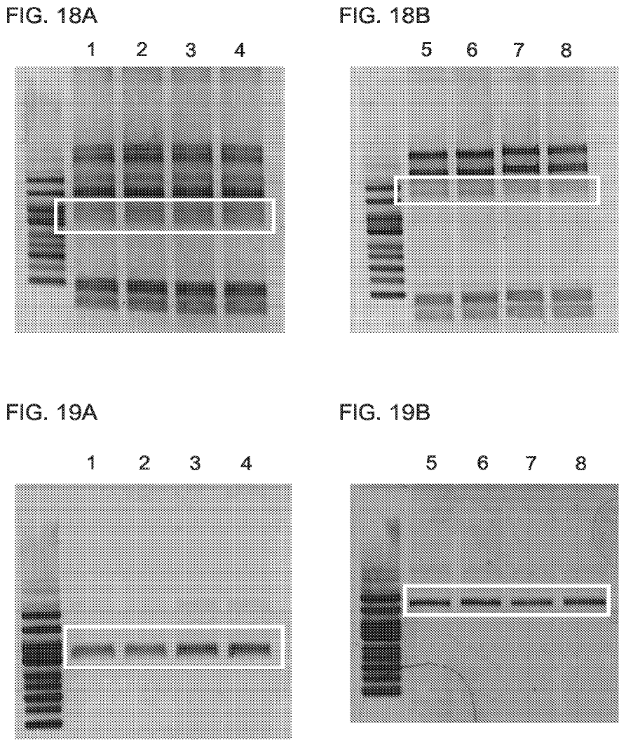

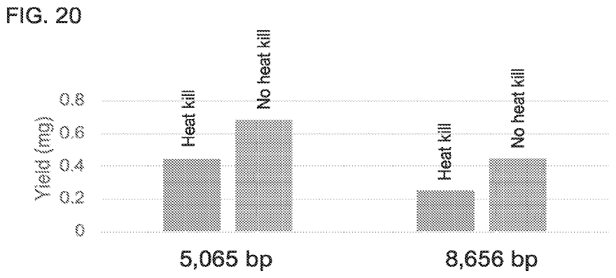

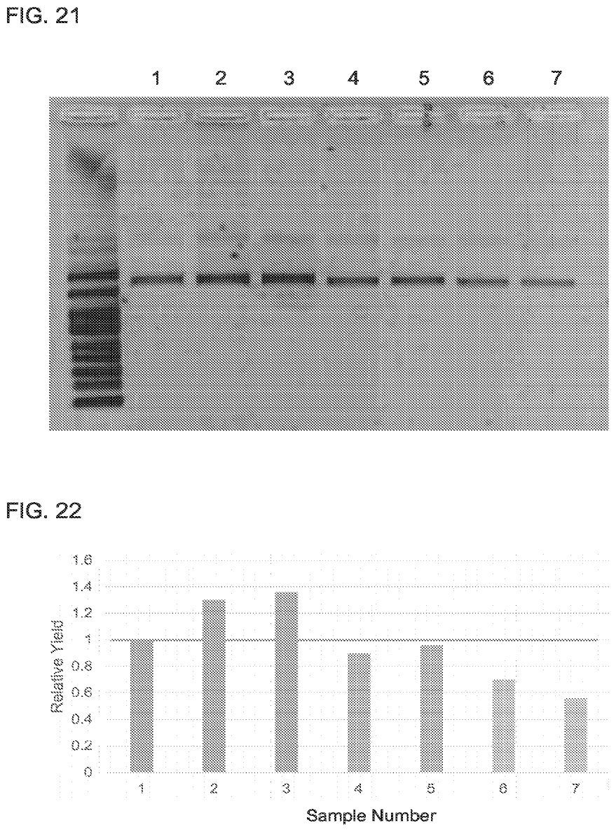

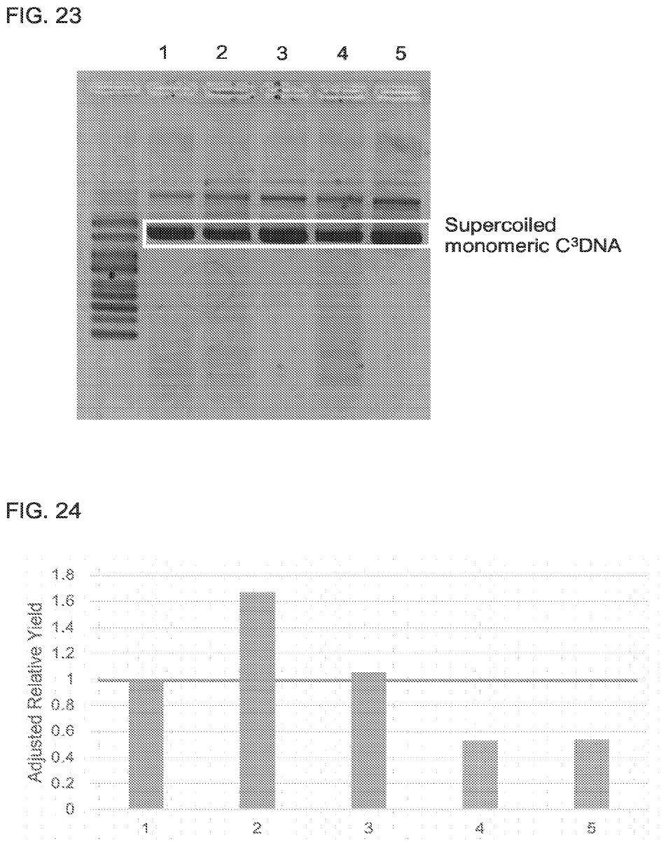

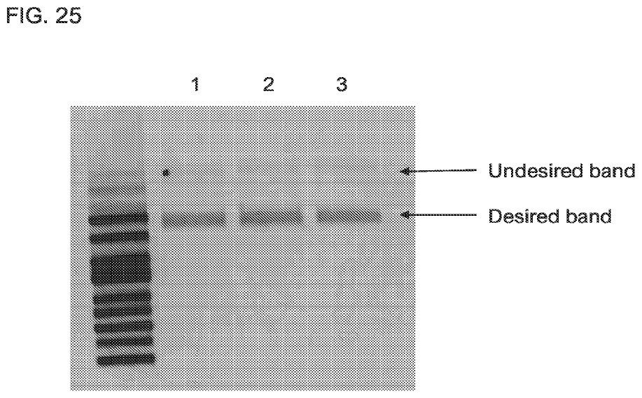

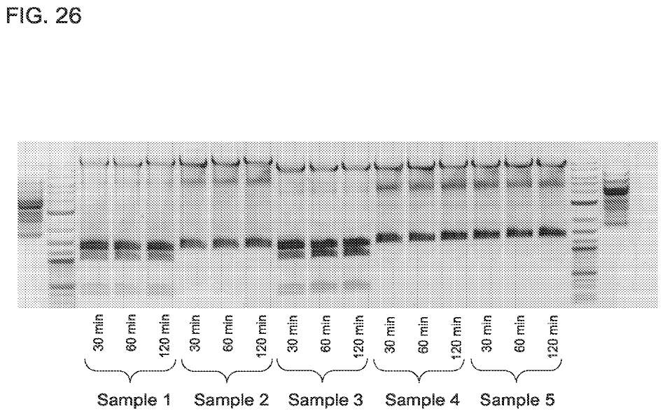

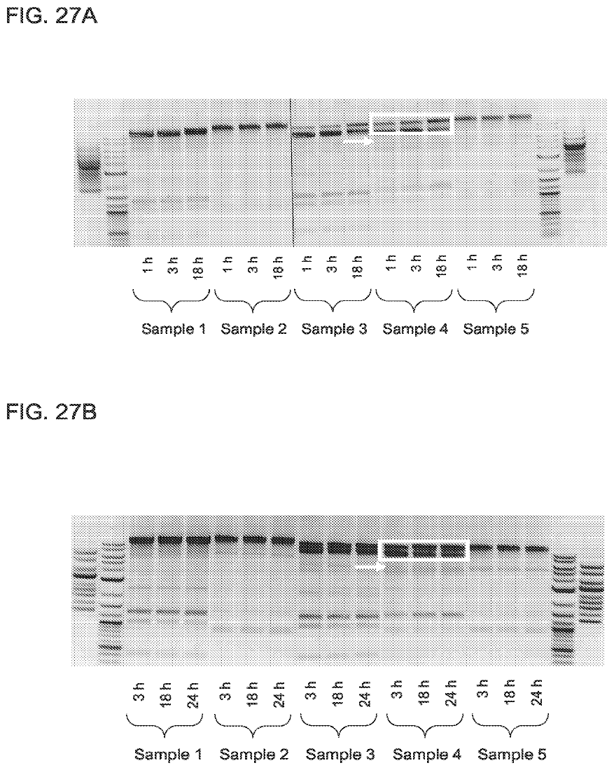

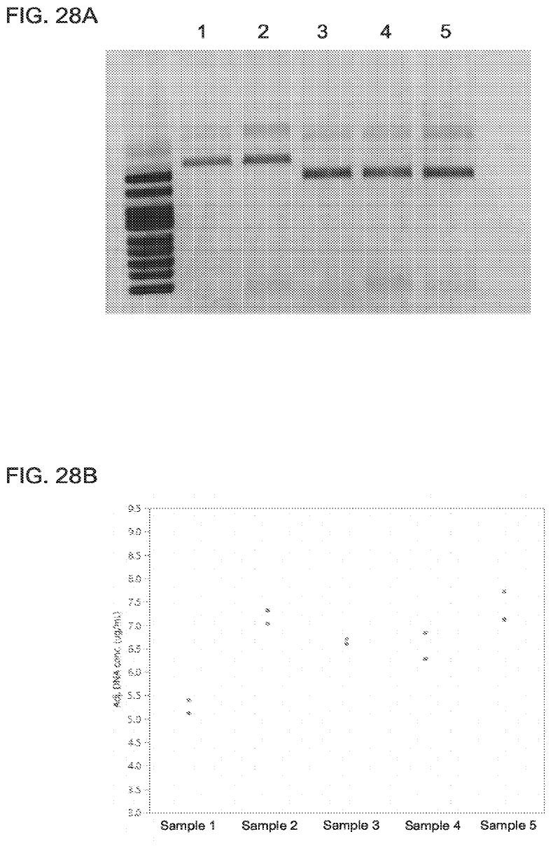

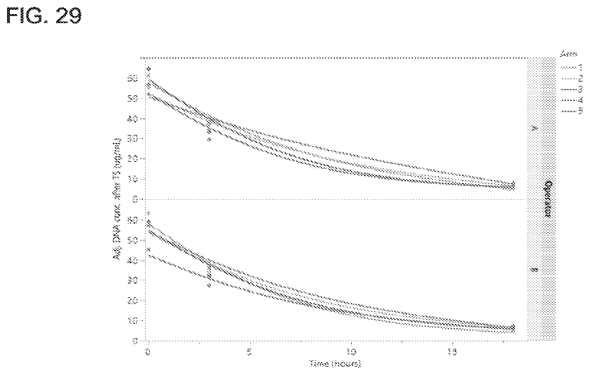

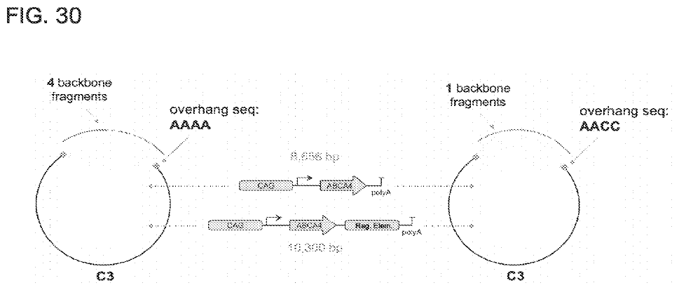

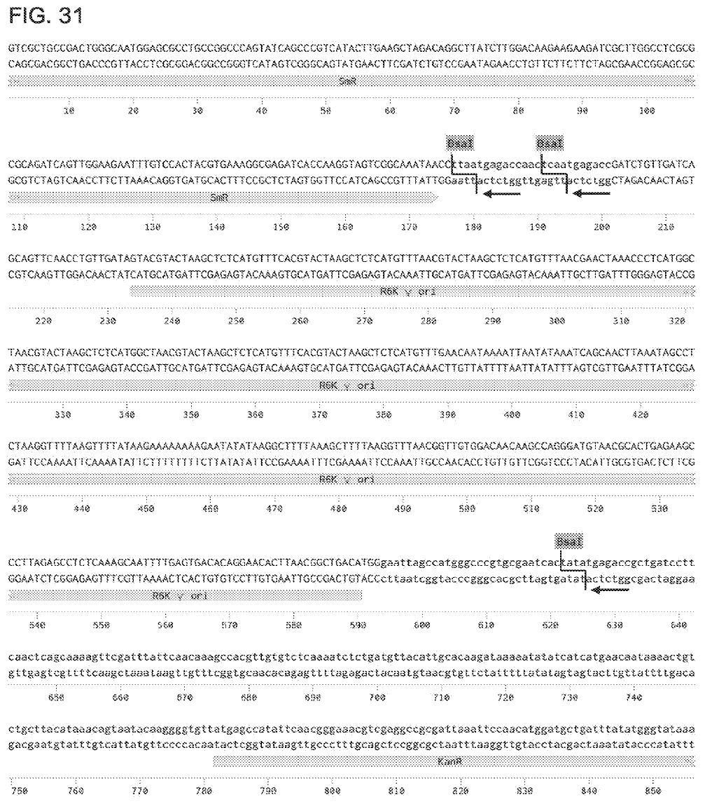

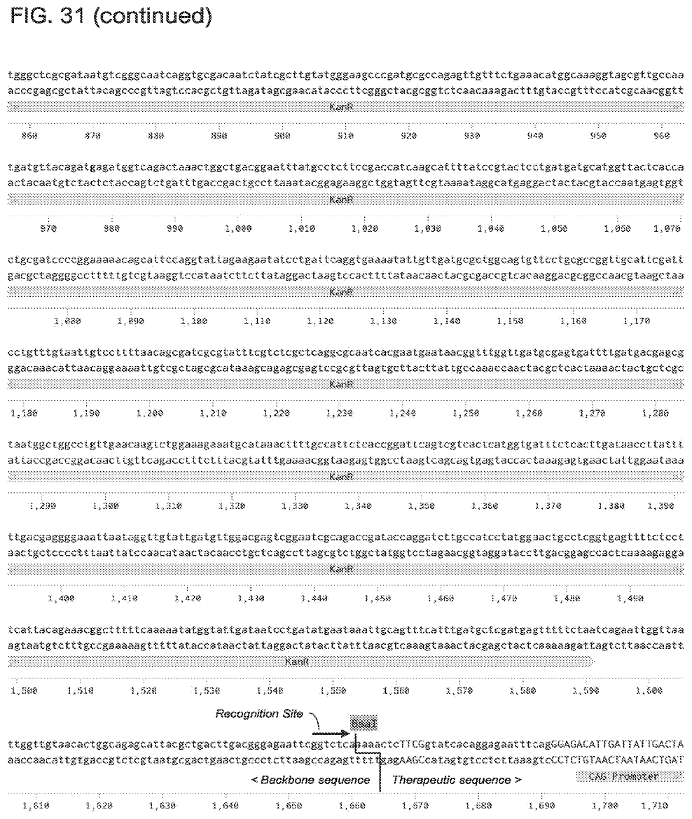

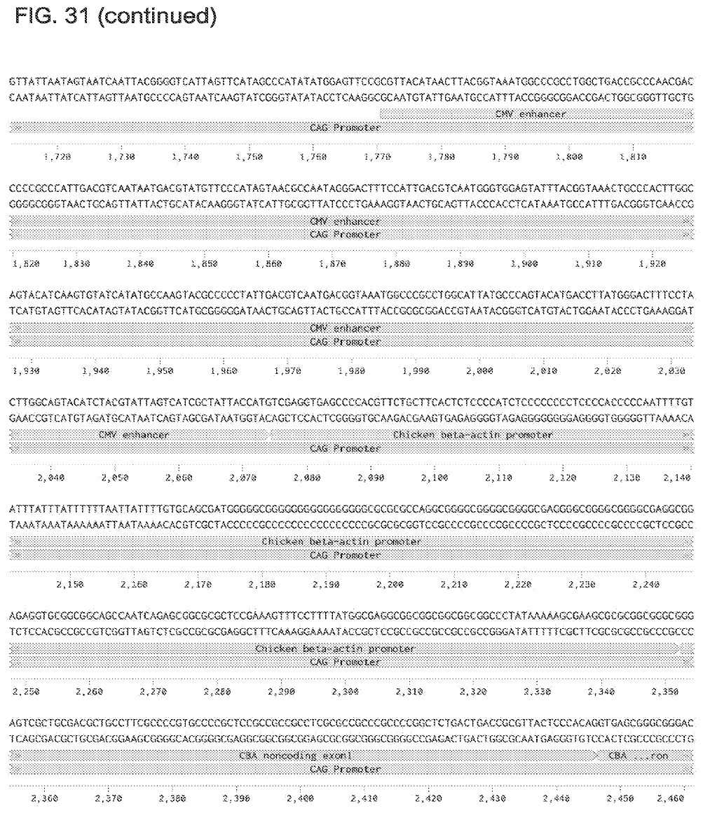

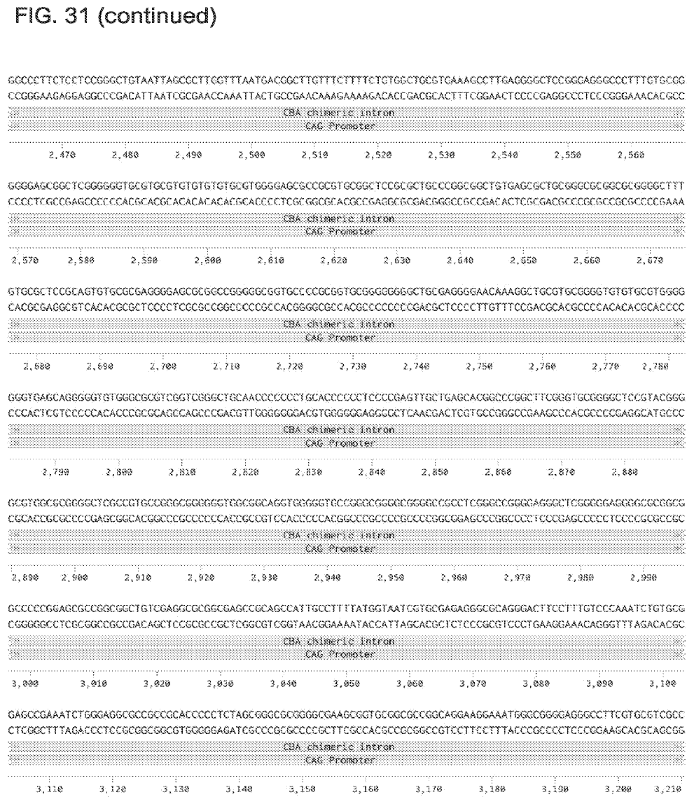

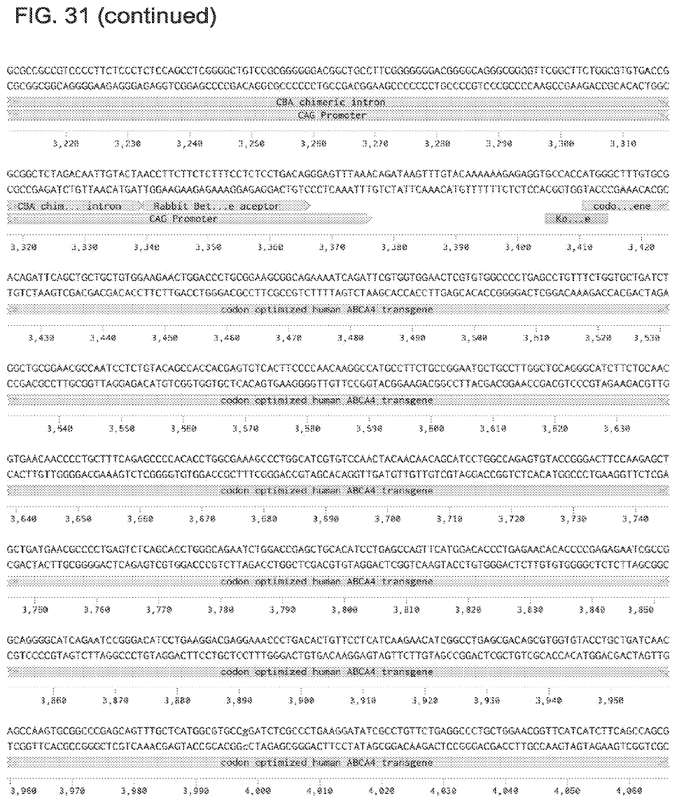

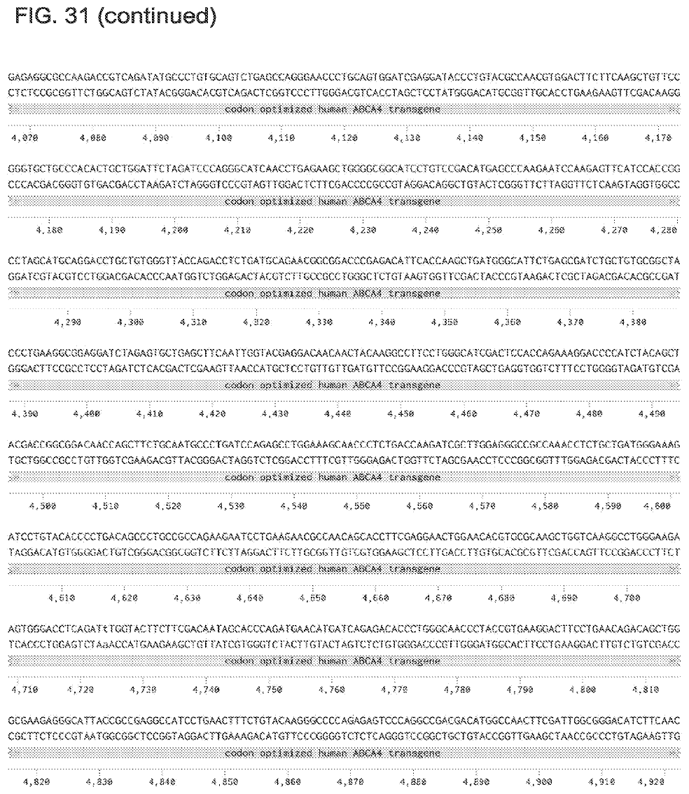

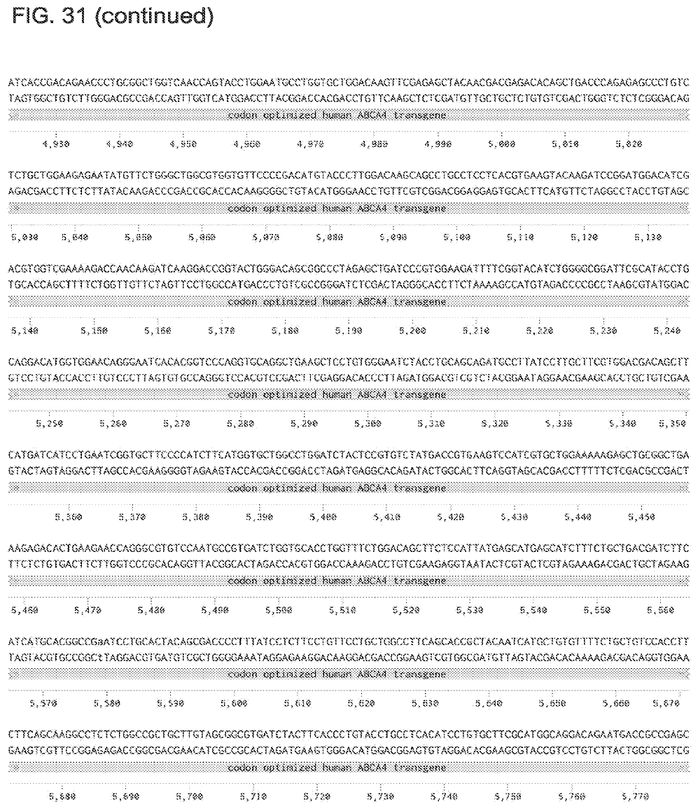

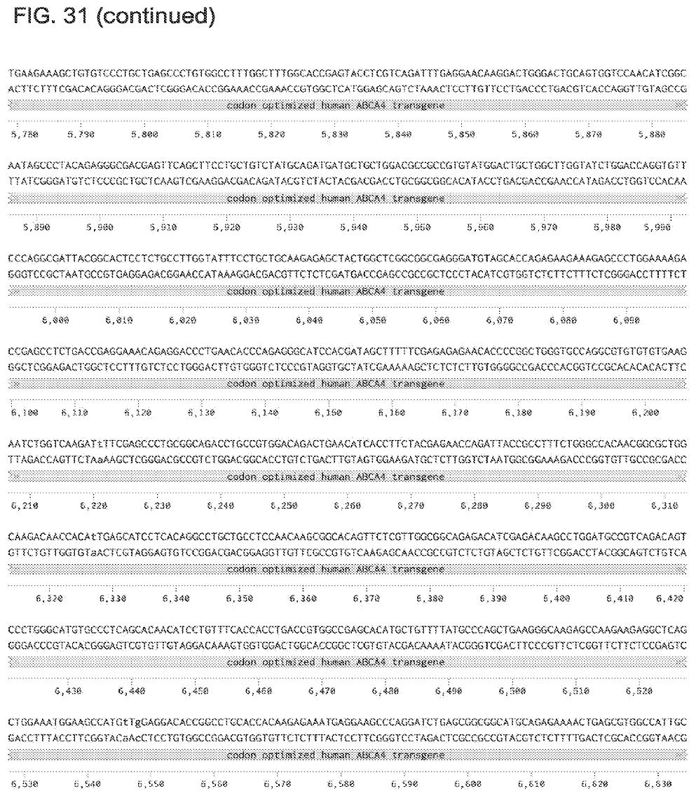

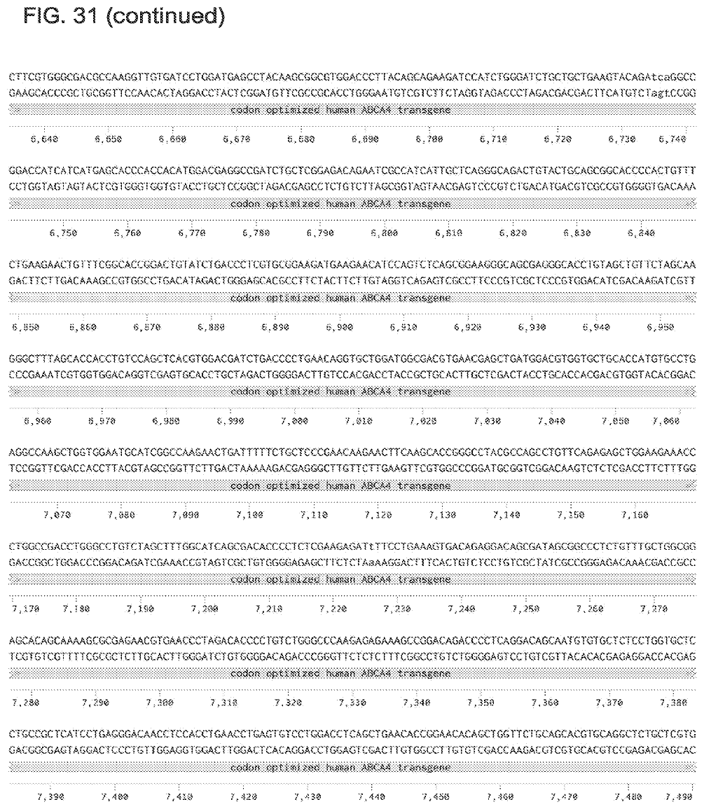

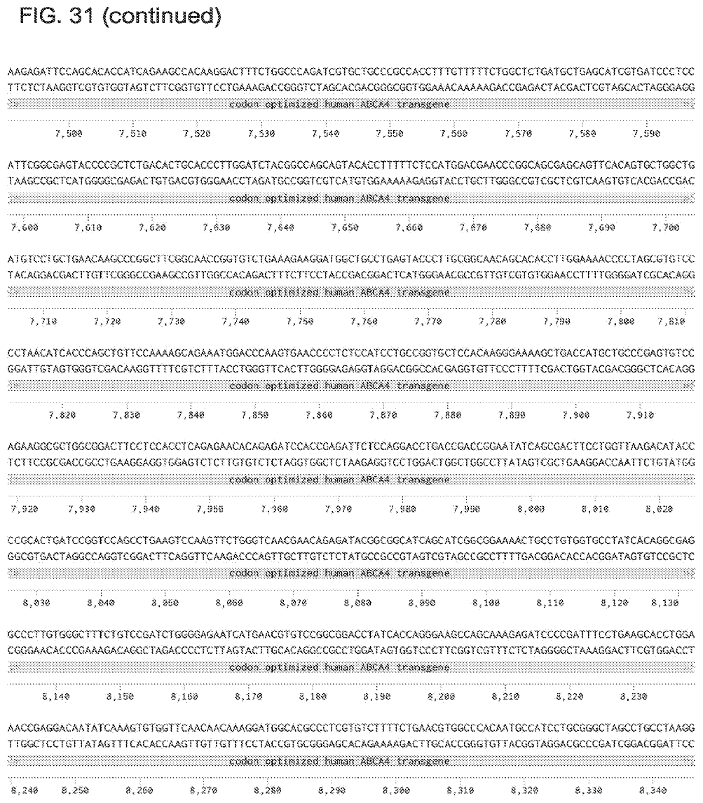

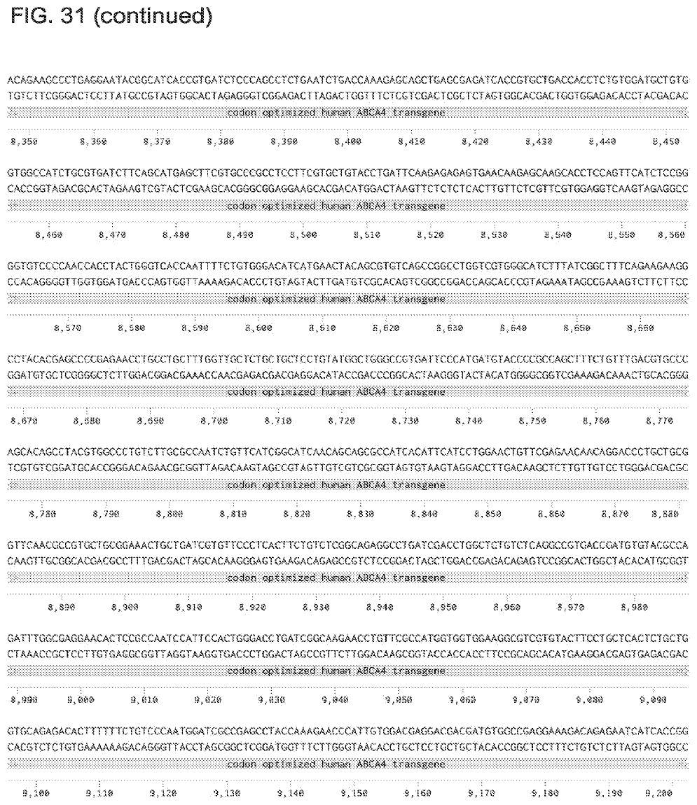

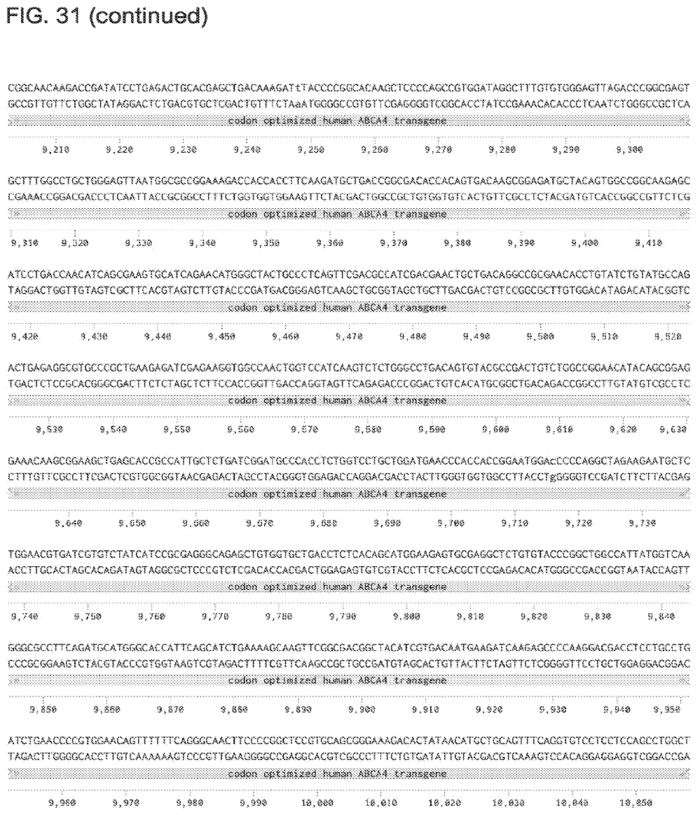

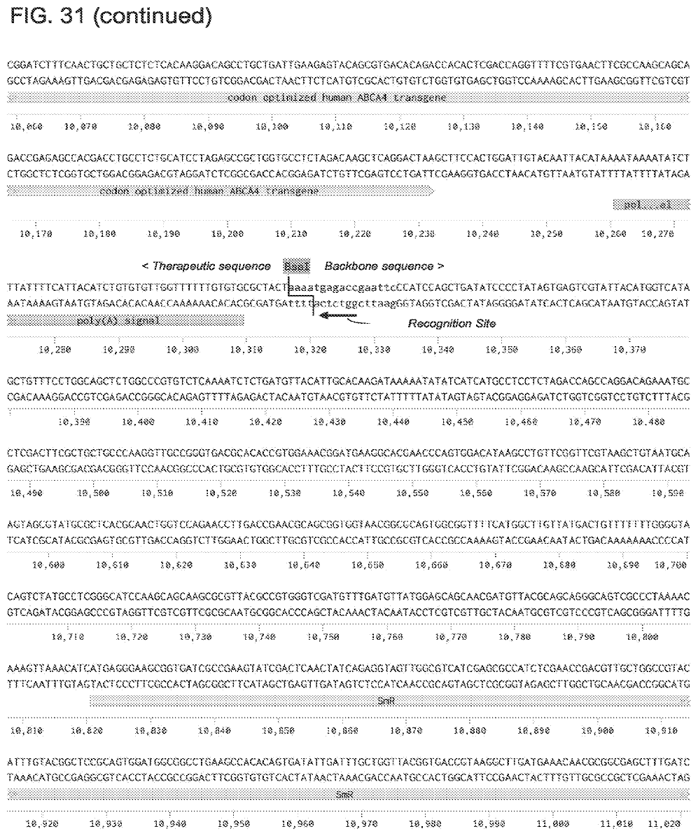

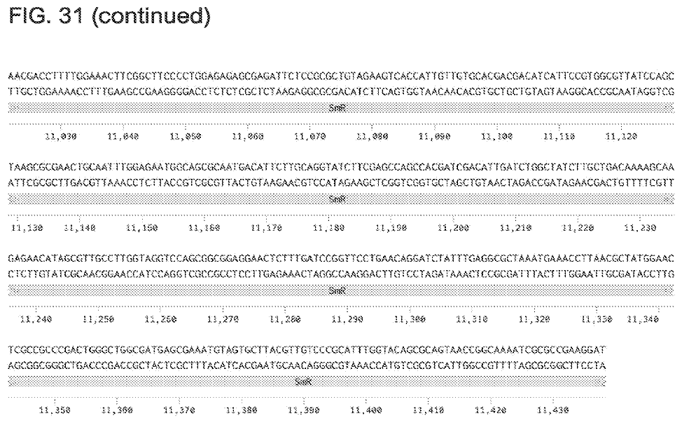

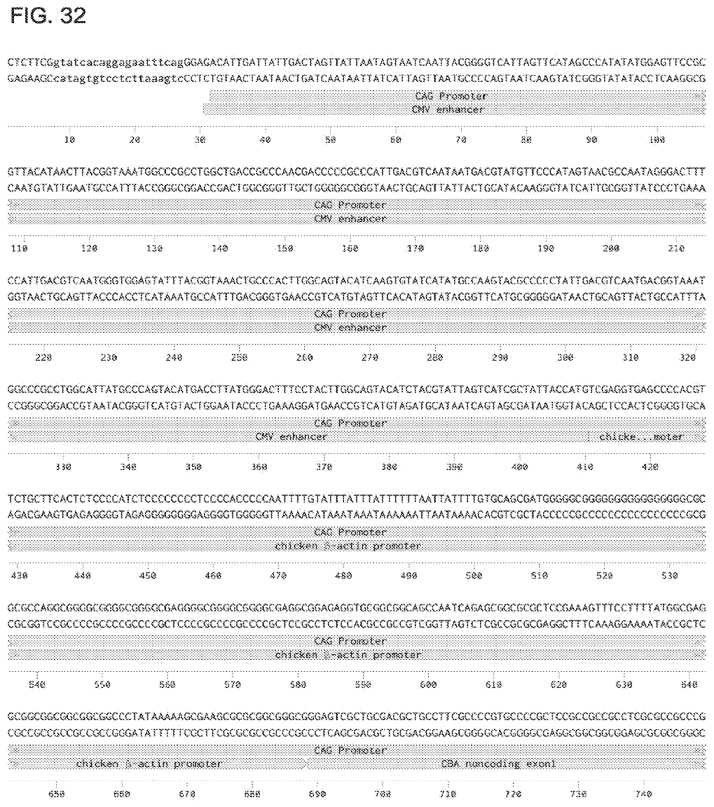

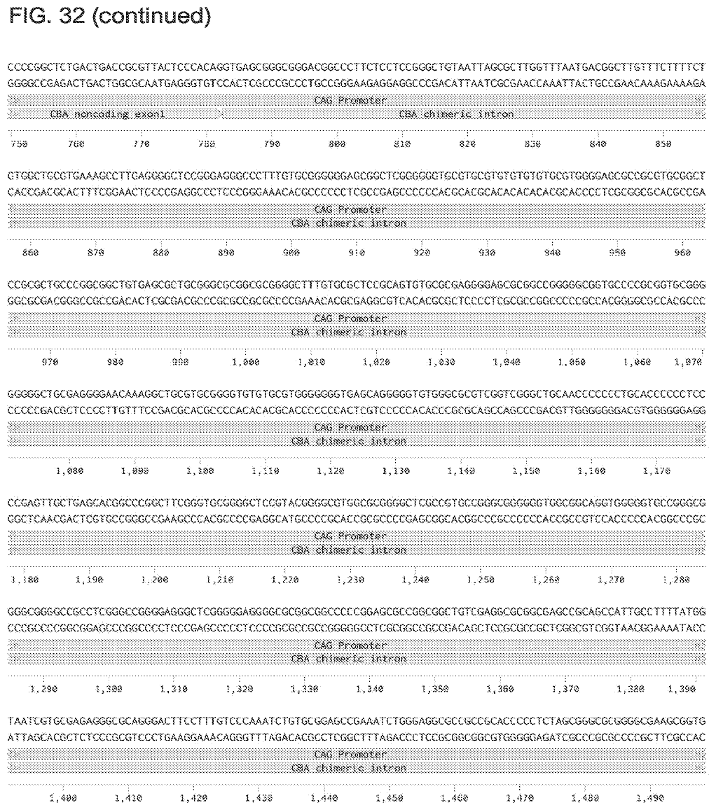

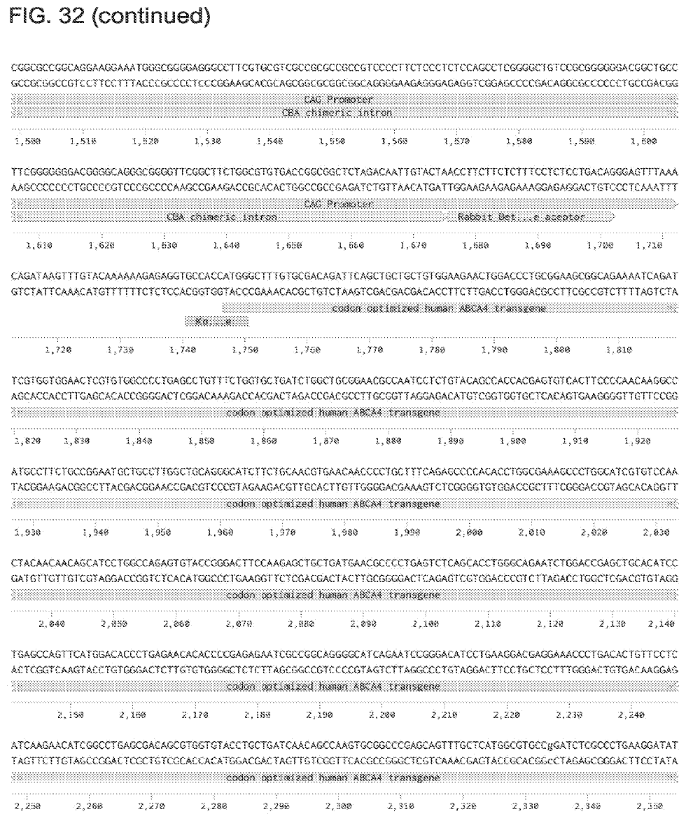

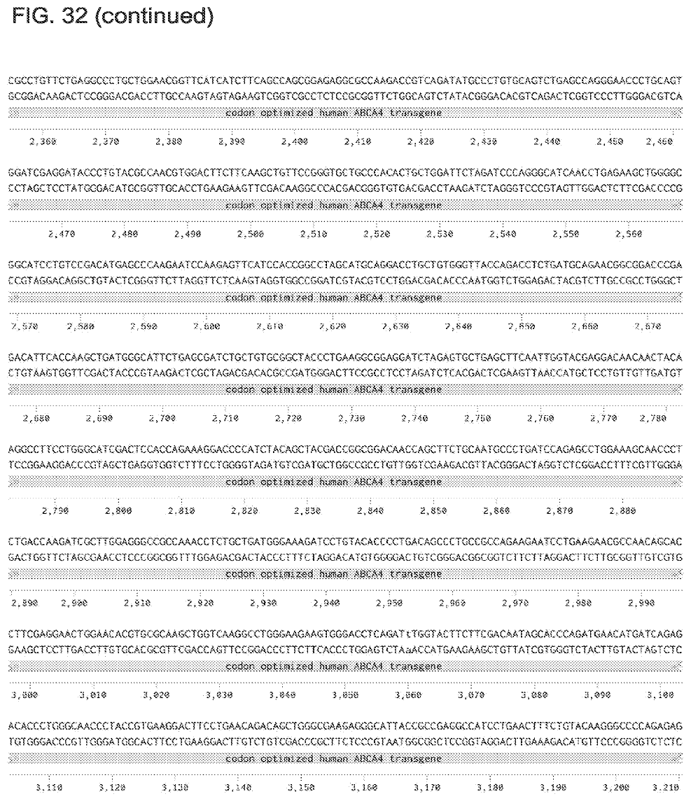

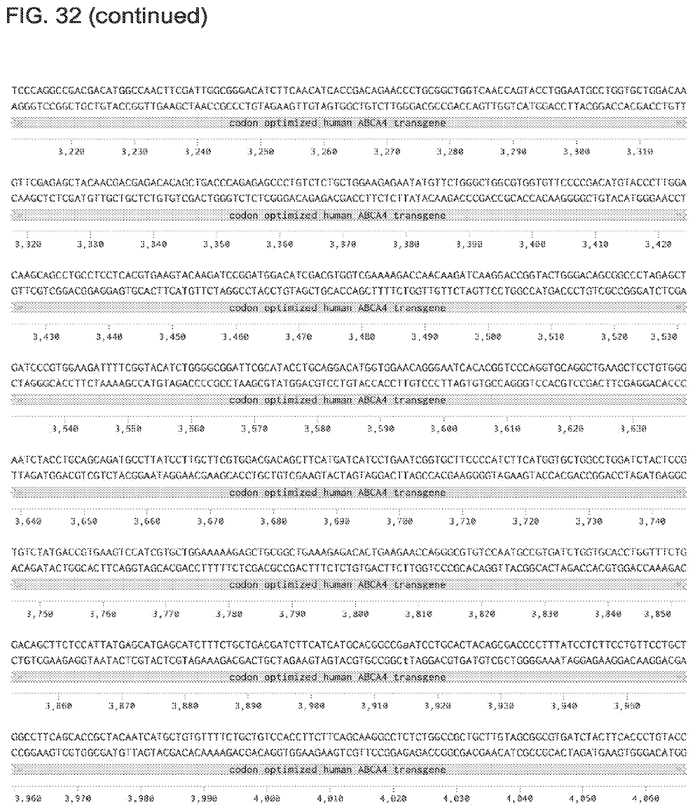

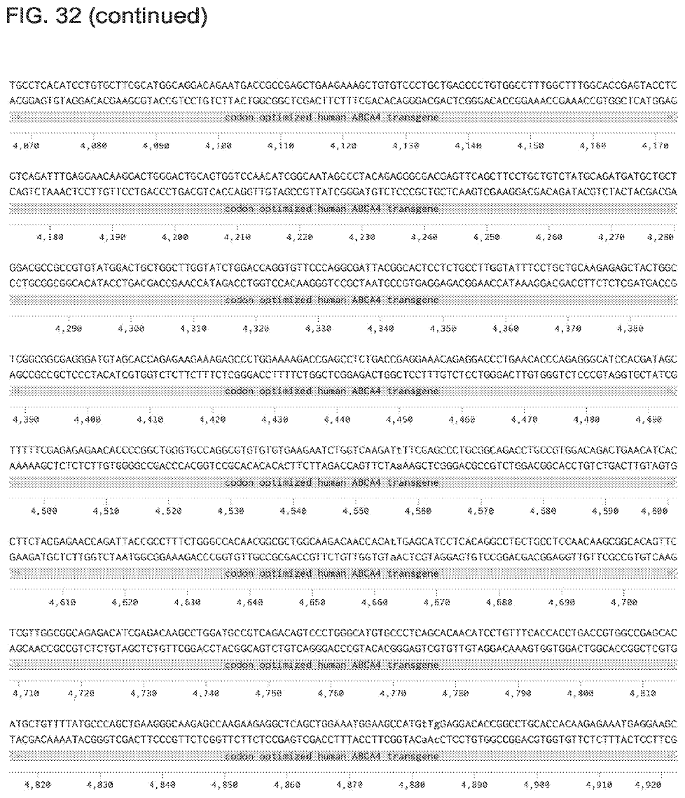

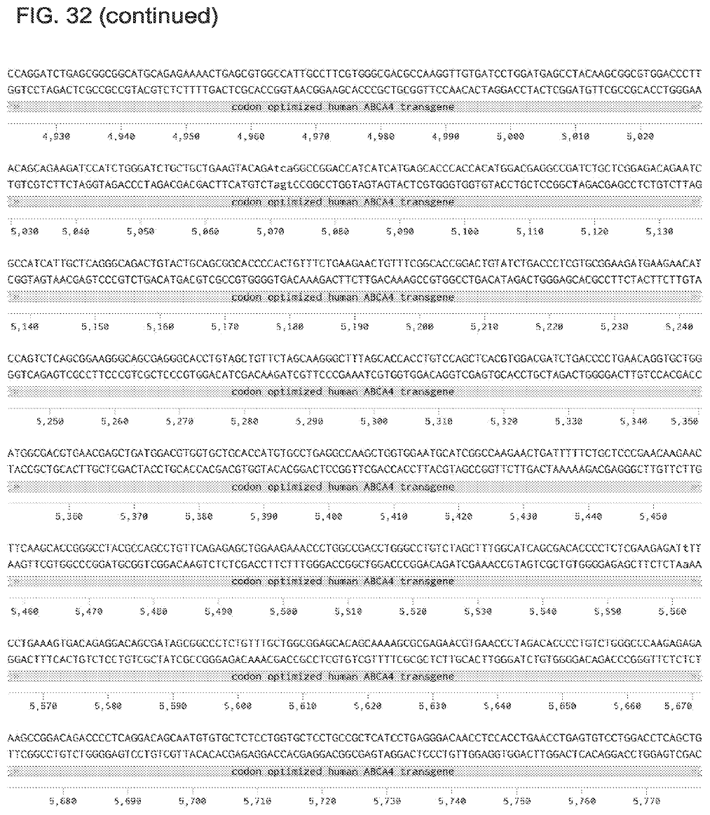

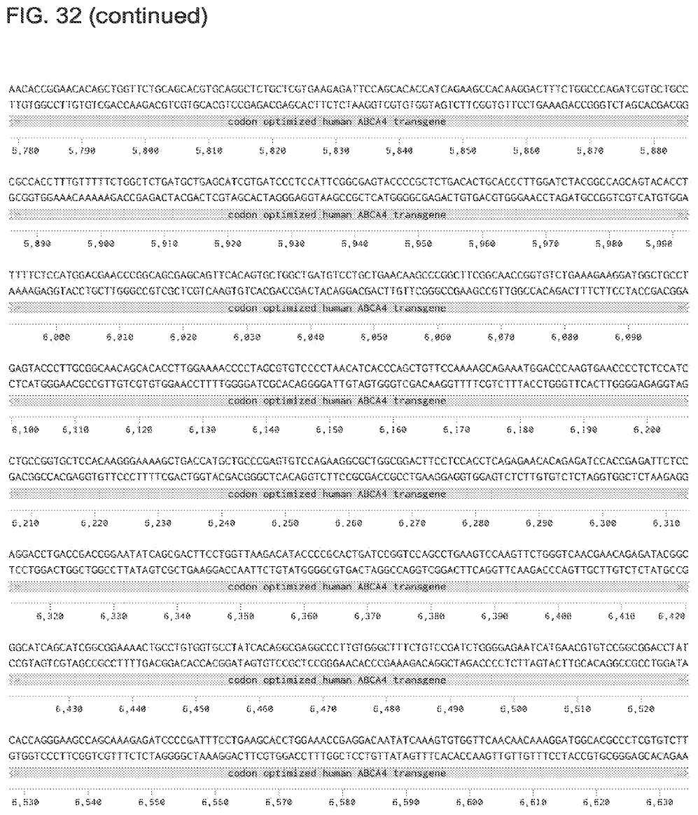

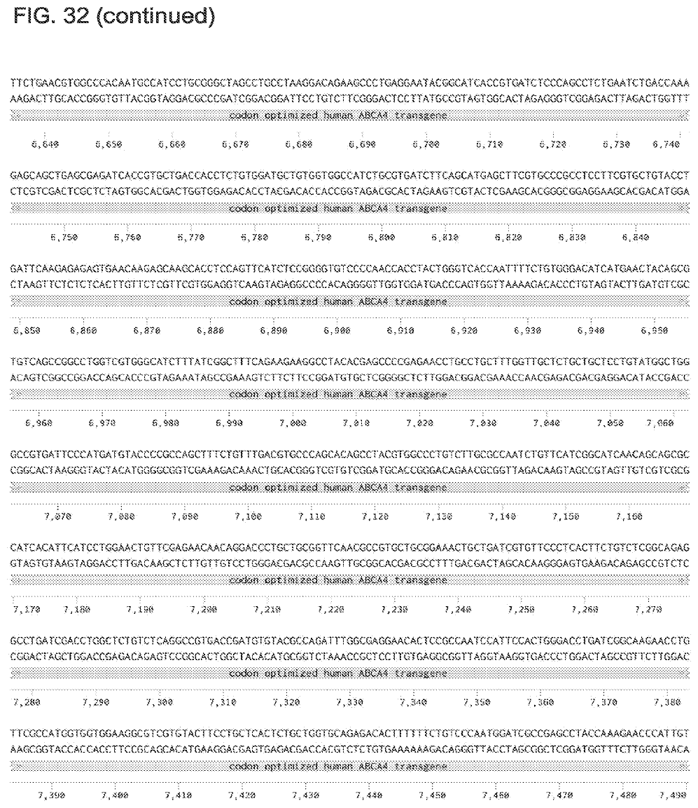

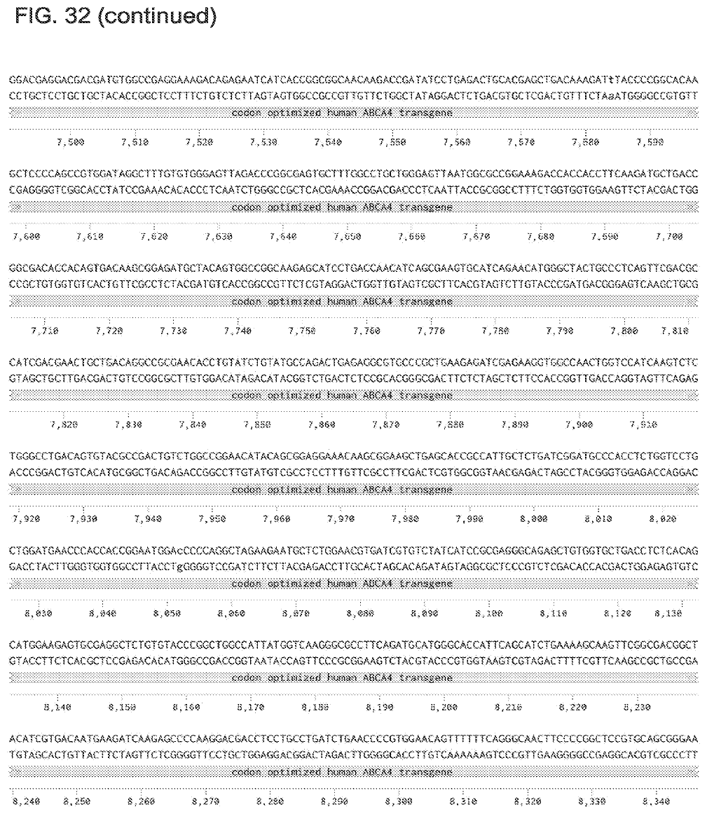

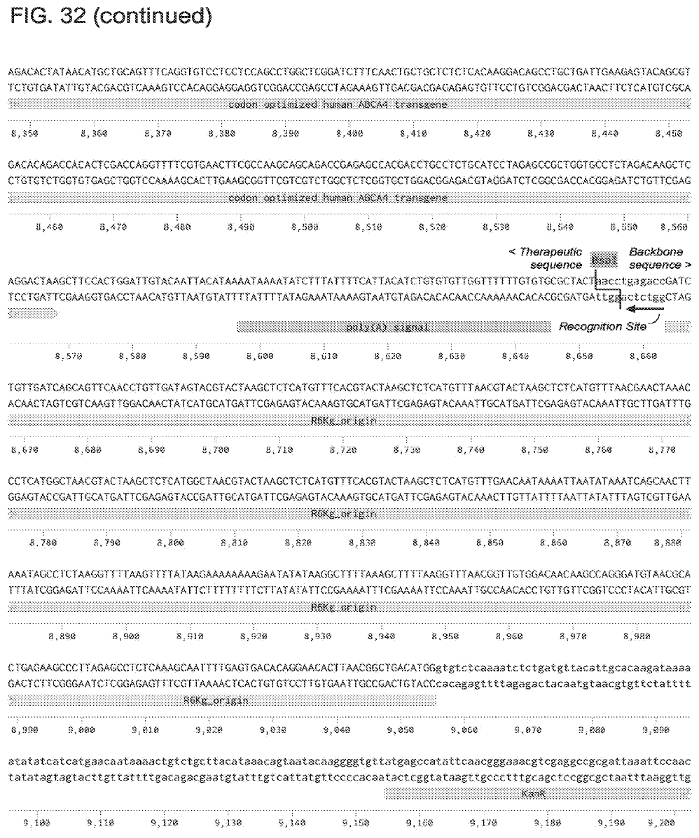

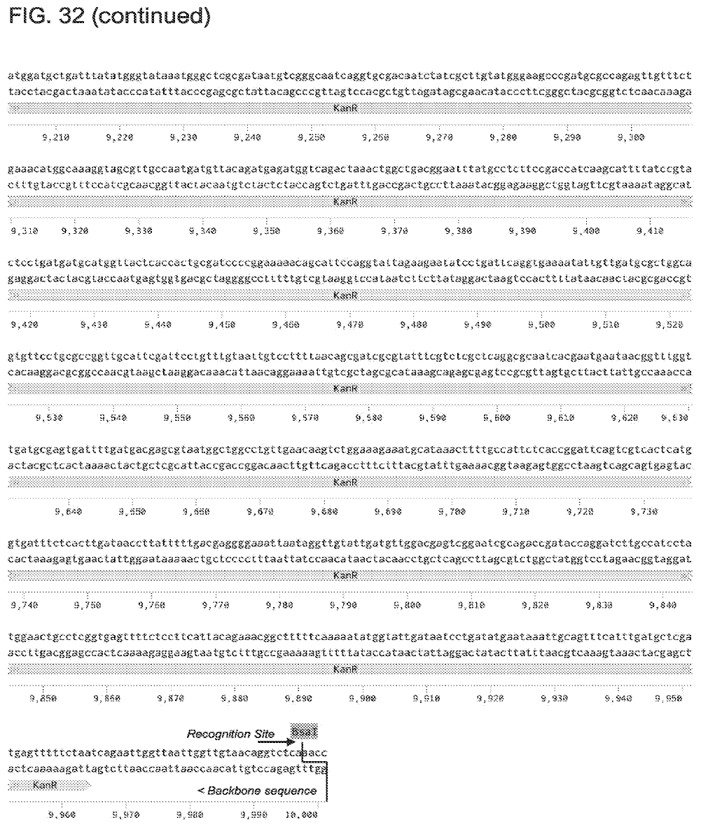

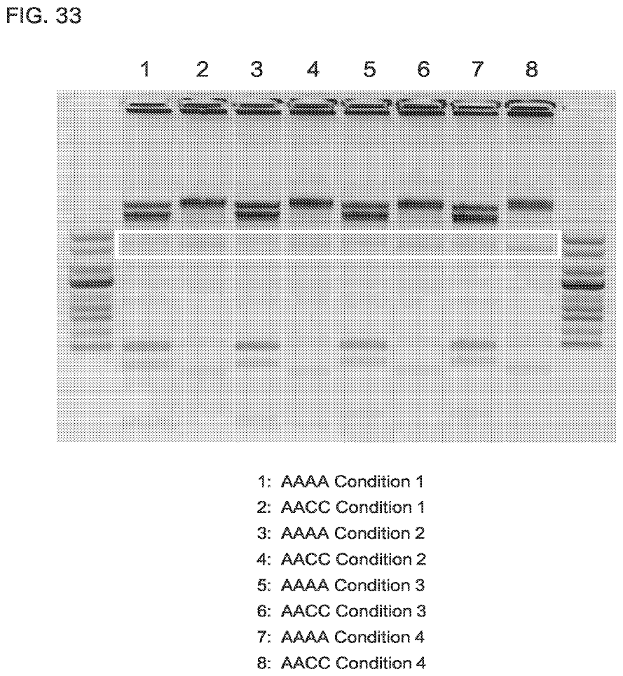

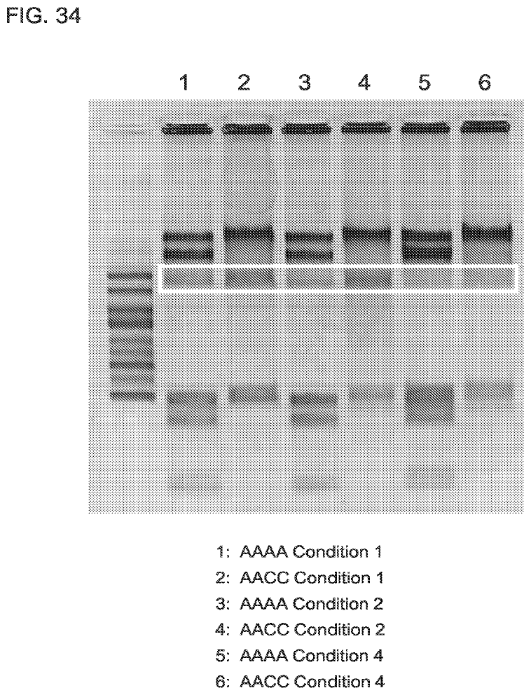

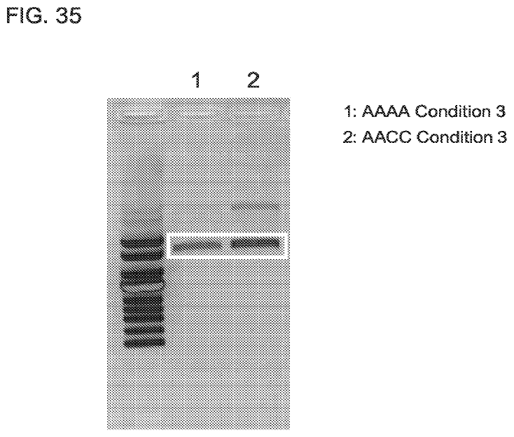

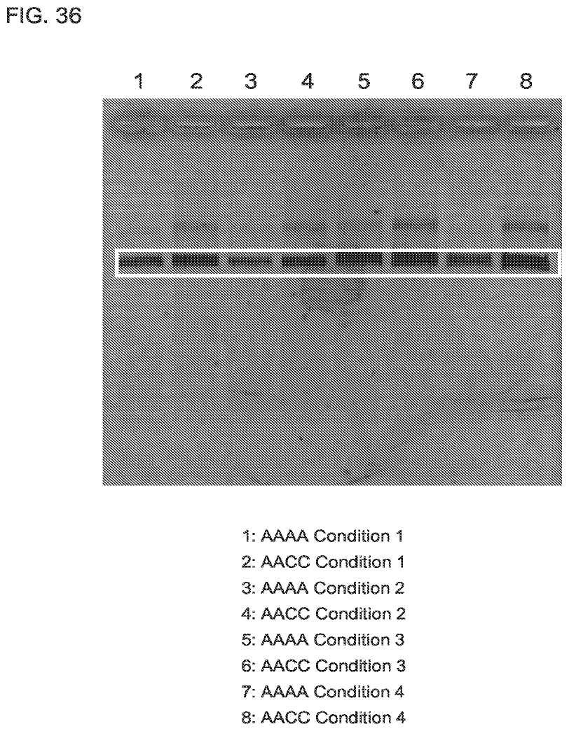

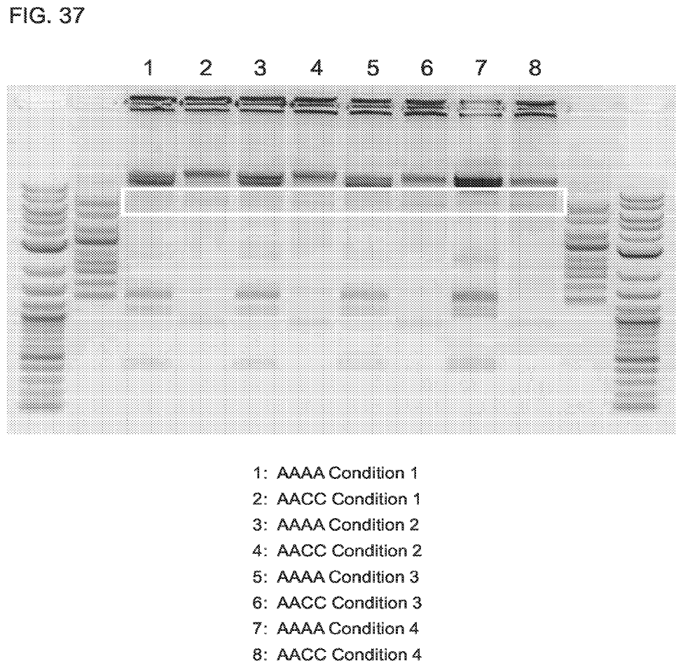

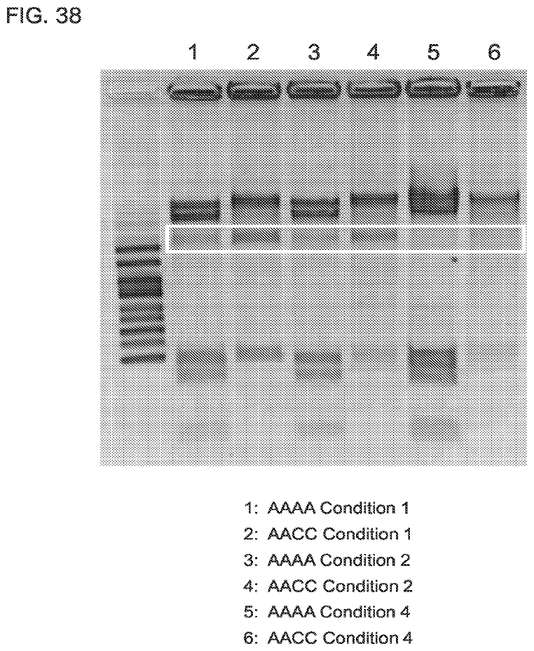

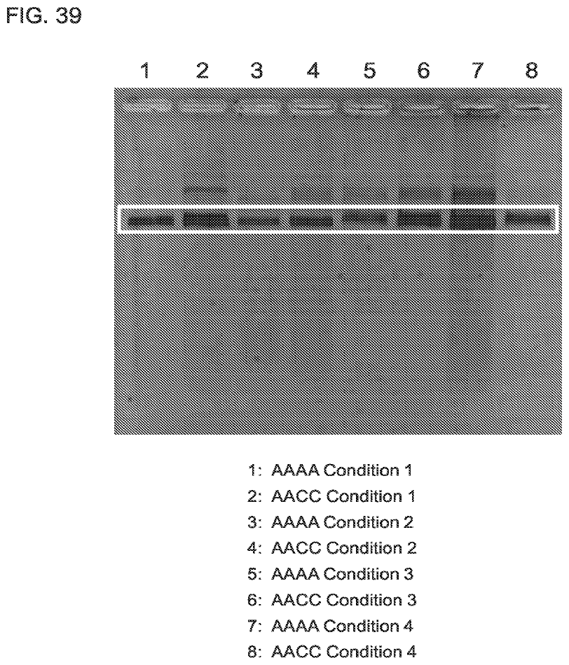

FIG. TA shows a plasmid DNA vector containing a therapeutic sequence (solid fill) and a backbone sequence (hatched fill). The backbone sequence contains two PvuII restriction sites therewithin. Two EcoRI restriction sites flank the backbone sequence and the therapeutic sequence. FIG. 1B shows products of a reaction of the plasmid DNA vector, or an amplified concatemer thereof, with EcoRI. A linear therapeutic fragment (solid fill) and a linear backbone fragment (hatched fill) are produced for each unit of plasmid DNA or linear concatemer. FIG. 1C shows circularized products of a reaction of the linear fragments with a ligase, i.e., a therapeutic circular DNA vector and a circularized backbone. FIG. 1D shows products of circularized products of FIG. 1C with PvuII. The circularized backbone is linearized and thereafter digestible with exonuclease. FIG. 1E shows a supercoiled therapeutic circular DNA vector resulting from a reaction of the therapeutic circular DNA vector with a topoisomerase, such as gyrase.

FIGS. 2A-2F are maps illustrating a process of removing a backbone from a plasmid DNA vector using BsaI to cut at two sites within the plasmid DNA vector. FIG. 2A shows the plasmid DNA vector, which contains a therapeutic sequence (“C3 region,” nucleotide bases of which are represented as N's in FIG. 2B for illustrative purposes) and a backbone sequence (which contains a replication origin and two BsaI recognition sites that are distal from the therapeutic sequence relative to their corresponding BsaI overhangs (cut sites)). FIG. 2B shows the linear sequence corresponding to FIG. 2A. FIG. 2C shows the therapeutic circular DNA vector resulting from the plasmid of FIG. 2A. The therapeutic circular DNA contains no replication origin—only a four base-pair BsaI overhang remains. FIG. 2D shows the linear sequence corresponding to FIG. 2C. FIG. 2E shows a circularized backbone resulting from the plasmid of FIG. 2A, which contains the BsaI recognition sites separated by a BsaI cut site (overhangs). FIG. 2F shows the linear sequence corresponding to FIG. 2F.

FIGS. 3A-3D are a schematic diagram showing a series of reaction steps as described herein, in which a single restriction enzyme is used in a cell-free process of producing c3DNA. FIG. 3A shows a plasmid DNA vector containing a therapeutic sequence (solid fill) and a backbone sequence (hatched fill). The plasmid DNA vector contains four BsaI restriction sites—two within the backbone sequence and two flanking both the backbone sequence and the therapeutic sequence. FIG. 3B shows products of a reaction of the plasmid DNA vector, or an amplified concatemer thereof, with BsaI. A linear therapeutic fragment (solid fill) and three linear backbone fragments (hatched fill) are produced for each unit of plasmid DNA or linear concatemer. FIG. 3C shows products of a reaction of the linear fragments with a ligase, i.e., a therapeutic circular DNA vector and linear backbone fragments, which are digestible with exonuclease. FIG. 3D shows a supercoiled therapeutic circular DNA vector resulting from a reaction of the therapeutic circular DNA vector with a topoisomerase, such as gyrase.

FIGS. 4A-4C are drawings showing three DNA vectors generated using the methods described herein. FIG. 4A is a single transcription unit (TU) DNA vector (1103) having, arranged in a 5′-to-3′ direction, a CMV promoter (PcMv), a coding sequence, and a polyA tail. FIG. 4B is a multi-TU DNA vector (1147) having, arranged in a 5′-to-3′ direction, a first TU having a first promoter, a first coding sequence, and a first polyA tail; a second TU having a second promoter, a second coding sequence, and a second polyA tail; a third TU having a third promoter, a third coding sequence, and a third polyA tail; and a fourth TU having a fourth promoter, a fourth coding sequence, and a fourth polyA tail. FIG. 4C is a single TU DNA vector (1258) having three coding sequences under control of a single promoter, followed by a polyA tail.

FIGS. 5A-5D are photographs of electrophoresis gels showing bands corresponding to DNA fragments after digestion. FIGS. 5A and 5B show actual and theoretical bands, respectively, after digestion. FIG. 5C shows band patterns post-ligation, and FIG. 5D shows band patterns after exonuclease digestion.

FIGS. 6A and 6B are schematic diagrams showing two variants of a process of reaction steps as described herein. FIG. 6A shows a process in which a first BsaI digest is followed by heat inactivation before ligation (Condition 1). FIG. 6B shows a streamlined process in which BsaI digest was combined with ligation (Condition 2).

FIGS. 7A and 7B are maps illustrating a process of removing a backbone from a plasmid DNA vector using BsaI to cut at five sites within the plasmid DNA vector. FIG. 7A shows the plasmid DNA vector, which contains a therapeutic sequence (C3 region, nucleotide bases of which are represented as N's in FIG. 7B for illustrative purposes) and a backbone sequence (which contains a replication origin and resistance genes). FIG. 7B shows the linear sequence corresponding to FIG. 7A.

FIGS. 8A and 8B are gels showing theoretical bands (FIG. 8A) or actual bands (FIG. 8B) following exonuclease digestion of the three constructs shown in FIGS. 4A-4C. Lanes 1-3 contain product from Condition 1, while lanes 4-6 contain product from Condition 2. Lanes 1 and 4 contain construct 1103 (1431 bp), lanes 2 and 5 contain construct 1147 (6293 bp), and lanes 3 and 6 contain construct 1258 (5065 bp).

FIG. 9 is a photograph of an electrophoresis gel showing recovery of a 12.75 kb c3DNA construct after T5 exonuclease digestion.

FIG. 10 is photograph of an electrophoresis gel showing a ligation reaction in which a linear fragment containing a reporter gene sequence was self-ligated to form a closed circular DNA vector.

FIG. 11 is a photograph of an electrophoresis gel showing a ligation reaction in which a linear fragment containing a reporter gene sequence was self-ligated to form a closed circular DNA vector. Lanes 1-3 show 20 μg/mL of DNA treated with 100 U/μg ligase, 20 U/μg ligase, and 5 U/μg ligase, respectively. Lanes 4-6 show 40 μg/mL of DNA treated with 100 U/μg ligase, 20 U/μg ligase, and 5 U/μg ligase, respectively. Lanes 7-9 show 100 μg/mL of DNA treated with 100 U/μg ligase, 20 U/μg ligase, and 5 U/μg ligase, respectively.

FIG. 12 is a photograph of a gel showing results of time courses of a T5 exonuclease digest from 0 hours (lane 2) to two hours (lane 6) or overnight hours (lane 11).

FIG. 13 is a photograph of an electrophoresis gel showing banding profiles for C³DNA after ligation for various ligase enzymes and DNA concentrations at ligation. The black box identifies the desired C³DNA vector band. Lane numbering corresponds with Sample Number identified in Table 3.

FIG. 14 is a photograph of an electrophoresis gel showing banding profiles for C³DNA after various durations of ligation with T4 ligase. The white box indicates the desired C³DNA band. Lane 1 is a control sample post-BsaI treatment; Lanes 2-5 show Sample 1 of Table 3 at t=0 (Lane 2), t=2 hour (Lane 3), t=5 hours (Lane 4), t=21.5 hours (Lane 5); Lanes 6-9 show Sample 2 of Table 3 at t=0 (Lane 6), t=2 hour (Lane 7), t=5 hours (Lane 8), t=21.5 hours (Lane 9); and Lanes 10-13 show Sample 2 of Table 3 at t=0 (Lane 10), t=2 hour (Lane 11), t=5 hours (Lane 12), t=21.5 hours (Lane 13).

FIG. 15 is a photograph of an electrophoresis gel showing banding profiles for C³DNA after various durations of ligation with T3 ligase (Lanes 2-9) and T7 ligase (Lanes 10-17). The white box indicates the desired C³DNA band. Lane 1 is a control sample post-BsaI treatment; Lanes 2-5 show Sample 4 of Table 3 at t=0 (Lane 2), t=2 hour (Lane 3), t=5 hours (Lane 4), t=21.5 hours (Lane 5); Lanes 6-9 show Sample 5 of Table 3 at t=0 (Lane 6), t=2 hour (Lane 7), t=5 hours (Lane 8), t=21.5 hours (Lane 9); Lanes 10-13 show Sample 6 of Table 3 at t=0 (Lane 10), t=2 hour (Lane 11), t=5 hours (Lane 12), t=21.5 hours (Lane 13); and Lanes 14-17 show Sample 7 of Table 3 at t=0 (Lane 14), t=2 hour (Lane 15), t=5 hours (Lane 16), t=21.5 hours (Lane 17).

FIG. 16 is a graph showing ligation kinetics as a reduction of linear DNA over time for Samples 1-7 of Table 3.

FIG. 17 is a photograph of an electrophoresis gel showing banding patters of various C³DNA construct preparations after ligation and before heat kill. Lanes correspond to Sample Numbers of Table 4. White boxes indicate desired C³DNA monomer band for each construct size.

FIGS. 18A and 18B are photographs of electrophoresis gels showing banding patters of various C³DNA construct preparations after supercoiling by gyrase treatment. Lanes correspond to Sample Numbers of Table 4. White boxes indicate desired C3DNA monomer band for each construct size.

FIGS. 19A and 19B are photographs of electrophoresis gels showing banding patters of various C³DNA construct preparations after exonuclease digest. Lanes correspond to Sample Numbers of Table 4. White boxes indicate desired C3DNA monomer band for each construct size.

FIG. 20 is a graph showing quantification of C³DNA monomer yield of C³DNA construct preparations after purification. The left-hand bar in each sample shows heat-killed samples.

FIG. 21 is a photograph of an electrophoresis gel showing banding patterns of C³DNA produced under the conditions identified in Table 6, through supercoiling and exonuclease digestion, before downstream column purification. Lane number corresponds to Sample Number of Table 6.

FIG. 22 is a graph showing relative quantification of C³DNA monomer yield for samples shown in FIG. 21.

FIG. 23 is a photograph of an electrophoresis gel showing banding patterns of C³DNA produced under the conditions identified in Table 6, through downstream purification, excluding 160 μg/mL DNA concentration samples.

FIG. 24 is a graph showing relative quantification of C³DNA monomer yield for samples shown in FIG. 23.

FIG. 25 is a photograph of an electrophoresis gel showing banding patterns of C³DNA produced under various gyrase concentrations identified in Table 8.

FIG. 26 is a photograph of an electrophoresis gel showing banding patterns of different sizes of C³DNA produced using different restriction processes (overhang sequences and number of cut sites), over various durations of BsaI digest steps. Sample numbers are provided in Table 9.

FIGS. 27A and 27B are photographs of electrophoresis gels showing banding patterns of different sizes of C³DNA produced using different restriction processes (overhang sequences and number of cut sites), over various durations of ligation steps carried out as part of the same study. FIG. 27A shows time points at 1 hour, 3 hours, and 18 hours. FIG. 27B shows time points at 3 hours, 18 hours, and 24 hours. Sample numbers are provided in Table 9. White arrows point to the desired bands. White boxes indicate byproduct bands.

FIG. 28A is a photograph of an electrophoresis gel showing post-exonuclease banding patterns of different sizes of C³DNA produced using different restriction processes (overhang sequences and number of cut sites), as indicated in Table 9.

FIG. 28B is a graph showing post-exonuclease C³DNA concentrations for each sample described in Table 9, quantified by Qubit.

FIG. 29 is a graph showing DNA concentrations of byproduct DNA over a time course of exonuclease treatment. Samples were tested in replicate (A and B).

FIG. 30 is a schematic showing two constructs tested in Example 11. Each construct was produced by a different restriction process (either AAAA or AACC overhangs, with backbones consisting of either one or four fragments).

FIG. 31 is a plasmid map of the 8.7 kb construct with AAAA overhang exemplified in Example 11. BsaI recognition sites (GGTCTC) are denoted with black arrows near the BsaI cut site (sticky ends delineated with black lines). At cut sites flanking the therapeutic sequence, each side of the cut site is marked as the therapeutic sequence or backbone sequence.

FIG. 32 is a plasmid map of the 8.7 kb construct with AACC overhang exemplified in Example 11. BsaI recognition sites (GGTCTC) are denoted with black arrows near the BsaI cut site (sticky ends delineated with black lines). Each side of each cut site is marked as the therapeutic sequence or backbone sequence.

FIG. 33 is a photograph of an electrophoresis gel showing 8.7 kb C3DNA banding patterns for samples identified in Table 14 at the end of the ligation step. The white box indicates the desired C³DNA monomer band.

FIG. 34 is a photograph of an electrophoresis gel showing 8.7 kb C³DNA banding patterns for Conditions 1, 2, and 4, at the end of the supercoiling step. The white box indicates the desired C³DNA monomer band.

FIG. 35 is a photograph of an electrophoresis gel showing 8.7 kb C³DNA banding patterns for Condition 3 at the end of the exonuclease digestion step. The white box indicates the desired C³DNA monomer band.

FIG. 36 is a photograph of an electrophoresis gel showing 8.7 kb C³DNA banding patterns for Conditions 1, 2, and 4 at the end of the exonuclease digestion step and Condition 3 at the end of the supercoiling step. The white box indicates the desired C³DNA monomer band.

FIG. 37 is a photograph of an electrophoresis gel showing 10.3 kb C³DNA banding patterns for samples identified in Table 16 at the end of the ligation step. The white box indicates the desired C³DNA monomer band.

FIG. 38 is a photograph of an electrophoresis gel showing 10.3 kb C³DNA banding patterns for Conditions 1, 2, and 4, at the end of the supercoiling step. The white box indicates the desired C³DNA monomer band.

FIG. 39 is a photograph of an electrophoresis gel showing 10.3 kb C³DNA banding patterns for Conditions 1, 2, and 4 at the end of the exonuclease digestion step and Condition 3 at the end of the supercoiling step. The white box indicates the desired C³DNA monomer band.

### DETAILED DESCRIPTION

The present invention features improved methods of producing non-viral DNA vectors, such as therapeutic circular DNA vectors. The invention is based, in part, on the development of a cell-free process to synthetically produce circular DNA by rolling-circle amplification and ligation-mediated circularization (e.g., as opposed to bacterial expression and/or site-specific recombination). The present methods allow for improved scalability and manufacturing efficiency in production of non-viral, circular DNA vectors and can reduce risks associated with bacterial processing. The invention allows production of circular DNA vectors with a therapeutic sequence that can be used for treating a disease or disorder, e.g., by transfecting a target cell.

Methods disclosed herein can provide enhanced purity and yield of desired products as compared to conventional methods. In particular, the use of steps that include treatment with certain restriction enzymes, (e.g., type IIS restriction enzymes), exonucleases, such as terminal exonucleases (e.g., T5 exonuclease), and/or a helicase or topoisomerase (e.g., type II topoisomerase, such as gyrase), produce products with enhanced yields and purity by reducing and/or degrading impurities, such as bacterial sequences. In certain embodiments, the present methods streamline the manufacturing process by performing a restriction digest and a ligation simultaneously by using a type IIS restriction enzyme.

Therapeutic circular DNA vectors, and pharmaceutical compositions thereof, generated by the present methods exhibit several advantageous properties. For instance, by eliminating or reducing bacterial plasmid DNA sequences, such as RNAPII arrest sites, transcriptional silencing of a therapeutic circular DNA vector can be reduced or eliminated, resulting in persistence of the therapeutic sequence in an individual. In particular embodiments of the present invention, immunogenic components (e.g., bacterial endotoxin, DNA, or RNA, or bacterial signatures, such as CpG motifs) are absent in the present therapeutic circular DNA vectors; therefore, the risk of stimulating a host immune response is reduced relative to conventional DNA vectors, such as plasmid DNA vectors.

Thus, the methods described herein produce DNA vectors that are substantially devoid of bacterial plasmid DNA sequences (e.g., RNAPII arrest sites, origins of replication, and/or resistance genes) and other bacterial signatures (e.g., immunogenic CpG motifs), and/or can be synthesized and amplified entirely in vitro (e.g., replication in bacteria is unnecessary; bacterial origins of replication and bacterial resistance genes are unnecessary; and recombination sites are unnecessary). These methods and steps are described in more detail below.

### I. Definitions

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. In the event of any conflicting definitions between those set forth herein and those of a referenced publication, the definition provided herein shall control.

As used herein, the term “circular DNA vector” refers to a DNA molecule in a circular form. Such circular form is typically capable of being amplified into concatemers by rolling circle amplification. A linear double-stranded nucleic acid having conjoined strands at its termini (e.g., covalently conjugated backbones, e.g., by hairpin loops or other structures) is not a circular vector, as used herein. The term “circular DNA vector” is used interchangeably herein with the terms “covalently closed and circular DNA vector” and “c3DNA.” A skilled artisan will understand that such circular vectors include vectors that are covalently closed with supercoiling and complex DNA topology, as is described herein. In particular embodiments, a circular DNA vector is supercoiled (e.g., monomeric supercoiled). In certain instances, a circular DNA vector lacks a bacterial origin of replication (e.g., in instances in which the circular DNA vector encodes a self-replicating RNA molecule, the circular DNA vector lacks a bacterial origin of replication and encodes an RNA origin of replication).

As used herein, a “cell-free” method of producing a circular DNA vector refers to a method that does not rely on containment of any of the DNA within a host cell, such as a bacterial (e.g., E. coli) host cell, to facilitate any step of the method, from providing the template DNA vector (e.g., plasmid DNA vector) through producing the therapeutic circular DNA vector. For example, a cell-free method occurs within one or more synthetic containers (e.g., glass or plastic tubes, bioreactors, vessels, tanks, or other suitable containers) within appropriate solutions (e.g., buffered solutions), to which enzymes and other agents may be added to facilitate DNA amplification, modification, and isolation. Cell-free production methods may use template DNA that has been produced within cells.

As used herein, the term “therapeutic sequence” refers to the portion of a DNA molecule (e.g., a plasmid DNA vector or a concatemer thereof) that contains any genetic material required for transcription in a target cell of one or more therapeutic moieties, which may include one or more coding sequences, promoters, terminators, introns, and/or other regulatory elements. A therapeutic moiety can be a therapeutic protein (e.g., a replacement protein (e.g., a protein that replaces a defective protein in the target cell) or an endogenous protein (e.g., a modulatory protein, such as a cytokine)) and/or a therapeutic nucleic acid (e.g., one or more microRNAs). In DNA vectors having more than one transcription unit, the therapeutic sequence contains the plurality of transcription units. A therapeutic sequence may include one or more genes (e.g., heterologous genes or transgenes) to be administered for a therapeutic purpose.

As used herein, the term “protein” refers to a plurality of amino acids attached to one another through peptide bonds (i.e., as a primary structure), including multimeric (e.g., dimeric, trimeric, etc.) proteins that are non-covalently associated (e.g., proteins having quaternary structure). Thus, the term “protein” encompasses peptides (e.g., polypeptides), native proteins, recombinant proteins, and fragments thereof. In some embodiments, a protein has a primary structure and no secondary, tertiary, or quaternary structure in physiological conditions. In some embodiments, a protein has a primary and secondary structure and no tertiary or quaternary structure in physiological conditions. In particular embodiments, a protein has a primary structure, a secondary structure, and a tertiary structure, but no quaternary structure in physiological conditions (e.g., a monomeric protein having one or more folded alpha-helices and/or beta sheets). In some embodiments, any of the proteins described herein have a length of at least 25 amino acids (e.g., 50 to 1,000 amino acids).

The term “therapeutic gene” refers to a transgene to be administered (e.g., as part of a DNA vector or self-replicating RNA molecule). A therapeutic gene can be a mammalian gene encoding a therapeutic protein.

As used herein, the term “therapeutic protein” refers to a protein that can treat a disease or disorder in a subject. In some embodiments, a therapeutic protein is a therapeutic replacement protein administered to replace a defective (e.g., mutated) protein in a subject. In some embodiments, a therapeutic protein is the same or functionally similar to a native protein that is not defective in a subject (e.g., a cytokine, chemokine, or growth factor). In some embodiments, a therapeutic protein is an antigen. In some embodiments, a therapeutic protein is an antigen-binding protein.

As used herein, the term “therapeutic replacement protein” refers to a protein that is structurally similar to (e.g., structurally identical to) a protein that is endogenously expressed by a normal (e.g., healthy) individual. A therapeutic replacement protein can be administered to an individual that suffers from a disorder associated with a dysfunction of (or lack of) the protein to be replaced. In some embodiments, the therapeutic replacement protein corrects a defect in a protein resulting from a mutation (e.g., a point mutation, an insertion mutation, a deletion mutation, or a splice variant mutation) in the gene encoding the protein. Therapeutic replacement proteins do not include non-endogenous proteins, such as proteins associated with a pathogen (e.g., as part of a vaccine). Therapeutic replacement proteins may include enzymes, growth factors, hormones, interleukins, interferons, cytokines, anti-apoptosis factors, anti-diabetic factors, coagulation factors, anti-tumor factors, liver-secreted proteins, or neuroprotective factors. In some instances, the therapeutic replacement protein is monogenic.

As used herein, the term “therapeutic nucleic acid” refers to a nucleic acid that binds to (e.g., hybridizes with) a molecule (e.g., protein or nucleic acid) in the subject to confer its therapeutic effect (i.e., without necessarily being transcribed or translated). Therapeutic nucleic acids can be DNA or RNA, such as small interfering RNA (siRNA), short hairpin RNA (shRNA), microRNA (miRNA), a CRISPR molecule (e.g., guide RNA (gRNA)), an oligonucleotide (e.g., an antisense oligonucleotide), an aptamer, or a DNA vaccine. In some embodiments, the therapeutic nucleic acid may be a non-inflammatory or a non-immunogenic therapeutic nucleic acid. In other embodiments, the therapeutic nucleic acid is recognizable by the immune system (e.g., adaptive immune system) and may induce an immune response (e.g., an innate immune response). Such therapeutic nucleic acids include toll-like receptor (TLR) agonists.

As used herein, the term “type IIs restriction enzyme” refers to an enzyme that recognizes a recognition site on a DNA molecule and cleaves the DNA molecule at a cut site that is outside the recognition site, thereby producing an overhang (sticky end) having a sequence that is unrelated to the recognition site. Type IIs restriction enzymes include natural type IIs restriction enzymes (e.g., BsaI, AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MboII, MlyI, MmeI, MnII, NmeAIII, PaqCI, PleI, SapI, and SfaNI) and synthetic type IIs restriction enzymes (e.g., as described in Lippow et al. Nucleic Acids Res. 2009, 37(9): 3061-3073, which is incorporated by reference in its entirety).

As used herein, the term “backbone sequence” refers to a portion of plasmid DNA outside the therapeutic sequence that includes one or more bacterial origins of replication or fragments thereof, one or more drug resistance genes or fragments thereof, one or more recombination sites, or any combination thereof. In some embodiments, the backbone sequence includes one or more bacterial origins of replication. Backbone sequences include truncated plasmid backbones of 20 base pairs or more (e.g., 31-40, e.g., 38 base pairs), which may include, e.g., a functional origin of replication.

As used herein, the term “recombination site” refers to a nucleic acid sequence that is a product of site-specific recombination, which includes a first sequence that corresponds to a portion of a first recombinase attachment site and a second sequence that corresponds to a portion of a second recombinase attachment site. One example of a hybrid recombination site is attR, which is a product of site-specific recombination and includes a first sequence that corresponds to a portion of attP and a second sequence that corresponds to a portion of attB. Alternatively, recombination sites can be generated from Cre/Lox recombination. Thus, a vector generated from Cre/Lox recombination (e.g., a vector including a LoxP site) includes a recombination site, as used herein. Other site-specific recombination events that generate recombination sites involve, e.g., lambda integrase, FLP recombinase, and Kw recombinase. Nucleic acid sequences that result from non-site-specific recombination events (e.g., ITR-mediated intermolecular recombination) are not recombination sites, as defined herein.

As used herein, the term “flank,” “flanking,” and “flanked” refer to a pair of regions or points on a nucleic acid molecule (e.g., a plasmid DNA vector) that are outside a reference region of the nucleic acid molecule. In some embodiments, a pair of regions or points flanking a reference region on a nucleic acid are adjacent to (i.e., abut) the reference region (i.e., there are no intervening bases between the reference point and the flanking point). In other embodiments, a pair of regions or points on a nucleic acid molecule that flank a reference region are separated from the reference region by one or more intervening bases (e.g., up to 1,000 intervening bases). For example, a first and second restriction site are said to flank a therapeutic sequence if the first restriction site is 200 bases upstream of the therapeutic sequence and the second restriction site is 100 bases downstream of the therapeutic sequence.

In some embodiments, all intervening sequences between a flanking region or point and a reference region are devoid of bacterial sequences. Thus, there are no bacterial sequences in a circular DNA vector produced by self-ligating a therapeutic sequence that was cut out of a plasmid DNA vector at restriction sites flanking the therapeutic sequence. For example, in such embodiments, a type IIs restriction enzyme that cuts sites flanking a therapeutic sequence may produce a therapeutic circular DNA vector having a sequence between the 5′ end and 3′ end of the therapeutic sequence; however, this region contains no bacterial sequences (e.g., bacterial origins of replication or drug-resistance genes). Such intervening sequences may be artifacts from sticky end ligation, e.g., corresponding to overhang bases generated by the type IIs restriction enzyme.

As used herein, steps are performed “simultaneously” when the steps overlap, wholly or partially. Thus, restriction digestion and ligation occur simultaneously in any of the following scenarios: (i) the restriction enzyme acts on the DNA at the same time as the ligase and both enzymes are inactivated at the same time; (ii) the restriction enzyme acts on the DNA at the same time as the ligase and the enzymes are inactivated at different times; (iii) the restriction enzyme acts on the DNA before the ligase and both enzymes are inactivated at the same time; or (iv) the restriction enzyme acts on the DNA before the ligase, the ligase acts on the DNA before the restriction enzyme is inactivated, and the restriction enzyme is inactivated before the ligase in inactivated.

As used herein, a step is said to “immediately” follow a preceding step if there are no intervening functional steps, such as purifications (e.g., purifications that reduce DNA yield, such as gel purifications or column purifications), enzymatic reactions, or enzyme inactivation steps (e.g., heat-inactivation steps, also referred to heat-kill steps). When proceeding from one step to an immediate subsequent step, it will be understood that transition conditions may occur, such as an increase or decrease in temperature and/or increases or decreases in reagent concentration. Whether such transition conditions occur instantaneously or gradually (e.g., over the course of seconds or minutes), a subsequent step is nevertheless said to “immediately” follow a preceding step if there are not intervening functional steps. For example, a 65° C. post-ligation heat inactivation step may be immediately proceeded by a 37° C. supercoiling step after a two-hour cooling period during which the temperature is reduced from 65° C. to 37° C.

As used herein, “large-scale production” means production of at least 2 mg therapeutic circular DNA vector per batch. Large-scale production enables therapeutically effective amounts of therapeutic circular DNA vector for one or more doses.

As used herein, the term “self-replicating RNA molecule” refers to a self-replicating genetic element comprising an RNA that replicates from one origin of replication. The terms “self-replicating RNA,” “replicon RNA,” and “self-amplifying replicon RNA” are used interchangeably herein.

As used herein, the term “operably linked” refers to an arrangement of elements, wherein the components so described are configured so as to perform their usual function. A nucleic acid is “operably linked” when it is placed into a functional relationship with another nucleic acid sequence. For example, a promoter is operably linked to one or more heterologous genes if it affects the transcription of the one or more heterologous genes. Further, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence, and the promoter sequence can still be considered “operably linked” to the coding sequence.

As used herein, the term “isolated” means artificially produced and not integrated into a native host genome. For example, an isolated nucleic acid vector includes nucleic acid vectors that are encapsulated in a lipid envelope (e.g., a liposome) or a polymer matrix. In some embodiments, the term “isolated” refers to a DNA vector that is: (i) amplified in vitro (e.g., in a cell-free environment), for example, by rolling-circle amplification or polymerase chain reaction (PCR); (ii) recombinantly produced by molecular cloning; (iii) purified, as by restriction endonuclease cleavage and gel electrophoretic fractionation, or column chromatography; or (iv) synthesized by, for example, chemical synthesis. An isolated nucleic acid vector is one which is readily manipulable by recombinant DNA techniques well-known in the art. Thus, a nucleotide sequence contained in a vector in which 5′ and 3′ restriction sites are known or for which polymerase chain reaction (PCR) primer sequences have been disclosed is considered isolated, but a nucleic acid sequence existing in its native state in its natural host is not. An isolated nucleic acid vector may be substantially purified, but need not be.

As used herein, the term “naked” refers to a nucleic acid molecule (e.g., a circular DNA vector) that is not encapsulated in a lipid envelope (e.g., a liposome) or a polymer matrix and is not physically associated with (e.g., covalently or non-covalently bound to) a solid structure (e.g., a particulate structure) upon administration to the individual. In some instances of the present invention, a pharmaceutical composition includes a naked circular DNA vector.

As used herein, a “vector” refers to a nucleic acid molecule capable of carrying a therapeutic sequence to which is it linked into a target cell in which the therapeutic sequence can then be transcribed, replicated, processed, and/or expressed in the target cell. After a target cell or host cell processes the therapeutic sequence of the vector, the therapeutic sequence is not considered a vector. One type of vector is a “plasmid”, which refers to a circular double stranded DNA loop containing a bacterial backbone into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors” or “expression vectors”).

As used herein, the terms “individual” and “subject” are used interchangeably and include any mammal in need of treatment or prophylaxis, e.g., by a therapeutic circular DNA vector, or pharmaceutical composition thereof, described herein. In some embodiments, the individual or subject is a human. In other embodiments, the individual or subject is a non-human mammal (e.g., a non-human primate (e.g., a monkey), a mouse, a pig, a rabbit, a cat, or a dog). The individual or subject may be male or female.

As used herein, an “effective amount” or “effective dose” of a therapeutic circular DNA vector, or pharmaceutical composition thereof, refers to an amount sufficient to achieve a desired biological, pharmacological, or therapeutic effect, e.g., when administered to the individual according to a selected administration form, route, and/or schedule. As will be appreciated by those of ordinary skill in this art, the absolute amount of a particular composition that is effective can vary depending on such factors as the desired biological or pharmacological endpoint, the agent to be delivered, the target tissue, etc. Those of ordinary skill in the art will further understand that an “effective amount” can be contacted with cells or administered to a subject in a single dose or through use of multiple doses. An effective amount of a composition to treat a disease may slow or stop disease progression or increase partial or complete response, relative to a reference population, e.g., an untreated or placebo population, or a population receiving the standard of care treatment.

As used herein, “treatment” (and grammatical variations thereof such as “treat” or “treating”) refers to clinical intervention in an attempt to alter the natural course of the individual being treated, which can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and improved prognosis. In some embodiments, therapeutic circular DNA vectors of the invention are used to delay development of a disease or to slow the progression of a disease.

The terms “level of expression” or “expression level” are used interchangeably and generally refer to the amount of a polynucleotide or an amino acid product or protein in a biological sample (e.g., retina). “Expression” generally refers to the process by which gene-encoded information is converted into the structures present and operating in the cell. Therefore, according to the invention, “expression” may refer to transcription into a polynucleotide, translation into a protein, or post-translational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

As used herein, the term “expression persistence” refers to the duration of time during which a therapeutic sequence, or a functional portion thereof (e.g., one or more coding sequences of a therapeutic DNA vector), is expressible in the cell in which it was transfected (“intra-cellular persistence”) or any progeny of the cell in which it was transfected (“trans-generational persistence”). A therapeutic sequence, or functional portion thereof, may be expressible if it is not silenced, e.g., by DNA methylation and/or histone methylation and compaction. Expression persistence can be assessed by detecting or quantifying (i) mRNA transcribed from the therapeutic sequence in the target cell or progeny thereof (e.g., through qPCR, RNA-seq, or any other suitable method) and (ii) protein translated from the therapeutic sequence in the target cell or progeny thereof (e.g., through Western blot, ELISA, or any other suitable method). In some instances, expression persistence is assessed by detecting or quantifying therapeutic DNA in the target cell or progeny thereof (e.g., the presence of therapeutic circular DNA vector in the target cell, e.g., through episomal DNA copy number analysis) in conjunction with either or both of (i) mRNA transcribed from the therapeutic sequence in the target cell or progeny thereof and (ii) protein translated from the therapeutic sequence in the target cell or progeny thereof. Expression persistence of a therapeutic sequence, or a functional portion thereof, can be quantified relative to a reference vector, such as a control vector produced in bacteria (e.g., a circular vector produced in bacteria or having one or more bacterial signatures not present in the vector of the invention (e.g., a plasmid)), using any gene expression characterization method known in the art. Expression persistence can be quantified at any given time point following administration of the vector. For example, in some embodiments, expression of a therapeutic circular DNA vector of the invention persists for at least two weeks after administration if it is detectable in the target cell, or progeny thereof, two weeks after administration of the therapeutic circular DNA vector. In some embodiments, expression of a gene “persists” in a target cell if it is detectable in the target cell at one week, two weeks, three weeks, four weeks, six weeks, two months, three months, four months, five months, six months, seven months, eight months, nine months, ten months, eleven months, one year, or longer after administration. In some embodiments, expression of a therapeutic sequence is said to persist for a given period after administration if any detectable fraction of the original expression level remains (e.g., at least 1%, at least 5%, at least 10%, at least 20%, at least 30%, at least 40%, at least 50%, at least 70%, or at least 100% of the original expression level) after the given period of time (e.g., one week, two weeks, three weeks, four weeks, six weeks, two months, three months, four months, five months, six months, seven months, eight months, nine months, ten months, eleven months, one year, or longer after administration).

As used herein, “intra-cellular persistence” refers to the duration of time during which a therapeutic sequence, or a functional portion thereof (e.g., one or more coding sequences of a therapeutic DNA vector), is expressible in the cell in which it was transfected (e.g., a target cell, such as a post-mitotic cell or a quiescent cell). Intra-cellular persistence can be assessed by detecting or quantifying (i) mRNA transcribed from the therapeutic sequence in the target cell and (ii) protein translated from the therapeutic sequence in the target cell. In some instances, intra-cellular persistence is assessed by detecting or quantifying therapeutic DNA in the target cell (e.g., the presence of therapeutic circular DNA vector in the target cell) in conjunction with either or both of (i) mRNA transcribed from the therapeutic sequence in the target cell and (ii) protein translated from the therapeutic sequence in the target cell. In some embodiments, the therapeutic circular DNA vector of the invention exhibits improved intra-cellular persistence relative to a reference vector (e.g., a plasmid DNA vector).

As used herein, “trans-generational persistence” refers to the duration of time during which a therapeutic sequence, or a functional portion thereof (e.g., one or more coding sequences of a therapeutic DNA vector), is expressible in progeny of the cell in which the gene was transfected (e.g., progeny of the target cell, such as first-generation, second-generation, third-generation, or fourth-generation descendants of the cell in which the gene was transfected, e.g., through a therapeutic circular DNA vector). Trans-generational persistence accounts for any dilution of a gene over cell divisions and may therefore be useful in measuring persistence of a vector in a dividing tissue over time. In some embodiments, the therapeutic circular DNA vector of the invention exhibits improved trans-generational persistence relative to a reference vector (e.g., a plasmid DNA vector). Trans-generational persistence can be assessed by detecting or quantifying (i) mRNA transcribed from the therapeutic sequence in progeny of the target cell and (ii) protein translated from the therapeutic sequence in progeny of the target cell. In some instances, intra-cellular persistence is assessed by detecting or quantifying therapeutic DNA in progeny of the target cell (e.g., the presence of therapeutic circular DNA vector in progeny of the target cell) in conjunction with either or both of (i) mRNA transcribed from the therapeutic sequence in progeny of the target cell and (ii) protein translated from the therapeutic sequence in progeny of the target cell. In some embodiments, the therapeutic circular DNA vector of the invention exhibits improved trans-generational persistence relative to a reference vector (e.g., a plasmid DNA vector).

The term “pharmaceutically acceptable” means safe for administration to a mammal, such as a human. In some embodiments, a pharmaceutically acceptable composition is approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which a vector or composition of the invention is administered. Examples of suitable pharmaceutical carriers are described in “Remington's Pharmaceutical Sciences,” Mack Publishing Co., Easton, PA., 23^{rd} edition, 2020.

The terms “a” and “an” mean “one or more of.” For example, “a gene” is understood to represent one or more such genes. As such, the terms “a” and “an,” “one or more of a (or an),” and “at least one of a (or an)” are used interchangeably herein.

As used herein, the term “about” refers to a value within +10% variability from the reference value, unless otherwise specified.

For any conflict in definitions between various sources or references, the definition provided herein shall control.

### II. Methods of Producing Therapeutic Circular DNA Vectors

The methods provided herein involve cell-free synthesis of therapeutic circular DNA vectors as alternative means to conventional production methods that are based on bacterial cell synthesis. Because the amplification of the bacterial plasmid DNA vector is feasible using a polymerase in cell-free conditions, the circular DNA vector can be isolated from the bacterial components of a plasmid in which it was cloned, and bacterial signatures are substantially absent from the isolated product vector. Cell-free synthesis therefore minimizes risk of bacterial impurities and offers purer compositions of resulting circular DNA vectors (i.e., synthetic circular DNA vectors) relative to bacterial-derived vectors (i.e., non-synthetic circular DNA vectors). The present methods are amenable to scale-up and provide improved efficiency in manufacturing. No gel extraction steps are required. Thus, in some embodiments, no gel purification (e.g., agarose gel purification) is performed as part of the production process (e.g., gel electrophoresis may be conducted in parallel for analytics purposes). In some embodiments, streamlined restriction digest schemes are provided. Therapeutic circular DNA vectors produced using such cell-free processes are referred to herein as “synthetic” vectors, reflecting the absence of bacterial cells in their production from templates.

In one aspect, the method includes providing a sample that includes a template DNA molecule (e.g., a template DNA vector (e.g., a plasmid DNA vector)) including a therapeutic gene sequence and amplifying the template DNA vector using a polymerase-mediated rolling-circle amplification to generate a linear concatemer. The linear concatemer is digested with a restriction enzyme that cuts at least two sites of the linear concatemer per unit of the bacterial plasmid DNA vector to generate linearized fragments of the DNA vector. The method further includes self-ligating the linearized fragment of the DNA vector that includes the therapeutic sequence to produce a therapeutic circular DNA vector. The method also includes treating the sample with a topoisomerase or a helicase. In some embodiments, the method further includes digesting the sample with an exonuclease (e.g., a terminal exonuclease). In some embodiments, the digesting and self-ligating are performed simultaneously.

In one aspect, the method includes providing a sample that includes a template DNA molecule (e.g., a template DNA vector (e.g., a plasmid DNA vector)) including a therapeutic sequence and amplifying the template DNA vector using a polymerase-mediated rolling-circle amplification to generate a linear concatemer. The method further includes digesting the linear concatemer with a restriction enzyme (e.g., a type IIs restriction enzyme, e.g., BsaI) to generate a linearized fragment of the DNA vector. The linear concatemer contains multiple copies of the template DNA vector, each copy having a unit length and the linear concatemer having multiple unit lengths of the vector. The restriction enzyme cuts at least two sites (e.g., two and only two sites, or more than two sites (e.g., three, four, five, or more sites)) of the linear concatemer per unit of the bacterial plasmid DNA vector. The method further includes self-ligating the linearized fragment of the DNA vector to produce a closed circular DNA vector (e.g., C³DNA). The method also includes digesting the sample with an exonuclease (e.g., a terminal exonuclease, e.g., T5 exonuclease). In some embodiments, the method further includes treating the sample with a topoisomerase (e.g., gyrase) or a helicase. In some embodiments, the digesting and self-ligating are performed simultaneously.

In another aspect, the method includes providing a sample having a template DNA molecule (e.g., a template DNA vector (e.g., a plasmid DNA vector)) including a therapeutic sequence and amplifying the template DNA vector using a polymerase-mediated rolling-circle amplification to generate a linear concatemer. The method further includes digesting the linear concatemer with a restriction enzyme (e.g., a type IIs restriction enzyme, e.g., BsaI) to generate a linearized fragment of the DNA vector. The restriction enzyme cuts at least two sites (e.g., two and only two sites, or more than two sites (e.g., three, four, five, or more sites)) of the linear concatemer per unit of the template DNA vector. The method further includes self-ligating the linearized fragment of the DNA vector to produce a closed circular DNA vector (e.g., C³DNA). The method may further include treating the sample with a topoisomerase (e.g., gyrase) or a helicase. The method may also include digesting the sample with an exonuclease (e.g., a terminal exonuclease, e.g., T5 exonuclease). In some embodiments, the digesting and self-ligating are performed simultaneously (in the same reaction conditions).

In some embodiments, the method utilizes a single restriction enzyme to generate overhangs such that the restriction digest step can be consolidated with the ligation step (e.g., the restriction digest step can overlap with the ligation step or occur simultaneously with the ligations step). For example, some embodiments of such a method include: (a) providing a sample comprising a template DNA vector comprising a therapeutic sequence and a backbone sequence; (b) amplifying the template DNA vector using a polymerase-mediated rolling-circle amplification to generate a linear concatemer; (c) digesting the linear concatemer with a restriction enzyme (e.g., type IIs restriction enzyme, e.g., BsaI) that cuts at least a first site, a second site, and a third site per unit of the linear concatemer, thereby producing a linear therapeutic fragment comprising the therapeutic sequence and at least two linear backbone fragments each comprising a portion of the backbone sequence; and (d) contacting the linear therapeutic fragment with a ligase to produce a therapeutic circular DNA vector in solution. The first and second sites flank the therapeutic sequence and form self-complementary overhangs, and the third site is within the backbone sequence and forms an overhang that is non-complementary to the first or second site.

Alternatively, a type Us restriction enzyme can be used to cut a template DNA molecule at two sites, thereby producing a single backbone fragment and a therapeutic fragment. By designing the template such that the type IIs recognition site (e.g., GGTCTC in embodiments involving BsaJ) is on the backbone fragment and not on the therapeutic fragment, self-ligation of the backbone fragment reconstitutes the type IIs restriction site on the circularized backbone, whereas self-ligation of the therapeutic fragment produces a therapeutic DNA vector lacking the type IIs restriction site. Thus, the backbone is subject to further digestion, while the therapeutic DNA vector is uncleaved.

In some embodiments, the restriction enzyme cuts a fourth site of the linear concatemer per unit, wherein the fourth site is within the backbone sequence and forms an overhang that is non-complementary to the first or second site, and wherein the digestion produces at least three linear backbone fragments each comprising a portion of the backbone sequence.

In some embodiments, no restriction enzyme inactivation step precedes step (d). For example, no heat inactivation of the restriction enzyme (e.g., type IIs restriction enzyme, e.g., BsaI) is performed before ligation. This allows for streamlined DNA production, enabled by the overhang design that allows for restriction digest and ligation steps to occur simultaneously, obviating the need to inactivate the restriction enzyme of step (c). It also permits use of single-use vessels not suitable for increased temperatures.

After step (d), the temperature of the solution containing the therapeutic circular DNA vector can be raised to about 65° C. to inactivate enzymes (e.g., restriction enzymes and/or ligase). Alternatively, no heat inactivation is performed after (e.g., immediately after) restriction enzyme digestion and/or ligation.

In another aspect, the invention provides a method of removing a backbone sequence from a DNA molecule (e.g., a template DNA vector) to produce a therapeutic circular DNA vector. The DNA molecule (e.g., template DNA vector) comprises the backbone sequence and a therapeutic sequence. The method involves the steps of (a) digesting the DNA molecule with one or more restriction enzymes (e.g., type IIs restriction enzymes) that cut at least a first site and a second site of the DNA molecule, wherein: (i) the first and second sites flank the therapeutic sequence and form self-complementary overhangs, and (ii) the recognition sites are within the backbone sequence, thereby producing a linear therapeutic fragment comprising the therapeutic sequence and a linear backbone fragments comprising the backbone sequence and the recognition sites (e.g., type IIs recognition sites); and (b) contacting the linear therapeutic fragment with a ligase to produce a therapeutic circular DNA vector in solution.

In another aspect, the invention provides a method of removing a backbone sequence from a DNA molecule (e.g., a template DNA vector) to produce a therapeutic circular DNA vector. The DNA molecule (e.g., template DNA vector) comprises the backbone sequence and a therapeutic sequence. The method involves the steps of (a) digesting the DNA molecule with one or more restriction enzymes that cut at least a first site, a second site, and a third site per unit of the DNA molecule, wherein: (i) the first and second sites flank the therapeutic sequence and form self-complementary overhangs, and (ii) the third site is within the backbone sequence and forms an overhang that is non-complementary to the first or second site, thereby producing a linear therapeutic fragment comprising the therapeutic sequence and at least two linear backbone fragments each comprising a portion of the backbone sequence; and (b) contacting the linear therapeutic fragment with a ligase to produce a therapeutic circular DNA vector in solution.

In some embodiments, the therapeutic circular DNA vector is contacted with topoisomerase (e.g., gyrase) or a helicase. Such reactions can be carried out at about 37° C. Additionally, or alternatively, the therapeutic circular DNA vector can be contacted with an exonuclease (e.g., a terminal exonuclease) (e.g., in a reaction carried out at about 37° C.). In particular embodiments, the therapeutic circular DNA vector (and reaction mixture thereof) is contact with a topoisomerase or a helicase and, without raising the reaction temperature to inactivate the topoisomerase or helicase, the therapeutic circular DNA vector (and reaction mixture thereof) is thereafter contacted with an exonuclease (e.g., a terminal exonuclease). Alternatively, the exonuclease digestion occurs before contact with the topoisomerase or a helicase.

In some embodiments, after contacting the therapeutic circular DNA vector with the topoisomerase or helicase and/or the terminal exonuclease, the method includes running the therapeutic circular DNA vector through a column (e.g., capture column). In some embodiments, the therapeutic circular DNA vector is then precipitated with isopropyl alcohol. In some embodiments, the methods include amplifying a template vector in vitro, digesting the amplified vector with a restriction enzyme, self-ligating the resultant fragment, and treating the sample with a terminal exonuclease and/or a helicase or topoisomerase, using any combination of the steps described in Sections A-G, below.

### A. Template

In general, production of a therapeutic circular DNA vector begins with providing a sample having a template DNA molecule (e.g., template DNA vector), such as a plasmid DNA vector, having a therapeutic sequence and a backbone sequence. By designing a template DNA vector to contain restriction sites (e.g., type IIs restriction sites, e.g., BsaI restriction sites, e.g., GGTCTC) flanking the therapeutic sequence (e.g., within the backbone sequence), the backbone sequence can be separated from the therapeutic sequence. The therapeutic sequence can then be self-ligated to produce a therapeutic circular DNA vector. Restriction sites can be designed in positions within the backbone sequence to allow for removal of the backbone sequence from the product without performing a yield-reducing step, such as gel purification, by further restriction digest and/or exonuclease digest. For example, a type IIs recognition site can be positioned distal to its corresponding cut site relative to the therapeutic sequence (see, e.g., FIG. 2A), i.e., the cut site is between the recognition site and the therapeutic sequence.

In some embodiments, the template contains, linked the following order: a first type IIs recognition site, a first type IIs cut site corresponding to the first type IIs recognition site, a therapeutic sequence, a second type IIs cut site, and a second type IIs recognition site corresponding to the second type IIs cut site. The second type IIs recognition site may be connected to the first type IIs recognition site by a backbone sequence (or portion thereof, where one or both of the first and second type IIs recognition sites are within the backbone sequence). In some instances, there are two and only two (i.e. no more than two) type IIs recognition sites on the template DNA molecule. In some instances, there are two and only two BsaI recognition sites on the template DNA molecule (e.g., as shown in FIG. 32). Such a design allows for both type IIs recognition sites to be positioned on a linear backbone sequence produced upon type IIs restriction enzyme digest and, upon, re-ligation, the type IIs restriction enzyme can cut the circularized backbone sequence. In some embodiments, the therapeutic sequence contains no type IIs recognition sites (e.g., BsaI recognition sites, e.g., GGTCTC). In some embodiments, the therapeutic sequence contains no restriction enzyme recognition sites.

In some embodiments in which a template is a plasmid DNA vector having a therapeutic sequence and a backbone sequence, the plasmid DNA contains at least three restriction sites (e.g., at least four restriction sites, or at least five restriction sites; e.g., three restriction sites, four restriction sites, or five restriction sites) that are recognized by the same restriction enzyme (e.g., a type IIs restriction enzyme, e.g., BsaI). Two of the at least three restriction sites flank the therapeutic sequence such that, upon restriction digest by the restriction enzyme (e.g., the type IIs restriction enzyme, e.g., BsaI), a linear therapeutic fragment is formed (e.g., a single linear therapeutic fragment is formed) having self-complementary overhangs at its termini. The at least one remaining restriction site is within the backbone sequence such that, upon digestion of the plasmid DNA vector with the restriction enzyme (e.g., the type II restriction enzyme, e.g., BsaI), at least two linear backbone fragments are produced, each of which includes a portion of the backbone sequence. The at least one remaining restriction site is positioned within the backbone sequence such that, upon digestion of the plasmid DNA vector with the restriction enzyme (e.g., the type II restriction enzyme, e.g., BsaI), the overhang produced by the restriction site within the backbone sequence is non-complementary to an overhang produced at the flanking ends of the therapeutic sequence.

In some embodiments in which the plasmid DNA contains four restriction sites recognized by a type Us restriction enzyme (e.g., BsaJ), the two remaining restriction sites within the backbone sequence are positioned such that, upon digestion of the plasmid DNA vector with the type IIs restriction enzyme (e.g., BsaI), three linear backbone fragments are produced, each of which includes a portion of the backbone sequence, and the overhangs produced by the restriction sites within backbone sequence are both non-complementary to an overhang produced at the flanking ends of the therapeutic sequence. In some such embodiments, the two type IIs restriction sites within the backbone sequence produce overhangs that are non-complementary to each other.

In embodiments in which two different restriction enzymes are used, the template (e.g., plasmid DNA vector) may contain at least three restriction sites (e.g., at least four restriction sites, or at least five restriction sites; e.g., three restriction sites, four restriction sites, or five restriction sites), wherein two of the restriction sites flank the therapeutic sequence and are recognized by a first restriction enzyme. The at least one remaining restriction site is within the backbone sequence and is recognized by a different, second restriction enzyme. In some embodiments, the restriction sites flanking the therapeutic sequence are EcoRI restriction sites and the first restriction enzyme is EcoRI. In some embodiments, the restriction sites flanking the therapeutic sequence are PvuII restriction sites and the first restriction enzyme is PvuII. In some embodiments, the restriction site within the backbone sequence is a PvuII restriction site and the second restriction enzyme is PvuII. In some embodiments, the restriction site within the backbone sequence is an EcoRI restriction site and the second restriction enzyme is EcoRI.

In some embodiments, the template (e.g., plasmid DNA vector) contains four restriction sites, wherein two restriction sites flank the therapeutic sequence and are recognized by a first restriction enzyme, and the two remaining restriction sites within the backbone sequence are recognized by a second restriction enzyme. In some embodiments, the restriction sites flanking the therapeutic sequence are EcoRI restriction sites and the first restriction enzyme is EcoRI. In some embodiments, the restriction sites flanking the therapeutic sequence are PvuII restriction sites and the first restriction enzyme is PvuII. In some embodiments, the restriction sites within the backbone sequence are PvuII restriction sites and the second restriction enzyme is PvuII. In some embodiments, the restriction sites within the backbone sequence are EcoRI restriction sites and the second restriction enzyme is EcoRI.

The sample containing the template DNA can be a lysate or other preparation from a cell or tissue (e.g., a mammalian cell or tissue or a bacterial cell) that includes a template DNA vector (e.g., bacterial plasmid DNA vector). Double stranded circular DNA can be obtained from the cells using standard DNA extraction/isolation techniques. In some embodiments, linear DNA is specifically degraded, e.g., using plasmid-safe DNase, to purify the plasmid DNA vector prior to further processing.

In other embodiments, template DNA lacks one or more bacterial elements of plasmid DNA vectors. In some instances of the methods described herein, synthetic DNA vectors described in International Publication No. WO 2021/055760, incorporated herein by reference in its entirety, are used as template DNA. Such synthetic DNA vectors can be amplified using rolling circle amplification and re-circularized by restriction digest and ligation. In embodiments in which synthetic DNA vectors lacking backbone sequences is used as a template, steps involving exonuclease digestion of linear backbone fragments are obviated and omitted from the present methods of production.

### B. Amplification

In some instances, cell-free synthesis of circular DNA vectors relies on effective amplification using a polymerase, such as a phage polymerase (e.g., Phi29 polymerase). The polymerase used herein can be, for example, a thermophilic polymerase having high processivity through GC-rich residues. In particular embodiments, the polymerase used to amplify the vector is Phi29 polymerase.

In some embodiments, the plasmid DNA vector is amplified in vitro, in a cell-free preparation, by incubating the DNA with a polymerase (e.g., a phage polymerase, e.g., Phi29 DNA polymerase; TempliPhi kit, GE Healthcare), primers (e.g., site-specific primers or random primers, e.g., random hexamer primers), and a nucleotide mixture (e.g., dNTP, e.g., dATP, dCTP, dGTP, and dTTP). The polymerase (e.g., phage polymerase, e.g., Phi29 polymerase) amplifies the template by rolling-circle amplification (e.g., isothermal rolling-circle amplification), generating a linear concatemer having a plurality of unit length copies of the template vector (e.g., plasmid DNA vector). Suitable polymerases include thermophilic polymerases and polymerases featuring high processivity.

A suitable polymerase concentration (e.g., Phi29 DNA polymerase concentration) can be from 10 U/mL to 2,000 U/mL (e.g., from 50 U/mL to 1,000 U/mL, from 100 U/mL to 500 U/mL, or from 150 U/mL to 300 U/mL, e.g., from 10 U/mL to 50 U/mL, from 50 U/mL to 100 U/mL, from 100 U/mL to 150 U/mL, from 150 U/mL to 200 U/mL, from 200 U/mL to 250 U/mL, from 250 U/mL to 300 U/mL, from 300 U/mL to 400 U/mL, from 400 U/mL to 500 U/mL, from 500 U/mL to 750 U/mL, from 750 U/mL to 1,000 U/mL, from 1,000 U/mL to 1,500 U/mL, or from 1,500 U/mL to 2,000 U/mL). In some embodiments, the polymerase (e.g., Phi29 DNA polymerase) concentration is about 200 U/mL.

Starting concentration of template DNA vector (e.g., plasmid DNA vector) can be from 10 ng/mL to 5 mg/mL (e.g., from 0.1 μg/mL to 1 mg/mL, from 0.2 μg/mL to 0.5 mg/mL, from 0.5 μg/mL to 0.1 mg/mL, from 1.0 μg/mL to 50 μg/mL, from 2.0 μg/mL to 25 μg/mL, from 4.0 g/mL to 10 μg/mL, or about 5.0 μg/mL; e.g., from 10 ng/mL to 50 ng/mL, from 50 ng/mL to 100 ng/mL, from 100 ng/mL to 500 ng/mL, from 500 ng/mL to 1 μg/mL, from 1 μg/mL to 2 g/mL, from 2 μg/mL to 3 μg/mL, from 3 μg/mL to 4 μg/mL, from 4 μg/mL to 5 μg/mL, from 5 g/mL to 6 μg/mL, from 6 μg/mL to 7 μg/mL, from 7 μg/mL to 8 μg/mL, from 8 μg/mL to 9 g/mL, from 9 μg/mL to 10 μg/mL, from 10 μg/mL to 20 μg/mL, from 20 μg/mL to 50 μg/mL, from 50 μg/mL to 100 μg/mL, from 100 μg/mL to 500 μg/mL, from 500 μg/mL to 1 mg/mL, or from 1 mg/mL to 5 mg/mL; e.g., about 0.5 μg/mL, about 1.0 μg/mL, about 2 μg/mL, about 3 g/mL, about 4 μg/mL, about 5 μg/mL, about 6 μg/mL, about 7 μg/mL, about 8 μg/mL, about 9 μg/mL, or about 10 μg/mL). In some embodiments, the starting concentration of template DNA vector (e.g., plasmid DNA vector) is from 1.0 μg/mL to 10 μg/mL (e.g., about 1.0 μg/mL, about 5.0 μg/mL, or about 10 μg/mL). In some embodiments, the starting concentration of template DNA vector (e.g., plasmid DNA vector) is 10 μg/mL. In some embodiments, the starting concentration of template DNA vector (e.g., plasmid DNA vector) is 1 μg/mL. In some embodiments, the starting concentration of template DNA vector (e.g., plasmid DNA vector) is 5 μg/mL.

Starting concentration of primers (e.g., random primers or specific primers) can be from 0.1 μM to 1.0 mM (e.g., from 0.5 μM to 500 μM, from 1.0 μM to 250 μM, from 2.0 μM to 200 μM, from 4 μM to 100 μM, or from 5 μM to 50 μM; e.g., from 0.1 μM to 0.5 μM, from 0.5 μM to 1.0 μM, from 1.0 μM to 2.0 μM, from 2.0 μM to 5.0 μM, from 5.0 μM to 10 μM, from 10 μM to 50 μM, from 50 μM to 100 μM, from 100 μM to 500 μM, or from 500 μM to 1.0 mM; e.g., about 1 μM, about 2 μM, about 5 μM, about 10 μM, about 20 μM, about 25 μM, about 50 μM, about 100 μM, about 200 μM, about 250 μM, about 300 μM, about 400 μM, about 500 μM, about 600 μM, about 700 μM, about 750 μM, about 800 μM, about 900 μM, or about 1.0 mM).

In some instances, the starting concentration of template DNA vector (e.g., plasmid DNA vector) at the start of amplification is from 1 μg/mL to 10 μg/mL and the starting concentration of primers is from 1 μM to 100 μM. In some instances, the starting concentration of plasmid DNA vector at the start of amplification is about 5 μg/mL and the starting concentration of primers is from 1 μM to 100 μM (e.g., about 50 μM).

Any suitable amplification buffer known in the art or described herein may be used in the present methods.

In some embodiments, the amplification reaction proceeds for a duration from about 1 hour to about 24 hours, e.g., about 18 hours. For example, the amplification reaction may be about 1 hour, about 2 hours, about 3 hours, about 4 hours, about 4 hours, about 5 hours, about 6 hours, about 7 hours, about 8 hours, about 9 hours, about 10 hours, about 11 hours, about 12 hours, 13 hours, 14 hours, 15 hours, 16 hours, 17 hours, 18 hours, 19 hours, 20 hours, 21 hours, 22 hours, 23 hours, or 24 hours. In some embodiments, the amplification reaction proceeds for about 18 hours.

In some embodiments, the amplification reaction is performed at a temperature from about 25° C. to about 42° C. (e.g., about 28° C. to about 40° C., e.g., about 29° C. to about 40° C., e.g., about 30° C.). For example, the amplification step may be performed at about 25° C., 26° C., 27° C., 28° C., 29° C., 30° C., 31° C., 32° C., 33° C., 34° C., 35° C., 36° C., 37° C., 38° C., 39° C., 40° C., 41° C., or 42° C. In some embodiments, the amplification step is performed at about 30° C.

In some instances, the total quantity of DNA present after amplification is at least five-fold the quantity (e.g., mass) of template DNA (e.g., plasmid DNA vector) present at the start of the amplification reaction (e.g., at least 10-fold, at least 15-fold, at least 20-fold, at least 25-fold, at least 30-fold, at least 40-fold, or at least 50-fold the quantity of template DNA present at the start of the amplification reaction; e.g., from 10-fold to 50-fold, from 10-fold to 40-fold, from 10-fold to 30-fold, or from 10-fold to 20-fold the quantity of template DNA present at the start of the amplification reaction; e.g., from 20-fold to 50-fold, from 20-fold to 40-fold, or from 20-fold to 30-fold the quantity of template DNA present at the start of the amplification reaction; e.g., from 30-fold to 50-fold or from 40-fold to 50-fold the quantity of template DNA present at the start of the amplification reaction).

In some embodiments, the total quantity of DNA present after amplification is at least 50-fold the quantity (e.g., mass) of template DNA (e.g., plasmid DNA vector) present at the start of the amplification reaction (e.g., from 50-fold to 300-fold, e.g., at least 82-fold, e.g., from 82-fold to 236-fold the quantity of template DNA present at the start of the amplification reaction.

In some instances, the restriction digest step occurs immediately after the amplification step (e.g., there is no heat inactivation step between amplification of the template DNA (e.g., plasmid DNA vector) and the restriction digest step).

In other instances, a heat inactivation step is performed after amplification (e.g., immediately after amplification). Heat inactivation may be conducted to inactivate the polymerase (e.g., phage polymerase, e.g., Phi29 polymerase) by raising the temperature to at least 50° C., at least 55° C., at least 60° C., at least 65° C., or at least 70° C. In some instances, the temperature is raised to at least 65° C. In some instances, the temperature of the heat inactivation after amplification is about 65° C. In some embodiments, restriction digest occurs immediately after heat inactivation.

Certain embodiments of the present methods allow for the manufacturing process to proceed from amplification to restriction digest without intervening steps that may compromise yield, such as in-process purification (e.g., gel purification (e.g., agarose gel extraction) or column purification). Thus, in some embodiments, there is no purification step (e.g., no gel purification step (e.g., no agarose gel extraction) or column purification step) between amplification and restriction digest. Additionally, or alternatively, in some embodiments, at least 90% of the amplified DNA product proceeds to restriction digest (e.g., at least 91%, at least 92%, at least 93%, at least 94%, at least 95%, at least 96%, at least 97%, at least 98%, at least 99%, or more of the amplified DNA product proceeds to restriction digest; e.g., from 90% to 95%, from 95% to 97%, from 97% to 98%, from 98% to 99%, or from 99% to 100% of the amplified DNA product proceeds to restriction digest).

### C. Restriction Digest

Template DNA vectors (e.g., plasmid DNA vectors), and/or concatemers thereof produced by rolling circle amplification, can be digested using restriction enzymes. In some embodiments, one or more restriction enzymes cut at least two sites (e.g., at least three sites or at least four sites) per unit of template DNA vector (e.g., plasmid DNA vector) to generate linear fragments of DNA, some of which include the therapeutic gene sequence.

In some embodiments, multiple restriction enzymes are used (e.g., two restriction enzymes are used). In such instances, a first restriction enzyme can be used to cut the therapeutic gene sequence from the backbone sequence (e.g., by designing the plasmid DNA vector such that the first restriction sites flank the therapeutic gene sequence). A second restriction enzyme can be used to cut the backbone sequence into one or more (e.g., two, three, or more) linear backbone fragments, which can then be degraded using an exonuclease (e.g., T5 exonuclease or Plasmid-Safe). Suitable restriction enzymes for such methods include, e.g., EcoRI and PvuII (e.g., EcoRI as the first restriction enzyme and PvuII as the second restriction enzyme).

In particular embodiments, a single restriction enzyme is used (i.e., the step comprises use of one and only one restriction enzyme). In such instances, type IIS restriction enzymes are suitable as a single restriction enzyme. Type IIS restriction enzymes may be particularly useful because they recognize a restriction site that is outside the cut site. Thus, following cleavage and ligation, the restriction site is no longer present in the ligated product (e.g., therapeutic circular DNA vector). This allows for the simultaneous treatment of a DNA fragment with the restriction enzyme and ligase.

In some instances, as discussed herein, type IIs restriction sites are positioned in a DNA molecule (e.g., a template DNA vector, e.g., plasmid DNA vector) outside the therapeutic sequence in such a way that a reaction containing a ligase and a type IIs restriction enzyme will drive the reaction forward to increase the relative concentration of therapeutic circular DNA vector to byproducts containing type IIs restriction sites (e.g., byproducts containing one or more backbone sequences and type IIs restriction sites).

In some embodiments, the type IIS restriction enzyme used in such embodiments is BsaI. Other suitable type IIS restriction enzymes that may be used in conjunction with the methods described herein include, for example, AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MboII, MlyI, MmeI, MnII, NmeAIII, PaqCI, PleI, SapI, and SfaNI.

In some embodiments, the restriction enzyme is provided at a concentration from about 0.5 U/μg DNA to about 20 U/μg DNA, e.g., from about 1 U/μg DNA to about 10 U/μg DNA, e.g., from about 2 U/μg DNA to about 5 U/μg DNA, e.g., about 2.5 U/μg DNA. For example, the restriction enzyme may be provided at a concentration of about 0.5 U/μg DNA, 1.0 U/μg DNA, 1.5 U/μg DNA, 2.0 U/μg DNA, 2.5 U/μg DNA, 3.0 U/μg DNA, 3.5 U/μg DNA, 4.0 U/μg DNA, 4.5 U/μg DNA, 5.0 U/μg DNA, 5.5 U/μg DNA, 6.0 U/μg DNA, 6.5 U/μg DNA, 7.0 U/μg DNA, 7.5 U/μg DNA, 8.0 U/μg DNA, 8.5 U/μg DNA, 9.0 U/μg DNA, 9.5 U/μg DNA, 10.0 U/μg DNA, 11 U/μg DNA, 12 U/μg DNA, 13 U/μg DNA, 14 U/μg DNA, 15 U/μg DNA, 16 U/μg DNA, 17 U/μg DNA, 18 U/μg DNA, 19 U/μg DNA, or 20 U/μg DNA. In some embodiments, the restriction enzyme is BsaI at a concentration of about 2.5 U/μg DNA.

In some embodiments, the restriction digest step is from about 1 hour to about 24 hours, e.g., about 1 hour to about 12 hours. For example, the digesting step can be about 1 hour, about 2 hours, about 3 hours, about 4 hours, about 4 hours, about 5 hours, about 6 hours, about 7 hours, about 8 hours, about 9 hours, about 10 hours, about 11 hours, about 12 hours, 13 hours, 14 hours, 15 hours, 16 hours, 17 hours, 18 hours, 19 hours, 20 hours, 21 hours, 22 hours, 23 hours, or 24 hours. In some embodiments, the digesting step is about 2 hours. In some embodiments, the digesting step is about 1 hour or less, e.g., about 30 minutes or less.

In some embodiments, the total quantity of DNA present after the restriction digest is at least 50-fold the quantity (e.g., mass) of template DNA (e.g., plasmid DNA vector) present at the start of the amplification reaction (e.g., from 50-fold to 300-fold, e.g., at least 82-fold, e.g., from 82-fold to 236-fold the quantity of template DNA present at the start of the amplification reaction.

Restriction digestion can be performed at a reaction temperature from about 30° C. to about 42° C. (e.g., about 32° C. to about 40° C., e.g., about 35° C. to about 40° C., e.g., about 37° C.). For example, in some instances, the restriction digestion step is performed at about 30° C., 31° C., 32° C., 33° C., 34° C., 35° C., 36° C., 37° C., 38° C., 39° C., 40° C., 41° C., or 42° C. In some embodiments, the restriction digestion step is performed at about 37° C.

Some embodiments of the present methods allow for the manufacturing process to proceed from restriction digest to ligation without intervening steps that may compromise yield, such as purification (e.g., gel purification (e.g., agarose gel extraction) or column purification). Thus, in some embodiments, there is no purification step (e.g., no gel purification step (e.g., no agarose gel extraction) or column purification step) between restriction digest and ligation. Additionally, or alternatively, in some embodiments, at least 90% of the total DNA present at or after restriction digest (including digested linear fragments (e.g., backbone and/or therapeutic sequences) and any undigested or circular DNA) proceeds to ligation (e.g., at least 91%, at least 92%, at least 93%, at least 94%, at least 95%, at least 96%, at least 97%, at least 98%, at least 99%, or more of the total DNA present at or after restriction digest proceeds to ligation; e.g., from 90% to 95%, from 95% to 97%, from 97% to 98%, from 98% to 99%, or from 99% to 100% of the total DNA present at or after restriction digest (including digested linear fragments (e.g., backbone and/or therapeutic sequences) and any undigested or circular DNA) proceeds to ligation).

In some instances, a heat inactivation step is performed after restriction digest and before ligation (e.g., a restriction digest involving one or more non-type II restriction enzymes, such as EcoRI and/or PvuII). Heat inactivation may be conducted to inactivate the restriction enzyme (e.g., a non-type II restriction enzyme, such as EcoRI and/or PvuII) by raising the temperature to at least 50° C., at least 55° C., at least 60° C., at least 65° C., or at least 70° C. In some instances, the temperature is raised to at least 65° C. In some instances, the temperature of the heat inactivation after amplification is about 65° C. In some embodiments, ligation occurs immediately after heat inactivation of the restriction enzyme(s).

Alternatively, no heat inactivation step is performed after (e.g., immediately after) ligation (e.g., no heat inactivation step is performed along the entire process).

In some embodiments involving a type II restriction enzyme, such as BsaI, no inactivation (e.g., heat inactivation) of restriction enzyme is necessary prior to ligation. In some embodiments, ligation occurs immediately after restriction digest (e.g., there is no heat inactivation step between restriction digest (e.g., restriction digest with a type II restriction enzyme, e.g., BsaI) and the ligation step). In some embodiments, ligation occurs, wholly or partially, during restriction digest. For example, the ligation reaction may be initiated at initiation of, or during, the restriction digest (e.g., ligase may be added to the DNA at the same time that the restriction enzyme (e.g., type II restriction enzyme, e.g., BsaI) is added or after the time of restriction enzyme is added (e.g., within one minute after the restriction enzyme is added, within five minutes after the restriction enzyme is added, within 10 minutes after the restriction enzyme is added, within 30 minutes after the restriction enzyme is added, within 60 minutes after the restriction enzyme is added, within 90 minutes after the restriction enzyme is added, or within 120 minutes after the restriction enzyme is added)). Alternatively, the ligation reaction may be initiated after the restriction digest is complete (e.g., after 2 hours from the time the restriction enzyme is added).

After restriction digest (e.g., immediately after restriction digest or immediately after heat inactivation, if performed), the reaction temperature can be adjusted to match the temperature of the aforementioned ligation reaction (e.g., 25° C.).

### D. Ligation

Self-ligation of the linear therapeutic fragment containing the therapeutic sequence results in a therapeutic circular DNA vector (e.g., a monomeric therapeutic circular DNA vector). In some embodiments, the self-ligating step includes providing a ligase (e.g., a DNA ligase) to the digested DNA sample to obtain a ligation solution. The ligase may be, e.g., T3 ligase, T4 ligase, or T7 ligase. In particular embodiments, T4 ligase is used. The ligation solution may contain additional components, such as ATP (e.g., ribo ATP) or other buffering agents at suitable concentrations known in the art or described herein. For example, some instances of the present methods involve preparing a ligation solution containing ligase in CUTSMART® or recombinant CUTSMART® (rCUTSMART®) buffer or equivalent buffer with ribo ATP, wherein the ribo ATP is at a concentration from 0.1 to 100 mM (e.g., about 10 mM).

In some embodiments, the total quantity of DNA contacted with ligase is 90% or more of the total quantity of DNA produced at the end of amplification (e.g., at least 91%, at least 92%, at least 93%, at least 94%, at least 95%, at least 96%, at least 97%, at least 98%, at least 99%, or more of the total quantity of DNA produced at the end of amplification; e.g., from 90% to 99%, from 91% to 99%, from 92% to 99%, from 93% to 99%, from 94% to 99%, from 95% to 99%, from 96% to 99%, from 97% to 99%, from 98% to 99%, from 90% to 98%, from 910% to 98%, from 92% to 98%, from 93% to 98%, from 94% to 98%, from 95% to 98%, from 96% to 98%, from 97% to 98%, from 90% to 97%, from 91% to 97%, from 92% to 97%, from 93% to 97%, from 94% to 97%, from 95% to 97%, from 96% to 97%, from 90% to 96%, from 91% to 96%, from 92% to 96%, from 93% to 96%, from 94% to 96%, from 95% to 96%, or from 90% to 95% the total quantity of DNA produced at the end of amplification).

Ligase (e.g., T4 ligase) can be present in the ligation solution at a concentration from about 0.5 U/μg DNA to about 20 U/μg DNA, e.g., from about 0.5 U/μg DNA to about 10 U/μg DNA, e.g., from about 1 U/μg DNA to about 10 U/μg DNA, e.g., from about 1 U/μg DNA to about 5 U/μg DNA, e.g., about 1.5 U/μg DNA, about 2.0 U/μg DNA, or about 2.5 U/μg DNA. For example, ligase (e.g., T4 ligase) may be provided at a concentration of about 0.5 U/μg DNA, 1.0 U/μg DNA, 1.5 U/μg DNA, 2.0 U/μg DNA, 2.5 U/μg DNA, 3.0 U/μg DNA, 3.5 U/μg DNA, 4.0 U/μg DNA, 4.5 U/μg DNA, 5.0 U/μg DNA, 5.5 U/μg DNA, 6.0 U/μg DNA, 6.5 U/μg DNA, 7.0 U/μg DNA, 7.5 U/μg DNA, 8.0 U/μg DNA, 8.5 U/μg DNA, 9.0 U/μg DNA, 9.5 U/μg DNA, 10.0 U/μg DNA, 11 U/μg DNA, 12 U/μg DNA, 13 U/μg DNA, 14 U/μg DNA, 15 U/μg DNA, 16 U/μg DNA, 17 U/μg DNA, 18 U/μg DNA, 19 U/μg DNA, or 20 U/μg DNA. In some embodiments, the ligase is provided at a concentration no greater than 50 U ligase per ag DNA (U/μg) (e.g., no greater than 40 U/μg DNA, no greater than 30 U/μg DNA, no greater than 25 U/μg DNA, no greater than 20 U/μg DNA, no greater than 15 U/μg DNA, no greater than 10 U/μg DNA, no greater than 5 U/μg DNA, no greater than 4 U/μg DNA, no greater than 3 U/μg DNA, no greater than 2.5 U/μg DNA, no greater than 2.0 U/μg DNA, no greater than 1.5 U/μg DNA, or no greater than 1.0 U/μg DNA; e.g., from 0.1 U/μg DNA to 20 U/μg DNA; e.g., from 0.1 U/μg DNA to 30 U/μg DNA, from 0.1 U/μg DNA to 20 U/μg DNA, from 0.2 U/μg DNA to 15 U/μg DNA, from 0.5 U/μg DNA to 12 U/μg DNA, or from 1 U/μg DNA to 10 U/μg DNA; e.g., from 0.1 U/μg DNA to 0.5 U/μg DNA, from 0.5 U/μg DNA to 1.0 U/μg DNA, from 1.0 U/μg DNA to 2.0 U/μg DNA, from 2.0 U/μg DNA to 3.0 U/μg DNA, from 3.0 U/μg DNA to 4.0 U/μg DNA, from 4.0 U/μg DNA to 5.0 U/μg DNA, from 5.0 to 6.0 U/μg DNA, from 6.0 U/μg DNA to 7.0 U/μg DNA, from 7.0 U/μg DNA to 8.0 U/μg DNA, from 8.0 U/μg DNA to 9.0 U/μg DNA, from 9.0 U/μg DNA to 11 U/μg DNA, from 11 U/μg DNA to 12 U/μg DNA, from 12 U/μg DNA to 15 U/μg DNA, from 15 U/μg DNA to 20 U/μg DNA, from 20 U/μg DNA to 25 U/μg DNA, from 25 U/μg DNA to 30 U/μg DNA, from 30 U/μg DNA to 35 U/μg DNA, from 35 U/μg DNA to 40 U/μg DNA, or from 40 U/μg DNA to 50 U/μg DNA). In some embodiments, the ligase (e.g., T4 ligase) is provided at a concentration no greater than 20 U/μg DNA (e.g., no greater than 15 U/μg DNA, no greater than 10 U/μg DNA, no greater than 5 U/μg DNA, no greater than 4 U/μg DNA, no greater than 3 U/μg DNA, no greater than 2.5 U/μg DNA, no greater than 2.0 U/μg DNA, no greater than 1.5 U/μg DNA, or no greater than 1.0 U/μg DNA; e.g., from 0.1 U/μg DNA to 20 U/μg DNA; e.g., from 0.2 U/μg DNA to 15 U/μg DNA, from 0.5 U/μg DNA to 12 U/μg DNA, or from 1 U/μg DNA to 10 U/μg DNA; e.g., from 0.1 U/μg DNA to 0.5 U/μg DNA, from 0.5 U/μg DNA to 1.0 U/μg DNA, from 1.0 U/μg DNA to 2.0 U/μg DNA, from 2.0 U/μg DNA to 3.0 U/μg DNA, from 3.0 U/μg DNA to 4.0 U/μg DNA, from 4.0 U/μg DNA to 5.0 U/μg DNA, from 5.0 to 6.0 U/μg DNA, from 6.0 U/μg DNA to 7.0 U/μg DNA, from 7.0 U/μg DNA to 8.0 U/μg DNA, from 8.0 U/μg DNA to 9.0 U/μg DNA, from 9.0 U/μg DNA to 11 U/μg DNA, from 11 U/μg DNA to 12 U/μg DNA, from 12 U/μg DNA to 15 U/μg DNA, or from 15 U/μg DNA to 20 U/μg DNA).

In particular embodiments, the linear therapeutic fragment is contacted with T4 ligase at a concentration from 5.0 U/μg DNA to 15 U/μg DNA (e.g., about 10 U/μg DNA).

In some embodiments, the ligation step is from about 30 minutes to about 24 hours, e.g., about 1 hour to about 12 hours. For example, the ligation step can be about 1 hour, about 2 hours, about 3 hours, about 4 hours, about 4 hours, about 5 hours, about 6 hours, about 7 hours, about 8 hours, about 9 hours, about 10 hours, about 11 hours, about 12 hours, 13 hours, 14 hours, 15 hours, 16 hours, 17 hours, 18 hours, 19 hours, 20 hours, 21 hours, 22 hours, 23 hours, or 24 hours. In some embodiments, the ligation step is about 2 hours. In some embodiments, the ligation step is about 18 to about 24 hours (e.g., about 18 hours, about 19 hours, about 20 hours, about 21 hours, about 22 hours, about 23 hours, or about 24 hours).

Ligation can be performed at a reaction temperature from about 20° C. to about 42° C. (e.g., about 20° C. to about 37° C., about 22° C. to about 30° C., or about 25° C.). For example, in some instances, the ligation step is performed at about 20° C., 21° C., 22° C., 23° C., 24° C., 25° C., 26° C., 27° C., 28° C., 29° C., or 30° C. In some embodiments, the ligation step is performed at about 25° C.

A heat inactivation step can be performed after ligation to inactivate the ligase. In processes involving a type II restriction enzyme (e.g., ligation simultaneous to or immediately after restriction digest) this post-ligation heat inactivation step may inactivate both the type II restriction enzyme (e.g., BsaI) and the ligase (e.g., T4 ligase). Heat inactivation may be conducted by raising the temperature to at least 50° C., at least 55° C., at least 60° C., at least 65° C., at least 70° C., or at least 80° C. In some instances, the temperature is raised to at least 65° C. In some instances, the temperature of the heat inactivation after amplification is about 65° C. In some embodiments, ligation occurs immediately after heat inactivation of the restriction enzyme(s). Heat inactivation may proceed for 10 minutes to 2 hours (e.g., from 30 minutes to 90 minutes, from 40 minutes to 60 minutes, or about 45 minutes). In some embodiments, the heat inactivation involves a post-ligation incubation at about 65° C. for about 45 minutes.

In some embodiments, after post-ligation heat inactivation, the method involves reducing the temperature of the solution to below 50° C. (e.g., from 20° C. to 40° C., from 25° C. to 37° C., or about 37° C.).

In other instances, no heat inactivation step is performed after (e.g., immediately after) ligation. In some instances, the temperature of the reaction after ligation (e.g., immediately after ligation) is less than 50° C. or less than 45° C. In some instances, the temperature is kept within (+/−) 10° C. of the ligation reaction temperature (e.g., within (+/−) 8° C., within (+/−) 5° C., or within (+/−) 2° C. of the ligation reaction temperature).

In some embodiments, the present methods allow for the manufacturing process to proceed from ligation to supercoiling and/or exonuclease digestion without intervening steps that may compromise yield, such as purification (e.g., gel purification (e.g., agarose gel extraction) or column purification). Thus, in some embodiments, there is no purification step (e.g., no gel purification step (e.g., no agarose gel extraction) or column purification step) between ligation and supercoiling and/or exonuclease digestion. Additionally, or alternatively, in some embodiments, at least 90% of the total DNA present at or after ligation (including therapeutic circular DNA, linear backbone fragments, and any unligated therapeutic fragments) proceeds to supercoiling and/or exonuclease digestion (e.g., at least 91%, at least 92%, at least 93%, at least 94%, at least 95%, at least 96%, at least 97%, at least 98%, at least 99%, or more of the total DNA present at or after ligation proceeds to supercoiling and/or exonuclease digestion; e.g., from 90% to 95%, from 95% to 97%, from 97% to 98%, from 98% to 99%, or from 99% to 100% of the total DNA present at or after ligation proceeds to supercoiling and/or exonuclease digestion).

### E. Supercoiling

Cell-free methods of producing therapeutic circular DNA vectors that are supercoiled (and pharmaceutical compositions thereof) may involve a step in which a relaxed circular DNA vector is contacted with a topoisomerase or a helicase under conditions suitable for supercoiling. In some embodiments, the therapeutic circular DNA vector produced by a method described herein is positively supercoiled. Methods described herein include any reagents and conditions known in the art or described herein to facilitate effective supercoiling.

For instance, an exemplary suitable buffer for a supercoiling reaction contains 35 mM Tris-HCl, 24 mM KCl, 4 mM MgCl2, 1 mM ATP, 2 mM DTT, 1.8 mM spermidine, 32% glycerol (w/v), and 100 μg/mL BSA.

In some embodiments, the total quantity of DNA contacted with topoisomerase or a helicase is 90% or more of the total quantity of DNA produced at the end of amplification or at the end of ligation (e.g., at least 91%, at least 92%, at least 93%, at least 94%, at least 95%, at least 96%, at least 97%, at least 98%, at least 99%, or more of the total quantity of DNA produced at the end of amplification or at the end of ligation; e.g., from 90% to 99%, from 91% to 99%, from 92% to 99%, from 93% to 99%, from 94% to 99%, from 95% to 99%, from 96% to 99%, from 97% to 99%, from 98% to 99%, from 90% to 98%, from 91% to 98%, from 92% to 98%, from 93% to 98%, from 94% to 98%, from 95% to 98%, from 96% to 98%, from 97% to 98%, from 90% to 97%, from 91% to 97%, from 92% to 97%, from 93% to 97%, from 94% to 97%, from 95% to 97%, from 96% to 97%, from 90% to 96%, from 91% to 96%, from 92% to 96%, from 93% to 96%, from 94% to 96%, from 95% to 96%, or from 90% to 95% the total quantity of DNA produced at the end of amplification or at the end of ligation).

In some embodiments, the topoisomerase is a type II topoisomerase. The type II topoisomerase may be, e.g., gyrase or topoisomerase IV.

In some embodiments, the topoisomerase (e.g., type II topoisomerase, e.g., topoisomerase IV or gyrase) or helicase is provided at a concentration of from about 0.5 U/μg DNA to about 20 U/μg DNA, e.g., from about 0.5 U/μg DNA to about 10 U/μg DNA, e.g., from about 1 U/μg DNA to about 10 U/μg DNA, e.g., from about 1 U/μg DNA to about 5 U/μg DNA, e.g., about 1.5 U/μg DNA, about 2.0 U/μg DNA, or about 2.5 U/μg DNA. For example, the topoisomerase (e.g., type II topoisomerase, e.g., topoisomerase IV or gyrase) or helicase may be provided at a concentration of about 0.5 U/μg DNA, 1.0 U/μg DNA, 1.5 U/μg DNA, 2.0 U/μg DNA, 2.5 U/μg DNA, 3.0 U/μg DNA, 3.5 U/μg DNA, 4.0 U/μg DNA, 4.5 U/μg DNA, 5.0 U/μg DNA, 5.5 U/μg DNA, 6.0 U/μg DNA, 6.5 U/μg DNA, 7.0 U/μg DNA, 7.5 U/μg DNA, 8.0 U/μg DNA, 8.5 U/μg DNA, 9.0 U/μg DNA, 9.5 U/μg DNA, 10.0 U/μg DNA, 11 U/μg DNA, 12 U/μg DNA, 13 U/μg DNA, 14 U/μg DNA, 15 U/μg DNA, 16 U/μg DNA, 17 U/μg DNA, 18 U/μg DNA, 19 U/μg DNA, or 20 U/μg DNA. In some embodiments, the topoisomerase (e.g., type II topoisomerase, e.g., topoisomerase IV or gyrase) or helicase is provided at a concentration no greater than 10 U/μg DNA (e.g., no greater than 5 U/μg DNA, no greater than 4 U/μg DNA, no greater than 3 U/μg DNA, no greater than 2.5 U/μg DNA, no greater than 2.0 U/μg DNA, no greater than 1.5 U/μg DNA, or no greater than 1.0 U/μg DNA; e.g., from 0.1 U/μg DNA to 10 U/μg DNA; e.g., from 0.5 U/μg DNA to 8 U/μg DNA, or from 1 U/μg DNA to 5 U/μg DNA; e.g., from 0.1 U/μg DNA to 0.5 U/μg DNA, from 0.5 U/μg DNA to 1.0 U/μg DNA, from 1.0 U/μg DNA to 2.0 U/μg DNA, from 2.0 U/μg DNA to 3.0 U/μg DNA, from 3.0 U/μg DNA to 4.0 U/μg DNA, from 4.0 U/μg DNA to 5.0 U/μg DNA, from 5.0 to 6.0 U/μg DNA, from 6.0 U/μg DNA to 7.0 U/μg DNA, from 7.0 U/μg DNA to 8.0 U/μg DNA, from 8.0 U/μg DNA to 9.0 U/μg DNA, or from 9.0 U/μg DNA to 10 U/μg DNA).

In particular embodiments, the relaxed circular DNA vector is contacted with gyrase at a concentration from 1.0 U/μg DNA to 2.5 U/μg DNA (e.g., about 1.0 U/μg DNA, about 1.5 U/μg DNA, or about 2.0 U/μg DNA). In embodiments in which the gyrase is contacted to the DNA after terminal exonuclease digestion (e.g., T5 exonuclease digestion), the gyrase is at a concentration from 0.1 U/μg DNA to 1.5 U/μg DNA (e.g., 0.2 U/μg DNA to 1.5 U/μg DNA, 0.5 U/μg DNA to 1.5 U/μg DNA, 0.5 U/μg DNA to 1.0 U/μg DNA, or 1.0 U/μg DNA to 1.5 U/μg DNA, e.g., about 0.1 U/μg DNA, about 0.2 U/μg DNA, about 0.3 U/μg DNA, about 0.4 U/μg DNA, about 0.5 U/μg DNA, about 1.0 U/μg DNA, or about 1.5 U/μg DNA).

In some embodiments, the step of contacting the circular DNA vector with the topoisomerase or helicase (e.g., type II topoisomerase, e.g., topoisomerase IV or gyrase) is from about 1 hour to about 24 hours, e.g., about 1 hour to about 12 hours. For example, the step of contacting the circular DNA vector with the topoisomerase or helicase (e.g., type II topoisomerase, e.g., topoisomerase IV or gyrase) is about 1 hour, about 2 hours, about 3 hours, about 4 hours, about 4 hours, about 5 hours, about 6 hours, about 7 hours, about 8 hours, about 9 hours, about 10 hours, about 11 hours, about 12 hours, 13 hours, 14 hours, 15 hours, 16 hours, 17 hours, 18 hours, 19 hours, 20 hours, 21 hours, 22 hours, 23 hours, or 24 hours. In some embodiments, the step of contacting the circular DNA vector with the topoisomerase or helicase (e.g., type II topoisomerase, e.g., topoisomerase IV or gyrase) is about 12 hours.

In some embodiments, the step of contacting the circular DNA vector with the topoisomerase or helicase (e.g., type II topoisomerase, e.g., topoisomerase IV or gyrase) is performed at a temperature from about 30° C. to about 42° C. (e.g., about 32° C. to about 40° C., e.g., about 35° C. to about 40° C., e.g., about 37° C.). For example, the digesting step may be performed at about 30° C., 31° C., 32° C., 33° C., 34° C., 35° C., 36° C., 37° C., 38° C., 39° C., 40° C., 41° C., or 42° C. In some embodiments, the step of contacting the circular DNA vector with the topoisomerase or helicase (e.g., type II topoisomerase, e.g., topoisomerase IV or gyrase) is performed at about 37° C.

### F. Terminal Exonuclease

Any of the methods of cell-free production of therapeutic circular DNA vectors described herein may involve a cleanup step in which undesired DNA (e.g., bacterial sequences, linear or nicked DNA byproducts, etc.) are enzymatically degraded. In particular instances, linear backbone fragments produced upon restriction digestion can be selectively degraded in a solution containing circularized therapeutic fragments (e.g., relaxed circular therapeutic DNA vector or supercoiled therapeutic DNA vector) using a terminal exonuclease at any suitable conditions known in the art or described herein. In some embodiments, the terminal exonuclease is T5 exonuclease.

Methods described herein include any reagents and conditions known in the art or described herein to facilitate effective terminal exonuclease activity. For instance, an exemplary suitable buffer for a terminal exonuclease reaction may be a potassium acetate buffer (e.g., from 10 mM to 100 mM potassium acetate, e.g., about 50 mM potassium acetate).

In some embodiments, the total quantity of DNA contacted with a terminal exonuclease is 90% or more of the total quantity of DNA produced at the end of amplification or at the end of ligation (e.g., at least 91%, at least 92%, at least 93%, at least 94%, at least 95%, at least 96%, at least 97%, at least 98%, at least 99%, or more of the total quantity of DNA produced at the end of amplification or at the end of ligation; e.g., from 90% to 99%, from 91% to 99%, from 92% to 99%, from 93% to 99%, from 94% to 99%, from 95% to 99%, from 96% to 99%, from 97% to 99%, from 98% to 99%, from 90% to 98%, from 91% to 98%, from 92% to 98%, from 93% to 98%, from 94% to 98%, from 95% to 98%, from 96% to 98%, from 97% to 98%, from 90% to 97%, from 91% to 97%, from 92% to 97%, from 93% to 97%, from 94% to 97%, from 95% to 97%, from 96% to 97%, from 90% to 96%, from 91% to 96%, from 92% to 96%, from 93% to 96%, from 94% to 96%, from 95% to 96%, or from 90% to 95% the total quantity of DNA produced at the end of amplification or at the end of ligation).

In some embodiments, the terminal exonuclease (e.g., T5 exonuclease) is provided at a concentration of from about 0.5 U/μg to about 20 U/μg, e.g., from about 0.5 U/μg to about 10 U/μg, e.g., from about 1 U/μg to about 10 U/μg, e.g., from about 2 U/μg to about 5 U/μg, e.g., about 2.5 U/μg. For example, the terminal exonuclease may be provided at a concentration of about 0.5 U/μg, 1.0 U/μg, 1.5 U/μg, 2.0 U/μg, 2.5 U/μg, 3.0 U/μg, 3.5 U/μg, 4.0 U/μg, 4.5 U/μg, 5.0 U/μg, 5.5 U/μg, 6.0 U/μg, 6.5 U/μg, 7.0 U/μg, 7.5 U/μg, 8.0 U/μg, 8.5 U/μg, 9.0 U/μg, 9.5 U/μg, 10.0 U/μg, 11 U/μg, 12 U/μg, 13 U/μg, 14 U/μg, 15 U/μg, 16 U/μg, 17 U/μg, 18 U/μg, 19 U/μg, or 20 U/μg.

In some embodiments, the digesting step with the terminal exonuclease is from about 1 hour to about 24 hours, e.g., about 1 hour to about 12 hours. For example, the digesting step is about 1 hour, about 2 hours, about 3 hours, about 4 hours, about 4 hours, about 5 hours, about 6 hours, about 7 hours, about 8 hours, about 9 hours, about 10 hours, about 11 hours, about 12 hours, 13 hours, 14 hours, 15 hours, 16 hours, 17 hours, 18 hours, 19 hours, 20 hours, 21 hours, 22 hours, 23 hours, or 24 hours. In some embodiments, the digesting step is between 2 hours and 12 hours (e.g., 2 to 5 hours, 2 to 4 hours, or 2 to 3 hours).

In some embodiments, digesting the sample with the terminal exonuclease is performed from about 30° C. to about 42° C. (e.g., about 32° C. to about 40° C., e.g., about 35° C. to about 40° C., e.g., about 37° C.). For example, the digesting step can be performed at about 30° C., 31° C., 32° C., 33° C., 34° C., 35° C., 36° C., 37° C., 38° C., 39° C., 40° C., 41° C., or 42° C. In some embodiments, digesting the sample with the terminal exonuclease is performed at about 37° C.

In some instances, no heat inactivation of the terminal exonuclease is performed immediately after the exonuclease digestion.

In some instances, terminal exonuclease digestion is conducted after supercoiling. (e.g., immediately after supercoiling). Alternatively, terminal exonuclease digestion is conducted before supercoiling (e.g., immediately before supercoiling). In some embodiments, terminal exonuclease digestion is conducted simultaneously with supercoiling.

### G. Purification Precipitation

In some embodiments of any of the methods described herein, the method further includes precipitating the therapeutic circular DNA vector, e.g., via isopropanol precipitation.

In some embodiments, prior to precipitation, solution containing therapeutic circular DNA vector (e.g., supercoiled circular DNA vector) is sterile filtered, e.g., through a 0.22 m filter. Solution can be reconstituted in buffer containing IPA according to methods known in the art and described herein. In some embodiments, a sterile filtrate from Section F above is reconstituted in IPA buffer to a final concentration of 760 mM NaCl, 50 mM MOPS, 15% isopropyl alcohol (IPA), and 0.15% Triton X-100 (v/v). Therapeutic circular DNA vector (e.g., supercoiled circular DNA vector) in IPA buffer is added to an equilibrated Qiagen-tip column (Qiagen plasmid kit), and the column is washed and contents eluted. 24.5 mL IPA buffer is added per 35 mL elution. IPA precipitation can then be performed in which the sample is centrifuged for 30 minutes at 15,000 g at 4° C. The dried pellet can be resuspended in water or desired final buffer.

In some embodiments, the quantity of therapeutic circular DNA vector obtained after purification/precipitation (e.g., a single purification/precipitation step, e.g., not more than one purification/precipitation step) is at least two-fold the number of therapeutic sequences (e.g., at least two-fold, at least three-fold, at least four-fold, at least five-fold, at least six-fold, at least seven-fold, at least eight-fold, at least nine-fold, at least ten-fold, at least 20-fold, at least 30-fold, at least 40-fold, at least 50-fold, at least 60-fold, at least 70-fold, at least 80-fold, at least 90-fold, at least 100-fold; e.g., from two-fold to 1,000-fold, from two-fold to 500-fold, from two-fold to 100-fold, from two-fold to 50-fold, from two-fold to 40-fold, from two-fold to 30-fold, from two-fold to 20-fold, or from two-fold to ten-fold; e.g., from five-fold to 1,000-fold, from five-fold to 500-fold, from five-fold to 100-fold, from five-fold to 50-fold, from five-fold to 40-fold, from five-fold to 30-fold, from five-fold to 20-fold, or from five-fold to ten-fold; e.g., from ten-fold to 1,000-fold, from ten-fold to 500-fold, from ten-fold to 100-fold, from ten-fold to 50-fold, from ten-fold to 40-fold, from ten-fold to 30-fold, or from ten-fold to 20-fold; e.g., from two-fold to five-fold, from five-fold to ten-fold, from ten-fold to 20-fold, from 20-fold to 30-fold, from 30-fold to 40-fold, from 40-fold to 50-fold, from 50-fold to 60-fold, from 60-fold to 70-fold, from 70-fold to 80-fold, from 80-fold to 90-fold, from 90-fold to 100-fold, from 100-fold to 200-fold, from 200-fold to 500-fold, or from 500-fold to 1,000-fold; e.g., about two-fold, about three-fold, about four-fold, about five-fold, about six-fold, about seven-fold, about eight-fold, about nine-fold, about 10-fold, about 15-fold, about 20-fold, about 25-fold, about 30-fold, about 40-fold, about 50-fold, about 60-fold, about 70-fold, about 80-fold, about 90-fold, or about 100-fold the number of therapeutic sequences) the quantity of template DNA vector (e.g., plasmid DNA vector) from which it was produced. In some embodiments, the quantity of therapeutic circular DNA vector obtained after purification/precipitation (e.g., a single purification/precipitation step, e.g., not more than one purification/precipitation step) is at least three-fold the quantity of template DNA vector (e.g., plasmid DNA vector) from which it was produced. Additionally, or alternatively, the quantity of therapeutic circular DNA vector obtained after purification/precipitation (e.g., a single purification/precipitation step, e.g., not more than one purification/precipitation step) is at least 1.0 mg (e.g., from 1.0 mg to 10 g, from 1.0 mg to 5.0 g, from 1.0 mg to 1.0 g, from 1.0 mg to 500 mg, from 1.0 mg to 200 mg, from 1.0 mg to 100 mg, from 1.0 mg to 50 mg, from 1.0 mg to 25 mg, from 1.0 mg to 20 mg, from 1.0 mg to 15 mg, from 1.0 mg to 10 mg, from 1.0 mg to 5.0 mg, from 2.0 mg to 10 g, from 2.0 mg to 5.0 g, from 2.0 mg to 1.0 g, from 2.0 mg to 500 mg, from 2.0 mg to 200 mg, from 2.0 mg to 100 mg, from 2.0 mg to 50 mg, from 2.0 mg to 25 mg, from 2.0 mg to 20 mg, from 2.0 mg to 15 mg, from 2.0 mg to 10 mg, from 2.0 mg to 5.0 mg, from 5.0 mg to 10 g, from 5.0 mg to 5.0 g, from 5.0 mg to 1.0 g, from 5.0 mg to 500 mg, from 5.0 mg to 200 mg, from 5.0 mg to 100 mg, from 5.0 mg to 50 mg, from 5.0 mg to 25 mg, from 5.0 mg to 20 mg, from 5.0 mg to 15 mg, from 5.0 mg to 10 mg, from 10 mg to 10 g, from 10 mg to 5.0 g, from 10 mg to 1.0 g, from 10 mg to 500 mg, from 10 mg to 200 mg, from 10 mg to 100 mg, from 10 mg to 50 mg, from 10 mg to 25 mg, from 10 mg to 20 mg, or from 10 mg to 15 mg). In some embodiments, the quantity of therapeutic circular DNA vector obtained after purification/precipitation (e.g., a single purification/precipitation step, e.g., not more than one purification/precipitation step) is at least 2.0 mg.

In some instances, the amount (mass) of therapeutic circular DNA vector produced by methods of the invention is at least twice the amount (mass) of template DNA (e.g., plasmid DNA vector) input in the production at amplification (e.g., at least three-fold, at least four-fold, at least five-fold, at least six-fold, at least seven-fold, at least eight-fold, at least nine-fold, or at least 10-fold the mass of template DNA (e.g., plasmid DNA vector) input in the production at amplification; e.g., from two-fold to 20-fold, from two-fold to 15-fold, from two-fold to 13-fold, from three-fold to 10-fold, or from four-fold to 8-fold the mass of template DNA (e.g., plasmid DNA vector) input in the production at amplification; e.g., about two-fold, about three-fold, about four-fold, about five-fold, about six-fold, about seven-fold, about eight-fold, about nine-fold, about 10-fold, about 11-fold, about 12-fold, or about 13-fold the mass of template DNA (e.g., plasmid DNA vector) input in the production at amplification).

### IV. Therapeutic Circular DNA Vectors

Provided herein are therapeutic circular DNA vectors produced by any of the methods of production described herein. In some instances, such therapeutic circular DNA vectors persist intracellularly (e.g., in dividing or in quiescent cells, such as post-mitotic cells) as episomes, e.g., in a manner similar to AAV vectors. In any of the embodiments, described herein, a therapeutic circular DNA vector may be a non-integrating vector. Therapeutic circular DNA vectors provided herein can be naked DNA vectors, devoid of components inherent to viral vectors (e.g., viral proteins) and bacterial plasmid DNA, such as immunogenic components (e.g., immunogenic bacterial signatures (e.g., CpG islands or CpG motifs)) or components additionally, or otherwise associated with reduced persistence (e.g., CpG islands or CpG motifs). The therapeutic circular DNA vectors produced as described herein feature one or more therapeutic sequences and may lack plasmid backbone elements (e.g., bacterial elements such as (i) a bacterial origin of replication and/or (ii) a drug resistance gene) and a recombination site.

Therapeutic circular DNA vectors provided herein can be naked DNA vectors, devoid of components inherent to viral vectors (e.g., viral proteins) and bacterial plasmid DNA, such as immunogenic components (e.g., immunogenic bacterial signatures (e.g., CpG motifs)) or components additionally or otherwise associated with reduced persistence (e.g., CpG islands). For example, in some embodiments, the vector contains DNA in which at least 50% (e.g., at least 60%, at least 70%, at least 80%, at least 90%, at least 95%, at least 97%, at least 99%, or essentially all) of the DNA lacks one or more elements of bacterial plasmid DNA, such as immunogenic components (e.g., immunogenic bacterial signatures (e.g., CpG motifs)) or components additionally or otherwise associated with reduced persistence (e.g., CpG islands). In some embodiments, at least 50% (e.g., at least 60%, at least 70%, at least 80%, at least 90%, at least 95%, at least 97%, at least 99%, or essentially all) of the DNA lacks CpG methylation. In some embodiments, the vector contains DNA in which at least 50% (e.g., at least 60%, at least 70%, at least 80%, at least 90%, at least 95%, at least 97%, at least 99%, or essentially all) of the DNA lacks bacterial methylation signatures, such as Dam methylation and Dcm methylation. For examples, in some embodiments, the vector contains DNA in which at least 50% (e.g., at least 60%, at least 70%, at least 80%, at least 90%, at least 95%, at least 97%, at least 99%, or essentially all) of the GATC sequences are unmethylated (e.g., by Dam methylase). Additionally, or alternatively, the vector contains DNA in which at least 50% (e.g., at least 60%, at least 70%, at least 80%, at least 90%, at least 95%, at least 97%, at least 99%, or essentially all) of the CCAGG sequences and/or CCTGG sequences are unmethylated (e.g., by Dcm methylase).

In some embodiments, the therapeutic circular DNA vector is persistent in vivo (e.g., the therapeutic circular DNA vector exhibits improved expression persistence (e.g., intra-cellular persistence and/or trans-generational persistence) and/or therapeutic persistence relative to a reference vector, e.g., a circular DNA vector produced in bacteria or having one or more bacterial signatures not present in the vector of the invention, e.g., plasmid DNA). In some embodiments, expression persistence of the therapeutic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, intra-cellular persistence of the therapeutic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, trans-generational persistence of the therapeutic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, therapeutic persistence of the therapeutic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, the reference vector is a circular vector or plasmid that (a) has the same therapeutic sequence as a therapeutic circular DNA vector to which it is being compared, and (b) is produced in bacteria and/or has one or more bacterial signatures not present in the therapeutic circular DNA vector to which it is being compared, which signatures may include, for example, an antibiotic resistance gene or a bacterial origin of replication.

In some embodiments, expression of a therapeutic circular DNA vector persists for one week, two weeks, three weeks, four weeks, six weeks, two months, three months, four months, five months, six months, seven months, eight months, nine months, ten months, eleven months, one year, or longer after administration. In particular embodiments, the therapeutic circular DNA vector exhibits intra-cellular persistence and/or trans-generational persistence of one week, two weeks, three weeks, four weeks, six weeks, two months, three months, four months, five months, six months, seven months, eight months, nine months, ten months, eleven months, one year, or longer after administration. In some embodiments, therapeutic persistence of a therapeutic circular DNA vector endures for one week, two weeks, three weeks, four weeks, six weeks, two months, three months, four months, five months, six months, seven months, eight months, nine months, ten months, eleven months, one year, or longer after administration.

In some embodiments, expression and/or therapeutic effect of the therapeutic circular DNA vector persists for one week to four weeks, from one month to four months, or from four months to one year (e.g., at least one week, at least two weeks, at least one month, or longer). In some embodiments, the expression level of the therapeutic circular DNA vector does not decrease by more than 90%, by more than 50%, or by more than 10% in the 1 week or more, e.g., 2 weeks, 3 weeks, 5 weeks, 7 weeks, 9 weeks or more, 13 weeks or more, 18 weeks or more following transfection from levels observed within the first 1, 2, or 3 days.

The therapeutic circular DNA vector may be monomeric, dimeric, trimeric, tetrameric, pentameric, hexameric, etc. In some preferred embodiments, the circular DNA vector is monomeric. In some embodiments, the DNA vector is supercoiled, e.g., following treatment with a topoisomerase (e.g., gyrase). In some embodiments, the therapeutic circular DNA vector is a monomeric, supercoiled circular DNA molecule. In some embodiments, the therapeutic circular DNA vector is nicked. In some embodiments, the therapeutic circular DNA vector is open circular. In some embodiments, the therapeutic circular DNA vector is double-stranded circular.

### Therapeutic Sequences

Therapeutic circular DNA vectors described herein contain a therapeutic sequence, which may include one or more protein-coding domain and/or one or more non-protein coding domains (e.g., a therapeutic nucleic acid).

In particular embodiments involving a therapeutic protein-coding therapeutic domain, the therapeutic sequence includes, linked in the 5′ to 3′ direction: a promoter and a single therapeutic protein-coding domain (e.g., a single transcription unit); a promoter and two or more therapeutic protein-coding domains (e.g., a multicistronic unit); or a first transcription unit and one or more additional transcription units (e.g., a multi-transcription unit). Any such protein-coding therapeutic sequences may further include non-protein coding domains, such as polyadenylation sites, control elements, enhancers, sequences to mark DNA (e.g., for antibody recognition), PCR amplification sites, sequences that define restriction enzyme sites, site-specific recombinase recognition sites, sequences that are recognized by a protein that binds to and/or modifies nucleic acids, linkers, splice sites, pre-mRNA binding domains, regulatory sequences, and/or a therapeutic nucleic acid (e.g., a microRNA-encoding sequence).

Therapeutic protein-coding domains can be full-length protein-coding domains (e.g., corresponding to a native gene or natural variant thereof) or a functional portion thereof, such as a truncated protein-coding domain (e.g., minigene).

In some embodiments, the therapeutic sequence encodes a monomeric protein (e.g., a monomeric protein with secondary structure under physiological conditions, e.g., a monomeric protein with secondary and tertiary structure under physiological conditions, e.g., a monomeric protein with secondary, tertiary, and quaternary structure under physiological conditions).

Additionally, or alternatively, the therapeutic sequence may encode a multimeric protein (e.g., a dimeric protein (e.g., a homodimeric protein or heterodimeric protein), a trimeric protein, etc.) In some embodiments, the therapeutic sequence encodes an antibody, or a portion, fragment, or variant thereof. Antibodies include fragments that are capable of binding to an antigen, such as Fv, single-chain Fv (scFv), Fab, Fab′, di-scFv, sdAb (single domain antibody), (Fab′)₂ (including a chemically linked F(ab′)₂), and nanobodies. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. Antibodies also include chimeric antibodies and humanized antibodies. Furthermore, for all antibody constructs provided herein, variants having the sequences from other organisms are also contemplated. Thus, if a human version of an antibody is disclosed, one of skill in the art will appreciate how to transform the human sequence-based antibody into a mouse, rat, cat, dog, horse, etc. sequence. Antibody fragments also include either orientation of single chain scFvs, tandem di-scFv, diabodies, tandem tri-sdcFv, minibodies, nanobodies, etc. In some embodiments, such as when an antibody is an scFv, a single polynucleotide of a therapeutic gene sequence encodes a single polypeptide comprising both a heavy chain and a light chain linked together. Antibody fragments also include nanobodies (e.g., sdAb, an antibody having a single, monomeric domain, such as a pair of variable domains of heavy chains, without a light chain). Multispecific antibodies (e.g., bispecific antibodies, trispecific antibodies, etc.) are known in the art and contemplated as expression products of the therapeutic gene sequences of the present invention.

In some instances, the therapeutic sequence encodes one or more proteins (e.g., a single protein, two proteins, three proteins, four proteins, or more), each having a length of at least 25 amino acids, at least 50 amino acids, at least 100 amino acids, at least 200 amino acids, at least 500 amino acids, at least 1,000 amino acids, at least 1,500 amino acids, at least 2,000 amino acids, at least 2,500 amino acids, at least 3,000 amino acids, or more (e.g., from 25 to 5,000 amino acids, from 50 to 5,000 amino acids, from 100 to 5,000 amino acids, from 200 to 5,000 amino acids, from 500 to 5,000 amino acids, from 1,000 to 5,000 amino acids, from 1,500 to 5,000 amino acids, or from 2,000 to 5,000 amino acids; e.g., from 25 to 4,000 amino acids, from 50 to 4,000 amino acids, from 100 to 4,000 amino acids, from 200 to 4,000 amino acids, from 500 to 4,000 amino acids, from 1,000 to 4,000 amino acids, from 1,500 to 4,000 amino acids, or from 2,000 to 4,000 amino acids; e.g., from 25 to 3,000 amino acids, from 50 to 3,000 amino acids, from 100 to 3,000 amino acids, from 200 to 3,000 amino acids, from 500 to 3,000 amino acids, from 1,000 to 3,000 amino acids, from 1,500 to 3,000 amino acids, or from 2,000 to 3,000 amino acids). In embodiments in which such therapeutic sequence encodes two or more proteins, the therapeutic sequence can be a multicistronic therapeutic sequence or a multi-transcription unit therapeutic sequence.

In some embodiments, the therapeutic sequence encodes an ocular protein. In particular embodiments, the ocular protein is ABCA4. An exemplary human ABCA4 sequence is provided as NCBI Reference Sequence: NG_009073 or NM_000350.

In embodiments involving a non-protein coding therapeutic sequence, the therapeutic sequence lacks a protein-coding domain (e.g., a therapeutic protein-coding domain). For instance, in some embodiments, a therapeutic sequence includes a non-protein-coding therapeutic nucleic acid, such as a short hairpin RNA (shRNA)-encoding sequence or an immune activating therapeutic nucleic acid (e.g., a TLR agonist).

In some embodiments, the therapeutic sequence is from 0.1 Kb to 100 Kb in length (e.g., the therapeutic gene sequence is from 0.2 Kb to 90 Kb, from 0.5 Kb to 80 Kb, from 1.0 Kb to 70 Kb, from 1.5 Kb to 60 Kb, from 2.0 Kb to 50 Kb, from 2.5 Kb to 45 Kb, from 3.0 Kb to 40 Kb, from 3.5 Kb to 35 Kb, from 4.0 Kb to 30 Kb, from 4.5 Kb to 25 Kb, from 4.6 Kb to 24 Kb, from 4.7 Kb to 23 Kb, from 4.8 Kb to 22 Kb, from 4.9 Kb to 21 Kb, from 5.0 Kb to 20 Kb, from 5.5 Kb to 18 Kb, from 6.0 Kb to 17 Kb, from 6.5 Kb to 16 Kb, from 7.0 Kb to 15 Kb, from 7.5 Kb to 14 Kb, from 8.0 Kb to 13 Kb, from 8.5 Kb to 12.5 Kb, from 9.0 Kb to 12.0 Kb, from 9.5 Kb to 11.5 Kb, or from 10.0 Kb to 11.0 Kb in length, e.g., from 0.1 Kb to 0.5 Kb, from 0.5 Kb to 1.0 Kb, from 1.0 Kb to 2.5 Kb, from 2.5 Kb to 4.5 Kb, from 4.5 Kb to 8 Kb, from 8 Kb to 10 Kb, from 10 Kb to 15 Kb, from 15 Kb to 20 Kb in length, or greater, e.g., from 0.1 Kb to 0.25 Kb, from 0.25 Kb to 0.5 Kb, from 0.5 Kb to 1.0 Kb, from 1.0 Kb to 1.5 Kb, from 1.5 Kb to 2.0 Kb, from 2.0 Kb to 2.5 Kb, from 2.5 Kb to 3.0 Kb, from 3.0 Kb to 3.5 Kb, from 3.5 Kb to 4.0 Kb, from 4.0 Kb to 4.5 Kb, from 4.5 Kb to 5.0 Kb, from 5.0 Kb to 5.5 Kb, from 5.5 Kb to 6.0 Kb, from 6.0 Kb to 6.5 Kb, from 6.5 Kb to 7.0 Kb, from 7.0 Kb to 7.5 Kb, from 7.5 Kb to 8.0 Kb, from 8.0 Kb to 8.5 Kb, from 8.5 Kb to 9.0 Kb, from 9.0 Kb to 9.5 Kb, from 9.5 Kb to 10 Kb, from 10 Kb to 10.5 Kb, from 10.5 Kb to 11 Kb, from 11 Kb to 11.5 Kb, from 11.5 Kb to 12 Kb, from 12 Kb to 12.5 Kb, from 12.5 Kb to 13 Kb, from 13 Kb to 13.5 Kb, from 13.5 Kb to 14 Kb, from 14 Kb to 14.5 Kb, from 14.5 Kb to 15 Kb, from 15 Kb to 15.5 Kb, from 15.5 Kb to 16 Kb, from 16 Kb to 16.5 Kb, from 16.5 Kb to 17 Kb, from 17 Kb to 17.5 Kb, from 17.5 Kb to 18 Kb, from 18 Kb to 18.5 Kb, from 18.5 Kb to 19 Kb, from 19 Kb to 19.5 Kb, from 19.5 Kb to 20 Kb, from 20 Kb to 21 Kb, from 21 Kb to 22 Kb, from 22 Kb to 23 Kb, from 23 Kb to 24 Kb, from 24 Kb to 25 Kb in length, or greater, e.g., about 4.5 Kb, about 5.0 Kb, about 5.5 Kb, about 6.0 Kb, about 6.5 Kb, about 7.0 Kb, about 7.5 Kb, about 8.0 Kb, about 8.5 Kb, about 9.0 Kb, about 9.5 Kb, about 10 Kb, about 11 Kb, about 12 Kb, about 13 Kb, about 14 Kb, about 15 Kb, about 16 Kb, about 17 Kb, about 18 Kb, about 19 Kb, about 20 Kb in length, or greater). In some embodiments, the therapeutic sequence is at least 10 Kb (e.g., from 10 Kb to 15 Kb, from 15 Kb to 20 Kb, or from 20 Kb to 30 Kb; e.g., from 10 Kb to 13 Kb, from 10 Kb to 12 Kb, or from 10 Kb to 11 Kb; e.g., from 10-11 Kb, from 11-12 Kb, from 12-13 Kb, from 13-14 Kb, or from 14-15 Kb). In some embodiments, the therapeutic sequence is at least 1,100 bp in length (e.g., from 1,100 bp to 10,000 bp, from 1,100 bp to 8,000 bp, or from 1,100 bp to 5,000 bp in length). In some embodiments, the therapeutic sequence is at least 2,500 bp in length (e.g., from 2,500 bp to 15,000 bp, from 2,500 bp to 10,000 bp, or from 2,500 bp to 5,000 bp in length; e.g., from 2,500 bp to 5,000 bp, from 5,000 bp to 7,500 bp, from 7,500 bp to 10,000 bp, from 10,000 bp to 12,500 bp, or from 12,500 bp to 15,000 bp). In some embodiments, the therapeutic sequence is at least 8,000 bp, at least 9,000 bp, at least 10,000 bp, at least 11,000 bp, at least 12,000 bp at least 13,000 bp, at least 14,000 bp, at least 15,000 bp, at least 16,000 bp (e.g., 11,000 bp to 16,000 bp, 12,000 bp to 16,000 bp, 13,000 bp to 16,000 bp, 14,000 bp to 16,000 bp, or 15,000 bp to 16,000 bp). In particular embodiments, the therapeutic sequence is sufficiently large to encode a protein and is not an oligonucleotide therapy (e.g., not an antisense, siRNA, shRNA therapy, etc.).

In some embodiments, the 3′ end of the therapeutic sequence is connected to the 5′ end of the therapeutic sequence in a therapeutic circular DNA vector by a non-bacterial sequence of no more than 30 bp (e.g., from 3 bp to 24 bp, from 4 bp to 18 bp, from 5 bp to 12 bp, or from 6 bp to 10 bp; e.g., from 3 bp to 5 bp, from 4 bp to 6 bp, from 8 bp to 12 bp, from 12 bp to 18 bp, from 18 bp to 24 bp, or from 24 bp to 30 bp; e.g., 3 bp, 4 bp, 5 bp, 6 bp, 7 bp, or 8 bp). For example, in any of the therapeutic circular DNA vectors generated using type IIs restriction enzymes described herein, the 3′ end of the therapeutic sequence may be connected to the 5′ end of the therapeutic sequence by a non-bacterial sequence corresponding to sticky end or overhang of the type IIs restriction enzyme cut site (e.g., TTTT, AAAA, or AACC). In some instances, the sticky end or overhang of the type IIs restriction enzyme cut site comprises (or consists of) four bases, wherein two and only two of the four bases are A or T (e.g., AACC or TTGG). In some instances, the sticky end or overhang of the type IIs restriction enzyme cut site comprises (or consists of) four bases, wherein two and only two of the four bases are A (e.g., AACC). In some instances, the sticky end or overhang of the type IIs restriction enzyme cut site comprises (or consists of) four bases, wherein two and only two of the four bases are T (e.g., TTGG).

In some embodiments, the therapeutic sequence includes a reporter sequence in addition to a therapeutic protein-encoding domain or a therapeutic non-protein encoding domain. Such reporter genes can be useful in verifying therapeutic gene sequence expression, for example, in specific cells and tissues. Reporter sequences that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

In some embodiments, the therapeutic sequence lacks a reporter sequence.

As part of the therapeutic sequence, therapeutic circular DNA vectors of the invention may include conventional control elements which modulate or improve transcription, translation, and/or expression in a target cell. Suitable control elements are described in International Publication No. WO 2021/055760, which is incorporated herein by reference in its entirety.

In some instances, any of the therapeutic circular DNA vectors of the invention encodes a self-replicating RNA molecule. Such self-replicating RNA molecules include replicase sequences derived from alphavirus, which are characterized as having positive-stranded replicons that are translated after delivery to a target cell into a replicase (or replicase-transcriptase). The replicase is translated as a polyprotein which auto-cleaves to provide a replication complex which creates genomic negative-strand copies of the positive-strand delivered RNA. These negative-strand transcripts can themselves be transcribed to give further copies of the positive-stranded parent RNA and also to give a subgenomic transcript (e.g., a modulatory sequence). Translation of the subgenomic transcript thus leads to in situ expression of the modulatory protein by the infected cell.

Non-limiting examples of alphaviruses from which replicase-encoding sequences of the present invention can be derived include Venezuelan equine encephalitis virus (VEE), Semliki Forest virus (SF), Sindbis virus (SIN), Eastern Equine Encephalitis virus (EEE), Western equine encephalitis virus (WEE), Everglades virus (EVE), Mucambo virus (MUC), Pixuna virus (PIX), Semliki Forest virus (SF), Middelburg virus (MID), Chikungunya virus (CHIK), O'Nyong-Nyong virus (ONN), Ross River virus (RR), Barmah Forest virus (BF), Getah virus (GET), Sagiyama virus (SAG), Bebaru virus (BEB), Mayaro virus (MAY), Una virus (UNA), Aura virus (AURA), Babanki virus (BAB), Highlands J virus (HJ), and Fort Morgan virus (FM). In particular instances of the invention, the self-replicating RNA molecule comprises a VEE replicase or a variant thereof.

Mutant or wild-type virus sequences can be used. For example, in some instances, the self-replicating RNA includes an attenuated TC83 mutant of VEE replicase. Other mutations in the replicase are contemplated herein, including replicase mutated replicases (e.g., mutated VEE replicases) obtained by in vitro evolution methods, e.g., as taught by Yingzhong et al., Sci Rep. 2019, 9: 6932, the methodology of which is incorporated herein by reference.

In some instances, a self-replicating RNA molecule includes (i) a replicase-encoding sequence (e.g., an RNA sequence that encodes an RNA-dependent RNA polymerase which can transcribe RNA from the self-replicating RNA molecule) and (ii) a heterologous modulatory gene. The polymerase can be an alphavirus replicase, e.g., an alphavirus replicase comprising one, two, three, or all four alphavirus nonstructural proteins nsP1, nsP2, nsP3, and nsP4. In some instances, the polymerase is a VEE replicase, e.g., a VEE replicase comprising one, two, three, or all four alphavirus nonstructural proteins nsP1, nsP2, nsP3, and nsP4.

In some instances of the present invention, a self-replicating RNA molecule does not encode alphavirus structural proteins (e.g., capsid proteins). Such self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins can be replaced by gene(s) encoding the heterologous modulatory protein(s) of interest, such that the subgenomic transcript encodes the heterologous modulatory protein(s) rather than the structural alphavirus virion proteins.

Accordingly, in some instances, a self-replicating RNA molecule of the invention can have two open reading frames. The first (5′) open reading frame encodes a replicase; the second (3′) open reading frame encodes one or more (e.g., two or three) therapeutic proteins. In some embodiments, the RNA may have additional (e.g. downstream) open reading frames, e.g., to encode further genes or to encode accessory polypeptides.

Suitable self-replicating RNA molecules can have various lengths. In some embodiments of the invention, the length of the self-replicating RNA molecule is from 5,000 to 50,000 nucleotides (i.e., 5 kb to 50 kb). In some instances, the self-replicating RNA molecule is 5 kb to 20 kb in length (e.g., from 6 kb to 18 kb, from 7 kb to 16 kb, from 8 kb to 14 kb, or from 9 kb to 12 kb in length, e.g., from 5 kb to 6 kb, from 6 kb to 7 kb, from 7 kb to 8 kb, from 8 kb to 9 kb, from 9 kb to 10 kb, from 10 kb to 11 kb, from 11 kb to 12 kb, from 12 kb to 13 kb, from 13 kb to 14 kb, from 14 kb to 15 kb, from 15 kb to 16 kb, from 16 kb to 18 kb, or from 18 kb to 20 kb in length, e.g., about 5 kb, about 6 kb, about 7 kb, about 8 kb, about 9 kb, about 10 kb, about 10.5 kb, about 11 kb, about 11.5 kb, about 12 kb, about 12.5 kb, about 13 kb, about 14 kb, about 15 kb, about 16 kb, about 17 kb, about 18 kb, about 19 kb, or about 20 kb in length).

A self-replicating RNA molecule may have a 3′ poly-A tail. Additionally, the self-replicating RNA molecule may include a poly-A polymerase recognition sequence (e.g., AAUAAA).

In a particular embodiment, the RNA according to the invention does not encode a reporter molecule, such as luciferase or a fluorescent protein, such as green fluorescent protein (GFP).

In some embodiments, the replicase encoded by the self-replicating RNA can be a variant of any of the replicases described herein. In some embodiments, the variant is a functional fragment (e.g., a fragment of the protein that is functionally similar or functionally equivalent to the protein).

### V. Pharmaceutical Compositions

Improvements in efficiency render the present methods particularly amenable to scalable manufacturing of pharmaceutical compositions containing therapeutic circular DNA vectors.

Any of the methods of producing therapeutic circular DNA vectors described herein can be adapted for production of pharmaceutical compositions containing the therapeutic circular DNA vector in a pharmaceutically acceptable carrier.

Provided herein are methods of producing a pharmaceutical formulation containing a therapeutic circular DNA vector (e.g., a supercoiled therapeutic circular DNA vector). In some embodiments, such methods include the following steps: First, a sample containing a plasmid DNA vector having a therapeutic gene sequence and a backbone sequence is provided. The plasmid DNA vector is amplified using polymerase-mediated rolling-circle amplification to generate a linear concatemer. Next, the linear concatemer is digested with a restriction enzyme that cuts at least a first site and a second site of the linear concatemer per unit, wherein the first and second sites flank the therapeutic sequence. This digestion produces a linear therapeutic fragment containing the therapeutic sequence and a linear backbone fragment containing the backbone sequence. The linear therapeutic fragment is then self-ligated to produce a relaxed circular DNA vector, which is then contacted with a topoisomerase or a helicase to produce a supercoiled circular DNA vector. In some embodiments, the linear bacterial fragments are digested with a terminal exonuclease.

In certain instances, methods of producing a pharmaceutical formulation containing a therapeutic circular DNA vector include the following steps: A sample containing a plasmid DNA vector having a therapeutic sequence and a backbone sequence is provided. The plasmid DNA vector is amplified using polymerase-mediated rolling-circle amplification to generate a linear concatemer. Next, the linear concatemer is digested with a restriction enzyme that cuts at least a first site and a second site of the linear concatemer per unit, wherein the first and second sites flank the therapeutic sequence. This digestion produces a linear therapeutic fragment containing the therapeutic sequence and a linear backbone fragment containing the backbone sequence. The linear therapeutic fragment is then self-ligated to produce a circular DNA vector, and the linear backbone fragment is digested with a terminal nuclease. In some embodiments, the circular DNA vector is contacted with a topoisomerase or a helicase to produce a supercoiled circular DNA vector.

In some instances, methods of producing a pharmaceutical formulation containing a therapeutic circular DNA vector include the following steps: A sample comprising a plasmid DNA vector comprising a therapeutic sequence and a backbone sequence is provided. The plasmid DNA vector is amplified using a polymerase-mediated rolling-circle amplification to generate a linear concatemer. Next, the linear concatemer is digested with a restriction enzyme (e.g., type IIs restriction enzyme, e.g., BsaI) that cuts at least a first site, a second site, and a third site per unit of the linear concatemer. The first and second sites flank the therapeutic sequence and form self-complementary overhangs, and the third site is within the backbone sequence and forms an overhang that is non-complementary to the first or second site. The digestion produces a linear therapeutic fragment having the therapeutic sequence and at least two linear backbone fragments each including a portion of the backbone sequence. The linear therapeutic fragment is contacted with a ligase to produce a therapeutic circular DNA vector in solution.

In some embodiments, the restriction enzyme cuts a fourth site of the linear concatemer per unit, wherein the fourth site is within the backbone sequence and forms an overhang that is non-complementary to the first or second site, and wherein the digestion produces at least three linear backbone fragments each comprising a portion of the backbone sequence.

In some embodiments, the therapeutic circular DNA vector is contacted with topoisomerase or a helicase. Such reactions can be carried out at about 37° C. Additionally, or alternatively, the therapeutic circular DNA vector can be contacted with a terminal exonuclease (e.g., in a reaction carried out at about 37° C.). In particular embodiments, the therapeutic circular DNA vector is contact with a topoisomerase or a helicase and, without raising the reaction temperature to inactivate the topoisomerase or helicase, the therapeutic circular DNA vector is thereafter contacted with a terminal exonuclease.

In some embodiments, after contacting the therapeutic circular DNA vector with the topoisomerase or helicase and/or the terminal exonuclease, the method includes running the therapeutic circular DNA vector through a column (e.g., a capture column). In some embodiments, the therapeutic circular DNA vector is then precipitated with isopropyl alcohol.

The aforementioned methods can produce pharmaceutical formulations containing a high quantity and purity of therapeutic circular DNA vector (e.g., supercoiled therapeutic circular DNA vector). Thus, the invention includes any of the pharmaceutical formulations described herein. In some embodiments, a pharmaceutical formulation of the invention contains at least two-fold the number of therapeutic sequences as the sample of plasmid DNA vector from which it was produced. In some embodiments, the pharmaceutical formulation contains at least five-fold the number of therapeutic sequences as the sample of plasmid DNA vector. In some embodiments, the pharmaceutical formulation contains at least ten-fold the number of therapeutic sequences as the sample of plasmid DNA vector.

In particular embodiments, a pharmaceutical formulation of the invention (e.g., the pharmaceutical composition produced by any of the methods described herein) contains at least two-fold the number of therapeutic sequences (e.g., at least two-fold, at least three-fold, at least four-fold, at least five-fold, at least six-fold, at least seven-fold, at least eight-fold, at least nine-fold, at least ten-fold, at least 20-fold, at least 30-fold, at least 40-fold, at least 50-fold, at least 60-fold, at least 70-fold, at least 80-fold, at least 90-fold, at least 100-fold; e.g., from two-fold to 1,000-fold, from two-fold to 500-fold, from two-fold to 100-fold, from two-fold to 50-fold, from two-fold to 40-fold, from two-fold to 30-fold, from two-fold to 20-fold, or from two-fold to ten-fold; e.g., from five-fold to 1,000-fold, from five-fold to 500-fold, from five-fold to 100-fold, from five-fold to 50-fold, from five-fold to 40-fold, from five-fold to 30-fold, from five-fold to 20-fold, or from five-fold to ten-fold; e.g., from ten-fold to 1,000-fold, from ten-fold to 500-fold, from ten-fold to 100-fold, from ten-fold to 50-fold, from ten-fold to 40-fold, from ten-fold to 30-fold, or from ten-fold to 20-fold; e.g., from two-fold to five-fold, from five-fold to ten-fold, from ten-fold to 20-fold, from 20-fold to 30-fold, from 30-fold to 40-fold, from 40-fold to 50-fold, from 50-fold to 60-fold, from 60-fold to 70-fold, from 70-fold to 80-fold, from 80-fold to 90-fold, from 90-fold to 100-fold, from 100-fold to 200-fold, from 200-fold to 500-fold, or from 500-fold to 1,000-fold; e.g., about two-fold, about three-fold, about four-fold, about five-fold, about six-fold, about seven-fold, about eight-fold, about nine-fold, about 10-fold, about 15-fold, about 20-fold, about 25-fold, about 30-fold, about 40-fold, about 50-fold, about 60-fold, about 70-fold, about 80-fold, about 90-fold, or about 100-fold the number of therapeutic sequences) as the sample of template DNA vector (e.g., plasmid DNA vector) from which it was produced.

In some embodiments, a pharmaceutical formulation of the invention (e.g., the pharmaceutical composition produced by any of the methods described herein) contains at least 1.0 mg therapeutic circular DNA vector in a pharmaceutically acceptable carrier (e.g., from 1.0 mg to 10 g, from 1.0 mg to 5.0 g, from 1.0 mg to 1.0 g, from 1.0 mg to 500 mg, from 1.0 mg to 200 mg, from 1.0 mg to 100 mg, from 1.0 mg to 50 mg, from 1.0 mg to 25 mg, from 1.0 mg to 20 mg, from 1.0 mg to 15 mg, from 1.0 mg to 10 mg, from 1.0 mg to 5.0 mg, from 2.0 mg to 10 g, from 2.0 mg to 5.0 g, from 2.0 mg to 1.0 g, from 2.0 mg to 500 mg, from 2.0 mg to 200 mg, from 2.0 mg to 100 mg, from 2.0 mg to 50 mg, from 2.0 mg to 25 mg, from 2.0 mg to 20 mg, from 2.0 mg to 15 mg, from 2.0 mg to 10 mg, from 2.0 mg to 5.0 mg, from 5.0 mg to 10 g, from 5.0 mg to 5.0 g, from 5.0 mg to 1.0 g, from 5.0 mg to 500 mg, from 5.0 mg to 200 mg, from 5.0 mg to 100 mg, from 5.0 mg to 50 mg, from 5.0 mg to 25 mg, from 5.0 mg to 20 mg, from 5.0 mg to 15 mg, from 5.0 mg to 10 mg, from 10 mg to 10 g, from 10 mg to 5.0 g, from 10 mg to 1.0 g, from 10 mg to 500 mg, from 10 mg to 200 mg, from 10 mg to 100 mg, from 10 mg to 50 mg, from 10 mg to 25 mg, from 10 mg to 20 mg, or from 10 mg to 15 mg).

In some embodiments, a pharmaceutical formulation of the invention (e.g., the pharmaceutical composition produced by any of the methods described herein) contains at least 2.0 mg therapeutic circular DNA vector in a pharmaceutically acceptable carrier. In some embodiments, a pharmaceutical formulation produced by any of the methods described herein contains at least 5.0 mg therapeutic circular DNA vector in a pharmaceutically acceptable carrier. In some embodiments, a pharmaceutical formulation produced by any of the methods described herein contains at least 10.0 mg therapeutic circular DNA vector in a pharmaceutically acceptable carrier.

In some embodiments, a pharmaceutical formulation of the invention (e.g., the pharmaceutical composition produced by a method described herein (e.g., a method involving contacting a therapeutic circular DNA vector with a topoisomerase or helicase)) contains therapeutic circular DNA vector that is at least 60% supercoiled monomer, at least 70% supercoiled monomer, at least 80% supercoiled monomer, or at least 90% supercoiled monomer (e.g., 60% to 80% supercoiled monomer, 60% to 90% supercoiled monomer, 60% to 95% supercoiled monomer, 60% to 99% supercoiled monomer, 60% to 99.5% supercoiled monomer, 60% to 99.9% supercoiled monomer, 65% to 80% supercoiled monomer, 65% to 90% supercoiled monomer, 65% to 95% supercoiled monomer, 65% to 99% supercoiled monomer, 65% to 99.5% supercoiled monomer, 65% to 99.9% supercoiled monomer, 70% to 80% supercoiled monomer, 70% to 90% supercoiled monomer, 70% to 95% supercoiled monomer, 70% to 99% supercoiled monomer, 70% to 99.5% supercoiled monomer, 70% to 99.9% supercoiled monomer, 75% to 80% supercoiled monomer, 75% to 90% supercoiled monomer, 75% to 95% supercoiled monomer, 75% to 99% supercoiled monomer, 75% to 99.5% supercoiled monomer, 75% to 99.9% supercoiled monomer, 80% to 85% supercoiled monomer, 80% to 90% supercoiled monomer, 80% to 95% supercoiled monomer, 80% to 99% supercoiled monomer, 80% to 99.5% supercoiled monomer, 80% to 99.9% supercoiled monomer, 85% to 90% supercoiled monomer, 85% to 95% supercoiled monomer, 85% to 99% supercoiled monomer, 85% to 99.5% supercoiled monomer, 85% to 99.9% supercoiled monomer, 90% to 95% supercoiled monomer, 90% to 99% supercoiled monomer, 90% to 99.5% supercoiled monomer, 90% to 99.9% supercoiled monomer, 95% to 99% supercoiled monomer, 95% to 99.5% supercoiled monomer, 95% to 99.9% supercoiled monomer, 98% to 99% supercoiled monomer, 98% to 99.5% supercoiled monomer, or 98% to 99.9% supercoiled monomer; e.g., about 60% supercoiled monomer, about 65% supercoiled monomer, about 70% supercoiled monomer, about 75% supercoiled monomer, about 80% supercoiled monomer, about 85% supercoiled monomer, about 90% supercoiled monomer, about 95% supercoiled monomer, about 96% supercoiled monomer, about 97% supercoiled monomer, about 98% supercoiled monomer, about 99% supercoiled monomer, or about 99.9% supercoiled monomer). In any of these instances, supercoiled monomer is calculated using densitometry analysis of gel electrophoresis (e.g., as described in Example 5, below).

In other embodiments, a pharmaceutical formulation of the invention (e.g., the pharmaceutical composition produced by a method described herein (e.g., a method in which the therapeutic circular DNA vector is not contacted with a topoisomerase or helicase)) contains therapeutic circular DNA vector that is not supercoiled (i.e., relaxed circular DNA).

In some embodiments, percent supercoiled monomer is determined by agarose gel electrophoresis or capillary electrophoresis. Additionally, or alternatively, percent supercoiled monomer is determined by anion exchange-HPLC.

In some embodiments, the pharmaceutical formulation of the invention (e.g., the pharmaceutical composition produced by a method described herein) is substantially devoid of impurities. For instance, in some embodiments, the pharmaceutical formulation contains <1.0% protein content by mass (e.g., <0.9%, <0.8%, <0.7%, <0.6%, <0.5%, <0.4%, <0.3%, <0.2%, <0.1%, <0.05%, or <0.01% protein content by mass). In some instances, protein content is determined by bicinchoninic acid assay. Additionally or alternatively, protein content is determined by ELISA.

In some instances, the pharmaceutical formulation of the invention (e.g., the pharmaceutical composition produced by a method described herein) contains <1.0% RNA content by mass (e.g., <0.9%, <0.8%, <0.7%, <0.6%, <0.5%, <0.4%, <0.3%, <0.2%, <0.1%, <0.05%, or <0.01% RNA content by mass). In some embodiments, the RNA content is determined by agarose gel electrophoresis. In some embodiments, the RNA content is determined by quantitative PCR. In some embodiments, the RNA content is determined by fluorescence assay (e.g., Ribogreen).

In some embodiments, the pharmaceutical formulation of the invention (e.g., the pharmaceutical composition produced by a method described herein) contains <1.0% gDNA content by mass (e.g., <0.9%, <0.8%, <0.7%, <0.6%, <0.5%, <0.4%, <0.3%, <0.2%, <0.1%, <0.05%, or <0.01% gDNA content by mass). In some embodiments, the gDNA content is determined by agarose gel electrophoresis or capillary electrophoresis. In some embodiments, the gDNA content is determined by quantitative PCR. In some embodiments, the gDNA content is determined by Southern blot.

In some embodiments, the pharmaceutical formulation of the invention (e.g., the pharmaceutical composition produced by a method described herein) contains <40 EU/mg endotoxin. In some embodiments, the pharmaceutical formulation contains <20 EU/mg endotoxin. In some embodiments, the pharmaceutical formulation contains <10 EU/mg endotoxin. In some embodiments, the pharmaceutical formulation contains <5 EU/mg endotoxin (e.g., <4 EU/mg endotoxin, <3 EU/mg endotoxin, <2 EU/mg endotoxin, <1 EU/mg endotoxin, <0.5 EU/mg endotoxin), e.g., as measured by Limulus Ameobocyte Lysate (LAL) assay.

Pharmaceutical compositions provided herein may include one or more pharmaceutically acceptable carriers, such as excipients and/or stabilizers that are nontoxic to the individual being treated (e.g., human patient) at the dosages and concentrations employed. In some embodiments, the pharmaceutically acceptable carrier is an aqueous pH buffered solution. Examples of pharmaceutically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as tween, polyethylene glycol (PEG), and pluronics.

If the pharmaceutical composition is provided in liquid form, the pharmaceutically acceptable carrier may be water (e.g., pyrogen-free water), isotonic saline, or a buffered aqueous solution, e.g., a phosphate buffered solution or a citrate buffered solution. Injection of the pharmaceutical composition may be carried out in water or a buffer, such as an aqueous buffer, e.g., containing a sodium salt (e.g., at least 50 mM of a sodium salt), a calcium salt (e.g., at least 0.01 mM of a calcium salt), or a potassium salt (e.g., at least 3 mM of a potassium salt). According to a particular embodiment, the sodium, calcium, or potassium salt may occur in the form of their halogenides, e.g., chlorides, iodides, or bromides, in the form of their hydroxides, carbonates, hydrogen carbonates, or sulfates, etc. Without being limited thereto, examples of sodium salts include NaCl, NaI, NaBr, Na₂CO₂, NaHCO₂, and Na₂SO₄. Examples of potassium salts include, e.g., KCl, KI, KBr, K₂CO₂, KHCO₂, and K₂SO₄. Examples of calcium salts include, e.g., CaCl₂), CaI₂, CaBr₂, CaCO₂, CaSO₄, and Ca(OH)₂. Additionally, organic anions of the aforementioned cations may be contained in the buffer. According to a particular embodiment, the buffer suitable for injection purposes as defined above, may contain salts selected from sodium chloride (NaCl), calcium chloride (CaCl₂)) or potassium chloride (KCl), wherein further anions may be present. CaCl₂) can also be replaced by another salt, such as KCl. In some embodiments, salts in the injection buffer are present in a concentration of at least 50 mM sodium chloride (NaCl), at least 3 mM potassium chloride (KCl), and at least 0.01 mM calcium chloride (CaCl₂)). The injection buffer may be hypertonic, isotonic, or hypotonic with reference to the specific reference medium, i.e., the buffer may have a higher, identical or lower salt content with reference to the specific reference medium, wherein preferably such concentrations of the afore mentioned salts may be used, which do not lead to damage of cells due to osmosis or other concentration effects. Reference media can be liquids such as blood, lymph, cytosolic liquids, other body liquids, or common buffers. Such common buffers or liquids are known to a skilled person. Ringer-Lactate solution is particularly preferred as a liquid basis.

One or more compatible solid or liquid fillers, diluents, or encapsulating compounds may be suitable for administration to a person. The constituents of the pharmaceutical composition according to the invention are capable of being mixed with the nucleic acid vector according to the invention as defined herein, in such a manner that no interaction occurs, which would substantially reduce the pharmaceutical effectiveness of the (pharmaceutical) composition according to the invention under typical use conditions. Pharmaceutically acceptable carriers, fillers and diluents can have sufficiently high purity and sufficiently low toxicity to make them suitable for administration to an individual being treated. Some examples of compounds which can be used as pharmaceutically acceptable carriers, fillers, or constituents thereof are sugars, such as lactose, glucose, trehalose, and sucrose; starches, such as corn starch or potato starch; dextrose; cellulose and its derivatives, such as sodium carboxymethylcellulose, ethylcellulose, cellulose acetate; powdered tragacanth; malt; gelatin; tallow; solid glidants, such as stearic acid, magnesium stearate; calcium sulfate; vegetable oils, such as groundnut oil, cottonseed oil, sesame oil, olive oil, corn oil and oil from theobroma; polyols, such as polypropylene glycol, glycerol, sorbitol, mannitol, and polyethylene glycol; or alginic acid.

The choice of a pharmaceutically acceptable carrier can be determined, according to the manner in which the pharmaceutical composition is administered.

Suitable unit dose forms for injection include sterile solutions of water, physiological saline, and mixtures thereof. The pH of such solutions may be adjusted to about 7.4. Suitable carriers for injection include hydrogels, devices for controlled or delayed release, polylactic acid, and collagen matrices. Suitable pharmaceutically acceptable carriers for topical application include those which are suitable for use in lotions, creams, gels and the like. If the pharmaceutical composition is to be administered perorally, tablets, capsules and the like are the preferred unit dose form.

Further additives which may be included in the pharmaceutical composition are emulsifiers, such as tween; wetting agents, such as sodium lauryl sulfate; coloring agents; pharmaceutical carriers; stabilizers; antioxidants; and preservatives.

The pharmaceutical composition according to the present invention may be provided in liquid or in dry (e.g. lyophilized) form. In a particular embodiment, the nucleic acid vector of the pharmaceutical composition is provided in lyophilized form. Lyophilized compositions including nucleic acid vector of the invention may be reconstituted in a suitable buffer, advantageously based on an aqueous carrier, prior to administration, e.g. Ringer-Lactate solution, Ringer solution, or a phosphate buffer solution.

In certain embodiments of the invention, any of the therapeutic circular DNA vectors of the invention can be complexed with one or more cationic or polycationic compounds, e.g., cationic or polycationic polymers, cationic or polycationic peptides or proteins, e.g. protamine, cationic or polycationic polysaccharides, and/or cationic or polycationic lipids.

According to a particular embodiment, the therapeutic circular DNA vector of the invention may be complexed with lipids to form one or more liposomes, lipoplexes, or lipid nanoparticles. Therefore, in one embodiment, the pharmaceutical composition comprises liposomes, lipoplexes, and/or lipid nanoparticles comprising a therapeutic circular DNA vector.

Lipid-based formulations can be effective delivery systems for nucleic acid vectors due to their biocompatibility and their ease of large-scale production. Cationic lipids have been widely studied as synthetic materials for delivery of nucleic acids. After mixing together, nucleic acids are condensed by cationic lipids to form lipid/nucleic acid complexes known as lipoplexes. These lipid complexes are able to protect genetic material from the action of nucleases and deliver it into cells by interacting with the negatively charged cell membrane. Lipoplexes can be prepared by directly mixing positively charged lipids at physiological pH with negatively charged nucleic acids.

Conventional liposomes include of a lipid bilayer that can be composed of cationic, anionic, or neutral phospholipids and cholesterol, which encloses an aqueous core. Both the lipid bilayer and the aqueous space can incorporate hydrophobic or hydrophilic compounds, respectively. Liposome characteristics and behavior in vivo can be modified by addition of a hydrophilic polymer coating, e.g. polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains.

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm.

Cationic liposomes can serve as delivery systems for therapeutic circular DNA vectors. Cationic lipids, such as MAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for nucleic acid vector delivery as e.g., neutral 1,2-dioleoyl-sn-glycero-3-phosphatidylcholine (DOPC)-based nanoliposomes are available.

Thus, in one embodiment of the invention, the therapeutic circular DNA vector of the invention is complexed with cationic lipids and/or neutral lipids and thereby forms liposomes, lipid nanoparticles, lipoplexes or neutral lipid-based nanoliposomes in the present pharmaceutical compositions.

In a particular embodiment, a pharmaceutical composition according to the invention comprises the therapeutic circular DNA vector of the invention that is formulated together with a cationic or polycationic compound and/or with a polymeric carrier. Accordingly, in a further embodiment of the invention, the therapeutic circular DNA vector as defined herein is associated with or complexed with a cationic or polycationic compound or a polymeric carrier, optionally in a weight ratio selected from a range of about 5:1 (w/w) to about 0.25:1 (w/w), e.g., from about 5:1 (w/w) to about 0.5:1 (w/w), e.g., from about 4:1 (w/w) to about 1:1 (w/w) or of about 3:1 (w/w) to about 1:1 (w/w), e.g., from about 3:1 (w/w) to about 2:1 (w/w) of nucleic acid vector to cationic or polycationic compound and/or with a polymeric carrier; or optionally in a nitrogen/phosphate (N/P) ratio of nucleic acid vector to cationic or polycationic compound and/or polymeric carrier in the range of about 0.1-10, e.g., in a range of about 0.3-4 or 0.3-1, e.g., in a range of about 0.5-1 or 0.7-1, e.g., in a range of about 0.3-0.9 or 0.5-0.9. For example, the N/P ratio of the therapeutic circular DNA vector to the one or more polycations is in the range of about 0.1 to 10, including a range of about 0.3 to 4, of about 0.5 to 2, of about 0.7 to 2 and of about 0.7 to 1.5.

The nucleic acid vectors described herein can also be associated with a vehicle, transfection or complexation agent for increasing the transfection efficiency and/or the expression of the modulatory gene according to the invention.

In some instances, the therapeutic circular DNA vector according to the invention is complexed with one or more polycations, preferably with protamine or oligofectamine. Further cationic or polycationic compounds, which can be used as transfection or complexation agent may include cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPE, LEAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, MAP: dioleoyloxy-3-(trimethylammonio)propane, DC-6-14: O,O-ditetradecanoyl-N-(α-trimethylammonioacetyl)diethanolamine chloride, CLIP1: rac-[(2,3-dioctadecyloxypropyl)(2-hydroxyethyl)]-dimethylammonium chloride, CLIP6: rac-[2(2,3-dihexadecyloxypropyl-oxymethyloxy)ethyl]trimethylammonium, CLIP9: rac-[2(2,3-dihexadecyloxypropyl-oxysuccinyloxy)ethyl]-trimethylammonium, oligofectamine, or cationic or polycationic polymers, e.g. modified polyaminoacids, such as Q3-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., block polymers consisting of a combination of one or more cationic blocks (e.g. selected from a cationic polymer as mentioned above) and of one or more hydrophilic or hydrophobic blocks (e.g. polyethyleneglycole); etc.

According to a particular embodiment, the pharmaceutical composition of the invention includes the therapeutic circular DNA vector encapsulated within or attached to a polymeric carrier. A polymeric carrier used according to the invention might be a polymeric carrier formed by disulfide-crosslinked cationic components. The disulfide-crosslinked cationic components may be the same or different from each other. The polymeric carrier can also contain further components. It is also particularly preferred that the polymeric carrier used according to the present invention comprises mixtures of cationic peptides, proteins or polymers and optionally further components as defined herein, which are crosslinked by disulfide bonds as described herein. In this context, the disclosure of WO 2012/013326 is incorporated herein by reference. In this context, the cationic components that form basis for the polymeric carrier by disulfide-crosslinkage are typically selected from any suitable cationic or polycationic peptide, protein or polymer suitable for this purpose, particular any cationic or polycationic peptide, protein or polymer capable of complexing the nucleic acid vector as defined herein or a further nucleic acid comprised in the composition, and thereby preferably condensing the nucleic acid vector. The cationic or polycationic peptide, protein or polymer, may be a linear molecule; however, branched cationic or polycationic peptides, proteins or polymers may also be used.

Every disulfide-crosslinking cationic or polycationic protein, peptide or polymer of the polymeric carrier, which may be used to complex the therapeutic circular DNA vector according to the invention included as part of the pharmaceutical composition of the invention may contain at least one SH moiety (e.g., at least one cysteine residue or any further chemical group exhibiting an SH moiety) capable of forming a disulfide linkage upon condensation with at least one further cationic or polycationic protein, peptide or polymer as cationic component of the polymeric carrier as mentioned herein.

Such polymeric carriers used to complex the therapeutic circular DNA vector of the present invention may be formed by disulfide-crosslinked cationic (or polycationic) components. In particular, such cationic or polycationic peptides or proteins or polymers of the polymeric carrier, which comprise or are additionally modified to comprise at least one SH moiety, can be selected from proteins, peptides, and polymers as a complexation agent.

In other embodiments, the therapeutic circular DNA vector according to the invention may be administered naked in a suitable buffer without being associated with any further vehicle, transfection, or complexation agent.

### VI. Methods of Use

Provided herein are methods of inducing expression (e.g., persistent expression) of a therapeutic sequence in a subject in need thereof (e.g., as part of a gene therapy regimen) by administering to the subject any of the therapeutic circular DNA vectors, or pharmaceutical compositions thereof, described herein. Target cells or tissues of a subject can be characterized by examining a nucleic acid sequence (e.g., an RNA sequence, e.g., an mRNA sequence) of the host cell, such as by Southern Blotting or PCR analysis, to detect or quantify the presence (e.g., persistence) of the therapeutic sequence delivered. Alternatively, expression of the therapeutic sequence in the subject can be characterized (e.g., quantitatively or qualitatively) by monitoring the progress of a disease being treated by delivery of the therapeutic sequence (e.g., associated with a defect or mutation targeted by the therapeutic sequence). In some embodiments, transcription or expression (e.g., persistent transcription or persistent expression) of the therapeutic sequence is confirmed by observing a decline in one or more symptoms associated with the disease.

Accordingly, the invention provides methods of treating a disease in a subject by administering to the subject any of the therapeutic circular DNA vectors, or pharmaceutical compositions thereof, described herein. Any of the therapeutic circular DNA vectors, or pharmaceutical compositions thereof, described herein can be administered to a subject in a dosage from 1 μg to 10 mg of DNA (e.g., from 5 μg to 5.0 mg, from 10 μg to 2.0 mg, or from 100 μg to 1.0 mg of DNA, e.g., from 10 μg to 20 μg, from 20 μg to 30 μg, from 30 μg to 40 μg, from 40 μg to 50 μg, from 50 μg to 75 μg, from 75 μg to 100 μg, from 100 μg to 200 μg, from 200 μg to 300 μg, from 300 μg to 400 μg, from 400 μg to 500 μg, from 500 μg to 1.0 mg, from 1.0 mg to 5.0 mg, or from 5.0 mg to 10 mg of DNA, e.g., about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 600 μg, about 700 μg, about 750 μg, about 1.0 mg, about 2.0 mg, about 2.5 mg, about 5.0 mg, about 7.5 mg, or about 10 mg of DNA).

In some embodiments, administration of a therapeutic circular DNA vector of the invention, or a pharmaceutical composition thereof, is less likely to induce an immune response in a subject compared with administration of other gene therapy vectors (e.g., plasmid DNA vectors and viral vectors).

In some instances, the therapeutic circular DNA vectors, and pharmaceutical compositions thereof, provided herein are amenable to repeat dosing due to their ability to transfect target cells without triggering an immune response or inducing a reduced immune response relative to a reference vector, such as a plasmid DNA vector or an AAV vector, as discussed above. Thus, the invention provides methods of repeatedly administering the therapeutic circular DNA vectors and pharmaceutical compositions described herein. Any of the aforementioned dosing quantities may be repeated at a suitable frequency and duration. In some embodiments, the subject receives a dose about twice per day, about once per day, about five times per week, about four times per week, about three times per week, about twice per week, about once per week, about twice per month, about once per month, about once every six weeks, about once every two months, about once every three months, about once every four months, twice per year, once yearly, or less frequently. In some embodiments, the number and frequency of doses corresponds with the rate of turnover of the target cell. It will be understood that in long-lived post-mitotic target cells transfected using the vectors described herein, a single dose of vector may be sufficient to maintain expression of the heterologous gene within the target cell for a substantial period of time. Thus, in other embodiments, a therapeutic circular DNA vector provided herein may be administered to a subject in a single dose. The number of occasions in which a therapeutic circular DNA vector is delivered to the subject can be that which is required to maintain a clinical (e.g., therapeutic) benefit.

Methods of the invention include administration of a therapeutic circular DNA vector or pharmaceutical composition thereof through any suitable route. The therapeutic circular DNA vector or pharmaceutical composition thereof can be administered systemically or locally, e.g., intravenously, ocularly (e.g., intravitreally, subretinally, by eye drop, intraocularly, intraorbitally), intramuscularly, intravitreally (e.g., by intravitreal injection), intradermally, intrahepatically, intracerebrally, intramuscularly, percutaneously, intraarterially, intraperitoneally, intralesionally, intracranially, intraarticularly, intraprostatically, intrapleurally, intratracheally, intrathecally, intranasally, intravaginally, intrarectally, intratumorally, subcutaneously, subconjunctivally, intravesicularly, mucosally, intrapericardially, intraumbilically, orally, topically, transdermally, by inhalation, by aerosolization, by injection (e.g., by jet injection), by electroporation, by implantation, by infusion (e.g., by continuous infusion), by localized perfusion bathing target cells directly, by catheter, by lavage, in creams, or in lipid compositions.

Therapeutic circular DNA vectors described herein can be delivered into cells via in vivo electrotransfer (e.g., in vivo electroporation). In vivo electroporation has been demonstrated in certain tissues, such as eye, skin, skeletal muscle, certain tumor types, and lung epithelium. Delivery of naked DNA into cells by in vivo electroporation involves administration of the DNA into target tissue, followed by application of electrical field to temporarily increase cell membrane permeability within the tissue by generating pores, allowing the DNA molecules to cross cell membranes. As an example, delivery to skin using in vivo electroporation is described in Cha & Daud Hum. Vaccin. Immunother. 2012, 8(11):1734-1738, which is incorporated by reference in its entirety. In vivo electroporation of skeletal muscle is described in Sokolowska & Blachnio-Zabielska, Int. J Molecular Sci. 2019, 20:2776, which is incorporated by reference in its entirety. Intratumoral delivery using in vivo electroporation is described in Aung et al. Gene Therapy 2009, 16:830-839, which is incorporated by reference in its entirety. In vivo electroporation of DNA into lung cells is described in Pringle et al. J. Gene Med. 2007, 9:369-380, which is incorporated by reference in its entirety. In vivo electrotransfer of circular DNA vectors to cells in the eye (e.g., retinal cells and/or photoreceptor cells) is described in International Patent Publication No. WO 2022/198138, which is incorporated by reference in its entirety. In some instances, after administration of the circular DNA vector to the eye, an electrode can be positioned within the interior of the eye (e.g., within about 1 mm from the retina), and an electric field can be transmitted through the electrode into a target ocular tissue at conditions suitable for electrotransfer of the circular DNA vector into the target cell (e.g., by applying six to ten pulses from 10-100 V each). Devices and systems having electrodes suitable for transmitting electric fields in mammalian tissues are commercially available and can be useful in the methods disclosed herein. In some instances, the electric field is transmitted through an electrode comprising a needle (e.g., a needle positioned within the vitreous humor or in the subretinal space). Suitable needle electrodes include CLINIPORATOR® electrodes marketed by IGEA® and needle electrodes marketed by AMBU®. Methods of the invention include administration of any of the therapeutic circular DNA vectors described herein, or pharmaceutical compositions thereof, to skin, skeletal muscle, tumors (including, e.g., melanomas), eye, and lung via in vivo electrotransfer.

Additionally, or alternatively, therapeutic circular DNA vectors or pharmaceutical compositions thereof can be administered to host cells ex vivo, such as by cells explanted from an individual patient, followed by reimplantation of the host cells into a patient, e.g., after selection for cells which have incorporated the vector. Thus, in some aspects, the disclosure provides transfected host cells and methods of administration thereof for treating a disease.

Additionally or alternatively, the present invention includes methods of treating a subject having a disease or disorder by administering to the subject the isolated DNA vector (or a composition thereof) of the invention.

Assessment of the efficiency of transfection of any of the therapeutic circular DNA vectors described herein can be performed using any method known in the art or described herein. Isolating a transfected cell can also be performed in accordance with standard techniques. For example, a cell comprising a therapeutic gene can express a visible marker, such as a fluorescent protein (e.g., GFP) or other reporter protein, encoded by the sequence of the heterologous gene that aids in the identification and isolation of a cell or cells comprising the heterologous gene. Cells containing a therapeutic gene can also be characterized by examining the nucleic acid sequence (e.g., an RNA sequence, e.g., an mRNA sequence) of the host cell, such as by Southern Blotting or PCR analysis, to assay for the presence of the heterologous gene contained in the vector.

Accordingly, methods of the present invention include, after administering any of the therapeutic circular DNA vectors described herein to a subject, subsequently detecting the expression of the heterologous gene in the subject. Expression can be detected one week to four weeks after administration, one month to four months after administration, four months to one year after administration, one year to five years after administration, or five years to twenty years after administration (e.g., at least one week, at least two weeks, at least one month, at least four months, at least one year, at least two years, at least five years, at least ten years after administration). At any of these detection timepoints, persistence (e.g., episomal persistence) of the DNA vector may be observed. In some embodiments, the persistence of the circular DNA vector is from 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector (e.g., a circular vector produced in bacteria or having one or more bacterial signatures not present in the vector of the invention).

Additionally, or alternatively, any of the therapeutic circular DNA vectors of the invention can be administered to host cells ex vivo, such as cells explanted from an individual patient, followed by reimplantation of the host cells into a patient, e.g., after selection for cells which have incorporated the vector. Thus, in some aspects, the disclosure provides transfected host cells (e.g., electrotransfected host cells), methods of transfecting host cells, and methods of administering host cells to a subject, e.g., for treating a disease in the subject. In some instances, any of the therapeutic circular DNA vectors described herein can be transfected into host cells by electroporation using known methods and devices (e.g., by NEON transfection (Thermo Fisher) or flow electroporation chambers (e.g., as described in U.S. Pat. No. 9,546,350 or U.S. Patent Publication No. 2020/0131500, each of which is incorporated by reference)).

### VII. Kits and Articles of Manufacture

In another aspect of the invention, an article of manufacture or a kit containing any of the therapeutic circular DNA vectors, or pharmaceutical compositions thereof, described herein. The article of manufacture includes a container and a label or package insert on or associated with the container. Suitable containers include, for example, bottles, vials, syringes, IV solution bags, etc. The containers may be formed from a variety of materials, such as glass or plastic. The container holds a composition which is by itself or combined with another composition effective for treating, preventing and/or diagnosing a condition and may have a sterile access port (for example the container may be an intravenous solution bag or a vial having a stopper pierceable by a hypodermic injection needle). At least one active agent in the composition is a therapeutic circular DNA vector of the invention or a pharmaceutical composition comprising the therapeutic circular DNA vector. The label or package insert indicates that the composition is used for treating the condition treatable by its contents. Moreover, the article of manufacture may comprise (a) a first container with a composition contained therein, wherein the composition comprises a therapeutic circular DNA vector, or pharmaceutical composition thereof, and (b) a second container with a composition contained therein, wherein the composition comprises an additional therapeutic agent. The article of manufacture may further comprise a package insert indicating that the compositions can be used to treat a particular condition. Alternatively, or additionally, the article of manufacture may further comprise a second (or third) container comprising a pharmaceutically acceptable carrier, such as bacteriostatic water for injection (BWFI), phosphate-buffered saline, Ringer's solution, dextrose solution, or any of the pharmaceutically acceptable carriers disclosed above. It may further include other materials desirable from a commercial and user standpoint, including other buffers, diluents, filters, needles, syringes, or other delivery devices.

In some instances, kits provided herein include any of the therapeutic circular DNA vectors or compositions thereof (e.g., pharmaceutical compositions) described herein (or produced by the methods described herein) and instructions for expressing the therapeutic circular DNA vector in a cell, or a culture of cells, using electroporation (e.g., in vitro or ex vivo electroporation) or electrotransfer (e.g., in vivo electrotransfer).

### EXAMPLES

### Example 1. Generation and Purification of Closed Circular DNA Using Two Restriction Enzymes

Therapeutic circular DNA vectors can be produced through a cell-free process using plasmid DNA vectors as a template, which can be amplified into concatemers by rolling circle amplification. This Example compares a process involving gel extraction of digested DNA with a streamlined process in which no gel extraction is required. In the streamlined process, restriction digestion is followed immediately by ligation (i.e., without gel extraction), and undesired products are purified using a second restriction enzyme in lieu of gel extraction.

The process described in this Example involves two different restriction enzymes: a first restriction enzyme to cut two sites located between the plasmid backbone and the desired DNA sequence, and a second restriction enzyme to cut at least once within the plasmid backbone to digest the plasmid backbone.

FIGS. 1A-1E illustrate such a dual-enzyme process. First, the plasmid DNA containing two EcoRI restriction sites flanking the closed circular DNA sequence and two PvuII restriction sites within the plasmid DNA sequence is amplified by Phi29 in a rolling circle amplification reaction. The sample is heat-inactivated at 65° C. Next, EcoRI is added to the sample to cut out the desired closed circular DNA insert and separate it from the plasmid DNA backbone. The reaction is stopped by another heat inactivation step at 65° C. Next, both linear products are intramolecularly self-ligated using T4 ligase, and yet another heat inactivation step is performed at 65° C. The sample is then contacted with PvuII to digest the plasmid backbone, leaving the closed circular DNA intact. Closed circular DNA can be supercoiled using gyrase and purified of plasmid backbone fragments using an exonuclease, such as T5 exonuclease or Plasmid-Safe (Lucigen).

### Example 2. Process for Generation and Purification of Closed Circular DNA Using a Single, Type ITs Restriction Enzyme to Cut Two Sites on a Template DNA

Therapeutic circular DNA vectors are generated from plasmid DNA using a single, type ITs restriction enzyme to cut two sites flanking the therapeutic sequence. FIGS. 2A-2F illustrate such a single-enzyme process.

A plasmid DNA is used as a template. The plasmid contains a therapeutic sequence (represented in FIGS. 2A and 2B as C3 region/Payload) and a backbone sequence (depicted in FIGS. 2A and 2B as the sequence containing the Rep origin, resistance genes, and BsaI recognition sites). Prior to restriction digest, the template can be amplified by rolling circle amplification (e.g., using Phi29 polymerase).

Next, BsaI and ligase are added. BsaI enzymes recognize the two BsaI recognition sites within the backbone and each cuts the template (which can be a circular template or an amplified concatemer resulting from rolling circle amplification) at a cut site between the therapeutic sequence and the recognition site to generate a linear therapeutic fragment and a linear backbone fragment. The linear therapeutic fragment contains the therapeutic sequence, and the linear backbone fragment contains the backbone sequence and the two BsaI recognition sites. Upon ligation, the linear therapeutic fragment circularizes into a therapeutic circular DNA vector as shown in FIGS. 2C and 2D, and the linear backbone fragment circularizes as shown in FIGS. 2E and 2F. Because the circular backbone fragment contains BsaI sites and ligation occurs in the presence of the BsaI enzyme, BsaI can cut the backbone and does not cut the therapeutic circular DNA vector, thereby driving the reaction forward toward a purer therapeutic circular DNA product. Exonuclease is added to digest the remaining linear backbone, and gyrase is added to supercoil the therapeutic circular DNA vector.

### Example 3. Process for Generation and Purification of Closed Circular DNA Using a Single, Type IIs Restriction Enzyme to Cut Four Sites on a Template DNA

Therapeutic circular DNA vectors were generated from plasmid DNA using a single, type IIs restriction enzyme to cut (1) two sites flanking the desired DNA sequence and (2) twice within the plasmid backbone to digest the plasmid backbone.

FIGS. 3A-3D illustrate such a single-enzyme process. First, the plasmid DNA containing two BsaI restriction sites flanking the closed circular DNA sequence and two PvuII restriction sites within the plasmid DNA sequence were amplified by Phi29 in a rolling circle amplification reaction. The sample was heat-inactivated at 65° C. Next, BsaI was added to the sample to cut at four sites within each unit amplicon to cut out the desired closed circular DNA insert and separate it from the plasmid DNA backbone and to digest the plasmid backbone. The reaction was stopped by heat inactivation at 80° C. T4 ligase was added to self-ligate the closed circular DNA. During self-ligation of closed circular DNA, fragments of plasmid DNA self-ligate. Accordingly, after 65° C. heat inactivation of the ligation reaction, a final BsaI digest step was carried out to digest the plasmid backbone. Closed circular DNA was then supercoiled using gyrase and purified of plasmid backbone fragments using Plasmid-Safe.

FIG. 4A-4C depict the three closed circular DNA constructs generated as described above. Construct 1103 is a 1431 bp single transcription unit (TU) vector, including a single CMV promoter (PcMv), a transgene, and a polyA tail. Construct 1147 is a 6293 bp multi-TU vector, including four transgenes, each flanked by a promoter and a polyA tail. Construct 1258 is a 5065 bp multi-cistronic vector, including three transgenes flanked by a single promoter and a single polyA tail.

Various reaction conditions were tested on the three constructs. Animal-free BsaI (New England Biolabs) was compared to standard BsaI. In addition, random hexamer primers were tested alongside specific primers, although primer quantities were not equivalent (random primers were used in greater quantity than specific primers). Digestion controls were included (lanes 7 and 10), in which a PvuII cut site was included in the closed circular DNA transgene. Table 1, below, describes the reaction conditions for each well.

TABLE 1

| Reaction conditions | | | |
|---|---|---|---|
| | Construct | | |
| Lane | ID | Primer | Enzyme |
| 1 | 1103 | Random | Standard Bsal |
| 2 | 1103 | Random | Animal-free BsaI |
| 3 | 1103 | Specific | Standard Bsal |
| 4 | 1103 | Specific | Animal-free BsaI |
| 5 | 1147 | Random | Standard BsaI |
| 6 | 1147 | Random | Animal-free BsaI |
| 7* | 1147 | Random | EcoRI |
| 8 | 1147 | Specific | Standard BsaI |
| 9 | 1147 | Specific | Animal-free Bsal |
| 10* | 1147 | Specific | EcoRI |
| 11 | 1258 | Random | Standard BsaI |
| 12 | 1258 | Random | Animal-free Bsal |

A simulation gel showing theoretical bands after the digestion step is shown in FIG. 5B, and its corresponding actual gel is shown in FIG. 5A. Lane 1 of the simulation corresponds to the banding pattern expected in lanes 1-4 of the actual gel; lane 2 of the simulation gel corresponds to the banding pattern expected in lanes 5, 6, 8, and 9 of the actual gel; lane 3 of the simulation gel corresponds to the banding pattern expected in lanes 7 and 10 of the actual gel; and lane 4 of the simulation gel corresponds to the banding pattern expected in lanes 11 and 12 of the actual gel. All banding patterns appeared as expected.

A gel showing banding patterns following ligation in vitro is shown in FIG. 5C. Intermediate ligation products were observed in each well.

FIG. 5D shows banding patterns after exonuclease digestion. Each of lanes 1-4 contained a single prominent band corresponding to a DNA size of 1431 bp, the expected size of closed circular DNA of sample 1103. Each of lanes 5 and 6 contained a single prominent band corresponding to a DNA size of 6293 bp, the expected size of closed circular DNA of sample 1147. Each of lanes 11 and 12 contained a single prominent band corresponding to a DNA size of 5065 bp, the expected size of closed circular DNA of sample 1258. Digestion controls in lanes 7 and 10 did not contain a prominent single band, as expected due to the presence of a PvuII cut site within the closed circular DNA transgene.

### Example 4. Streamlined Process for Generating and Purifying c3DNA Using a Single, Type IIs Restriction Enzyme

Closed circular DNA was generated from plasmid DNA using a single, type IIs restriction enzyme as described in Example 3 (referred to in this Example as “Condition 1”) and compared to a streamlined variation of the process in which the BsaI restriction digest was combined with the ligation step (“Condition 2”). Conditions 1 and 2 are shown in FIG. 6.

The streamlined process was achieved using a type IIs restriction enzyme, BsaI, by leveraging its ability to cut outside the recognition sequence. Applicant exploited this property to ensure that, upon self-ligation of the closed circular DNA, no recognition site was present in the closed circular DNA. An exemplary template plasmid DNA vector for such a process is shown in FIGS. 7A and 7B. Upon self-ligation of plasmid backbone, a BsaI sites are limited to backbone byproducts, leading to enzymatic cleavage of the plasmid backbone. As a result, the combined digestion/ligation reaction proceeds to digest the plasmid backbone, while self-ligated closed circular DNA accumulates without further digestion.

This streamlined process precludes the need to heat-inactivate the restriction digest prior to ligation. Instead, heat-inactivation was performed after ligation. T4 ligase was inactivated while leaving BsaI activity intact by raising the reaction temperature to 65° C., which is sufficient to inactivate T4 ligase but insufficient to inactivate BsaI.

In the present experiment, random primers and animal-free BsaI were used. Heat inactivation was carried out for ten minutes, followed by addition of DTT to final concentration of 1 mM and T5 exonuclease. FIG. 8A shows a simulated banding pattern, and FIG. 8B shows the actual gel. Lanes 1-3 contain product from Condition 1, while lanes 4-6 contain product from Condition 2. Lanes 1 and 4 contain construct 1103 (1431 bp), lanes 2 and 5 contain construct 1147 (6293 bp), and lanes 3 and 6 contain construct 1258 (5065 bp). 1 Kb Plus DNA Ladder (Left marker, Invitrogen, Waltham, MA.) and supercoiled DNA ladder (right marker, New England Biolabs, Ipswich, MA.) were used. Both conditions resulted in production of correctly sized c3DNA for all three constructs tested. Thus, by utilizing unique properties of type IIs restriction enzymes, such as BsaI, it was confirmed that c3DNA production prior to supercoiling was carried out in a single reaction vessel without buffer exchange, which makes it simpler and allows for higher throughput.

### Example 5. Cell-Free Production of c3DNA Using a Single Restriction Digest

### A. Methods

The following reagents were mixed in 1×Phi29 buffer (New England Biolabs) to prepare the rolling circle amplification (RCA) solution: plasmid DNA (5 μg/mL final concentration); random primers (50 μM final concentration); NaOH (10 mM final concentration); dNTPs (2 mM final concentration); bovine serum albumin (BSA) (0.2 mg/mL final concentration); Phi29 DNA polymerase (200 U/mL final concentration); and pyrophosphatase stock (New England Biolabs; 0.4 mU/mL final concentration). The RCA solution was continuously mixed for 18 hours at 30° C.

After incubation, the RCA solution was heat-inactivated by raising the temperature to 65° C. for 45 minutes. The temperature of the inactivated RCA solution was then reduced to 25° C.

To produce the BsaI solution, the inactivated RCA solution (0.2 mg DNA/mL final concentration) was added to rCutSmart buffer (New England Biolabs; 1× final concentration) containing BsaI (2.5 U/μg DNA final concentration). The BsaI solution was continuously mixed for two hours at 37° C. No heat-inactivation was carried out on the BsaI solution. The temperature of the digested BsaI solution was reduced to 25° C.

To produce the ligation solution, the digested BsaI solution (40 μg DNA/mL final concentration) was added to rCutSmart buffer containing T4 ligase (10 U T4 ligase per μg DNA) and ribo ATP (1 mM final concentration). The ligation solution was incubated for two hours at 25° C.

After incubation, the ligation solution was heat-inactivated by raising the temperature to 65° C. for 45 minutes. The temperature of the inactivated ligation solution was then reduced to 37° C.

To produce the supercoiling solution, the ligation solution was added to gyrase buffer containing DNA gyrase (1.5 U gyrase per g DNA). Gyrase buffer contains 35 mM Tris-HCl, 24 mM KCl, 4 mM MgCl2, 1 mM ATP, 2 mM DTT, 1.8 mM spermidine, 6.5% glycerol (w/v), and 100 μg/mL BSA. The supercoiling solution was continuously mixed for at least two hours at 37° C. No heat-inactivation was carried out on the supercoiling solution.

Next, the supercoiling solution was added to potassium acetate buffer (50 mM potassium acetate, final concentration) containing T5 exonuclease (2.5 U T5 exonuclease per μg DNA) to produce the cleanup solution. The cleanup solution was continuously mixed for at least two hours at 37° C. No heat-inactivation was carried out on the cleanup solution.

The cleanup solution was then sterile-filtered through a 0.22 m filter and diluted 1:1 in a buffer containing 1.5 M NaCl, 100 mM MOPS, 30% isopropyl alcohol (IPA), and 0.3% Triton X-100 (v/v) to achieve a final concentration of 750 mM NaCl, 50 mM MOPS, 15% isopropyl alcohol (IPA), and 0.15% Triton X-100 (v/v). Diluted cleanup solution was added to Qiagen plasmid prep columns, DNA was washed with QC buffer, and DNA was eluted with QN buffer. Eluate was diluted with IPA (40% v/v) and centrifuged at 4° C. for 30 minutes at 15,000 g. The pellet was washed with 70% EtOH and centrifuged again at 4° C. for 30 minutes at 15,000 g. After the second centrifugation, the pellet was resuspended in PBS at concentrations from 1.0 mg/mL to 2.0 mg/mL.

For scaled-up production of c3DNA, discussed below, supercoiled monomer was calculated by densitometry analysis of gel electrophoresis preparations using Image Lab software (BIO-RAD®). 200 ng c3DNA sample was loaded into tris-acetate-EDTA gels and electrophoresis was run at 110 V for 40 minutes prior to staining with 1% EtBr for 20 minutes. For each c3DNA sample, the target band was identified according to its size compared to supercoiled ladder, and “Band Detection Sensitivity” was set as a “Custom Sensitivity” at a value of 50 in “Detection Settings.”

### B. Results

Production of 12.75 kb c3DNA

The methods described above were adapted to a bench-scale production of a 12.75 kb c3DNA construct, starting with 0.15 mg of template, in which two lots of gyrase were tested. FIG. 9 shows bands corresponding to 12.75 kb c3DNA recovered after T5 exonuclease digestion for both gyrase lots (lanes 1 and 2).

Large-Scale Production of c3DNA

One advantage of the streamlined, cell-free processes of embodiments disclosed herein, such as those that do not include in-process gel extraction, is that they are suitable for scalability to large-scale production of therapeutic circular DNA vector (i.e., producing at least 2 mg circular DNA vector per batch). Reaction volumes in large-scale production methods can be at least 100 mL, at least 1 L, at least 10 L, at least 100 L, at least 500 L, or greater, and the yield of therapeutic circular DNA vector can be at least 2 mg per liter of initial reaction volume (e.g., rolling circle amplification solution).

The methods described in Example 5A above were scaled up to generate larger quantities of c3DNA containing a variety of therapeutic sequences, ranging in size, number of transcription units, and number of regulatory elements. An exemplary large-scale production involves volumes of 150 mL of RCA solution, 500 mL of BsaI solution, 2.5 L of ligation solution, about 3.2 L of supercoiling solution, about 3.2 L of cleanup solution, and 2-20 mL of c3DNA product.

After a single purification step, the quantity of c3DNA in each lot exceeded 2 mg, corresponding to at least three-fold greater quantity of c3DNA product relative to the plasmid DNA vector from which it was produced. Each lot of c3DNA was verified for protein expression in vitro (data not shown), endotoxin level of no greater than 0.5 EU/mL, and percent supercoiled monomer of 70% or higher.

TABLE 2
Summary of c3DNA constructs produced by cell-free
production using a single restriction digest

| Composition | Size (bp) |
|---|---|
| [P]-[RD] | 2744 |
| [P]-[RD] | 3677 |
| [P]-[RD] | 2383 |
| [P]-[TD] | 8482 |
| [P]-[TD] | 7985 |
| [P]-[TD]-[TD]-[TD] | 4537 |
| [P]-[TD]-[A]-[P]-[TD]- | 8035 |
| [A]-[P]-[TD] | |
| [P]-[TD] | 3466 |
| [P]-[TD] | 8656 |
| [P]-[RD]-[TD]-[R] | 10927 |
| [P]-[RD]-[TD]-[R] | 8425 |
| [R]-[P]-[R]-[RD]-[R]-[A] | 5162 |
| [P]-[R]-[RD]-[A] | 2991 |

[P] = Promoter;
[TD] = Therapeutic protein-encoding domain;
[RD] = Reporter protein-encoding domain;
[A] = Poly-adenylation site;
[R] = Regulatory element

### Example 6. Effects of Varying Ligation Conditions

Following digestion with a restriction enzyme, a ligation reaction was performed on linear DNA in which the total DNA concentration was varied from 40 ng/μL to 100 ng/μL. As shown in FIG. 10, 20 ng/μL of plasmid DNA (pDNA) was treated with ligase as a reference (lanes 2 and 3), and 20 ng/μL of closed circular DNA was treated with ligase as a reference (lanes 4 and 5). Lanes 6 and 7 show 40 ng/μL linear DNA, lanes 8 and 9 show 100 ng/μL linear DNA, and lanes 10 and 11 show 40 ng/μL linear DNA without buffer. As shown in lanes 8 and 9, a large undesired band appears, indicative of an undesired intermolecular ligation. In contrast, lanes 6 and 7 show less undesired product, indicating that reduced linear DNA concentration reduced intermolecular ligation (undesired) and increased intramolecular self-ligation (desired).

FIG. 11 shows another ligation experiment in which the amount of ligase enzyme was varied. On the left of the gel is a marker and a control reaction of the linear DNA previously treated with a restriction enzyme. Lanes 1-3 show 20 μg/mL of DNA treated with 100 U/μg ligase, 20 U/μg ligase, and 5 U/μg ligase, respectively. Lanes 4-6 show 40 μg/mL of DNA treated with 100 U/μg ligase, 20 U/μg ligase, and 5 U/μg ligase, respectively. Lanes 7-9 show 100 μg/mL of DNA treated with 100 U/μg ligase, 20 U/μg ligase, and 5 U/μg ligase, respectively. Samples 4-9 were diluted to 20 μg/mL prior to sample loaded, so that lanes 1-9 all contain the same amount of total DNA. As shown in lanes 3, 6, and 9, a decrease in ligase concentration to 5 U/μg ligase produced the largest amount of desired intramolecular ligation product, denoted as the desired band.

### Example 7. Ligase Composition Comparison

In this study, three different ligase enzymes, T3, T4, and T7, were purchased from New England Biolabs for comparison as reagents for synthetic C³DNA production. BsaI digestion was performed with a BsaI concentration of 2.5 U BsaI per g DNA (500 U/mL) for 3 hours and 42 minutes. Ligation reactions were carried out on BsaI-digested DNA samples (construct size of 9,542 bp) in rCUTSMART® buffer containing ATP (1 mM), without polyethylene glycol (PEG). Conditions for each sample are summarized in Table 3, below:

TABLE 3
Sample conditions for ligase comparison study

| Sample | | DNA | Ligase |
|---|---|---|---|
| Number | Ligase | concentration | concentration |
| 1 | T4 | 40 μg/mL | 10 U/μg DNA |
| 2 | T4 | 80 μg/mL | 5 U/μg DNA |
| 3 | T4 | 80 μg/mL | 10 U/μg DNA |
| 4 | T3 | 40 μg/mL | 10 U/μg DNA |
| 5 | T3 | 80 μg/mL | 5 U/μg DNA |
| 6 | T7 | 40 μg/mL | 10 U/μg DNA |
| 7 | T7 | 80 μg/mL | 5 U/μg DNA |

Ligase reactions were carried out over various time courses for each sample, and samples were exposed to enzyme heat-inactivation at the end of ligation. Samples were collected at various timepoints and for analysis after gyrase treatment and a subsequent T5 exonuclease digest.

FIG. 13 shows gel profiles of each sample after ligation. Desired therapeutic vector (C³DNA) band is shown within the black box. Samples 1-5 (T3 ligase and T4 ligase) showed similar gel profiles, whereas Samples 6 and 7 (T7 ligase) show fewer bands and nearly invisible desired bands.

FIGS. 14 and 15 show gel profiles for time course studies for T4 ligase (FIG. 14) and T3 and T7 ligases (FIG. 15). Results are plotted in FIG. 16 to show ligation kinetics as a reduction of linear DNA over time for each sample. This study suggests that T4 ligase exhibited the fastest ligation kinetics, and T7 ligase exhibited minimal ligation activity. T3 exhibited effective ligase activity, albeit with slower kinetics than T4 ligase. Together, these results show that both T3 and T4 were suitable ligases for self-ligation of synthetic circular DNA.

### Example 8. Maintenance of High Efficiency Ligation with Streamline Modifications

In an endeavor to improve manufacturing efficiency by reducing reaction volume and decreasing process duration, Applicant systematically studied the impact on product yield resulting from two process modifications—(1) increasing DNA concentration in the ligation reaction; and (2) removing post-ligation heat inactivation. Specifically, Applicant sought to (1) reduce the volume of the ligation reaction by increasing the concentration of DNA in a smaller ligation reaction volume and (2) expedite the process by removing the heat inactivation step after ligation (a time-consuming step requiring up to two hours). Each of these two process modifications was expected to adversely impact yield of ligated DNA. Surprisingly, adopting both of these process modifications had no substantial adverse impact on yield, indicating that a process incorporating both modifications could substantially improve manufacturability without sacrificing efficiency. Study details are provided below:

In the first experiment within this study, the effect of post-ligation heat-kill was assessed on constructs of different sizes—5,065 kb and 8,656 kb (SEQ ID NO: 1). Briefly, the production was carried out as follows: DNA was amplified using Phi29 rolling circle amplification starting with plasmid DNA at a concentration of 5 μg/mL, Phi29 at 200 U/mL, and dNTPs at 2 mM, for 18 hours, 14 minutes. BsaI digestion was carried out for 2 hours and 10 minutes with BsaI at a concentration of 2.5 U/ug (500 U/mL), and ligation was performed using 10 U/ug T4 ligase on 40 μg/mL DNA. Heat-inactivation was performed after ligation on only the samples indicated. All samples were then supercoiled with gyrase, followed by T5 exonuclease digestion and purification according to methods described above. Each construct was tested with and without heat-kill immediately after ligation, and samples were tested in duplicate. Sample identities are summarized in Table 4 below:

TABLE 4
Sample summary for heat kill removal

| Sample | Construct | Heat |
|---|---|---|
| Number | Size | Kill? |
| 1 | 5,065 | Yes |
| 2 | 5,065 | Yes |
| 3 | 5,065 | No |
| 4 | 5,065 | No |
| 5 | 8,656 | No |
| 6 | 8,656 | No |
| 7 | 8,656 | Yes |
| 8 | 8,656 | Yes |

FIG. 17 is a gel showing banding patterns after ligation, before heat kill. Desired bands are indicated by boxes, and no differences were observed among each construct type, as expected.

Post-gyrase gels are shown in FIG. 18A for samples 1-4 and FIG. 18B for samples 5-8, and post-exonuclease gels are shown in FIG. 19A for samples 1-4 and FIG. 19B for samples 5-8. Results of post-purification gels were quantified and are shown below in Table 5 and in FIG. 20. Yield was calculated by dividing the mass of total C³DNA product by the mass of total DNA post-BsaI digest.

TABLE 5
Results of heat kill removal study

| | | Post- | |
|---|---|---|---|
| | | Ligase | |
| Sample | | Heat | |
| Number | Yield (mg) | Kill? | % Yield |
| 1 | 0.515 | Yes | 7.0 |
| 2 | 0.518 | Yes | 7.1 |
| 3 | 0.843 | No | 11.5 |
| 4 | 0.805 | No | 10.9 |
| 5 | 0.687 | No | 9.3 |
| 6 | 0.410 | No | 5.6 |
| 7 | 0.290 | Yes | 3.9 |
| 8 | 0.284 | Yes | 3.9 |

Removal of heat kill had no adverse effect on yield. In fact, yield unexpectedly improved across every sample in which heat kill was removed. Specifically, yield of the 5,065 bp construct was improved by 59%, and yield of the 8,656 construct was improved by 91% (taking mean values across duplicates). This result shows that removal of heat kill from the synthetic C³DNA production process could improve manufacturing efficiency by meaningfully reducing process time (heat kill had previously taken 1.5-2 hours).

Next, the effect of increasing DNA concentration in the ligation reaction (ligation intensification) was assessed using the 8,656 kb construct as a model construct. Relative order of supercoiling by gyrase and exonuclease digestion with T5 exonuclease (i.e., gyrase before T5 exonuclease vs T5 exonuclease before gyrase) was also compared within each DNA concentration. Other conditions (amplification, BsaI digestion, and ligation) were the same as in the heat kill removal study with the exception of the DNA concentration at ligation, which is shown in Table 6, below, for each sample. DNA was diluted to each given concentration from 133 μg/mL, the DNA concentration immediately after BsaI digestion, measured by Qubit.

TABLE 6
Sample identification for DNA concentration

| | DNA | | |
|---|---|---|---|
| Sample | concentration at | Post-Ligase | T5/Gyrase |
| Number | ligase reaction | Heat-kill? | Order |
| 1 | 40 μg/mL | Yes | Gyrase → T5 |
| 2 | 40 μg/mL | No | Gyrase → T5 |
| 3 | 40 μg/mL | No | T5 → Gyrase |
| 4 | 80 μg/mL | No | Gyrase → T5 |
| 5 | 80 μg/mL | No | T5 → Gyrase |
| 6 | 160 μg/mL | No | Gyrase → T5 |
| 7 | 160 μg/mL | No | T5 → Gyrase |

Samples were carried through production through gyrase/exonuclease processing and run on gels prior to purification. Supercoiled monomer percent as measured post-exonuclease/pre-purification for each sample is shown in Table 7, below:

TABLE 7
Purity as measured pre-purification

| Sample | % Supercoiled |
|---|---|
| Number | monomer |
| 1 | 86% |
| 2 | 78% |
| 3 | 78% |
| 4 | 78% |
| 5 | 84% |
| 6 | 77% |
| 7 | 83% |

As shown in the gel profiles (FIG. 21) and relative quantification of yield (FIG. 22), doubling DNA concentration from 40 μg/mL to 80 μg/mL at the ligation step had only a minor effect on yield and purity relative to Sample 1; however, a second doubling of DNA concentration to 160 μg/mL had a more substantial (negative) impact on yield (see Samples 6 and 7, FIG. 22). Moreover, placing T5 exonuclease digest prior to gyrase had minimal impact on yield (FIG. 22), while increasing purity (Table 7).

Next, select samples were carried through downstream purification to produce drug substance. As shown in the gel profiles (FIG. 23) and relative quantification of yield (FIG. 24), removal of heat-inactivation resulted in substantial increase in yield of drug substance. By running the T5 exonuclease step prior to gyrase-mediated supercoiling, the previous purity was maintained with full removal of low molecular weight species.

Together, these results show, surprisingly, that purity and yield can be maintained despite (a) increasing the DNA concentration in the ligation reaction to reduce the necessary reaction volume; and (b) removing the time-consuming post-ligation heat inactivation step. A process involving these modifications (and, optionally, performing exonuclease digest before supercoiling) therefore offers benefits in manufacturability (e.g., smaller required reactors and less processing time, and ability to use single-use vessels incompatible with heat-kill) without sacrificing product quality, representing a substantial improvement in synthetic DNA manufacturing.

### Example 9: Improved Gyrase Efficiency

In processes described above that involve supercoiling before exonuclease digestion, the lowest concentration of gyrase used was 1.5 U/ug. This example describes a titration study intended to determine whether lower concentrations of gyrase were feasible in view of new process modifications, such as performing exonuclease digestion before supercoiling.

The production was carried out as follows: Amplified DNA was produced using Phi29 rolling circle amplification using a starting plasmid DNA concentration of 5 μg/mL, Phi29 concentration of 200 U/mL, dNTP concentration of 2 mM, and a duration of 18 hours, 49 minutes. BsaI digestion was carried out using a BsaI concentration of 2.5 U/μg DNA (500 U/mL) and a DNA concentration of 200 μg/mL, for a duration of 4 hours, 5 minutes. Ligation was performed using 10 U/ug T4 ligase on 80 μg/mL DNA. No heat-inactivation was conducted after ligation. Rather, immediately after ligation, T5 exonuclease was added at a concentration of 2.5 U/ug DNA. Next, three concentrations of gyrase were tested; 1.5 U/ug, 1.0 U/μg, and 0.5 U/μg. Results were observed by gel electrophoresis (relative quantification and average adjusted concentration by Qubit).

Notably, no substantial change in desired product intensity or purity was observed at decreased gyrase concentrations (FIG. 25 and Table 8). Additionally, minimal change in undesired band intensity was observed across the three samples (FIG. 25), suggesting minimal impact to product quality at lower gyrase concentrations.

TABLE 8
Gyrase study results

| | | | Avg. Adj. Conc. | Purity (C³DNA |
|---|---|---|---|---|
| | Gyrase | Relative | (μg/mL) | monomer band |
| Lane | concentration | Quant | by Qubit | %) |
| 1 | 1.5 U/ug DNA | 1.00 | 2.96 | 92.4 |
| 2 | 1.0 U/ug DNA | 1.17 | 2.97 | 90.5 |
| 3 | 0.5 U/ug DNA | 1.02 | 3.11 | 89.3 |

Importantly, these results suggest that process efficiency can be meaningfully improved by reducing the minimal effective amount of gyrase usage when exonuclease digestion is performed before supercoiling.

### Example 10: Effect of Type IIs Overhang Sequence and Number of Cut Sites

In previous Examples, the overhang sequence AAAA was used as the BsaI overhang sequence flanking the therapeutic sequence. The overhang sequence AAAA was selected due to its low-efficiency, which, without being bound by theory, was hypothesized to be advantageous to skew the kinetics toward intramolecular ligation (self-ligation; desired) rather than intermolecular ligation (ligation with another therapeutic sequence; undesired). However, the type IIs restriction processes described herein allow for selection of desired overhang sequences. Accordingly, Applicant tested a second overhang sequence—AACC.

The present study also included an assessment of the effect of the number of BsaI cut sites within the template plasmid (i.e., whether the template plasmid contained only two BsaI cut sites flanking the therapeutic sequence or, alternatively, more than two BsaI cut sites, wherein additional cut sites are within the backbone).

Constructs having two different sizes were tested. The study design is shown in Table 9, below:

TABLE 9
Study design

| Sample | Construct | Overhang | Number of |
|---|---|---|---|
| Number | size | sequence | cut sites |
| 1 | 10,927 bp | AAAA | 5 |
| 2 | 10,927 bp | AACC | 2 |
| 3 | 8,425 bp | AAAA | 5 |
| 4 | 8,425 bp | AAAA | 2 |
| 5 | 8,425 bp | AACC | 2 |

For this study, the production was carried out generally as follows: Phi29 amplification (starting DNA concentration of 5 μg/mL, Phi29 concentration of 200 U/mL, dNTPs at a concentration of 2 mM, and primers at a concentration of 50 μM, for a duration of 18 hours, 55 minutes)→BsaI digestion (2.5 U/ug (500 U/mL), starting DNA concentration of 200 μg/mL, for a duration of 4 hours, 18 minutes)→ligation (DNA concentration of 40 μg/mL, T4 ligase at a concentration of 10 U/ug DNA)→heat kill→supercoiling→T5 exonuclease digestion→column purification. Samples were retained at various timepoints within the BsaI digestion step, ligation step, and T5 exonuclease step to compare the rate of reaction between samples. Percent supercoiled monomer was quantified on the final product at the end of the production, using gel analysis methods described above.

Results from the BsaI digestion time course show similar banding profiles and intensities between 30-minute, 60-minute, and 120-minute timepoints (FIG. 26). No differences in digestion kinetics were observed from this gel. These results indicate that the BsaI reaction was highly efficient, occurring primarily within the first 30 minutes of the reaction.

Results from the ligation time course study (FIGS. 27A and 27B) show that constructs having two BsaI cut sites and AACC overhangs exhibited stronger desired monomer bands (indicated by white arrows) at the 18-hour timepoint (FIG. 27A). Sample 5 exhibited a high ligation efficiency, with similar banding profile between one-hour, three-hour, and 18-hour timepoints (FIG. 27A). Sample 4 showed similar banding profiles and ligation efficiencies as Sample 3 (FIG. 27A). Samples 1, 3, and 4 exhibited increased C³DNA monomer band intensity from three hours through 24 hours (FIG. 27B). In both construct sizes, samples with AACC ligase overhangs led to faster self-ligation kinetics than AAAA Samples 1, 3, and 4. There was no visually observed difference between three-hour and 24-hour timepoint for either sample containing AACC overhangs. These results indicate that AACC ligase overhangs conferred substantially faster self-ligation kinetics, which can allow for shorter overall process durations and increased C³DNA monomer yields.

An effect of number of cut sites was also observed, albeit to a lesser degree than the overhang sequence. The lower band in the white box on Sample 4 in FIG. 27B corresponds with its 8.4 kb linear C³DNA. For Sample 4 (AAAA with two cut sites), the intensity of the lower band decreases as the reaction proceeded from three hours to 18 hours to 24 hours. In contrast, the corresponding band of Sample 3 (AAAA with five cut sites) remained the same throughout the entire 24-hour time course. These results indicate that the reaction kinetics were faster for Sample 4 than Sample 3. The corresponding band was not visible in any timepoint for Sample 5 (AACC with two cut sites), indicating faster self-ligation kinetics than either Sample 3 or 4.

Table 10, below, shows relative quantifications of the C³DNA monomer band post-ligation at the 18-hour timepoint. Relative quantification was executed on the C³DNA monomer band, utilizing the five-cut site reference sequence as reference (Sample is the reference for Sample 2, and Sample 3 was the reference for Samples 4 and 5).

TABLE 10
Relative quantification of post-ligase C³DNA monomer band (18 hours)

| Sample | | Overhang | Number of | Relative |
|---|---|---|---|---|
| Number | Construct size | sequence | cut sites | Quantification |
| 1 | 10,927 bp | AAAA | 5 | 1.00 |
| 2 | 10,927 bp | AACC | 2 | 1.60 |
| 3 | 8,425 bp | AAAA | 5 | 1.0 |
| 4 | 8,425 bp | AAAA | 2 | 1.56 |
| 5 | 8,425 bp | AACC | 2 | 1.90 |

These results verify the visual observations that, in the first 18 hours of ligation reaction, (i) reduction to two BsaI cut sites moderately improved C3DNA self-ligation, and (ii) changing from AAAA to AACC ligase overhangs substantially boosted self-ligation formation.

FIG. 28A shows gel profiles of Samples 1-5 after T5 exonuclease treatment. Here, Sample 2 (AACC with two cut sites) had a substantially stronger C³DNA monomer band (darkest band in each lane) than Sample 1 (AAAA with five cut sites). Visual differences were less pronounced across Samples 3-5. Relative quantification was executed on the post-exonuclease samples and is shown in Table 11, below. References were taken as described previously.

TABLE 11
Relative quantification of post-exonuclease C3DNA monomer band

| Sample | Construct | Overhang | Number of | Replicate | Replicate |
|---|---|---|---|---|---|
| Number | size | sequence | cut sites | 1 | 2 |
| 1 | 10,927 bp | AAAA | 5 | 1.00 | 1.00 |
| 2 | 10,927 bp | AACC | 2 | 1.23 | 1.48 |
| 3 | 8,425 bp | AAAA | 5 | 1.00 | 1.00 |
| 4 | 8,425 bp | AAAA | 2 | 0.85 | 0.96 |
| 5 | 8,425 bp | AACC | 2 | 1.06 | 1.02 |

AACC overhang sequences conferred higher yield for both constructs, relative to AAAA overhangs.

At the post-exonuclease timepoint, results were further quantified by Qubit, with different operators producing results for each replicate (FIG. 28B). Results across operators were similar, showing replicability. Like the gel quantifications, Qubit results also showed that pivoting from ligase overhang AAAA to AACC yielded substantially higher counts following 18-hour exonuclease digest. Additionally, reducing the number of BsaI cut sites from five to two without altering ligase overhangs (Sample 4 vs Sample 3) produced similar counts, as measured by Qubit.

FIG. 29 shows Qubit results post-exonuclease treatment over the course of 18 hours, replicated by two operators A and B. Without being bound by theory, decreasing counts over time reflect consumption by exonuclease of non-supercoiled DNA (linear and nicked DNA) into nucleotides. Trendlines suggest that the rate of exonuclease digestion slowed (or plateaued) between three hours and 18 hours (36.7%-48.7% (excluding one outlier) decrease in counts within the first three hours versus >88% by 18 hours). Total count reduction at 18 hours exonuclease digest across operators is shown in Table 12, below:

TABLE 12
Linear and nicked DNA reduction efficiency at 18 hours
exonuclease digestion

| | | | | Operator A | Operator B |
|---|---|---|---|---|---|
| Sample | Construct | Overhang | Number of | % | % |
| Number | size | sequence | cut sites | reduction | reduction |
| 1 | 10,927 bp | AAAA | 5 | 91.2 | 91.9 |
| 2 | 10,927 bp | AACC | 2 | 87.2 | 87.2 |
| 3 | 8,425 bp | AAAA | 5 | 89.6 | 88.9 |
| 4 | 8,425 bp | AAAA | 2 | 88.0 | 86.1 |
| 5 | 8,425 bp | AACC | 2 | 85.2 | 87.6 |

Final product yields were measured across two operators. Mean and standard deviation for each samples are shown in Table 13, below.

TABLE 13
Final product total yields

| | | | | Mean | |
|---|---|---|---|---|---|
| Sample | Construct | Overhang | Number of | C³DNA | Standard |
| Number | size | sequence | cut sites | yield (mg) | Deviation |
| 1 | 10,927 bp | AAAA | 5 | 0.465 | 0.092 |
| 2 | 10,927 bp | AACC | 2 | 0.61 | 0.028 |
| 3 | 8,425 bp | AAAA | 5 | 0.68 | 0.014 |
| 4 | 8,425 bp | AAAA | 2 | 0.68 | 0.014 |
| 5 | 8,425 bp | AACC | 2 | 0.71 | 0.042 |

For both construct sizes, total C³DNA product yield was increased in samples containing the AACC overhang with two cut sites relative to the AAAA overhang with five cut sites. This effect was more pronounced in the 10,927 bp construct than the 8,425 bp construct (30% increase vs 4.400 increase).

Together, these results show that C³DNA having an AACC overhang sequence can be produced with unexpectedly faster kinetics and improved product yield, relative to AAAA overhang-containing C³DNA. Reducing the number of cut sites can also improve manufacturability, although the impact of this modification did not appear as substantial as the AACC overhang in this study.

### Example 11: Head-to-Head Comparison of AAAA+5 Cut Sites Vs. AACC+2 Cut Sites Across Various Conditions

In this study, two constructs of different sizes were each produced by two different restriction processes, with each restriction process tested across four different conditions. The two constructs correspond to C³DNA vector sizes of 8,656 bp (the “8.7 kb construct”) and 10,300 bp (the “10.3 kb construct”). Both constructs contain a CAG promoter and an ABCA4-encoding sequence; the 10.3 kb construct includes an additional regulatory element downstream of the ABCA-encoding sequence (FIG. 30). Each construct was made with the following two restriction processes: (1) BsaI overhang of AAAA with four backbone fragments (AAAA) and (2) BsaI overhang of AACC with one backbone fragment (AACC). Constructs and restriction processes are illustrated in FIG. 30. Plasmid maps are shown for the 8.7 kb construct with AAAA restriction process (FIG. 31; SEQ ID NO: 2) and the 8.7 kb construct with AACC restriction process (FIG. 32; SEQ ID NO: 4). Nucleic acid sequences of the final therapeutic circular C³DNA vector are provided for the 8.7 kb construct with AAAA restriction process (SEQ ID NO: 1) and the AACC restriction process (SEQ ID NO: 3).

The four conditions were:
(1) Phi29 amplification→BsaI digestion→ligation (40 μg/mL T4 ligase)→heat kill→supercoiling→T5 exonuclease digestion→column purification;
(2) Phi29 amplification→BsaI digestion→ligation (80 μg/mL T4 ligase) (no heat kill)→supercoiling→T5 exonuclease digestion→column purification;
(3) Phi29 amplification→BsaI digestion→ligation (40 μg/mL T4 ligase) (no heat kill)→T5 exonuclease digestion→supercoiling→column purification; and
(4) Phi29 amplification→BsaI digestion→ligation (40 μg/mL T4 ligase) (no heat kill)→supercoiling→T5 exonuclease digestion→column purification.

For each condition, Phi29 amplification was conducted with a starting plasmid DNA concentration of 5 μg/mL (90 μg starting plasmid DNA in each sample), random hexamer primers at 50 μM, dNTPs at 2 mM, Phi29 polymerase at 200 U/mL, and for a duration of about 19 hours; BsaI digestion was conducted with a BsaI concentration of 2.5 U/ug DNA (500 U/mL), for a duration of about three hours; supercoiling was conducted with 1.5 U gyrase per g DNA, DNA concentration from 3-10 μg/mL, for four hours; and T5 exonuclease digestion was conducted with 2.5 U T5 exonuclease per g DNA for about 18 hours.

In addition to the differences noted above, Condition 4 included a smaller-scale amplification step relative to Conditions 1-3 (⅓ quantity of DNA template at the start of amplification) and included alternative buffer conditions (Conditions 1-3 include buffers as described in Example 5). Samples were taken after ligation, gyrase, and T5 exonuclease steps and run on gels to quantify yield.

### 8.7 kb Construct

Gel profiles are shown for the 8,656 bp construct at ligase end of run (EOR) in FIG. 33, gyrase EOR for Conditions 1, 2, and 4 in FIG. 34, T5 exonuclease EOR for Condition 3 in FIG. 35, and T5 exonuclease EOR for conditions 1, 2, and 4 and gyrase EOR for Condition 3 in FIG. 36. At each EOR, desired bands appear stronger for AACC than AAAA under all conditions (FIGS. 33-36). Band intensity for FIG. 36 was quantified, and yields are shown in Table 14, below:

TABLE 14
Yields for 8.7 kb construct across restriction processes
and conditions-desired band (monomer) intensity

| | | C³ Band | AACC yield |
|---|---|---|---|
| | | Adj. | improvement |
| Lane | Sample | Volume | factor |
| 1 | AAAA Condition 1 | 20170925 | n/a |
| 2 | AACC Condition 1 | 31481195 | 1.56 |
| 3 | AAAA Condition 2 | 13265610 | n/a |
| 4 | AACC Condition 2 | 22332305 | 1.68 |
| 5 | AAAA Condition 3 | 35522180 | n/a |
| 6 | AACC Condition 3 | 44448710 | 1.25 |
| 7 | AAAA Condition 4 | 23671225 | n/a |
| 8 | AACC Condition 4 | 47154175 | 1.99 |

Yield results were also quantified by Qubit assay for each of the samples. Mass values quantified by Qubit were multiplied by the Band % of Table 14 to calculate an improvement factor (of desired product), as shown in Table 15, below:

TABLE 15
Yields for 8.7 kb construct across restriction processes
and conditions-Qubit

| | | | Mass * | Yield |
|---|---|---|---|---|
| | | | Desired | Improvement |
| Lane | Sample | Mass (ug) | band % | factor |
| 1 | AAAA Condition 1 | 475 | 451 | n/a |
| 2 | AACC Condition 1 | 695 | 595 | 1.32 |
| 3 | AAAA Condition 2 | 453 | 453 | n/a |
| 4 | AACC Condition 2 | 663 | 550 | 1.22 |
| 5 | AAAA Condition 3 | 692 | 647 | n/a |
| 6 | AACC Condition 3 | 1082 | 898 | 1.39 |
| 7 | AAAA Condition 4 | 597 | 597 | n/a |
| 8 | AACC Condition 4 | 1507 | 1245 | 2.08 |

As shown in Table 15, yield improvements were also captured by Qubit assay with a similar range of improvement between AAAA and AACC restriction processes. The AACC restriction process exhibited increased yield of desired product (C³DNA) across all four conditions. Overall, AACC exhibited a 20-40% improvement in yield over AAAA across all conditions.

10.3 kb Construct Gel profiles are shown for the 10.3 kb construct at ligase HOR in FIG. 37, gyrase EOR for Conditions 1, 2, and 4 in FIG. 38, and T5 exonuclease EOR for Conditions 1, 2, and 4 and gyrase EOR for Condition 3 in FIG. 39. Generally, desired bands appear stronger for AACC than AAAA. Band intensity is for FIG. 39 was quantified, and yields are shown in Table 16, below:

TABLE 16
Yields for 10.3 kb construct across restriction processes
and conditions-desired band (monomer) intensity

| | | C³ Band | AACC Yield |
|---|---|---|---|
| | | Adj. | improvement |
| Lane | Sample | Volume | factor |
| 1 | AAAA Condition 1 | 16969848 | n/a |
| 2 | AACC Condition 1 | 37096248 | 2.19 |
| 3 | AAAA Condition 2 | 9172296 | n/a |
| 4 | AACC Condition 2 | 17876376 | 1.95 |
| 5 | AAAA Condition 3 | 11740764 | n/a |
| 6 | AACC Condition 3 | 28915320 | 2.46 |
| 7 | AAAA Condition 4 | 42832440 | n/a |
| 8 | AACC Condition 4 | 15498924 | 0.36 |

Qubit assays were performed for each of the 10.3 kb samples. Mass values quantified by Qubit were multiplied by the Band 00 of Table 16 to calculate an improvement factor (of desired product), as shown in Table 17, below:

TABLE 17
Yields for 8.7 kb construct across restriction processes
and conditions-Qubit

| | | | Mass * | Yield |
|---|---|---|---|---|
| | | Mass | Desired | Improvement |
| Lane | Sample | (ug) | band % | factor |
| 1 | AAAA Condition 1 | 285 | 285 | n/a |
| 2 | AACC Condition 1 | 1198 | 1076 | 3.77 |
| 3 | AAAA Condition 2 | 270 | 270 | n/a |
| 4 | AACC Condition 2 | 790 | 660 | 2.44 |
| 5 | AAAA Condition 3 | 223 | 189 | n/a |
| 6 | AACC Condition 3 | 919 | 778 | 4.12 |
| 7 | AAAA Condition 4 | 883 | 747 | n/a |
| 8 | AACC Condition 4 | 401 | 401 | 0.54 |

As shown in Table 17, and similar to the 8.7 kb construct, 10.3 kb construct yield 10 improvements were also captured by Qubit assay with a similar range of improvement between AAAA and AACC restriction processes. The AACC restriction process exhibited increased yield of desired product (C³DNA monomer) across Conditions 1-3.

DNA quantities for each construct and condition were quantified at various points along the production process. Each sample had an initial DNA quantity (plasmid DNA) of 90 μg. C³DNA mass was measured at the end of the process, and C³DNA mass was divided by initial plasmid DNA mass to show the ratio of C³DNA product to initial quantity. Results are shown in Table 18, below.

TABLE 18
DNA quantities summary

| | | Initial | Post-BsaI | | C³DNA:initial |
|---|---|---|---|---|---|
| | | DNA | digestion | C³DNA | plasmid DNA |
| Construct | Condition | (mg) | DNA (mg) | (mg) | (mass/mass) |
| 8.7 kb | 1 | 0.090 | 12.2 | 0.45 | 5.01 |
| AAAA | 2 | 0.090 | 12.9 | 0.45 | 5.03 |
| | 3 | 0.090 | 12.0 | 0.65 | 7.19 |
| | 4 | 0.090 | 18.7 | 0.60 | 6.64 |
| 8.7 kb | 1 | 0.090 | 9.9 | 0.59 | 6.61 |
| AACC | 2 | 0.090 | 9.6 | 0.55 | 6.11 |
| | 3 | 0.090 | 9.7 | 0.90 | 9.98 |
| | 4 | 0.090 | 14.0 | 1.24 | 13.83 |
| 10.3 kb | 1 | 0.090 | 11.4 | 0.29 | 3.17 |
| AAAA | 2 | 0.090 | 11.3 | 0.27 | 3.01 |
| | 3 | 0.090 | 9.5 | 0.19 | 2.1 |
| | 4 | 0.090 | 21.3 | 0.75 | 8.3 |
| 10.3 kb | 1 | 0.090 | 9.5 | 1.08 | 11.95 |
| AACC | 2 | 0.090 | 8.9 | 0.66 | 7.34 |
| | 3 | 0.090 | 8.9 | 0.78 | 8.65 |
| | 4 | 0.090 | 7.4 | 0.40 | 4.45 |

Together, these results, which span both 8.7 kb and 10.3 kb constructs and various conditions described throughout the present specification, are consistent with observations of Example 10 and further suggest that the AACC restriction process (AACC overhang with 1 backbone fragment) can increase C³DNA yield, offering an unexpected and useful improvement in synthetic circular DNA manufacturability.

### Numerated Paragraphs

1. A method of producing a therapeutic circular DNA vector, the method comprising:
(a) providing a sample comprising a template DNA vector comprising a therapeutic sequence and a backbone sequence;
(b) amplifying the template DNA vector using a polymerase-mediated rolling-circle amplification to generate a linear concatemer;
(c) digesting the linear concatemer with a type IIs restriction enzyme that cuts a first site and a second site per unit of the linear concatemer, wherein the first and second sites flank the therapeutic sequence and form self-complementary overhangs, thereby producing a linear therapeutic fragment and a linear backbone fragment, wherein the linear therapeutic fragment comprises the therapeutic sequence and the linear backbone fragment comprises at least a portion of the backbone sequence and a type IIs restriction site; and
(d) contacting the linear backbone fragment and the linear therapeutic fragment with a ligase to produce a circular backbone comprising the type IIs restriction site and a therapeutic circular DNA vector lacking a type IIs restriction site.

2. The method of paragraph 1, wherein the type IIs restriction enzyme cuts the circular backbone and does not cut the therapeutic circular DNA vector.

3. A method of producing a therapeutic circular DNA vector, the method comprising:
(a) providing a sample comprising a template DNA vector comprising a therapeutic sequence and a backbone sequence;
(b) amplifying the template DNA vector using a polymerase-mediated rolling-circle amplification to generate a linear concatemer;
(c) digesting the linear concatemer with one or more restriction enzymes that cut at least a first site, a second site, and a third site per unit of the linear concatemer, wherein: (i) the first and second sites flank the therapeutic sequence and form self-complementary overhangs, and (ii) the third site is within the backbone sequence and forms an overhang that is non-complementary to the first or second site, thereby producing a linear therapeutic fragment comprising the therapeutic sequence and at least two linear backbone fragments each comprising a portion of the backbone sequence; and
(d) contacting the linear therapeutic fragment with a ligase to produce a therapeutic circular DNA vector in solution.

4. The method of paragraph 3, wherein the linear concatemer is digested with a single restriction enzyme that cuts the first site, the second site, and the third site.

5. The method of paragraph 3, wherein the one or more restriction enzymes cut a fourth site of the linear concatemer per unit, wherein the fourth site is within the backbone sequence and forms an overhang that is non-complementary to the first or second site, and wherein the digestion produces at least three linear backbone fragments each comprising a portion of the backbone sequence.

6. The method of paragraph 5, wherein the single restriction enzyme cuts a fourth site of the linear concatemer per unit, wherein the fourth site is within the backbone sequence and forms an overhang that is non-complementary to the first or second site, and wherein the digestion produces at least three linear backbone fragments each comprising a portion of the backbone sequence.

7. The method of any one of paragraphs 1, 2, 4, and 6, wherein the restriction enzyme is a type IIs restriction enzyme.

8. The method of paragraph 7, wherein the type IIs restriction enzyme is BsaI.

9. The method of any one of paragraphs 1-8, wherein no restriction enzyme inactivation step precedes step (d).

10. The method of any one of paragraphs 1-9, wherein no temperature increase is performed between steps (c) and (d).

11. The method of any one of paragraphs 1-10, wherein steps (c) and (d) occur simultaneously.

12. The method of any one of paragraphs 1-11, further comprising raising the temperature of the solution containing the therapeutic circular DNA vector to about 65° C.

13. The method of any one of paragraphs 1-12, further comprising:
(e) contacting the therapeutic circular DNA vector with a topoisomerase or a helicase.

14. The method of paragraph 13, wherein step (e) is performed at about 37° C.

15. The method of any one of paragraphs 1-14, further comprising:
(f) contacting the linear backbone fragments with an exonuclease.

16. The method of paragraph 15 wherein step (f) is performed at about 37° C.

17. The method of any one of paragraphs 1-12, further comprising:
(e) contacting the therapeutic circular DNA vector with a topoisomerase or a helicase; and
(f) contacting the linear backbone fragments with an exonuclease, wherein no enzyme inactivation step is performed between steps (e) and (f).

18. The method of paragraph 17, wherein step (e) occurs before step (f).

19. The method of any one of paragraphs 1-18, wherein the restriction enzyme is provided at a concentration from about 0.5 U/μg to about 20 U/μg.

20. The method of paragraph 19, wherein the restriction enzyme is provided at a concentration of about 2.5 U/μg.

21. The method of any one of paragraphs 1-20, wherein step (c) comprises incubation from one to 12 hours.

22. The method of paragraph 21, wherein step (c) comprises incubation for about one hour.

23. The method of any one of paragraphs 1-22, wherein the ligase is provided at a concentration no greater than 20 U ligase per μg DNA (U/μg).

24. The method of any one of paragraphs 1-23, wherein the ligase is T4 ligase.

25. The method of any one of paragraphs 13-24, wherein the topoisomerase is provided at a concentration no greater than 10 U topoisomerase per g DNA (U/μg).

26. The method of any one of paragraphs 13-25, wherein the topoisomerase is a type II topoisomerase.

27. The method of any one of paragraphs 13-26, wherein the topoisomerase is gyrase or topoisomerase IV.

28. The method of any one of paragraphs 15-27, wherein the exonuclease is provided at a concentration from about 0.5 U/μg to about 20 U/μg.

29. The method of any one of paragraphs 15-28, wherein step (f) is performed two or more times.

30. The method of any one of paragraphs 15-29, wherein step (f) comprises incubation from one hour to 12 hours.

31. The method of any one of paragraphs 15-30, wherein the exonuclease is T5 exonuclease.

32. The method of any one of paragraphs 1-31, further comprising:
(g) running the therapeutic circular DNA vector through a column; and/or (h) precipitating the therapeutic circular DNA vector with isopropyl alcohol.

33. The method of any one of paragraphs 1-32, wherein step (b) is performed using site-specific primers.

34. The method of any one of paragraphs 1-33, wherein step (b) is performed using random primers.

35. The method of any one of paragraphs 1-34, wherein the quantity of therapeutic circular DNA vector produced is at least five-fold the quantity of plasmid DNA vector in the sample of step (a).

36. The method of any one of paragraphs 1-35, wherein no DNA purification or gel extraction step is performed before step (d).

37. The method of any one of paragraphs 1-36, wherein the amount of the therapeutic circular DNA in the solution of step (d) is at least 2.0% of the amount of the linear concatemer in step (b) by weight.

38. The method of any one of paragraphs 1-37, wherein the amount of the therapeutic circular DNA produced in step (d) is at least 1.0 mg.

39. The method of any one of paragraphs 1-38, wherein the concentration of the therapeutic circular DNA in the solution after step (d) is at least 5 μg/mL without any purification or concentration being performed.

40. The method of any one of paragraphs 1-39, wherein the volume of the solution of step (d) is at least five liters.

41. The method of any one of paragraphs 1-40, wherein steps (b) through (d) are performed in a reaction vessel having a volume of at least one liter.

42. The method of any one of paragraphs 1-41, wherein the amount of the therapeutic circular DNA produced in step (d) is at least five-fold the amount of the template DNA vector provided in step (a).

43. A method of removing a backbone sequence from a DNA molecule to produce a therapeutic circular DNA vector, wherein the DNA molecule comprises the backbone sequence and a therapeutic sequence, the method comprising:
(a) digesting the DNA molecule with a type IIs restriction enzyme that cuts a first site and a second site per unit of the linear concatemer, wherein the first and second sites flank the therapeutic sequence and form self-complementary overhangs, thereby producing a linear therapeutic fragment and a linear backbone fragment, wherein the linear therapeutic fragment comprises the therapeutic sequence and the linear backbone fragment comprises at least a portion of the backbone sequence and a type IIs restriction site; and
(b) contacting the linear backbone fragment and the linear therapeutic fragment with a ligase to produce a circular backbone comprising the type IIs restriction site and a therapeutic circular DNA vector lacking a type IIs restriction site.

44. A method of removing a backbone sequence from a DNA molecule to produce a therapeutic circular DNA vector, wherein the DNA molecule comprises the backbone sequence and a therapeutic sequence, the method comprising:
(a) digesting the DNA molecule with one or more restriction enzymes that cut at least a first site, a second site, and a third site per unit of the DNA molecule, wherein: (i) the first and second sites flank the therapeutic sequence and form self-complementary overhangs, and (ii) the third site is within the backbone sequence and forms an overhang that is non-complementary to the first or second site, thereby producing a linear therapeutic fragment comprising the therapeutic sequence and at least two linear backbone fragments each comprising a portion of the backbone sequence; and
(b) contacting the linear therapeutic fragment with a ligase to produce a therapeutic circular DNA vector in solution.

45. The method of paragraph 44, wherein the linear concatemer is digested with a single restriction enzyme that cuts the first site, the second site, and the third site.

46. The method of paragraph 44, wherein the one or more restriction enzymes cut a fourth site of the DNA molecule, wherein the fourth site is within the backbone sequence and forms an overhang that is non-complementary to the first or second site, and wherein the digestion produces at least three linear backbone fragments each comprising a portion of the backbone sequence.

47. The method of paragraph 45, wherein the single restriction enzyme cuts a fourth site of the DNA molecule, wherein the fourth site is within the backbone sequence and forms an overhang that is non-complementary to the first or second site, and wherein the digestion produces at least three linear backbone fragments each comprising a portion of the backbone sequence.

48. The method of any one of paragraphs 44-47, wherein the DNA molecule is a concatemer produced by amplification of a template DNA vector.

49. The method of any one of paragraphs 44-47, wherein the DNA molecule is a template DNA vector.

50. The method of paragraph 49, wherein the template DNA vector is a plasmid DNA vector.

51. The method of any one of paragraphs 43-50, wherein the restriction enzyme is a type IIs restriction enzyme.

52. The method of paragraph 51, wherein the type IIs restriction enzyme is BsaI.

53. The method of any one of paragraphs 43-52, wherein no restriction enzyme inactivation step precedes step (b).

54. The method of any one of paragraphs 43-53, wherein no temperature increase is performed between steps (a) and (b).

55. The method of any one of paragraphs 43-54, wherein steps (a) and (b) occur simultaneously.

56. The method of any one of paragraphs 43-55, further comprising raising the temperature of the solution containing the therapeutic circular DNA vector to about 65° C. 57. The method of any one of paragraphs 43-56, further comprising:
(c) contacting the therapeutic circular DNA vector with a topoisomerase or a helicase.

58. The method of paragraph 57, wherein step (c) is performed at about 37° C.

59. The method of any one of paragraphs 43-58, further comprising:
(d) contacting the linear backbone fragments with an exonuclease.

60. The method of paragraph 59, wherein step (d) is performed at about 37° C.

61. The method of any one of paragraphs 43-60, further comprising:
(c) contacting the therapeutic circular DNA vector with a topoisomerase or a helicase; and
(d) contacting the linear backbone fragments with an exonuclease, wherein no enzyme inactivation step is performed between steps (c) and (d).

62. The method of paragraph 61, wherein step (c) occurs before step (d).

63. The method of any one of paragraphs 43-62, wherein the restriction enzyme is provided at a concentration of from about 0.5 U/μg to about 20 U/μg.

64. The method of paragraph 63, wherein the restriction enzyme is provided at a concentration of about 2.5 U/μg.

65. The method of any one of paragraphs 43-64, wherein step (a) comprises incubation from one to 12 hours.

66. The method of paragraph 65, wherein step (a) comprises incubation for about one hour.

67. The method of any one of paragraphs 43-66, wherein the ligase is provided at a concentration no greater than 20 U ligase per μg DNA (U/μg).

68. The method of any one of paragraphs 43-67, wherein the ligase is T4 ligase.

69. The method of any one of paragraphs 57-68, wherein the topoisomerase is provided at a concentration no greater than 10 U topoisomerase per g DNA (U/μg).

70. The method of any one of paragraphs 57-69, wherein the topoisomerase is a type II topoisomerase.

71. The method of any one of paragraphs 57-70, wherein the topoisomerase is gyrase or topoisomerase IV.

72. The method of any one of paragraphs 59-71, wherein the exonuclease is provided at a concentration from about 0.5 U/μg to about 20 U/μg.

73. The method of any one of paragraphs 59-72, wherein step (d) is performed two or more times.

74. The method of any one of paragraphs 59-73, wherein step (d) comprises incubation from one hour to 12 hours.

75. The method of any one of paragraphs 59-74, wherein the exonuclease is T5 exonuclease.

76. The method of any one of paragraphs 43-75, further comprising:
(e) running the therapeutic circular DNA vector through a column; and/or
(f) precipitating the therapeutic circular DNA vector with isopropyl alcohol.

77. The method of any one of paragraphs 43-76, wherein the therapeutic circular DNA vector is produced in the absence of a gel extraction step.

78. A method for large-scale production of a therapeutic circular DNA vector, the method comprising:
(a) providing a sample of a plasmid DNA vector comprising a therapeutic sequence and a backbone sequence;
(b) amplifying the plasmid DNA vector in a reaction volume of at least 500 mL using a polymerase-mediated rolling-circle amplification to generate a linear concatemer;
(c) digesting the linear concatemer with one or more restriction enzymes that cut at least a first site, a second site, and a third site per unit of the linear concatemer, wherein: (i) the first and second sites flank the therapeutic sequence and form self-complementary overhangs, and (ii) the third site is within the backbone sequence and forms an overhang that is non-complementary to the first or second site, thereby producing a linear therapeutic fragment comprising the therapeutic sequence and at least two linear backbone fragments each comprising a portion of the backbone sequence; and
(d) contacting the linear therapeutic fragment with a ligase to produce a therapeutic circular DNA vector in solution.

79. The method of paragraph 78, wherein the amount of the plasmid DNA vector provided in step (a) is at least 1.0 mg.

80. The method of paragraph 78 or 79, wherein step (b) produces at least 100 mg of the linear concatemer.

81. The method of any one of paragraphs 78-90, wherein step (d) produces at least 2.0 mg of the therapeutic circular DNA vector.

82. The method of any one of paragraphs 78-81, wherein steps (c) and (d) occur simultaneously.

83. The method of any one of paragraph 78-82, wherein no DNA purification is performed during or between steps (b), (c), and (d).

84. The method of any one of paragraphs 78-83, wherein the amount of the therapeutic circular DNA in the solution of step (d) is at least 2.0% of the amount of the linear concatemer in step (b) by weight

85. The method of any one of paragraphs 78-84, wherein the amount of the therapeutic circular DNA produced in step (d) is at least twice the amount of the plasmid DNA vector provided in step (a).

86. A method producing a therapeutic circular DNA vector, the method comprising:
(a) providing a solution comprising DNA molecules, wherein each DNA molecule comprises a backbone sequence and a therapeutic sequence;
(b) adding a type IIs restriction enzyme to the solution to digest the DNA molecules, thereby separating the backbone sequences from the therapeutic sequences;
(c) adding a ligase to the solution to produce a reaction in a mixture comprising:
   (i) the ligase;
   (ii) the type IIs restriction enzyme;
   (iii) therapeutic circular DNA vectors each comprising a single therapeutic sequence, wherein the therapeutic circular DNA vectors each lack a type IIs recognition site; and
   (iv) byproducts, wherein each byproduct comprises one or more type IIs restriction sites,
wherein the ratio of the therapeutic circular DNA vectors to the byproducts comprising one or more type IIs restriction sites increases as the reaction proceeds.

87. The method of paragraph 86, wherein some or all of the byproducts comprise one or more backbone sequences.

88. The method of paragraph 87, wherein some or all of the byproducts further comprise two or more therapeutic sequences.

89. The method of any one of paragraphs 86-88, wherein some or all of the byproducts are circularized.

90. The method of any one of paragraphs 86-89, wherein the DNA molecules of (a) are concatemers.

91. The method of any one of paragraphs 86-90, wherein the method further comprises, prior to step (a), amplifying a template DNA vector using rolling circle amplification to generate concatemers.

92. The method of any one of paragraphs 86-91, wherein the type IIs restriction enzyme is BsaI.

93. The method of any one of paragraphs 86-92, wherein no restriction enzyme inactivation step precedes step (d).

94. The method of any one of paragraphs 86-93, wherein no temperature increase is performed between steps (b) and (c).

95. The method of any one of paragraphs 86-94, further comprising raising the temperature of the solution containing the therapeutic circular DNA vector to about 65° C.

96. The method of any one of paragraphs 86-95, further comprising:
(e) contacting the therapeutic circular DNA vector with a topoisomerase or a helicase.

97. The method of paragraph 96, wherein step (e) is performed at about 37° C.

98. The method of any one of paragraphs 86-97, further comprising:
(f) contacting linear byproducts with an exonuclease.

99. The method of paragraph 98, wherein step (f) is performed at about 37° C.

100. The method of any one of paragraphs 86-95, further comprising:
(e) contacting the therapeutic circular DNA vector with a topoisomerase or a helicase; and
(f) contacting linear byproducts with an exonuclease, wherein no enzyme inactivation step is performed between steps (e) and (f).

101. The method of paragraph 100, wherein step (e) occurs before step (f).

102. The method of any one of paragraphs 86-101, wherein the restriction enzyme is provided at a concentration from about 0.5 U/μg to about 20 U/μg.

103. The method of paragraph 102, wherein the restriction enzyme is provided at a concentration of about 2.5 U/μg.

104. The method of any one of paragraphs 86-103, wherein step (c) comprises incubation from one to 12 hours.

105. The method of paragraph 104, wherein step (c) comprises incubation for about one hour.

106. The method of any one of paragraphs 86-105, wherein the ligase is provided at a concentration no greater than 20 U ligase per μg DNA (U/μg).

107. The method of any one of paragraphs 86-106, wherein the ligase is T4 ligase.

108. The method of any one of paragraphs 96-107, wherein the topoisomerase is provided at a concentration no greater than 10 U topoisomerase per μg DNA (U/μg).

109. The method of any one of paragraphs 96-108, wherein the topoisomerase is a type II topoisomerase.

110. The method of any one of paragraphs 96-109, wherein the topoisomerase is gyrase or topoisomerase IV.

111. The method of any one of paragraphs 98-110, wherein the exonuclease is provided at a concentration from about 0.5 U/μg to about 20 U/μg.

112. The method of any one of paragraphs 98-111, wherein step (f) is performed two or more times.

113. The method of any one of paragraphs 98-112, wherein step (f) comprises incubation from one hour to 12 hours.

114. The method of any one of paragraphs 98-113, wherein the exonuclease is T5 exonuclease.

115. The method of any one of paragraphs 86-114, further comprising:
(g) running the therapeutic circular DNA vector through a column; and/or
(h) precipitating the therapeutic circular DNA vector with isopropyl alcohol.

116. The method of any one of paragraphs 86-115, wherein step (b) is performed using site-specific primers.

117. The method of any one of paragraphs 86-116, wherein step (b) is performed using random primers.

118. The method of any one of paragraphs 86-117, wherein no gel extraction step is performed before step (d).

119. The method of any one of paragraphs 86-118, wherein the amount of the therapeutic circular DNA in the solution of step (d) is at least 2.0% of the amount of the DNA molecule in step (a) by weight.

120. The method of any one of paragraphs 86-119, wherein the amount of the therapeutic circular DNA produced in step (d) is at least 2.0 mg.

121. The method of any one of paragraphs 86-120, wherein the concentration of the therapeutic circular DNA in the solution after step (d) is at least 5.0 μg/mL prior to any purification or concentration being performed.

122. The method of any one of paragraphs 86-121, wherein the volume of the solution of step (d) is at least 5.0 liters.

123. The method of any one of paragraphs 86-122, wherein steps (b) through (d) are performed in a reaction vessel having a volume of at least 1.0 liter.

124. The method of any one of paragraphs 1-123, wherein the therapeutic sequence is greater than 5 kb.

125. The method of any one of paragraphs 1-124, wherein the therapeutic sequence comprises two or more transcription units.

126. The method of any one of paragraphs 1-125, wherein the therapeutic sequence encodes one or more therapeutic proteins.

127. The method of paragraph 126, wherein the one or more therapeutic proteins is a multimeric protein.

128. The method of any one of paragraphs 1-127, wherein the therapeutic sequence encodes a therapeutic nucleic acid.

129. The method of paragraph 128, wherein the therapeutic nucleic acid is an RNA molecule.

130. The method of paragraph 129, wherein the RNA molecule is a self-replicating RNA molecule, a short hairpin RNA, or a microRNA.

131. The method of any one of paragraphs 1-130, wherein the therapeutic circular DNA vector is formulated as a pharmaceutical composition.

132. The method of any one of paragraphs 1-131, further comprising formulating the therapeutic circular DNA vector in a pharmaceutically acceptable carrier to produce a pharmaceutical composition.

133. The method of paragraph 131 or 132, wherein the pharmaceutical composition comprises at least 1.0 mg of the therapeutic circular DNA vector in a pharmaceutically acceptable carrier.

134. The method of paragraph 132 or 133, wherein the therapeutic circular DNA vector in the pharmaceutical composition is at least 70% supercoiled monomer.

135. The method of any one of paragraphs 131-134, wherein the pharmaceutical composition comprises no more than 1.0% of residual protein or backbone sequence.

136. The method of any one of paragraphs 131-135, wherein the pharmaceutical composition comprises <1.0% protein content by mass, less than <1.0% RNA content by mass, and less than <5 EU/mg endotoxin.

137. A pharmaceutical composition produced by the method of any one of paragraphs 131-136.

138. A method of expressing a therapeutic sequence in an individual, wherein the method comprises administering to the individual the pharmaceutical composition of paragraph 137.

139. A method of treating a disease or disorder in an individual in need thereof, the method comprising administering to the individual the pharmaceutical composition of paragraph 137.

140. The method of paragraph 138 or 139, wherein the method comprises in vivo electrotransfer.

141. The method of paragraph 140, wherein the in vivo electrotransfer induces expression of the therapeutic sequence in skin, skeletal muscle, tumor, eye, or lung of the individual.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

SEQUENCES

| SEQ | | |
|---|---|---|
| ID | | |
| NO: | Description | Sequence |
| 1 | 8,656 bp | AAAACTCTTCGGTATCACAGGAGAATTTCAGGGAGACAT |
| | C3DNA with | TGATTATTGACTAGTTATTAATAGTAATCAATTACGGGGT |
| | AAAA | CATTAGTTCATAGCCCATATATGGAGTTCCGCGTTACATA |
| | overhang | ACTTACGGTAAATGGCCCGCCTGGCTGACCGCCCAACGA |
| | | CCCCCGCCCATTGACGTCAATAATGACGTATGTTCCCATA |
| | | GTAACGCCAATAGGGACTTTCCATTGACGTCAATGGGTG |
| | | GAGTATTTACGGTAAACTGCCCACTTGGCAGTACATCAA |
| | | GTGTATCATATGCCAAGTACGCCCCCTATTGACGTCAATG |
| | | ACGGTAAATGGCCCGCCTGGCATTATGCCCAGTACATGA |
| | | CCTTATGGGACTTTCCTACTTGGCAGTACATCTACGTATT |
| | | AGTCATCGCTATTACCATGTCGAGGTGAGCCCCACGTTCT |
| | | GCTTCACTCTCCCCATCTCCCCCCCCTCCCCACCCCCAATT |
| | | TTGTATTTATTTATTTTTTAATTATTTTGTGCAGCGATGGG |
| | | GGCGGGGGGGGGGGGGGGGCGCGCGCCAGGCGGGGCGG |
| | | GGCGGGGCGAGGGGCGGGGGGGGCGAGGCGGAGAGGT |
| | | GCGGCGGCAGCCAATCAGAGCGGCGCGCTCCGAAAGTTT |
| | | CCTTTTATGGCGAGGCGGCGGCGGCGGCGGCCCTATAAA |
| | | AAGCGAAGCGCGCGGCGGGCGGGAGTCGCTGCGACGCT |
| | | GCCTTCGCCCCGTGCCCCGCTCCGCCGCCGCCTCGCGCCG |
| | | CCCGCCCCGGCTCTGACTGACCGCGTTACTCCCACAGGTG |
| | | AGCGGGCGGGACGGCCCTTCTCCTCCGGGCTGTAATTAG |
| | | CGCTTGGTTTAATGACGGCTTGTTTCTTTTCTGTGGCTGCG |
| | | TGAAAGCCTTGAGGGGCTCCGGGAGGGCCCTTTGTGCGG |
| | | GGGGAGCGGCTCGGGGGGTGCGTGCGTGTGTGTGTGCGT |
| | | GGGGAGCGCCGCGTGCGGCTCCGCGCTGCCCGGCGGCTG |
| | | TGAGCGCTGCGGGCGCGGCGCGGGGCTTTGTGCGCTCCG |
| | | CAGTGTGCGCGAGGGGAGCGCGGCCGGGGGCGGTGCCCC |
| | | GCGGTGCGGGGGGGGCTGCGAGGGGAACAAAGGCTGCG |
| | | TGCGGGGTGTGTGCGTGGGGGGGTGAGCAGGGGGTGTGG |
| | | GCGCGTCGGTCGGGCTGCAACCCCCCCTGCACCCCCCTCC |
| | | CCGAGTTGCTGAGCACGGCCCGGCTTCGGGTGCGGGGCT |
| | | CCGTACGGGGCGTGGCGCGGGGCTCGCCGTGCCGGGCGG |
| | | GGGGTGGCGGCAGGTGGGGGTGCCGGGCGGGGCGGGGC |
| | | CGCCTCGGGCCGGGGAGGGCTCGGGGGAGGGGCGCGGC |
| | | GGCCCCCGGAGCGCCGGCGGCTGTCGAGGCGCGGCGAGC |
| | | CGCAGCCATTGCCTTTTATGGTAATCGTGCGAGAGGGCG |
| | | CAGGGACTTCCTTTGTCCCAAATCTGTGCGGAGCCGAAAT |
| | | CTGGGAGGCGCCGCCGCACCCCCTCTAGCGGGCGCGGGG |
| | | CGAAGCGGTGCGGCGCCGGCAGGAAGGAAATGGGCGGG |
| | | GAGGGCCTTCGTGCGTCGCCGCGCCGCCGTCCCCTTCTCC |
| | | CTCTCCAGCCTCGGGGCTGTCCGCGGGGGGACGGCTGCC |
| | | TTCGGGGGGGACGGGGCAGGGCGGGGTTCGGCTTCTGGC |
| | | GTGTGACCGGCGGCTCTAGACAATTGTACTAACCTTCTTC |
| | | TCTTTCCTCTCCTGACAGGGAGTTTAAACAGATAAGTTTG |
| | | TACAAAAAAGAGAGGTGCCACCATGGGCTTTGTGCGACA |
| | | GATTCAGCTGCTGCTGTGGAAGAACTGGACCCTGCGGAA |
| | | GCGGCAGAAAATCAGATTCGTGGTGGAACTCGTGTGGCC |
| | | CCTGAGCCTGTTTCTGGTGCTGATCTGGCTGCGGAACGCC |
| | | AATCCTCTGTACAGCCACCACGAGTGTCACTTCCCCAACA |
| | | AGGCCATGCCTTCTGCCGGAATGCTGCCTTGGCTGCAGG |
| | | GCATCTTCTGCAACGTGAACAACCCCTGCTTTCAGAGCCC |
| | | CACACCTGGCGAAAGCCCTGGCATCGTGTCCAACTACAA |
| | | CAACAGCATCCTGGCCAGAGTGTACCGGGACTTCCAAGA |
| | | GCTGCTGATGAACGCCCCTGAGTCTCAGCACCTGGGCAG |
| | | AATCTGGACCGAGCTGCACATCCTGAGCCAGTTCATGGA |
| | | CACCCTGAGAACACACCCCGAGAGAATCGCCGGCAGGGG |
| | | CATCAGAATCCGGGACATCCTGAAGGACGAGGAAACCCT |
| | | GACACTGTTCCTCATCAAGAACATCGGCCTGAGCGACAG |
| | | CGTGGTGTACCTGCTGATCAACAGCCAAGTGCGGCCCGA |
| | | GCAGTTTGCTCATGGCGTGCCGGATCTCGCCCTGAAGGAT |
| | | ATCGCCTGTTCTGAGGCCCTGCTGGAACGGTTCATCATCT |
| | | TCAGCCAGCGGAGAGGCGCCAAGACCGTCAGATATGCCC |
| | | TGTGCAGTCTGAGCCAGGGAACCCTGCAGTGGATCGAGG |
| | | ATACCCTGTACGCCAACGTGGACTTCTTCAAGCTGTTCCG |
| | | GGTGCTGCCCACACTGCTGGATTCTAGATCCCAGGGCATC |
| | | AACCTGAGAAGCTGGGGCGGCATCCTGTCCGACATGAGC |
| | | CCAAGAATCCAAGAGTTCATCCACCGGCCTAGCATGCAG |
| | | GACCTGCTGTGGGTTACCAGACCTCTGATGCAGAACGGC |
| | | GGACCCGAGACATTCACCAAGCTGATGGGCATTCTGAGC |
| | | GATCTGCTGTGCGGCTACCCTGAAGGCGGAGGATCTAGA |
| | | GTGCTGAGCTTCAATTGGTACGAGGACAACAACTACAAG |
| | | GCCTTCCTGGGCATCGACTCCACCAGAAAGGACCCCATC |
| | | TACAGCTACGACCGGCGGACAACCAGCTTCTGCAATGCC |
| | | CTGATCCAGAGCCTGGAAAGCAACCCTCTGACCAAGATC |
| | | GCTTGGAGGGCCGCCAAACCTCTGCTGATGGGAAAGATC |
| | | CTGTACACCCCTGACAGCCCTGCCGCCAGAAGAATCCTG |
| | | AAGAACGCCAACAGCACCTTCGAGGAACTGGAACACGTG |
| | | CGCAAGCTGGTCAAGGCCTGGGAAGAAGTGGGACCTCAG |
| | | ATTTGGTACTTCTTCGACAATAGCACCCAGATGAACATGA |
| | | TCAGAGACACCCTGGGCAACCCTACCGTGAAGGACTTCC |
| | | TGAACAGACAGCTGGGCGAAGAGGGCATTACCGCCGAG |
| | | GCCATCCTGAACTTTCTGTACAAGGGCCCCAGAGAGTCC |
| | | CAGGCCGACGACATGGCCAACTTCGATTGGCGGGACATC |
| | | TTCAACATCACCGACAGAACCCTGCGGCTGGTCAACCAG |
| | | TACCTGGAATGCCTGGTGCTGGACAAGTTCGAGAGCTAC |
| | | AACGACGAGACACAGCTGACCCAGAGAGCCCTGTCTCTG |
| | | CTGGAAGAGAATATGTTCTGGGCTGGCGTGGTGTTCCCC |
| | | GACATGTACCCTTGGACAAGCAGCCTGCCTCCTCACGTG |
| | | AAGTACAAGATCCGGATGGACATCGACGTGGTCGAAAAG |
| | | ACCAACAAGATCAAGGACCGGTACTGGGACAGCGGCCCT |
| | | AGAGCTGATCCCGTGGAAGATTTTCGGTACATCTGGGGC |
| | | GGATTCGCATACCTGCAGGACATGGTGGAACAGGGAATC |
| | | ACACGGTCCCAGGTGCAGGCTGAAGCTCCTGTGGGAATC |
| | | TACCTGCAGCAGATGCCTTATCCTTGCTTCGTGGACGACA |
| | | GCTTCATGATCATCCTGAATCGGTGCTTCCCCATCTTCAT |
| | | GGTGCTGGCCTGGATCTACTCCGTGTCTATGACCGTGAAG |
| | | TCCATCGTGCTGGAAAAAGAGCTGCGGCTGAAAGAGACA |
| | | CTGAAGAACCAGGGCGTGTCCAATGCCGTGATCTGGTGC |
| | | ACCTGGTTTCTGGACAGCTTCTCCATTATGAGCATGAGCA |
| | | TCTTTCTGCTGACGATCTTCATCATGCACGGCCGAATCCT |
| | | GCACTACAGCGACCCCTTTATCCTCTTCCTGTTCCTGCTG |
| | | GCCTTCAGCACCGCTACAATCATGCTGTGTTTTCTGCTGT |
| | | CCACCTTCTTCAGCAAGGCCTCTCTGGCCGCTGCTTGTAG |
| | | CGGCGTGATCTACTTCACCCTGTACCTGCCTCACATCCTG |
| | | TGCTTCGCATGGCAGGACAGAATGACCGCCGAGCTGAAG |
| | | AAAGCTGTGTCCCTGCTGAGCCCTGTGGCCTTTGGCTTTG |
| | | GCACCGAGTACCTCGTCAGATTTGAGGAACAAGGACTGG |
| | | GACTGCAGTGGTCCAACATCGGCAATAGCCCTACAGAGG |
| | | GCGACGAGTTCAGCTTCCTGCTGTCTATGCAGATGATGCT |
| | | GCTGGACGCCGCCGTGTATGGACTGCTGGCTTGGTATCTG |
| | | GACCAGGTGTTCCCAGGCGATTACGGCACTCCTCTGCCTT |
| | | GGTATTTCCTGCTGCAAGAGAGCTACTGGCTCGGCGGCG |
| | | AGGGATGTAGCACCAGAGAAGAAAGAGCCCTGGAAAAG |
| | | ACCGAGCCTCTGACCGAGGAAACAGAGGACCCTGAACAC |
| | | CCAGAGGGCATCCACGATAGCTTTTTCGAGAGAGAACAC |
| | | CCCGGCTGGGTGCCAGGCGTGTGTGTGAAGAATCTGGTC |
| | | AAGATTTTCGAGCCCTGCGGCAGACCTGCCGTGGACAGA |
| | | CTGAACATCACCTTCTACGAGAACCAGATTACCGCCTTTC |
| | | TGGGCCACAACGGCGCTGGCAAGACAACCACATTGAGCA |
| | | TCCTCACAGGCCTGCTGCCTCCAACAAGCGGCACAGTTCT |
| | | CGTTGGCGGCAGAGACATCGAGACAAGCCTGGATGCCGT |
| | | CAGACAGTCCCTGGGCATGTGCCCTCAGCACAACATCCT |
| | | GTTTCACCACCTGACCGTGGCCGAGCACATGCTGTTTTAT |
| | | GCCCAGCTGAAGGGCAAGAGCCAAGAAGAGGCTCAGCT |
| | | GGAAATGGAAGCCATGTTGGAGGACACCGGCCTGCACCA |
| | | CAAGAGAAATGAGGAAGCCCAGGATCTGAGCGGCGGCA |
| | | TGCAGAGAAAACTGAGCGTGGCCATTGCCTTCGTGGGCG |
| | | ACGCCAAGGTTGTGATCCTGGATGAGCCTACAAGCGGCG |
| | | TGGACCCTTACAGCAGAAGATCCATCTGGGATCTGCTGCT |
| | | GAAGTACAGATCAGGCCGGACCATCATCATGAGCACCCA |
| | | CCACATGGACGAGGCCGATCTGCTCGGAGACAGAATCGC |
| | | CATCATTGCTCAGGGCAGACTGTACTGCAGCGGCACCCC |
| | | ACTGTTTCTGAAGAACTGTTTCGGCACCGGACTGTATCTG |
| | | ACCCTCGTGCGGAAGATGAAGAACATCCAGTCTCAGCGG |
| | | AAGGGCAGCGAGGGCACCTGTAGCTGTTCTAGCAAGGGC |
| | | TTTAGCACCACCTGTCCAGCTCACGTGGACGATCTGACCC |
| | | CTGAACAGGTGCTGGATGGCGACGTGAACGAGCTGATGG |
| | | ACGTGGTGCTGCACCATGTGCCTGAGGCCAAGCTGGTGG |
| | | AATGCATCGGCCAAGAACTGATTTTTCTGCTCCCGAACAA |
| | | GAACTTCAAGCACCGGGCCTACGCCAGCCTGTTCAGAGA |
| | | GCTGGAAGAAACCCTGGCCGACCTGGGCCTGTCTAGCTT |
| | | TGGCATCAGCGACACCCCTCTCGAAGAGATTTTCCTGAA |
| | | AGTGACAGAGGACAGCGATAGCGGCCCTCTGTTTGCTGG |
| | | CGGAGCACAGCAAAAGCGCGAGAACGTGAACCCTAGAC |
| | | ACCCCTGTCTGGGCCCAAGAGAGAAAGCCGGACAGACCC |
| | | CTCAGGACAGCAATGTGTGCTCTCCTGGTGCTCCTGCCGC |
| | | TCATCCTGAGGGACAACCTCCACCTGAACCTGAGTGTCCT |
| | | GGACCTCAGCTGAACACCGGAACACAGCTGGTTCTGCAG |
| | | CACGTGCAGGCTCTGCTCGTGAAGAGATTCCAGCACACC |
| | | ATCAGAAGCCACAAGGACTTTCTGGCCCAGATCGTGCTG |
| | | CCCGCCACCTTTGTTTTTCTGGCTCTGATGCTGAGCATCG |
| | | TGATCCCTCCATTCGGCGAGTACCCCGCTCTGACACTGCA |
| | | CCCTTGGATCTACGGCCAGCAGTACACCTTTTTCTCCATG |
| | | GACGAACCCGGCAGCGAGCAGTTCACAGTGCTGGCTGAT |
| | | GTCCTGCTGAACAAGCCCGGCTTCGGCAACCGGTGTCTG |
| | | AAAGAAGGATGGCTGCCTGAGTACCCTTGCGGCAACAGC |
| | | ACACCTTGGAAAACCCCTAGCGTGTCCCCTAACATCACCC |
| | | AGCTGTTCCAAAAGCAGAAATGGACCCAAGTGAACCCCT |
| | | CTCCATCCTGCCGGTGCTCCACAAGGGAAAAGCTGACCA |
| | | TGCTGCCCGAGTGTCCAGAAGGCGCTGGCGGACTTCCTC |
| | | CACCTCAGAGAACACAGAGATCCACCGAGATTCTCCAGG |
| | | ACCTGACCGACCGGAATATCAGCGACTTCCTGGTTAAGA |
| | | CATACCCCGCACTGATCCGGTCCAGCCTGAAGTCCAAGTT |
| | | CTGGGTCAACGAACAGAGATACGGCGGCATCAGCATCGG |
| | | CGGAAAACTGCCTGTGGTGCCTATCACAGGCGAGGCCCT |
| | | TGTGGGCTTTCTGTCCGATCTGGGGAGAATCATGAACGTG |
| | | TCCGGCGGACCTATCACCAGGGAAGCCAGCAAAGAGATC |
| | | CCCGATTTCCTGAAGCACCTGGAAACCGAGGACAATATC |
| | | AAAGTGTGGTTCAACAACAAAGGATGGCACGCCCTCGTG |
| | | TCTTTTCTGAACGTGGCCCACAATGCCATCCTGCGGGCTA |
| | | GCCTGCCTAAGGACAGAAGCCCTGAGGAATACGGCATCA |
| | | CCGTGATCTCCCAGCCTCTGAATCTGACCAAAGAGCAGC |
| | | TGAGCGAGATCACCGTGCTGACCACCTCTGTGGATGCTGT |
| | | GGTGGCCATCTGCGTGATCTTCAGCATGAGCTTCGTGCCC |
| | | GCCTCCTTCGTGCTGTACCTGATTCAAGAGAGAGTGAAC |
| | | AAGAGCAAGCACCTCCAGTTCATCTCCGGGGTGTCCCCA |
| | | ACCACCTACTGGGTCACCAATTTTCTGTGGGACATCATGA |
| | | ACTACAGCGTGTCAGCCGGCCTGGTCGTGGGCATCTTTAT |
| | | CGGCTTTCAGAAGAAGGCCTACACGAGCCCCGAGAACCT |
| | | GCCTGCTTTGGTTGCTCTGCTGCTCCTGTATGGCTGGGCC |
| | | GTGATTCCCATGATGTACCCCGCCAGCTTTCTGTTTGACG |
| | | TGCCCAGCACAGCCTACGTGGCCCTGTCTTGCGCCAATCT |
| | | GTTCATCGGCATCAACAGCAGCGCCATCACATTCATCCTG |
| | | GAACTGTTCGAGAACAACAGGACCCTGCTGCGGTTCAAC |
| | | GCCGTGCTGCGGAAACTGCTGATCGTGTTCCCTCACTTCT |
| | | GTCTCGGCAGAGGCCTGATCGACCTGGCTCTGTCTCAGGC |
| | | CGTGACCGATGTGTACGCCAGATTTGGCGAGGAACACTC |
| | | CGCCAATCCATTCCACTGGGACCTGATCGGCAAGAACCT |
| | | GTTCGCCATGGTGGTGGAAGGCGTCGTGTACTTCCTGCTC |
| | | ACTCTGCTGGTGCAGAGACACTTTTTTCTGTCCCAATGGA |
| | | TCGCCGAGCCTACCAAAGAACCCATTGTGGACGAGGACG |
| | | ACGATGTGGCCGAGGAAAGACAGAGAATCATCACCGGC |
| | | GGCAACAAGACCGATATCCTGAGACTGCACGAGCTGACA |
| | | AAGATTTACCCCGGCACAAGCTCCCCAGCCGTGGATAGG |
| | | CTTTGTGTGGGAGTTAGACCCGGCGAGTGCTTTGGCCTGC |
| | | TGGGAGTTAATGGCGCCGGAAAGACCACCACCTTCAAGA |
| | | TGCTGACCGGCGACACCACAGTGACAAGCGGAGATGCTA |
| | | CAGTGGCCGGCAAGAGCATCCTGACCAACATCAGCGAAG |
| | | TGCATCAGAACATGGGCTACTGCCCTCAGTTCGACGCCAT |
| | | CGACGAACTGCTGACAGGCCGCGAACACCTGTATCTGTA |
| | | TGCCAGACTGAGAGGCGTGCCCGCTGAAGAGATCGAGAA |
| | | GGTGGCCAACTGGTCCATCAAGTCTCTGGGCCTGACAGT |
| | | GTACGCCGACTGTCTGGCCGGAACATACAGCGGAGGAAA |
| | | CAAGCGGAAGCTGAGCACCGCCATTGCTCTGATCGGATG |
| | | CCCACCTCTGGTCCTGCTGGATGAACCCACCACCGGAAT |
| | | GGACCCCCAGGCTAGAAGAATGCTCTGGAACGTGATCGT |
| | | GTCTATCATCCGCGAGGGCAGAGCTGTGGTGCTGACCTCT |
| | | CACAGCATGGAAGAGTGCGAGGCTCTGTGTACCCGGCTG |
| | | GCCATTATGGTCAAGGGCGCCTTCAGATGCATGGGCACC |
| | | ATTCAGCATCTGAAAAGCAAGTTCGGCGACGGCTACATC |
| | | GTGACAATGAAGATCAAGAGCCCCAAGGACGACCTCCTG |
| | | CCTGATCTGAACCCCGTGGAACAGTTTTTTCAGGGCAACT |
| | | TCCCCGGCTCCGTGCAGCGGGAAAGACACTATAACATGC |
| | | TGCAGTTTCAGGTGTCCTCCTCCAGCCTGGCTCGGATCTT |
| | | TCAACTGCTGCTCTCTCACAAGGACAGCCTGCTGATTGAA |
| | | GAGTACAGCGTGACACAGACCACACTCGACCAGGTTTTC |
| | | GTGAACTTCGCCAAGCAGCAGACCGAGAGCCACGACCTG |
| | | CCTCTGCATCCTAGAGCCGCTGGTGCCTCTAGACAAGCTC |
| | | AGGACTAAGCTTCCACTGGATTGTACAATTACATAAAAT |
| | | AAAATATCTTTATTTTCATTACATCTGTGTGTTGGTTTTTT |
| | | GTGTGCGCTACT |
| 2 | Plasmid | GTCGCTGCCGACTGGGCAATGGAGCGCCTGCCGGCCCAG |
| | template for | TATCAGCCCGTCATACTTGAAGCTAGACAGGCTTATCTTG |
| | 8,656 bp | GACAAGAAGAAGATCGCTTGGCCTCGCGCGCAGATCAGT |
| | C3DNA with | TGGAAGAATTTGTCCACTACGTGAAAGGCGAGATCACCA |
| | AAAA | AGGTAGTCGGCAAATAACCTTAATGAGACCAACTCAATG |
| | overhang | AGACCGATCTGTTGATCAGCAGTTCAACCTGTTGATAGTA |
| | | CGTACTAAGCTCTCATGTTTCACGTACTAAGCTCTCATGT |
| | | TTAACGTACTAAGCTCTCATGTTTAACGAACTAAACCCTC |
| | | ATGGCTAACGTACTAAGCTCTCATGGCTAACGTACTAAG |
| | | CTCTCATGTTTCACGTACTAAGCTCTCATGTTTGAACAAT |
| | | AAAATTAATATAAATCAGCAACTTAAATAGCCTCTAAGG |
| | | TTTTAAGTTTTATAAGAAAAAAAAGAATATATAAGGCTTT |
| | | TAAAGCTTTTAAGGTTTAACGGTTGTGGACAACAAGCCA |
| | | GGGATGTAACGCACTGAGAAGCCCTTAGAGCCTCTCAAA |
| | | GCAATTTTGAGTGACACAGGAACACTTAACGGCTGACAT |
| | | GGGAATTAGCCATGGGCCCGTGCGAATCACTATATGAGA |
| | | CCGCTGATCCTTCAACTCAGCAAAAGTTCGATTTATTCAA |
| | | CAAAGCCACGTTGTGTCTCAAAATCTCTGATGTTACATTG |
| | | CACAAGATAAAAATATATCATCATGAACAATAAAACTGT |
| | | CTGCTTACATAAACAGTAATACAAGGGGTGTTATGAGCC |
| | | ATATTCAACGGGAAACGTCGAGGCCGCGATTAAATTCCA |
| | | ACATGGATGCTGATTTATATGGGTATAAATGGGCTCGCG |
| | | ATAATGTCGGGCAATCAGGTGCGACAATCTATCGCTTGT |
| | | ATGGGAAGCCCGATGCGCCAGAGTTGTTTCTGAAACATG |
| | | GCAAAGGTAGCGTTGCCAATGATGTTACAGATGAGATGG |
| | | TCAGACTAAACTGGCTGACGGAATTTATGCCTCTTCCGAC |
| | | CATCAAGCATTTTATCCGTACTCCTGATGATGCATGGTTA |
| | | CTCACCACTGCGATCCCCGGAAAAACAGCATTCCAGGTA |
| | | TTAGAAGAATATCCTGATTCAGGTGAAAATATTGTTGATG |
| | | CGCTGGCAGTGTTCCTGCGCCGGTTGCATTCGATTCCTGT |
| | | TTGTAATTGTCCTTTTAACAGCGATCGCGTATTTCGTCTC |
| | | GCTCAGGCGCAATCACGAATGAATAACGGTTTGGTTGAT |
| | | GCGAGTGATTTTGATGACGAGCGTAATGGCTGGCCTGTT |
| | | GAACAAGTCTGGAAAGAAATGCATAAACTTTTGCCATTC |
| | | TCACCGGATTCAGTCGTCACTCATGGTGATTTCTCACTTG |
| | | ATAACCTTATTTTTGACGAGGGGAAATTAATAGGTTGTAT |
| | | TGATGTTGGACGAGTCGGAATCGCAGACCGATACCAGGA |
| | | TCTTGCCATCCTATGGAACTGCCTCGGTGAGTTTTCTCCTT |
| | | CATTACAGAAACGGCTTTTTCAAAAATATGGTATTGATAA |
| | | TCCTGATATGAATAAATTGCAGTTTCATTTGATGCTCGAT |
| | | GAGTTTTTCTAATCAGAATTGGTTAATTGGTTGTAACACT |
| | | GGCAGAGCATTACGCTGACTTGACGGGAGAATTCGGTCT |
| | | CAAAAACTCTTCGGTATCACAGGAGAATTTCAGGGAGAC |
| | | ATTGATTATTGACTAGTTATTAATAGTAATCAATTACGGG |
| | | GTCATTAGTTCATAGCCCATATATGGAGTTCCGCGTTACA |
| | | TAACTTACGGTAAATGGCCCGCCTGGCTGACCGCCCAAC |
| | | GACCCCCGCCCATTGACGTCAATAATGACGTATGTTCCCA |
| | | TAGTAACGCCAATAGGGACTTTCCATTGACGTCAATGGG |
| | | TGGAGTATTTACGGTAAACTGCCCACTTGGCAGTACATCA |
| | | AGTGTATCATATGCCAAGTACGCCCCCTATTGACGTCAAT |
| | | GACGGTAAATGGCCCGCCTGGCATTATGCCCAGTACATG |
| | | ACCTTATGGGACTTTCCTACTTGGCAGTACATCTACGTAT |
| | | TAGTCATCGCTATTACCATGTCGAGGTGAGCCCCACGTTC |
| | | TGCTTCACTCTCCCCATCTCCCCCCCCTCCCCACCCCCAAT |
| | | TTTGTATTTATTTATTTTTTAATTATTTTGTGCAGCGATGG |
| | | GGGCGGGGGGGGGGGGGGGGCGCGCGCCAGGCGGGGCG |
| | | GGGCGGGGCGAGGGGCGGGGCGGGGCGAGGCGGAGAGG |
| | | TGCGGCGGCAGCCAATCAGAGCGGCGCGCTCCGAAAGTT |
| | | TCCTTTTATGGCGAGGCGGCGGCGGCGGCGGCCCTATAA |
| | | AAAGCGAAGCGCGCGGCGGGCGGGAGTCGCTGCGACGC |
| | | TGCCTTCGCCCCGTGCCCCGCTCCGCCGCCGCCTCGCGCC |
| | | GCCCGCCCCGGCTCTGACTGACCGCGTTACTCCCACAGGT |
| | | GAGCGGGCGGGACGGCCCTTCTCCTCCGGGCTGTAATTA |
| | | GCGCTTGGTTTAATGACGGCTTGTTTCTTTTCTGTGGCTGC |
| | | GTGAAAGCCTTGAGGGGCTCCGGGAGGGCCCTTTGTGCG |
| | | GGGGGAGCGGCTCGGGGGGTGCGTGCGTGTGTGTGTGCG |
| | | TGGGGAGCGCCGCGTGCGGCTCCGCGCTGCCCGGCGGCT |
| | | GTGAGCGCTGCGGGCGCGGCGCGGGGCTTTGTGCGCTCC |
| | | GCAGTGTGCGCGAGGGGAGCGCGGCCGGGGGCGGTGCC |
| | | CCGCGGTGCGGGGGGGGCTGCGAGGGGAACAAAGGCTG |
| | | CGTGCGGGGTGTGTGCGTGGGGGGGTGAGCAGGGGGTGT |
| | | GGGCGCGTCGGTCGGGCTGCAACCCCCCCTGCACCCCCC |
| | | TCCCCGAGTTGCTGAGCACGGCCCGGCTTCGGGTGCGGG |
| | | GCTCCGTACGGGGCGTGGCGCGGGGCTCGCCGTGCCGGG |
| | | CGGGGGGTGGCGGCAGGTGGGGGTGCCGGGCGGGGCGG |
| | | GGCCGCCTCGGGCCGGGGAGGGCTCGGGGGAGGGGCGC |
| | | GGCGGCCCCCGGAGCGCCGGCGGCTGTCGAGGCGCGGCG |
| | | AGCCGCAGCCATTGCCTTTTATGGTAATCGTGCGAGAGG |
| | | GCGCAGGGACTTCCTTTGTCCCAAATCTGTGCGGAGCCG |
| | | AAATCTGGGAGGCGCCGCCGCACCCCCTCTAGCGGGCGC |
| | | GGGGCGAAGCGGTGCGGCGCCGGCAGGAAGGAAATGGG |
| | | CGGGGAGGGCCTTCGTGCGTCGCCGCGCCGCCGTCCCCTT |
| | | CTCCCTCTCCAGCCTCGGGGCTGTCCGCGGGGGGACGGC |
| | | TGCCTTCGGGGGGGACGGGGCAGGGCGGGGTTCGGCTTC |
| | | TGGCGTGTGACCGGCGGCTCTAGACAATTGTACTAACCTT |
| | | CTTCTCTTTCCTCTCCTGACAGGGAGTTTAAACAGATAAG |
| | | TTTGTACAAAAAAGAGAGGTGCCACCATGGGCTTTGTGC |
| | | GACAGATTCAGCTGCTGCTGTGGAAGAACTGGACCCTGC |
| | | GGAAGCGGCAGAAAATCAGATTCGTGGTGGAACTCGTGT |
| | | GGCCCCTGAGCCTGTTTCTGGTGCTGATCTGGCTGCGGAA |
| | | CGCCAATCCTCTGTACAGCCACCACGAGTGTCACTTCCCC |
| | | AACAAGGCCATGCCTTCTGCCGGAATGCTGCCTTGGCTGC |
| | | AGGGCATCTTCTGCAACGTGAACAACCCCTGCTTTCAGA |
| | | GCCCCACACCTGGCGAAAGCCCTGGCATCGTGTCCAACT |
| | | ACAACAACAGCATCCTGGCCAGAGTGTACCGGGACTTCC |
| | | AAGAGCTGCTGATGAACGCCCCTGAGTCTCAGCACCTGG |
| | | GCAGAATCTGGACCGAGCTGCACATCCTGAGCCAGTTCA |
| | | TGGACACCCTGAGAACACACCCCGAGAGAATCGCCGGCA |
| | | GGGGCATCAGAATCCGGGACATCCTGAAGGACGAGGAA |
| | | ACCCTGACACTGTTCCTCATCAAGAACATCGGCCTGAGC |
| | | GACAGCGTGGTGTACCTGCTGATCAACAGCCAAGTGCGG |
| | | CCCGAGCAGTTTGCTCATGGCGTGCCGGATCTCGCCCTGA |
| | | AGGATATCGCCTGTTCTGAGGCCCTGCTGGAACGGTTCAT |
| | | CATCTTCAGCCAGCGGAGAGGCGCCAAGACCGTCAGATA |
| | | TGCCCTGTGCAGTCTGAGCCAGGGAACCCTGCAGTGGAT |
| | | CGAGGATACCCTGTACGCCAACGTGGACTTCTTCAAGCT |
| | | GTTCCGGGTGCTGCCCACACTGCTGGATTCTAGATCCCAG |
| | | GGCATCAACCTGAGAAGCTGGGGCGGCATCCTGTCCGAC |
| | | ATGAGCCCAAGAATCCAAGAGTTCATCCACCGGCCTAGC |
| | | ATGCAGGACCTGCTGTGGGTTACCAGACCTCTGATGCAG |
| | | AACGGCGGACCCGAGACATTCACCAAGCTGATGGGCATT |
| | | CTGAGCGATCTGCTGTGCGGCTACCCTGAAGGCGGAGGA |
| | | TCTAGAGTGCTGAGCTTCAATTGGTACGAGGACAACAAC |
| | | TACAAGGCCTTCCTGGGCATCGACTCCACCAGAAAGGAC |
| | | CCCATCTACAGCTACGACCGGCGGACAACCAGCTTCTGC |
| | | AATGCCCTGATCCAGAGCCTGGAAAGCAACCCTCTGACC |
| | | AAGATCGCTTGGAGGGCCGCCAAACCTCTGCTGATGGGA |
| | | AAGATCCTGTACACCCCTGACAGCCCTGCCGCCAGAAGA |
| | | ATCCTGAAGAACGCCAACAGCACCTTCGAGGAACTGGAA |
| | | CACGTGCGCAAGCTGGTCAAGGCCTGGGAAGAAGTGGGA |
| | | CCTCAGATTTGGTACTTCTTCGACAATAGCACCCAGATGA |
| | | ACATGATCAGAGACACCCTGGGCAACCCTACCGTGAAGG |
| | | ACTTCCTGAACAGACAGCTGGGCGAAGAGGGCATTACCG |
| | | CCGAGGCCATCCTGAACTTTCTGTACAAGGGCCCCAGAG |
| | | AGTCCCAGGCCGACGACATGGCCAACTTCGATTGGCGGG |
| | | ACATCTTCAACATCACCGACAGAACCCTGCGGCTGGTCA |
| | | ACCAGTACCTGGAATGCCTGGTGCTGGACAAGTTCGAGA |
| | | GCTACAACGACGAGACACAGCTGACCCAGAGAGCCCTGT |
| | | CTCTGCTGGAAGAGAATATGTTCTGGGCTGGCGTGGTGTT |
| | | CCCCGACATGTACCCTTGGACAAGCAGCCTGCCTCCTCAC |
| | | GTGAAGTACAAGATCCGGATGGACATCGACGTGGTCGAA |
| | | AAGACCAACAAGATCAAGGACCGGTACTGGGACAGCGG |
| | | CCCTAGAGCTGATCCCGTGGAAGATTTTCGGTACATCTGG |
| | | GGCGGATTCGCATACCTGCAGGACATGGTGGAACAGGGA |
| | | ATCACACGGTCCCAGGTGCAGGCTGAAGCTCCTGTGGGA |
| | | ATCTACCTGCAGCAGATGCCTTATCCTTGCTTCGTGGACG |
| | | ACAGCTTCATGATCATCCTGAATCGGTGCTTCCCCATCTT |
| | | CATGGTGCTGGCCTGGATCTACTCCGTGTCTATGACCGTG |
| | | AAGTCCATCGTGCTGGAAAAAGAGCTGCGGCTGAAAGAG |
| | | ACACTGAAGAACCAGGGCGTGTCCAATGCCGTGATCTGG |
| | | TGCACCTGGTTTCTGGACAGCTTCTCCATTATGAGCATGA |
| | | GCATCTTTCTGCTGACGATCTTCATCATGCACGGCCGAAT |
| | | CCTGCACTACAGCGACCCCTTTATCCTCTTCCTGTTCCTGC |
| | | TGGCCTTCAGCACCGCTACAATCATGCTGTGTTTTCTGCT |
| | | GTCCACCTTCTTCAGCAAGGCCTCTCTGGCCGCTGCTTGT |
| | | AGCGGCGTGATCTACTTCACCCTGTACCTGCCTCACATCC |
| | | TGTGCTTCGCATGGCAGGACAGAATGACCGCCGAGCTGA |
| | | AGAAAGCTGTGTCCCTGCTGAGCCCTGTGGCCTTTGGCTT |
| | | TGGCACCGAGTACCTCGTCAGATTTGAGGAACAAGGACT |
| | | GGGACTGCAGTGGTCCAACATCGGCAATAGCCCTACAGA |
| | | GGGCGACGAGTTCAGCTTCCTGCTGTCTATGCAGATGATG |
| | | CTGCTGGACGCCGCCGTGTATGGACTGCTGGCTTGGTATC |
| | | TGGACCAGGTGTTCCCAGGCGATTACGGCACTCCTCTGCC |
| | | TTGGTATTTCCTGCTGCAAGAGAGCTACTGGCTCGGCGGC |
| | | GAGGGATGTAGCACCAGAGAAGAAAGAGCCCTGGAAAA |
| | | GACCGAGCCTCTGACCGAGGAAACAGAGGACCCTGAACA |
| | | CCCAGAGGGCATCCACGATAGCTTTTTCGAGAGAGAACA |
| | | CCCCGGCTGGGTGCCAGGCGTGTGTGTGAAGAATCTGGT |
| | | CAAGATTTTCGAGCCCTGCGGCAGACCTGCCGTGGACAG |
| | | ACTGAACATCACCTTCTACGAGAACCAGATTACCGCCTTT |
| | | CTGGGCCACAACGGCGCTGGCAAGACAACCACATTGAGC |
| | | ATCCTCACAGGCCTGCTGCCTCCAACAAGCGGCACAGTT |
| | | CTCGTTGGCGGCAGAGACATCGAGACAAGCCTGGATGCC |
| | | GTCAGACAGTCCCTGGGCATGTGCCCTCAGCACAACATC |
| | | CTGTTTCACCACCTGACCGTGGCCGAGCACATGCTGTTTT |
| | | ATGCCCAGCTGAAGGGCAAGAGCCAAGAAGAGGCTCAG |
| | | CTGGAAATGGAAGCCATGTTGGAGGACACCGGCCTGCAC |
| | | CACAAGAGAAATGAGGAAGCCCAGGATCTGAGCGGCGG |
| | | CATGCAGAGAAAACTGAGCGTGGCCATTGCCTTCGTGGG |
| | | CGACGCCAAGGTTGTGATCCTGGATGAGCCTACAAGCGG |
| | | CGTGGACCCTTACAGCAGAAGATCCATCTGGGATCTGCT |
| | | GCTGAAGTACAGATCAGGCCGGACCATCATCATGAGCAC |
| | | CCACCACATGGACGAGGCCGATCTGCTCGGAGACAGAAT |
| | | CGCCATCATTGCTCAGGGCAGACTGTACTGCAGCGGCAC |
| | | CCCACTGTTTCTGAAGAACTGTTTCGGCACCGGACTGTAT |
| | | CTGACCCTCGTGCGGAAGATGAAGAACATCCAGTCTCAG |
| | | CGGAAGGGCAGCGAGGGCACCTGTAGCTGTTCTAGCAAG |
| | | GGCTTTAGCACCACCTGTCCAGCTCACGTGGACGATCTGA |
| | | CCCCTGAACAGGTGCTGGATGGCGACGTGAACGAGCTGA |
| | | TGGACGTGGTGCTGCACCATGTGCCTGAGGCCAAGCTGG |
| | | TGGAATGCATCGGCCAAGAACTGATTTTTCTGCTCCCGAA |
| | | CAAGAACTTCAAGCACCGGGCCTACGCCAGCCTGTTCAG |
| | | AGAGCTGGAAGAAACCCTGGCCGACCTGGGCCTGTCTAG |
| | | CTTTGGCATCAGCGACACCCCTCTCGAAGAGATTTTCCTG |
| | | AAAGTGACAGAGGACAGCGATAGCGGCCCTCTGTTTGCT |
| | | GGCGGAGCACAGCAAAAGCGCGAGAACGTGAACCCTAG |
| | | ACACCCCTGTCTGGGCCCAAGAGAGAAAGCCGGACAGAC |
| | | CCCTCAGGACAGCAATGTGTGCTCTCCTGGTGCTCCTGCC |
| | | GCTCATCCTGAGGGACAACCTCCACCTGAACCTGAGTGT |
| | | CCTGGACCTCAGCTGAACACCGGAACACAGCTGGTTCTG |
| | | CAGCACGTGCAGGCTCTGCTCGTGAAGAGATTCCAGCAC |
| | | ACCATCAGAAGCCACAAGGACTTTCTGGCCCAGATCGTG |
| | | CTGCCCGCCACCTTTGTTTTTCTGGCTCTGATGCTGAGCA |
| | | TCGTGATCCCTCCATTCGGCGAGTACCCCGCTCTGACACT |
| | | GCACCCTTGGATCTACGGCCAGCAGTACACCTTTTTCTCC |
| | | ATGGACGAACCCGGCAGCGAGCAGTTCACAGTGCTGGCT |
| | | GATGTCCTGCTGAACAAGCCCGGCTTCGGCAACCGGTGT |
| | | CTGAAAGAAGGATGGCTGCCTGAGTACCCTTGCGGCAAC |
| | | AGCACACCTTGGAAAACCCCTAGCGTGTCCCCTAACATC |
| | | ACCCAGCTGTTCCAAAAGCAGAAATGGACCCAAGTGAAC |
| | | CCCTCTCCATCCTGCCGGTGCTCCACAAGGGAAAAGCTG |
| | | ACCATGCTGCCCGAGTGTCCAGAAGGCGCTGGCGGACTT |
| | | CCTCCACCTCAGAGAACACAGAGATCCACCGAGATTCTC |
| | | CAGGACCTGACCGACCGGAATATCAGCGACTTCCTGGTT |
| | | AAGACATACCCCGCACTGATCCGGTCCAGCCTGAAGTCC |
| | | AAGTTCTGGGTCAACGAACAGAGATACGGCGGCATCAGC |
| | | ATCGGCGGAAAACTGCCTGTGGTGCCTATCACAGGCGAG |
| | | GCCCTTGTGGGCTTTCTGTCCGATCTGGGGAGAATCATGA |
| | | ACGTGTCCGGCGGACCTATCACCAGGGAAGCCAGCAAAG |
| | | AGATCCCCGATTTCCTGAAGCACCTGGAAACCGAGGACA |
| | | ATATCAAAGTGTGGTTCAACAACAAAGGATGGCACGCCC |
| | | TCGTGTCTTTTCTGAACGTGGCCCACAATGCCATCCTGCG |
| | | GGCTAGCCTGCCTAAGGACAGAAGCCCTGAGGAATACGG |
| | | CATCACCGTGATCTCCCAGCCTCTGAATCTGACCAAAGA |
| | | GCAGCTGAGCGAGATCACCGTGCTGACCACCTCTGTGGA |
| | | TGCTGTGGTGGCCATCTGCGTGATCTTCAGCATGAGCTTC |
| | | GTGCCCGCCTCCTTCGTGCTGTACCTGATTCAAGAGAGAG |
| | | TGAACAAGAGCAAGCACCTCCAGTTCATCTCCGGGGTGT |
| | | CCCCAACCACCTACTGGGTCACCAATTTTCTGTGGGACAT |
| | | CATGAACTACAGCGTGTCAGCCGGCCTGGTCGTGGGCAT |
| | | CTTTATCGGCTTTCAGAAGAAGGCCTACACGAGCCCCGA |
| | | GAACCTGCCTGCTTTGGTTGCTCTGCTGCTCCTGTATGGC |
| | | TGGGCCGTGATTCCCATGATGTACCCCGCCAGCTTTCTGT |
| | | TTGACGTGCCCAGCACAGCCTACGTGGCCCTGTCTTGCGC |
| | | CAATCTGTTCATCGGCATCAACAGCAGCGCCATCACATTC |
| | | ATCCTGGAACTGTTCGAGAACAACAGGACCCTGCTGCGG |
| | | TTCAACGCCGTGCTGCGGAAACTGCTGATCGTGTTCCCTC |
| | | ACTTCTGTCTCGGCAGAGGCCTGATCGACCTGGCTCTGTC |
| | | TCAGGCCGTGACCGATGTGTACGCCAGATTTGGCGAGGA |
| | | ACACTCCGCCAATCCATTCCACTGGGACCTGATCGGCAA |
| | | GAACCTGTTCGCCATGGTGGTGGAAGGCGTCGTGTACTTC |
| | | CTGCTCACTCTGCTGGTGCAGAGACACTTTTTTCTGTCCC |
| | | AATGGATCGCCGAGCCTACCAAAGAACCCATTGTGGACG |
| | | AGGACGACGATGTGGCCGAGGAAAGACAGAGAATCATC |
| | | ACCGGCGGCAACAAGACCGATATCCTGAGACTGCACGAG |
| | | CTGACAAAGATTTACCCCGGCACAAGCTCCCCAGCCGTG |
| | | GATAGGCTTTGTGTGGGAGTTAGACCCGGCGAGTGCTTT |
| | | GGCCTGCTGGGAGTTAATGGCGCCGGAAAGACCACCACC |
| | | TTCAAGATGCTGACCGGCGACACCACAGTGACAAGCGGA |
| | | GATGCTACAGTGGCCGGCAAGAGCATCCTGACCAACATC |
| | | AGCGAAGTGCATCAGAACATGGGCTACTGCCCTCAGTTC |
| | | GACGCCATCGACGAACTGCTGACAGGCCGCGAACACCTG |
| | | TATCTGTATGCCAGACTGAGAGGCGTGCCCGCTGAAGAG |
| | | ATCGAGAAGGTGGCCAACTGGTCCATCAAGTCTCTGGGC |
| | | CTGACAGTGTACGCCGACTGTCTGGCCGGAACATACAGC |
| | | GGAGGAAACAAGCGGAAGCTGAGCACCGCCATTGCTCTG |
| | | ATCGGATGCCCACCTCTGGTCCTGCTGGATGAACCCACCA |
| | | CCGGAATGGACCCCCAGGCTAGAAGAATGCTCTGGAACG |
| | | TGATCGTGTCTATCATCCGCGAGGGCAGAGCTGTGGTGCT |
| | | GACCTCTCACAGCATGGAAGAGTGCGAGGCTCTGTGTAC |
| | | CCGGCTGGCCATTATGGTCAAGGGCGCCTTCAGATGCAT |
| | | GGGCACCATTCAGCATCTGAAAAGCAAGTTCGGCGACGG |
| | | CTACATCGTGACAATGAAGATCAAGAGCCCCAAGGACGA |
| | | CCTCCTGCCTGATCTGAACCCCGTGGAACAGTTTTTTCAG |
| | | GGCAACTTCCCCGGCTCCGTGCAGCGGGAAAGACACTAT |
| | | AACATGCTGCAGTTTCAGGTGTCCTCCTCCAGCCTGGCTC |
| | | GGATCTTTCAACTGCTGCTCTCTCACAAGGACAGCCTGCT |
| | | GATTGAAGAGTACAGCGTGACACAGACCACACTCGACCA |
| | | GGTTTTCGTGAACTTCGCCAAGCAGCAGACCGAGAGCCA |
| | | CGACCTGCCTCTGCATCCTAGAGCCGCTGGTGCCTCTAGA |
| | | CAAGCTCAGGACTAAGCTTCCACTGGATTGTACAATTAC |
| | | ATAAAATAAAATATCTTTATTTTCATTACATCTGTGTGTT |
| | | GGTTTTTTGTGTGCGCTACTAAAATGAGACCGAATTCCCA |
| | | TCCAGCTGATATCCCCTATAGTGAGTCGTATTACATGGTC |
| | | ATAGCTGTTTCCTGGCAGCTCTGGCCCGTGTCTCAAAATC |
| | | TCTGATGTTACATTGCACAAGATAAAAATATATCATCATG |
| | | CCTCCTCTAGACCAGCCAGGACAGAAATGCCTCGACTTC |
| | | GCTGCTGCCCAAGGTTGCCGGGTGACGCACACCGTGGAA |
| | | ACGGATGAAGGCACGAACCCAGTGGACATAAGCCTGTTC |
| | | GGTTCGTAAGCTGTAATGCAAGTAGCGTATGCGCTCACG |
| | | CAACTGGTCCAGAACCTTGACCGAACGCAGCGGTGGTAA |
| | | CGGCGCAGTGGCGGTTTTCATGGCTTGTTATGACTGTTTT |
| | | TTTGGGGTACAGTCTATGCCTCGGGCATCCAAGCAGCAA |
| | | GCGCGTTACGCCGTGGGTCGATGTTTGATGTTATGGAGCA |
| | | GCAACGATGTTACGCAGCAGGGCAGTCGCCCTAAAACAA |
| | | AGTTAAACATCATGAGGGAAGCGGTGATCGCCGAAGTAT |
| | | CGACTCAACTATCAGAGGTAGTTGGCGTCATCGAGCGCC |
| | | ATCTCGAACCGACGTTGCTGGCCGTACATTTGTACGGCTC |
| | | CGCAGTGGATGGCGGCCTGAAGCCACACAGTGATATTGA |
| | | TTTGCTGGTTACGGTGACCGTAAGGCTTGATGAAACAAC |
| | | GCGGCGAGCTTTGATCAACGACCTTTTGGAAACTTCGGCT |
| | | TCCCCTGGAGAGAGCGAGATTCTCCGCGCTGTAGAAGTC |
| | | ACCATTGTTGTGCACGACGACATCATTCCGTGGCGTTATC |
| | | CAGCTAAGCGCGAACTGCAATTTGGAGAATGGCAGCGCA |
| | | ATGACATTCTTGCAGGTATCTTCGAGCCAGCCACGATCGA |
| | | CATTGATCTGGCTATCTTGCTGACAAAAGCAAGAGAACA |
| | | TAGCGTTGCCTTGGTAGGTCCAGCGGCGGAGGAACTCTTT |
| | | GATCCGGTTCCTGAACAGGATCTATTTGAGGCGCTAAAT |
| | | GAAACCTTAACGCTATGGAACTCGCCGCCCGACTGGGCT |
| | | GGCGATGAGCGAAATGTAGTGCTTACGTTGTCCCGCATTT |
| | | GGTACAGCGCAGTAACCGGCAAAATCGCGCCGAAGGAT |
| 3 | 8,656 bp | AACC |
| | C3DNA w | CTCTTCGGTATCACAGGAGAATTTCAGGGAGACATTGATT |
| | AACC | ATTGACTAGTTATTAATAGTAATCAATTACGGGGTCATTA |
| | overhang | GTTCATAGCCCATATATGGAGTTCCGCGTTACATAACTTA |
| | | CGGTAAATGGCCCGCCTGGCTGACCGCCCAACGACCCCC |
| | | GCCCATTGACGTCAATAATGACGTATGTTCCCATAGTAAC |
| | | GCCAATAGGGACTTTCCATTGACGTCAATGGGTGGAGTA |
| | | TTTACGGTAAACTGCCCACTTGGCAGTACATCAAGTGTAT |
| | | CATATGCCAAGTACGCCCCCTATTGACGTCAATGACGGT |
| | | AAATGGCCCGCCTGGCATTATGCCCAGTACATGACCTTAT |
| | | GGGACTTTCCTACTTGGCAGTACATCTACGTATTAGTCAT |
| | | CGCTATTACCATGTCGAGGTGAGCCCCACGTTCTGCTTCA |
| | | CTCTCCCCATCTCCCCCCCCTCCCCACCCCCAATTTTGTAT |
| | | TTATTTATTTTTTAATTATTTTGTGCAGCGATGGGGGCGG |
| | | GGGGGGGGGGGGGGCGCGCGCCAGGCGGGGCGGGGCGG |
| | | GGCGAGGGGCGGGGCGGGGCGAGGCGGAGAGGTGCGGC |
| | | GGCAGCCAATCAGAGCGGCGCGCTCCGAAAGTTTCCTTT |
| | | TATGGCGAGGCGGCGGCGGCGGCGGCCCTATAAAAAGCG |
| | | AAGCGCGCGGCGGGCGGGAGTCGCTGCGACGCTGCCTTC |
| | | GCCCCGTGCCCCGCTCCGCCGCCGCCTCGCGCCGCCCGCC |
| | | CCGGCTCTGACTGACCGCGTTACTCCCACAGGTGAGCGG |
| | | GCGGGACGGCCCTTCTCCTCCGGGCTGTAATTAGCGCTTG |
| | | GTTTAATGACGGCTTGTTTCTTTTCTGTGGCTGCGTGAAA |
| | | GCCTTGAGGGGCTCCGGGAGGGCCCTTTGTGCGGGGGGA |
| | | GCGGCTCGGGGGGTGCGTGCGTGTGTGTGTGCGTGGGGA |
| | | GCGCCGCGTGCGGCTCCGCGCTGCCCGGCGGCTGTGAGC |
| | | GCTGCGGGCGCGGCGCGGGGCTTTGTGCGCTCCGCAGTG |
| | | TGCGCGAGGGGAGCGCGGCCGGGGGCGGTGCCCCGCGGT |
| | | GCGGGGGGGGCTGCGAGGGGAACAAAGGCTGCGTGCGG |
| | | GGTGTGTGCGTGGGGGGGTGAGCAGGGGGTGTGGGCGCG |
| | | TCGGTCGGGCTGCAACCCCCCCTGCACCCCCCTCCCCGAG |
| | | TTGCTGAGCACGGCCCGGCTTCGGGTGCGGGGCTCCGTA |
| | | CGGGGCGTGGCGCGGGGCTCGCCGTGCCGGGCGGGGGGT |
| | | GGCGGCAGGTGGGGGTGCCGGGCGGGGCGGGGCCGCCT |
| | | CGGGCCGGGGAGGGCTCGGGGGAGGGGCGCGGCGGCCC |
| | | CCGGAGCGCCGGCGGCTGTCGAGGCGCGGCGAGCCGCAG |
| | | CCATTGCCTTTTATGGTAATCGTGCGAGAGGGCGCAGGG |
| | | ACTTCCTTTGTCCCAAATCTGTGCGGAGCCGAAATCTGGG |
| | | AGGCGCCGCCGCACCCCCTCTAGCGGGCGCGGGGCGAAG |
| | | CGGTGCGGCGCCGGCAGGAAGGAAATGGGCGGGGAGGG |
| | | CCTTCGTGCGTCGCCGCGCCGCCGTCCCCTTCTCCCTCTC |
| | | CAGCCTCGGGGCTGTCCGCGGGGGGACGGCTGCCTTCGG |
| | | GGGGGACGGGGCAGGGCGGGGTTCGGCTTCTGGCGTGTG |
| | | ACCGGCGGCTCTAGACAATTGTACTAACCTTCTTCTCTTT |
| | | CCTCTCCTGACAGGGAGTTTAAACAGATAAGTTTGTACA |
| | | AAAAAGAGAGGTGCCACCATGGGCTTTGTGCGACAGATT |
| | | CAGCTGCTGCTGTGGAAGAACTGGACCCTGCGGAAGCGG |
| | | CAGAAAATCAGATTCGTGGTGGAACTCGTGTGGCCCCTG |
| | | AGCCTGTTTCTGGTGCTGATCTGGCTGCGGAACGCCAATC |
| | | CTCTGTACAGCCACCACGAGTGTCACTTCCCCAACAAGG |
| | | CCATGCCTTCTGCCGGAATGCTGCCTTGGCTGCAGGGCAT |
| | | CTTCTGCAACGTGAACAACCCCTGCTTTCAGAGCCCCACA |
| | | CCTGGCGAAAGCCCTGGCATCGTGTCCAACTACAACAAC |
| | | AGCATCCTGGCCAGAGTGTACCGGGACTTCCAAGAGCTG |
| | | CTGATGAACGCCCCTGAGTCTCAGCACCTGGGCAGAATC |
| | | TGGACCGAGCTGCACATCCTGAGCCAGTTCATGGACACC |
| | | CTGAGAACACACCCCGAGAGAATCGCCGGCAGGGGCATC |
| | | AGAATCCGGGACATCCTGAAGGACGAGGAAACCCTGACA |
| | | CTGTTCCTCATCAAGAACATCGGCCTGAGCGACAGCGTG |
| | | GTGTACCTGCTGATCAACAGCCAAGTGCGGCCCGAGCAG |
| | | TTTGCTCATGGCGTGCCGGATCTCGCCCTGAAGGATATCG |
| | | CCTGTTCTGAGGCCCTGCTGGAACGGTTCATCATCTTCAG |
| | | CCAGCGGAGAGGCGCCAAGACCGTCAGATATGCCCTGTG |
| | | CAGTCTGAGCCAGGGAACCCTGCAGTGGATCGAGGATAC |
| | | CCTGTACGCCAACGTGGACTTCTTCAAGCTGTTCCGGGTG |
| | | CTGCCCACACTGCTGGATTCTAGATCCCAGGGCATCAACC |
| | | TGAGAAGCTGGGGCGGCATCCTGTCCGACATGAGCCCAA |
| | | GAATCCAAGAGTTCATCCACCGGCCTAGCATGCAGGACC |
| | | TGCTGTGGGTTACCAGACCTCTGATGCAGAACGGCGGAC |
| | | CCGAGACATTCACCAAGCTGATGGGCATTCTGAGCGATC |
| | | TGCTGTGCGGCTACCCTGAAGGCGGAGGATCTAGAGTGC |
| | | TGAGCTTCAATTGGTACGAGGACAACAACTACAAGGCCT |
| | | TCCTGGGCATCGACTCCACCAGAAAGGACCCCATCTACA |
| | | GCTACGACCGGCGGACAACCAGCTTCTGCAATGCCCTGA |
| | | TCCAGAGCCTGGAAAGCAACCCTCTGACCAAGATCGCTT |
| | | GGAGGGCCGCCAAACCTCTGCTGATGGGAAAGATCCTGT |
| | | ACACCCCTGACAGCCCTGCCGCCAGAAGAATCCTGAAGA |
| | | ACGCCAACAGCACCTTCGAGGAACTGGAACACGTGCGCA |
| | | AGCTGGTCAAGGCCTGGGAAGAAGTGGGACCTCAGATTT |
| | | GGTACTTCTTCGACAATAGCACCCAGATGAACATGATCA |
| | | GAGACACCCTGGGCAACCCTACCGTGAAGGACTTCCTGA |
| | | ACAGACAGCTGGGCGAAGAGGGCATTACCGCCGAGGCC |
| | | ATCCTGAACTTTCTGTACAAGGGCCCCAGAGAGTCCCAG |
| | | GCCGACGACATGGCCAACTTCGATTGGCGGGACATCTTC |
| | | AACATCACCGACAGAACCCTGCGGCTGGTCAACCAGTAC |
| | | CTGGAATGCCTGGTGCTGGACAAGTTCGAGAGCTACAAC |
| | | GACGAGACACAGCTGACCCAGAGAGCCCTGTCTCTGCTG |
| | | GAAGAGAATATGTTCTGGGCTGGCGTGGTGTTCCCCGAC |
| | | ATGTACCCTTGGACAAGCAGCCTGCCTCCTCACGTGAAGT |
| | | ACAAGATCCGGATGGACATCGACGTGGTCGAAAAGACCA |
| | | ACAAGATCAAGGACCGGTACTGGGACAGCGGCCCTAGAG |
| | | CTGATCCCGTGGAAGATTTTCGGTACATCTGGGGCGGATT |
| | | CGCATACCTGCAGGACATGGTGGAACAGGGAATCACACG |
| | | GTCCCAGGTGCAGGCTGAAGCTCCTGTGGGAATCTACCT |
| | | GCAGCAGATGCCTTATCCTTGCTTCGTGGACGACAGCTTC |
| | | ATGATCATCCTGAATCGGTGCTTCCCCATCTTCATGGTGC |
| | | TGGCCTGGATCTACTCCGTGTCTATGACCGTGAAGTCCAT |
| | | CGTGCTGGAAAAAGAGCTGCGGCTGAAAGAGACACTGA |
| | | AGAACCAGGGCGTGTCCAATGCCGTGATCTGGTGCACCT |
| | | GGTTTCTGGACAGCTTCTCCATTATGAGCATGAGCATCTT |
| | | TCTGCTGACGATCTTCATCATGCACGGCCGAATCCTGCAC |
| | | TACAGCGACCCCTTTATCCTCTTCCTGTTCCTGCTGGCCTT |
| | | CAGCACCGCTACAATCATGCTGTGTTTTCTGCTGTCCACC |
| | | TTCTTCAGCAAGGCCTCTCTGGCCGCTGCTTGTAGCGGCG |
| | | TGATCTACTTCACCCTGTACCTGCCTCACATCCTGTGCTTC |
| | | GCATGGCAGGACAGAATGACCGCCGAGCTGAAGAAAGC |
| | | TGTGTCCCTGCTGAGCCCTGTGGCCTTTGGCTTTGGCACC |
| | | GAGTACCTCGTCAGATTTGAGGAACAAGGACTGGGACTG |
| | | CAGTGGTCCAACATCGGCAATAGCCCTACAGAGGGCGAC |
| | | GAGTTCAGCTTCCTGCTGTCTATGCAGATGATGCTGCTGG |
| | | ACGCCGCCGTGTATGGACTGCTGGCTTGGTATCTGGACCA |
| | | GGTGTTCCCAGGCGATTACGGCACTCCTCTGCCTTGGTAT |
| | | TTCCTGCTGCAAGAGAGCTACTGGCTCGGCGGCGAGGGA |
| | | TGTAGCACCAGAGAAGAAAGAGCCCTGGAAAAGACCGA |
| | | GCCTCTGACCGAGGAAACAGAGGACCCTGAACACCCAGA |
| | | GGGCATCCACGATAGCTTTTTCGAGAGAGAACACCCCGG |
| | | CTGGGTGCCAGGCGTGTGTGTGAAGAATCTGGTCAAGAT |
| | | TTTCGAGCCCTGCGGCAGACCTGCCGTGGACAGACTGAA |
| | | CATCACCTTCTACGAGAACCAGATTACCGCCTTTCTGGGC |
| | | CACAACGGCGCTGGCAAGACAACCACATTGAGCATCCTC |
| | | ACAGGCCTGCTGCCTCCAACAAGCGGCACAGTTCTCGTT |
| | | GGCGGCAGAGACATCGAGACAAGCCTGGATGCCGTCAGA |
| | | CAGTCCCTGGGCATGTGCCCTCAGCACAACATCCTGTTTC |
| | | ACCACCTGACCGTGGCCGAGCACATGCTGTTTTATGCCCA |
| | | GCTGAAGGGCAAGAGCCAAGAAGAGGCTCAGCTGGAAA |
| | | TGGAAGCCATGTTGGAGGACACCGGCCTGCACCACAAGA |
| | | GAAATGAGGAAGCCCAGGATCTGAGCGGCGGCATGCAG |
| | | AGAAAACTGAGCGTGGCCATTGCCTTCGTGGGCGACGCC |
| | | AAGGTTGTGATCCTGGATGAGCCTACAAGCGGCGTGGAC |
| | | CCTTACAGCAGAAGATCCATCTGGGATCTGCTGCTGAAG |
| | | TACAGATCAGGCCGGACCATCATCATGAGCACCCACCAC |
| | | ATGGACGAGGCCGATCTGCTCGGAGACAGAATCGCCATC |
| | | ATTGCTCAGGGCAGACTGTACTGCAGCGGCACCCCACTG |
| | | TTTCTGAAGAACTGTTTCGGCACCGGACTGTATCTGACCC |
| | | TCGTGCGGAAGATGAAGAACATCCAGTCTCAGCGGAAGG |
| | | GCAGCGAGGGCACCTGTAGCTGTTCTAGCAAGGGCTTTA |
| | | GCACCACCTGTCCAGCTCACGTGGACGATCTGACCCCTG |
| | | AACAGGTGCTGGATGGCGACGTGAACGAGCTGATGGACG |
| | | TGGTGCTGCACCATGTGCCTGAGGCCAAGCTGGTGGAAT |
| | | GCATCGGCCAAGAACTGATTTTTCTGCTCCCGAACAAGA |
| | | ACTTCAAGCACCGGGCCTACGCCAGCCTGTTCAGAGAGC |
| | | TGGAAGAAACCCTGGCCGACCTGGGCCTGTCTAGCTTTG |
| | | GCATCAGCGACACCCCTCTCGAAGAGATTTTCCTGAAAG |
| | | TGACAGAGGACAGCGATAGCGGCCCTCTGTTTGCTGGCG |
| | | GAGCACAGCAAAAGCGCGAGAACGTGAACCCTAGACAC |
| | | CCCTGTCTGGGCCCAAGAGAGAAAGCCGGACAGACCCCT |
| | | CAGGACAGCAATGTGTGCTCTCCTGGTGCTCCTGCCGCTC |
| | | ATCCTGAGGGACAACCTCCACCTGAACCTGAGTGTCCTG |
| | | GACCTCAGCTGAACACCGGAACACAGCTGGTTCTGCAGC |
| | | ACGTGCAGGCTCTGCTCGTGAAGAGATTCCAGCACACCA |
| | | TCAGAAGCCACAAGGACTTTCTGGCCCAGATCGTGCTGC |
| | | CCGCCACCTTTGTTTTTCTGGCTCTGATGCTGAGCATCGT |
| | | GATCCCTCCATTCGGCGAGTACCCCGCTCTGACACTGCAC |
| | | CCTTGGATCTACGGCCAGCAGTACACCTTTTTCTCCATGG |
| | | ACGAACCCGGCAGCGAGCAGTTCACAGTGCTGGCTGATG |
| | | TCCTGCTGAACAAGCCCGGCTTCGGCAACCGGTGTCTGA |
| | | AAGAAGGATGGCTGCCTGAGTACCCTTGCGGCAACAGCA |
| | | CACCTTGGAAAACCCCTAGCGTGTCCCCTAACATCACCCA |
| | | GCTGTTCCAAAAGCAGAAATGGACCCAAGTGAACCCCTC |
| | | TCCATCCTGCCGGTGCTCCACAAGGGAAAAGCTGACCAT |
| | | GCTGCCCGAGTGTCCAGAAGGCGCTGGCGGACTTCCTCC |
| | | ACCTCAGAGAACACAGAGATCCACCGAGATTCTCCAGGA |
| | | CCTGACCGACCGGAATATCAGCGACTTCCTGGTTAAGAC |
| | | ATACCCCGCACTGATCCGGTCCAGCCTGAAGTCCAAGTTC |
| | | TGGGTCAACGAACAGAGATACGGCGGCATCAGCATCGGC |
| | | GGAAAACTGCCTGTGGTGCCTATCACAGGCGAGGCCCTT |
| | | GTGGGCTTTCTGTCCGATCTGGGGAGAATCATGAACGTGT |
| | | CCGGCGGACCTATCACCAGGGAAGCCAGCAAAGAGATCC |
| | | CCGATTTCCTGAAGCACCTGGAAACCGAGGACAATATCA |
| | | AAGTGTGGTTCAACAACAAAGGATGGCACGCCCTCGTGT |
| | | CTTTTCTGAACGTGGCCCACAATGCCATCCTGCGGGCTAG |
| | | CCTGCCTAAGGACAGAAGCCCTGAGGAATACGGCATCAC |
| | | CGTGATCTCCCAGCCTCTGAATCTGACCAAAGAGCAGCT |
| | | GAGCGAGATCACCGTGCTGACCACCTCTGTGGATGCTGT |
| | | GGTGGCCATCTGCGTGATCTTCAGCATGAGCTTCGTGCCC |
| | | GCCTCCTTCGTGCTGTACCTGATTCAAGAGAGAGTGAAC |
| | | AAGAGCAAGCACCTCCAGTTCATCTCCGGGGTGTCCCCA |
| | | ACCACCTACTGGGTCACCAATTTTCTGTGGGACATCATGA |
| | | ACTACAGCGTGTCAGCCGGCCTGGTCGTGGGCATCTTTAT |
| | | CGGCTTTCAGAAGAAGGCCTACACGAGCCCCGAGAACCT |
| | | GCCTGCTTTGGTTGCTCTGCTGCTCCTGTATGGCTGGGCC |
| | | GTGATTCCCATGATGTACCCCGCCAGCTTTCTGTTTGACG |
| | | TGCCCAGCACAGCCTACGTGGCCCTGTCTTGCGCCAATCT |
| | | GTTCATCGGCATCAACAGCAGCGCCATCACATTCATCCTG |
| | | GAACTGTTCGAGAACAACAGGACCCTGCTGCGGTTCAAC |
| | | GCCGTGCTGCGGAAACTGCTGATCGTGTTCCCTCACTTCT |
| | | GTCTCGGCAGAGGCCTGATCGACCTGGCTCTGTCTCAGGC |
| | | CGTGACCGATGTGTACGCCAGATTTGGCGAGGAACACTC |
| | | CGCCAATCCATTCCACTGGGACCTGATCGGCAAGAACCT |
| | | GTTCGCCATGGTGGTGGAAGGCGTCGTGTACTTCCTGCTC |
| | | ACTCTGCTGGTGCAGAGACACTTTTTTCTGTCCCAATGGA |
| | | TCGCCGAGCCTACCAAAGAACCCATTGTGGACGAGGACG |
| | | ACGATGTGGCCGAGGAAAGACAGAGAATCATCACCGGC |
| | | GGCAACAAGACCGATATCCTGAGACTGCACGAGCTGACA |
| | | AAGATTTACCCCGGCACAAGCTCCCCAGCCGTGGATAGG |
| | | CTTTGTGTGGGAGTTAGACCCGGCGAGTGCTTTGGCCTGC |
| | | TGGGAGTTAATGGCGCCGGAAAGACCACCACCTTCAAGA |
| | | TGCTGACCGGCGACACCACAGTGACAAGCGGAGATGCTA |
| | | CAGTGGCCGGCAAGAGCATCCTGACCAACATCAGCGAAG |
| | | TGCATCAGAACATGGGCTACTGCCCTCAGTTCGACGCCAT |
| | | CGACGAACTGCTGACAGGCCGCGAACACCTGTATCTGTA |
| | | TGCCAGACTGAGAGGCGTGCCCGCTGAAGAGATCGAGAA |
| | | GGTGGCCAACTGGTCCATCAAGTCTCTGGGCCTGACAGT |
| | | GTACGCCGACTGTCTGGCCGGAACATACAGCGGAGGAAA |
| | | CAAGCGGAAGCTGAGCACCGCCATTGCTCTGATCGGATG |
| | | CCCACCTCTGGTCCTGCTGGATGAACCCACCACCGGAAT |
| | | GGACCCCCAGGCTAGAAGAATGCTCTGGAACGTGATCGT |
| | | GTCTATCATCCGCGAGGGCAGAGCTGTGGTGCTGACCTCT |
| | | CACAGCATGGAAGAGTGCGAGGCTCTGTGTACCCGGCTG |
| | | GCCATTATGGTCAAGGGCGCCTTCAGATGCATGGGCACC |
| | | ATTCAGCATCTGAAAAGCAAGTTCGGCGACGGCTACATC |
| | | GTGACAATGAAGATCAAGAGCCCCAAGGACGACCTCCTG |
| | | CCTGATCTGAACCCCGTGGAACAGTTTTTTCAGGGCAACT |
| | | TCCCCGGCTCCGTGCAGCGGGAAAGACACTATAACATGC |
| | | TGCAGTTTCAGGTGTCCTCCTCCAGCCTGGCTCGGATCTT |
| | | TCAACTGCTGCTCTCTCACAAGGACAGCCTGCTGATTGAA |
| | | GAGTACAGCGTGACACAGACCACACTCGACCAGGTTTTC |
| | | GTGAACTTCGCCAAGCAGCAGACCGAGAGCCACGACCTG |
| | | CCTCTGCATCCTAGAGCCGCTGGTGCCTCTAGACAAGCTC |
| | | AGGACTAAGCTTCCACTGGATTGTACAATTACATAAAAT |
| | | AAAATATCTTTATTTTCATTACATCTGTGTGTTGGTTTTTT |
| | | GTGTGCGCTACT |
| 4 | Plasmid | GGTATCACAGGAGAATTTCAGGGAGACATTGATTATTGAC |
| | template for | TAGTTATTAATAGTAATCAATTACGGGGTCATTAGTTCAT |
| | 8,656 bp | AGCCCATATATGGAGTTCCGCGTTACATAACTTACGGTAA |
| | C3DNA with | ATGGCCCGCCTGGCTGACCGCCCAACGACCCCCGCCCAT |
| | AACC | TGACGTCAATAATGACGTATGTTCCCATAGTAACGCCAAT |
| | overhang | AGGGACTTTCCATTGACGTCAATGGGTGGAGTATTTACG |
| | | GTAAACTGCCCACTTGGCAGTACATCAAGTGTATCATATG |
| | | CCAAGTACGCCCCCTATTGACGTCAATGACGGTAAATGG |
| | | CCCGCCTGGCATTATGCCCAGTACATGACCTTATGGGACT |
| | | TTCCTACTTGGCAGTACATCTACGTATTAGTCATCGCTAT |
| | | TACCATGTCGAGGTGAGCCCCACGTTCTGCTTCACTCTCC |
| | | CCATCTCCCCCCCCTCCCCACCCCCAATTTTGTATTTATTT |
| | | ATTTTTTAATTATTTTGTGCAGCGATGGGGGCGGGGGGGG |
| | | GGGGGGGGCGCGCGCCAGGCGGGGCGGGGCGGGGCGAG |
| | | GGGCGGGGCGGGGCGAGGCGGAGAGGTGCGGCGGCAGC |
| | | CAATCAGAGCGGCGCGCTCCGAAAGTTTCCTTTTATGGCG |
| | | AGGCGGCGGCGGCGGCGGCCCTATAAAAAGCGAAGCGC |
| | | GCGGCGGGCGGGAGTCGCTGCGACGCTGCCTTCGCCCCG |
| | | TGCCCCGCTCCGCCGCCGCCTCGCGCCGCCCGCCCCGGCT |
| | | CTGACTGACCGCGTTACTCCCACAGGTGAGCGGGCGGGA |
| | | CGGCCCTTCTCCTCCGGGCTGTAATTAGCGCTTGGTTTAA |
| | | TGACGGCTTGTTTCTTTTCTGTGGCTGCGTGAAAGCCTTG |
| | | AGGGGCTCCGGGAGGGCCCTTTGTGCGGGGGGAGCGGCT |
| | | CGGGGGGTGCGTGCGTGTGTGTGTGCGTGGGGAGCGCCG |
| | | CGTGCGGCTCCGCGCTGCCCGGCGGCTGTGAGCGCTGCG |
| | | GGCGCGGCGCGGGGCTTTGTGCGCTCCGCAGTGTGCGCG |
| | | AGGGGAGCGCGGCCGGGGGCGGTGCCCCGCGGTGCGGG |
| | | GGGGGCTGCGAGGGGAACAAAGGCTGCGTGCGGGGTGT |
| | | GTGCGTGGGGGGGTGAGCAGGGGGTGTGGGCGCGTCGGT |
| | | CGGGCTGCAACCCCCCCTGCACCCCCCTCCCCGAGTTGCT |
| | | GAGCACGGCCCGGCTTCGGGTGCGGGGCTCCGTACGGGG |
| | | CGTGGCGCGGGGCTCGCCGTGCCGGGCGGGGGGTGGCGG |
| | | CAGGTGGGGGTGCCGGGCGGGGCGGGGCCGCCTCGGGCC |
| | | GGGGAGGGCTCGGGGGAGGGGCGCGGCGGCCCCCGGAG |
| | | CGCCGGCGGCTGTCGAGGCGCGGCGAGCCGCAGCCATTG |
| | | CCTTTTATGGTAATCGTGCGAGAGGGCGCAGGGACTTCCT |
| | | TTGTCCCAAATCTGTGCGGAGCCGAAATCTGGGAGGCGC |
| | | CGCCGCACCCCCTCTAGCGGGCGCGGGGCGAAGCGGTGC |
| | | GGCGCCGGCAGGAAGGAAATGGGCGGGGAGGGCCTTCG |
| | | TGCGTCGCCGCGCCGCCGTCCCCTTCTCCCTCTCCAGCCT |
| | | CGGGGCTGTCCGCGGGGGGACGGCTGCCTTCGGGGGGGA |
| | | CGGGGCAGGGCGGGGTTCGGCTTCTGGCGTGTGACCGGC |
| | | GGCTCTAGACAATTGTACTAACCTTCTTCTCTTTCCTCTCC |
| | | TGACAGGGAGTTTAAACAGATAAGTTTGTACAAAAAAGA |
| | | GAGGTGCCACCATGGGCTTTGTGCGACAGATTCAGCTGC |
| | | TGCTGTGGAAGAACTGGACCCTGCGGAAGCGGCAGAAAA |
| | | TCAGATTCGTGGTGGAACTCGTGTGGCCCCTGAGCCTGTT |
| | | TCTGGTGCTGATCTGGCTGCGGAACGCCAATCCTCTGTAC |
| | | AGCCACCACGAGTGTCACTTCCCCAACAAGGCCATGCCT |
| | | TCTGCCGGAATGCTGCCTTGGCTGCAGGGCATCTTCTGCA |
| | | ACGTGAACAACCCCTGCTTTCAGAGCCCCACACCTGGCG |
| | | AAAGCCCTGGCATCGTGTCCAACTACAACAACAGCATCC |
| | | TGGCCAGAGTGTACCGGGACTTCCAAGAGCTGCTGATGA |
| | | ACGCCCCTGAGTCTCAGCACCTGGGCAGAATCTGGACCG |
| | | AGCTGCACATCCTGAGCCAGTTCATGGACACCCTGAGAA |
| | | CACACCCCGAGAGAATCGCCGGCAGGGGCATCAGAATCC |
| | | GGGACATCCTGAAGGACGAGGAAACCCTGACACTGTTCC |
| | | TCATCAAGAACATCGGCCTGAGCGACAGCGTGGTGTACC |
| | | TGCTGATCAACAGCCAAGTGCGGCCCGAGCAGTTTGCTC |
| | | ATGGCGTGCCGGATCTCGCCCTGAAGGATATCGCCTGTTC |
| | | TGAGGCCCTGCTGGAACGGTTCATCATCTTCAGCCAGCG |
| | | GAGAGGCGCCAAGACCGTCAGATATGCCCTGTGCAGTCT |
| | | GAGCCAGGGAACCCTGCAGTGGATCGAGGATACCCTGTA |
| | | CGCCAACGTGGACTTCTTCAAGCTGTTCCGGGTGCTGCCC |
| | | ACACTGCTGGATTCTAGATCCCAGGGCATCAACCTGAGA |
| | | AGCTGGGGCGGCATCCTGTCCGACATGAGCCCAAGAATC |
| | | CAAGAGTTCATCCACCGGCCTAGCATGCAGGACCTGCTG |
| | | TGGGTTACCAGACCTCTGATGCAGAACGGCGGACCCGAG |
| | | ACATTCACCAAGCTGATGGGCATTCTGAGCGATCTGCTGT |
| | | GCGGCTACCCTGAAGGCGGAGGATCTAGAGTGCTGAGCT |
| | | TCAATTGGTACGAGGACAACAACTACAAGGCCTTCCTGG |
| | | GCATCGACTCCACCAGAAAGGACCCCATCTACAGCTACG |
| | | ACCGGCGGACAACCAGCTTCTGCAATGCCCTGATCCAGA |
| | | GCCTGGAAAGCAACCCTCTGACCAAGATCGCTTGGAGGG |
| | | CCGCCAAACCTCTGCTGATGGGAAAGATCCTGTACACCC |
| | | CTGACAGCCCTGCCGCCAGAAGAATCCTGAAGAACGCCA |
| | | ACAGCACCTTCGAGGAACTGGAACACGTGCGCAAGCTGG |
| | | TCAAGGCCTGGGAAGAAGTGGGACCTCAGATTTGGTACT |
| | | TCTTCGACAATAGCACCCAGATGAACATGATCAGAGACA |
| | | CCCTGGGCAACCCTACCGTGAAGGACTTCCTGAACAGAC |
| | | AGCTGGGCGAAGAGGGCATTACCGCCGAGGCCATCCTGA |
| | | ACTTTCTGTACAAGGGCCCCAGAGAGTCCCAGGCCGACG |
| | | ACATGGCCAACTTCGATTGGCGGGACATCTTCAACATCA |
| | | CCGACAGAACCCTGCGGCTGGTCAACCAGTACCTGGAAT |
| | | GCCTGGTGCTGGACAAGTTCGAGAGCTACAACGACGAGA |
| | | CACAGCTGACCCAGAGAGCCCTGTCTCTGCTGGAAGAGA |
| | | ATATGTTCTGGGCTGGCGTGGTGTTCCCCGACATGTACCC |
| | | TTGGACAAGCAGCCTGCCTCCTCACGTGAAGTACAAGAT |
| | | CCGGATGGACATCGACGTGGTCGAAAAGACCAACAAGAT |
| | | CAAGGACCGGTACTGGGACAGCGGCCCTAGAGCTGATCC |
| | | CGTGGAAGATTTTCGGTACATCTGGGGCGGATTCGCATA |
| | | CCTGCAGGACATGGTGGAACAGGGAATCACACGGTCCCA |
| | | GGTGCAGGCTGAAGCTCCTGTGGGAATCTACCTGCAGCA |
| | | GATGCCTTATCCTTGCTTCGTGGACGACAGCTTCATGATC |
| | | ATCCTGAATCGGTGCTTCCCCATCTTCATGGTGCTGGCCT |
| | | GGATCTACTCCGTGTCTATGACCGTGAAGTCCATCGTGCT |
| | | GGAAAAAGAGCTGCGGCTGAAAGAGACACTGAAGAACC |
| | | AGGGCGTGTCCAATGCCGTGATCTGGTGCACCTGGTTTCT |
| | | GGACAGCTTCTCCATTATGAGCATGAGCATCTTTCTGCTG |
| | | ACGATCTTCATCATGCACGGCCGAATCCTGCACTACAGC |
| | | GACCCCTTTATCCTCTTCCTGTTCCTGCTGGCCTTCAGCAC |
| | | CGCTACAATCATGCTGTGTTTTCTGCTGTCCACCTTCTTCA |
| | | GCAAGGCCTCTCTGGCCGCTGCTTGTAGCGGCGTGATCTA |
| | | CTTCACCCTGTACCTGCCTCACATCCTGTGCTTCGCATGG |
| | | CAGGACAGAATGACCGCCGAGCTGAAGAAAGCTGTGTCC |
| | | CTGCTGAGCCCTGTGGCCTTTGGCTTTGGCACCGAGTACC |
| | | TCGTCAGATTTGAGGAACAAGGACTGGGACTGCAGTGGT |
| | | CCAACATCGGCAATAGCCCTACAGAGGGCGACGAGTTCA |
| | | GCTTCCTGCTGTCTATGCAGATGATGCTGCTGGACGCCGC |
| | | CGTGTATGGACTGCTGGCTTGGTATCTGGACCAGGTGTTC |
| | | CCAGGCGATTACGGCACTCCTCTGCCTTGGTATTTCCTGC |
| | | TGCAAGAGAGCTACTGGCTCGGCGGCGAGGGATGTAGCA |
| | | CCAGAGAAGAAAGAGCCCTGGAAAAGACCGAGCCTCTG |
| | | ACCGAGGAAACAGAGGACCCTGAACACCCAGAGGGCAT |
| | | CCACGATAGCTTTTTCGAGAGAGAACACCCCGGCTGGGT |
| | | GCCAGGCGTGTGTGTGAAGAATCTGGTCAAGATTTTCGA |
| | | GCCCTGCGGCAGACCTGCCGTGGACAGACTGAACATCAC |
| | | CTTCTACGAGAACCAGATTACCGCCTTTCTGGGCCACAAC |
| | | GGCGCTGGCAAGACAACCACATTGAGCATCCTCACAGGC |
| | | CTGCTGCCTCCAACAAGCGGCACAGTTCTCGTTGGCGGC |
| | | AGAGACATCGAGACAAGCCTGGATGCCGTCAGACAGTCC |
| | | CTGGGCATGTGCCCTCAGCACAACATCCTGTTTCACCACC |
| | | TGACCGTGGCCGAGCACATGCTGTTTTATGCCCAGCTGAA |
| | | GGGCAAGAGCCAAGAAGAGGCTCAGCTGGAAATGGAAG |
| | | CCATGTTGGAGGACACCGGCCTGCACCACAAGAGAAATG |
| | | AGGAAGCCCAGGATCTGAGCGGCGGCATGCAGAGAAAA |
| | | CTGAGCGTGGCCATTGCCTTCGTGGGCGACGCCAAGGTT |
| | | GTGATCCTGGATGAGCCTACAAGCGGCGTGGACCCTTAC |
| | | AGCAGAAGATCCATCTGGGATCTGCTGCTGAAGTACAGA |
| | | TCAGGCCGGACCATCATCATGAGCACCCACCACATGGAC |
| | | GAGGCCGATCTGCTCGGAGACAGAATCGCCATCATTGCT |
| | | CAGGGCAGACTGTACTGCAGCGGCACCCCACTGTTTCTG |
| | | AAGAACTGTTTCGGCACCGGACTGTATCTGACCCTCGTGC |
| | | GGAAGATGAAGAACATCCAGTCTCAGCGGAAGGGCAGC |
| | | GAGGGCACCTGTAGCTGTTCTAGCAAGGGCTTTAGCACC |
| | | ACCTGTCCAGCTCACGTGGACGATCTGACCCCTGAACAG |
| | | GTGCTGGATGGCGACGTGAACGAGCTGATGGACGTGGTG |
| | | CTGCACCATGTGCCTGAGGCCAAGCTGGTGGAATGCATC |
| | | GGCCAAGAACTGATTTTTCTGCTCCCGAACAAGAACTTCA |
| | | AGCACCGGGCCTACGCCAGCCTGTTCAGAGAGCTGGAAG |
| | | AAACCCTGGCCGACCTGGGCCTGTCTAGCTTTGGCATCAG |
| | | CGACACCCCTCTCGAAGAGATTTTCCTGAAAGTGACAGA |
| | | GGACAGCGATAGCGGCCCTCTGTTTGCTGGCGGAGCACA |
| | | GCAAAAGCGCGAGAACGTGAACCCTAGACACCCCTGTCT |
| | | GGGCCCAAGAGAGAAAGCCGGACAGACCCCTCAGGACA |
| | | GCAATGTGTGCTCTCCTGGTGCTCCTGCCGCTCATCCTGA |
| | | GGGACAACCTCCACCTGAACCTGAGTGTCCTGGACCTCA |
| | | GCTGAACACCGGAACACAGCTGGTTCTGCAGCACGTGCA |
| | | GGCTCTGCTCGTGAAGAGATTCCAGCACACCATCAGAAG |
| | | CCACAAGGACTTTCTGGCCCAGATCGTGCTGCCCGCCACC |
| | | TTTGTTTTTCTGGCTCTGATGCTGAGCATCGTGATCCCTCC |
| | | ATTCGGCGAGTACCCCGCTCTGACACTGCACCCTTGGATC |
| | | TACGGCCAGCAGTACACCTTTTTCTCCATGGACGAACCCG |
| | | GCAGCGAGCAGTTCACAGTGCTGGCTGATGTCCTGCTGA |
| | | ACAAGCCCGGCTTCGGCAACCGGTGTCTGAAAGAAGGAT |
| | | GGCTGCCTGAGTACCCTTGCGGCAACAGCACACCTTGGA |
| | | AAACCCCTAGCGTGTCCCCTAACATCACCCAGCTGTTCCA |
| | | AAAGCAGAAATGGACCCAAGTGAACCCCTCTCCATCCTG |
| | | CCGGTGCTCCACAAGGGAAAAGCTGACCATGCTGCCCGA |
| | | GTGTCCAGAAGGCGCTGGCGGACTTCCTCCACCTCAGAG |
| | | AACACAGAGATCCACCGAGATTCTCCAGGACCTGACCGA |
| | | CCGGAATATCAGCGACTTCCTGGTTAAGACATACCCCGC |
| | | ACTGATCCGGTCCAGCCTGAAGTCCAAGTTCTGGGTCAA |
| | | CGAACAGAGATACGGCGGCATCAGCATCGGCGGAAAACT |
| | | GCCTGTGGTGCCTATCACAGGCGAGGCCCTTGTGGGCTTT |
| | | CTGTCCGATCTGGGGAGAATCATGAACGTGTCCGGCGGA |
| | | CCTATCACCAGGGAAGCCAGCAAAGAGATCCCCGATTTC |
| | | CTGAAGCACCTGGAAACCGAGGACAATATCAAAGTGTGG |
| | | TTCAACAACAAAGGATGGCACGCCCTCGTGTCTTTTCTGA |
| | | ACGTGGCCCACAATGCCATCCTGCGGGCTAGCCTGCCTA |
| | | AGGACAGAAGCCCTGAGGAATACGGCATCACCGTGATCT |
| | | CCCAGCCTCTGAATCTGACCAAAGAGCAGCTGAGCGAGA |
| | | TCACCGTGCTGACCACCTCTGTGGATGCTGTGGTGGCCAT |
| | | CTGCGTGATCTTCAGCATGAGCTTCGTGCCCGCCTCCTTC |
| | | GTGCTGTACCTGATTCAAGAGAGAGTGAACAAGAGCAAG |
| | | CACCTCCAGTTCATCTCCGGGGTGTCCCCAACCACCTACT |
| | | GGGTCACCAATTTTCTGTGGGACATCATGAACTACAGCGT |
| | | GTCAGCCGGCCTGGTCGTGGGCATCTTTATCGGCTTTCAG |
| | | AAGAAGGCCTACACGAGCCCCGAGAACCTGCCTGCTTTG |
| | | GTTGCTCTGCTGCTCCTGTATGGCTGGGCCGTGATTCCCA |
| | | TGATGTACCCCGCCAGCTTTCTGTTTGACGTGCCCAGCAC |
| | | AGCCTACGTGGCCCTGTCTTGCGCCAATCTGTTCATCGGC |
| | | ATCAACAGCAGCGCCATCACATTCATCCTGGAACTGTTCG |
| | | AGAACAACAGGACCCTGCTGCGGTTCAACGCCGTGCTGC |
| | | GGAAACTGCTGATCGTGTTCCCTCACTTCTGTCTCGGCAG |
| | | AGGCCTGATCGACCTGGCTCTGTCTCAGGCCGTGACCGAT |
| | | GTGTACGCCAGATTTGGCGAGGAACACTCCGCCAATCCA |
| | | TTCCACTGGGACCTGATCGGCAAGAACCTGTTCGCCATG |
| | | GTGGTGGAAGGCGTCGTGTACTTCCTGCTCACTCTGCTGG |
| | | TGCAGAGACACTTTTTTCTGTCCCAATGGATCGCCGAGCC |
| | | TACCAAAGAACCCATTGTGGACGAGGACGACGATGTGGC |
| | | CGAGGAAAGACAGAGAATCATCACCGGCGGCAACAAGA |
| | | CCGATATCCTGAGACTGCACGAGCTGACAAAGATTTACC |
| | | CCGGCACAAGCTCCCCAGCCGTGGATAGGCTTTGTGTGG |
| | | GAGTTAGACCCGGCGAGTGCTTTGGCCTGCTGGGAGTTA |
| | | ATGGCGCCGGAAAGACCACCACCTTCAAGATGCTGACCG |
| | | GCGACACCACAGTGACAAGCGGAGATGCTACAGTGGCCG |
| | | GCAAGAGCATCCTGACCAACATCAGCGAAGTGCATCAGA |
| | | ACATGGGCTACTGCCCTCAGTTCGACGCCATCGACGAAC |
| | | TGCTGACAGGCCGCGAACACCTGTATCTGTATGCCAGAC |
| | | TGAGAGGCGTGCCCGCTGAAGAGATCGAGAAGGTGGCCA |
| | | ACTGGTCCATCAAGTCTCTGGGCCTGACAGTGTACGCCG |
| | | ACTGTCTGGCCGGAACATACAGCGGAGGAAACAAGCGG |
| | | AAGCTGAGCACCGCCATTGCTCTGATCGGATGCCCACCTC |
| | | TGGTCCTGCTGGATGAACCCACCACCGGAATGGACCCCC |
| | | AGGCTAGAAGAATGCTCTGGAACGTGATCGTGTCTATCA |
| | | TCCGCGAGGGCAGAGCTGTGGTGCTGACCTCTCACAGCA |
| | | TGGAAGAGTGCGAGGCTCTGTGTACCCGGCTGGCCATTA |
| | | TGGTCAAGGGCGCCTTCAGATGCATGGGCACCATTCAGC |
| | | ATCTGAAAAGCAAGTTCGGCGACGGCTACATCGTGACAA |
| | | TGAAGATCAAGAGCCCCAAGGACGACCTCCTGCCTGATC |
| | | TGAACCCCGTGGAACAGTTTTTTCAGGGCAACTTCCCCGG |
| | | CTCCGTGCAGCGGGAAAGACACTATAACATGCTGCAGTT |
| | | TCAGGTGTCCTCCTCCAGCCTGGCTCGGATCTTTCAACTG |
| | | CTGCTCTCTCACAAGGACAGCCTGCTGATTGAAGAGTAC |
| | | AGCGTGACACAGACCACACTCGACCAGGTTTTCGTGAAC |
| | | TTCGCCAAGCAGCAGACCGAGAGCCACGACCTGCCTCTG |
| | | CATCCTAGAGCCGCTGGTGCCTCTAGACAAGCTCAGGAC |
| | | TAAGCTTCCACTGGATTGTACAATTACATAAAATAAAAT |
| | | ATCTTTATTTTCATTACATCTGTGTGTTGGTTTTTTGTGTG |
| | | CGCTACTAACCTGAGACCGATCTGTTGATCAGCAGTTCAA |
| | | CCTGTTGATAGTACGTACTAAGCTCTCATGTTTCACGTAC |
| | | TAAGCTCTCATGTTTAACGTACTAAGCTCTCATGTTTAAC |
| | | GAACTAAACCCTCATGGCTAACGTACTAAGCTCTCATGG |
| | | CTAACGTACTAAGCTCTCATGTTTCACGTACTAAGCTCTC |
| | | ATGTTTGAACAATAAAATTAATATAAATCAGCAACTTAA |
| | | ATAGCCTCTAAGGTTTTAAGTTTTATAAGAAAAAAAAGA |
| | | ATATATAAGGCTTTTAAAGCTTTTAAGGTTTAACGGTTGT |
| | | GGACAACAAGCCAGGGATGTAACGCACTGAGAAGCCCTT |
| | | AGAGCCTCTCAAAGCAATTTTGAGTGACACAGGAACACT |
| | | TAACGGCTGACATGGGTGTCTCAAAATCTCTGATGTTACA |
| | | TTGCACAAGATAAAAATATATCATCATGAACAATAAAAC |
| | | TGTCTGCTTACATAAACAGTAATACAAGGGGTGTTATGA |
| | | GCCATATTCAACGGGAAACGTCGAGGCCGCGATTAAATT |
| | | CCAACATGGATGCTGATTTATATGGGTATAAATGGGCTC |
| | | GCGATAATGTCGGGCAATCAGGTGCGACAATCTATCGCT |
| | | TGTATGGGAAGCCCGATGCGCCAGAGTTGTTTCTGAAAC |
| | | ATGGCAAAGGTAGCGTTGCCAATGATGTTACAGATGAGA |
| | | TGGTCAGACTAAACTGGCTGACGGAATTTATGCCTCTTCC |
| | | GACCATCAAGCATTTTATCCGTACTCCTGATGATGCATGG |
| | | TTACTCACCACTGCGATCCCCGGAAAAACAGCATTCCAG |
| | | GTATTAGAAGAATATCCTGATTCAGGTGAAAATATTGTTG |
| | | ATGCGCTGGCAGTGTTCCTGCGCCGGTTGCATTCGATTCC |
| | | TGTTTGTAATTGTCCTTTTAACAGCGATCGCGTATTTCGTC |
| | | TCGCTCAGGCGCAATCACGAATGAATAACGGTTTGGTTG |
| | | ATGCGAGTGATTTTGATGACGAGCGTAATGGCTGGCCTG |
| | | TTGAACAAGTCTGGAAAGAAATGCATAAACTTTTGCCAT |
| | | TCTCACCGGATTCAGTCGTCACTCATGGTGATTTCTCACT |
| | | TGATAACCTTATTTTTGACGAGGGGAAATTAATAGGTTGT |
| | | ATTGATGTTGGACGAGTCGGAATCGCAGACCGATACCAG |
| | | GATCTTGCCATCCTATGGAACTGCCTCGGTGAGTTTTCTC |
| | | CTTCATTACAGAAACGGCTTTTTCAAAAATATGGTATTGA |
| | | TAATCCTGATATGAATAAATTGCAGTTTCATTTGATGCTC |
| | | GATGAGTTTTTCTAATCAGAATTGGTTAATTGGTTGTAAC |
| | | AGGTCTCAAACC |

What is claimed is:

1. A method of producing a therapeutic circular DNA vector, the method comprising:
(a) providing a sample comprising a template DNA vector comprising a therapeutic sequence and a backbone sequence;
(b) amplifying the template DNA vector using a polymerase-mediated rolling-circle amplification to generate a linear concatemer;
(c) digesting the linear concatemer with a type IIs restriction enzyme that cuts a first site and a second site per unit of the linear concatemer, wherein the first and second sites flank the therapeutic sequence and form self-complementary overhangs, thereby producing a linear therapeutic fragment and a linear backbone fragment, wherein the linear therapeutic fragment comprises the therapeutic sequence and the linear backbone fragment comprises the backbone sequence, or a portion thereof; and
(d) contacting the linear backbone fragment and the linear therapeutic fragment with a ligase to produce a circular backbone and a therapeutic circular DNA vector lacking a type IIs restriction site.

2. The method of claim 1, wherein the linear backbone fragment of (c) comprises a type IIs restriction site, the circular backbone of (d) comprises the type IIs restriction site, and the type IIs restriction enzyme cuts the circular backbone and does not cut the therapeutic circular DNA vector.

3. A method of producing a therapeutic circular DNA vector, the method comprising:
(a) providing a sample comprising a template DNA vector comprising a therapeutic sequence and a backbone sequence;
(b) amplifying the template DNA vector using a polymerase-mediated rolling-circle amplification to generate a linear concatemer;
(c) digesting the linear concatemer with one or more restriction enzymes that cut at least a first site, a second site, and a third site per unit of the linear concatemer, wherein: (i) the first and second sites flank the therapeutic sequence and form self-complementary overhangs, and (ii) the third site is within the backbone sequence and forms an overhang that is non-complementary to the first or second site, thereby producing a linear therapeutic fragment comprising the therapeutic sequence and at least two linear backbone fragments each comprising a portion of the backbone sequence; and
(d) contacting the linear therapeutic fragment with a ligase to produce a therapeutic circular DNA vector in solution.

4. The method of any one of claims 1-3, wherein the method further comprises diluting the DNA between steps (c) and step (d).

5. The method of any one of claims 1-4, wherein the DNA concentration at the beginning of step (d) is greater than or equal to 20 μg/mL but less than 160 μg/mL.

6. The method of claim 5, wherein the DNA concentration at the beginning of step (d) is about 40 μg/mL.

7. The method of claim 5, wherein the DNA concentration at the beginning of step (d) is about 80 μg/mL.

8. The method of any one of claims 1-7, wherein the ligase concentration in step (d) is from about 10 to about 20 U ligase per g DNA.

9. The method of any one of claims 1-8, wherein the ligase is a T4 ligase.

10. The method of any one of claims 1-9, wherein no temperature increase is performed immediately after step (d).

11. The method of any one of claims 3-10, wherein the linear concatemer is digested with a single restriction enzyme that cuts the first site, the second site, and the third site.

12. The method of claim 11, wherein the one or more restriction enzymes cut a fourth site of the linear concatemer per unit, wherein the fourth site is within the backbone sequence and forms an overhang that is non-complementary to the first or second site, and wherein the digestion produces at least three linear backbone fragments each comprising a portion of the backbone sequence.

13. The method of claim 12, wherein the single restriction enzyme cuts a fourth site of the linear concatemer per unit, wherein the fourth site is within the backbone sequence and forms an overhang that is non-complementary to the first or second site, and wherein the digestion produces at least three linear backbone fragments each comprising a portion of the backbone sequence.

14. The method of any one of claims 1, 2, 4-11, and 13, wherein the restriction enzyme is a type IIs restriction enzyme.

15. The method of claim 14, wherein the type IIs restriction enzyme is BsaI.

16. The method of any one of claims 1-15, wherein no restriction enzyme inactivation step precedes step (d).

17. The method of any one of claims 1-16, wherein no temperature increase is performed between steps (c) and (d).

18. The method of any one of claims 1-17, wherein no temperature increase is performed immediately after step (d).

19. The method of any one of claims 1-18, wherein steps (c) and (d) occur simultaneously.

20. The method of any one of claims 1-19, further comprising raising the temperature of the solution containing the therapeutic circular DNA vector to about 65° C.

21. The method of any one of claims 1-20, further comprising:
(e) contacting the therapeutic circular DNA vector with a topoisomerase or a helicase.

22. The method of claim 21, wherein step (e) is performed at about 37° C.

23. The method of any one of claims 1-22, further comprising:
(f) contacting the linear backbone fragments with an exonuclease.

24. The method of claim 23, wherein step (f) is performed at about 37° C.

25. The method of any one of claims 1-20, further comprising:
(e) contacting the therapeutic circular DNA vector with a topoisomerase or a helicase; and
(f) contacting the linear backbone fragments with an exonuclease,
wherein no enzyme inactivation step is performed between steps (e) and (f).

26. The method of claim 25, wherein step (e) occurs before step (f).

27. The method of claim 25, wherein step (f) occurs before step (d).

28. The method of any one of claims 1-27, wherein the restriction enzyme is provided at a concentration from about 0.5 U/μg to about 20 U/μg.

29. The method of claim 28, wherein the restriction enzyme is provided at a concentration from about 0.5 U/μg to about 2.5 U/μg.

30. The method of claim 29, wherein the restriction enzyme is provided at a concentration of about 2.5 U/μg.

31. The method of any one of claims 1-30, wherein step (c) comprises incubation from one to 12 hours.

32. The method of any one of claims 1-30, wherein step (c) comprises incubation for one hour or less.

33. The method of claim 32, wherein step (c) comprises incubation for about one hour.

34. The method of any one of claims 1-33, wherein the ligase is provided at a concentration no greater than 20 U ligase per g DNA (U/μg).

35. The method of claim 34, wherein the ligase is provided at a concentration of about 10 U/μg.

36. The method of any one of claims 1-35, wherein the ligase is T4 ligase.

37. The method of any one of claims 21-36, wherein the topoisomerase is provided at a concentration no greater than 10 U topoisomerase per g DNA (U/μg).

38. The method of any one of claims 21-37, wherein the topoisomerase is a type II topoisomerase.

39. The method of any one of claims 21-38, wherein the topoisomerase is gyrase or topoisomerase IV.

40. The method of any one of claims 23-39, wherein the exonuclease is provided at a concentration from about 0.5 U/μg to about 20 U/μg.

41. The method of any one of claims 23-40, wherein step (f) is performed two or more times.

42. The method of any one of claims 23-41, wherein step (f) comprises incubation from one hour to 18 hours.

43. The method of claim 42, wherein step (f) comprises incubation from 3-18 hours.

44. The method of any one of claims 23-43, wherein the exonuclease is T5 exonuclease.

45. The method of any one of claims 1-44, further comprising:
(g) running the therapeutic circular DNA vector through a column; and/or
(h) precipitating the therapeutic circular DNA vector with isopropyl alcohol.

46. The method of any one of claims 1-45, wherein step (b) is performed using site-specific primers.

47. The method of any one of claims 1-46, wherein step (b) is performed using random primers.

48. The method of any one of claims 1-47, wherein the quantity of therapeutic circular DNA vector produced is at least five-fold the quantity of plasmid DNA vector in the sample of step (a).

49. The method of any one of claims 1-48, wherein no DNA purification or gel extraction step is performed before step (d).

50. The method of any one of claims 1-49, wherein the amount of the therapeutic circular DNA in the solution of step (d) is at least 2.0% of the amount of the linear concatemer in step (b) by weight.

51. The method of any one of claims 1-50, wherein the amount of the therapeutic circular DNA produced in step (d) is at least 1.0 mg.

52. The method of any one of claims 1-51, wherein the concentration of the therapeutic circular DNA in the solution after step (d) is at least 5 μg/mL without any purification or concentration being performed.

53. The method of any one of claims 1-52, wherein the volume of the solution of step (d) is at least five liters.

54. The method of any one of claims 1-53, wherein steps (b) through (d) are performed in a reaction vessel having a volume of at least one liter.

55. The method of any one of claims 1-54, wherein the amount of the therapeutic circular DNA produced in step (d) is at least five-fold the amount of the template DNA vector provided in step (a).

56. A method of removing a backbone sequence from a DNA molecule to produce a therapeutic circular DNA vector, wherein the DNA molecule comprises the backbone sequence and a therapeutic sequence, the method comprising:
(a) digesting the DNA molecule with a type IIs restriction enzyme that cuts a first site and a second site per unit of the linear concatemer, wherein the first and second sites flank the therapeutic sequence and form self-complementary overhangs, thereby producing a linear therapeutic fragment and a linear backbone fragment, wherein the linear therapeutic fragment comprises the therapeutic sequence and the linear backbone fragment comprises at least a portion of the backbone sequence and a type IIs restriction site; and
(b) contacting the linear backbone fragment and the linear therapeutic fragment with a ligase to produce a circular backbone comprising the type IIs restriction site and a therapeutic circular DNA vector lacking a type IIs restriction site.

57. A method of removing a backbone sequence from a DNA molecule to produce a therapeutic circular DNA vector, wherein the DNA molecule comprises the backbone sequence and a therapeutic sequence, the method comprising:
(a) digesting the DNA molecule with one or more restriction enzymes that cut at least a first site, a second site, and a third site per unit of the DNA molecule, wherein: (i) the first and second sites flank the therapeutic sequence and form self-complementary overhangs, and (ii) the third site is within the backbone sequence and forms an overhang that is non-complementary to the first or second site, thereby producing a linear therapeutic fragment comprising the therapeutic sequence and at least two linear backbone fragments each comprising a portion of the backbone sequence; and
(b) contacting the linear therapeutic fragment with a ligase to produce a therapeutic circular DNA vector in solution.

58. The method of claim 57, wherein the linear concatemer is digested with a single restriction enzyme that cuts the first site, the second site, and the third site.

59. The method of claim 57, wherein the one or more restriction enzymes cut a fourth site of the DNA molecule, wherein the fourth site is within the backbone sequence and forms an overhang that is non-complementary to the first or second site, and wherein the digestion produces at least three linear backbone fragments each comprising a portion of the backbone sequence.

60. The method of claim 58, wherein the single restriction enzyme cuts a fourth site of the DNA molecule, wherein the fourth site is within the backbone sequence and forms an overhang that is non-complementary to the first or second site, and wherein the digestion produces at least three linear backbone fragments each comprising a portion of the backbone sequence.

61. The method of any one of claims 57-60, wherein the DNA molecule is a concatemer produced by amplification of a template DNA vector.

62. The method of any one of claims 57-60, wherein the DNA molecule is a template DNA vector.

63. The method of claim 62, wherein the template DNA vector is a plasmid DNA vector.

64. The method of any one of claims 56-63, wherein the restriction enzyme is a type IIs restriction enzyme.

65. The method of claim 64, wherein the type IIs restriction enzyme is BsaI.

66. The method of any one of claims 56-65, wherein no restriction enzyme inactivation step precedes step (b).

67. The method of any one of claims 56-66, wherein no temperature increase is performed between steps (a) and (b).

68. The method of any one of claims 56-67, wherein steps (a) and (b) occur simultaneously.

69. The method of any one of claims 56-68, further comprising raising the temperature of the solution containing the therapeutic circular DNA vector to about 65° C.

70. The method of any one of claims 56-69, further comprising:
(c) contacting the therapeutic circular DNA vector with a topoisomerase or a helicase.

71. The method of claim 70, wherein step (c) is performed at about 37° C.

72. The method of any one of claims 56-71, further comprising:
(d) contacting the linear backbone fragments with an exonuclease.

73. The method of claim 72, wherein step (d) is performed at about 37° C.

74. The method of any one of claims 56-73, further comprising:
(c) contacting the therapeutic circular DNA vector with a topoisomerase or a helicase; and
(d) contacting the linear backbone fragments with an exonuclease,
wherein no enzyme inactivation step is performed between steps (c) and (d).

75. The method of claim 74, wherein step (c) occurs before step (d).

76. The method of claim 74, wherein step (d) occurs before step (c).

77. The method of any one of claims 56-76, wherein the restriction enzyme is provided at a concentration of from about 0.5 U/μg to about 20 U/μg.

78. The method of claim 77, wherein the restriction enzyme is provided at a concentration of about 2.5 U/μg.

79. The method of any one of claims 56-78, wherein step (a) comprises incubation from one to 12 hours.

80. The method of claim 79, wherein step (a) comprises incubation for about one hour.

81. The method of any one of claims 56-80, wherein the ligase is provided at a concentration no greater than 20 U ligase per μg DNA (U/μg).

82. The method of claim 81, wherein the ligase is provided at a concentration of about 10 U/μg.

83. The method of any one of claims 56-82, wherein the ligase is T4 ligase.

84. The method of any one of claims 70-83, wherein the topoisomerase is provided at a concentration no greater than 10 U topoisomerase per g DNA (U/μg).

85. The method of any one of claims 70-84, wherein the topoisomerase is a type II topoisomerase.

86. The method of any one of claims 70-85, wherein the topoisomerase is gyrase or topoisomerase IV.

87. The method of any one of claims 70-86, wherein the exonuclease is provided at a concentration from about 0.5 U/μg to about 20 U/μg.

88. The method of any one of claims 70-87, wherein step (d) is performed two or more times.

89. The method of any one of claims 70-88, wherein step (d) comprises incubation from one hour to 12 hours.

90. The method of any one of claims 70-89, wherein the exonuclease is T5 exonuclease.

91. The method of any one of claims 56-90, further comprising:
(e) running the therapeutic circular DNA vector through a column; and/or
(f) precipitating the therapeutic circular DNA vector with isopropyl alcohol.

92. The method of any one of claims 56-91, wherein the therapeutic circular DNA vector is produced in the absence of a gel extraction step.

93. A method of producing a supercoiled therapeutic circular DNA vector, the method comprising:
(a) providing a sample comprising a template DNA vector comprising a therapeutic sequence and a backbone sequence;
(b) amplifying the template DNA vector using a polymerase-mediated rolling-circle amplification to generate a linear concatemer;
(c) digesting the linear concatemer with a type IIs restriction enzyme that cuts a first site and a second site per unit of the linear concatemer, wherein the first and second sites flank the therapeutic sequence and form self-complementary overhangs, thereby producing a linear therapeutic fragment and a linear backbone fragment, wherein the linear therapeutic fragment comprises the therapeutic sequence and the linear backbone fragment comprises at least a portion of the backbone sequence; and
(d) diluting the linear therapeutic fragment and the linear backbone fragment to a cumulative DNA concentration from 20 μg/mL to 160 μg/mL;
(e) contacting the diluted linear backbone fragment and the linear therapeutic fragment with a ligase to produce a circular backbone and a therapeutic circular DNA vector lacking a type Us restriction site;
(f) contacting the therapeutic circular DNA vector with gyrase at a concentration of about 1.5 U per μg DNA to produce a mixture of supercoiled therapeutic circular DNA vectors and linear backbone fragments; and
(g) after step (f), digesting the linear backbone fragments with an exonuclease.

94. A method of producing a supercoiled therapeutic circular DNA vector, the method comprising:
(a) providing a sample comprising a template DNA vector comprising a therapeutic sequence and a backbone sequence;
(b) amplifying the template DNA vector using a polymerase-mediated rolling-circle amplification to generate a linear concatemer;
(c) digesting the linear concatemer with a type IIs restriction enzyme that cuts a first site and a second site per unit of the linear concatemer, wherein the first and second sites flank the therapeutic sequence and form self-complementary overhangs, thereby producing a linear therapeutic fragment and a linear backbone fragment, wherein the linear therapeutic fragment comprises the therapeutic sequence and the linear backbone fragment comprises at least a portion of the backbone sequence; and
(d) diluting the linear therapeutic fragment and the linear backbone fragment to a cumulative DNA concentration from 20 μg/mL to 160 μg/mL;
(e) contacting the diluted linear backbone fragment and the linear therapeutic fragment with a ligase to produce a circular backbone and a therapeutic circular DNA vector lacking a type IIs restriction site;
(f) digesting the linear backbone fragment with an exonuclease; and
(g) after step (f), supercoiling the therapeutic circular DNA vector with gyrase at a concentration of less than 1.5 U per g DNA.

95. The method of claim 93 or 94, wherein the ligase of step (e) is at a concentration from 10 to 20 U ligase per μg DNA.

96. The method of any one of claims 93-95, wherein the diluted cumulative DNA concentration of step (d) is about 10% to about 80% of cumulative DNA concentration immediately after step (c).

97. The method of claim 96, wherein the cumulative DNA concentration immediately after step (c) is between 100 μg/mL and 300 μg/mL.

98. The method of any one of claims 1-97, wherein the first or second cut sites flanking the therapeutic sequence comprises AAAA or AACC.

99. A method for large-scale production of a therapeutic circular DNA vector, the method comprising:
(a) providing a sample of a plasmid DNA vector comprising a therapeutic sequence and a backbone sequence;
(b) amplifying the plasmid DNA vector in a reaction volume of at least 500 mL using a polymerase-mediated rolling-circle amplification to generate a linear concatemer;
(c) digesting the linear concatemer with one or more restriction enzymes that cut at least a first site, a second site, and a third site per unit of the linear concatemer, wherein: (i) the first and second sites flank the therapeutic sequence and form self-complementary overhangs, and (ii) the third site is within the backbone sequence and forms an overhang that is non-complementary to the first or second site, thereby producing a linear therapeutic fragment comprising the therapeutic sequence and at least two linear backbone fragments each comprising a portion of the backbone sequence; and
(d) contacting the linear therapeutic fragment with a ligase to produce a therapeutic circular DNA vector in solution.

100. The method of claim 99, wherein the amount of the plasmid DNA vector provided in step (a) is at least 1.0 mg.

101. The method of claim 99 or 100, wherein step (b) produces at least 100 mg of the linear concatemer.

102. The method of any one of claims 99-101, wherein step (d) produces at least 2.0 mg of the therapeutic circular DNA vector.

103. The method of any one of claims 99-102, wherein steps (c) and (d) occur simultaneously.

104. The method of any one of claim 99-103, wherein no DNA purification is performed during or between steps (b), (c), and (d).

105. The method of any one of claims 99-104, wherein the amount of the therapeutic circular DNA in the solution of step (d) is at least 2.0% of the amount of the linear concatemer in step (b) by weight

106. The method of any one of claims 99-105, wherein the amount of the therapeutic circular DNA produced in step (d) is at least twice the amount of the plasmid DNA vector provided in step (a).

107. The method of any one of claims 99-106, wherein the DNA concentration at the beginning of step (d) is greater than or equal to 20 μg/mL but less than 160 μg/mL.

108. The method of claim 107, wherein the DNA concentration at the beginning of step (d) is from about 40 μg/mL to about 80 μg/mL.

109. The method of claim 108, wherein the DNA concentration at the beginning of step (d) is about 40 μg/mL.

110. The method of claim 108, wherein the DNA concentration at the beginning of step (d) is about 80 μg/mL.

111. The method of any one of claims 99-110, wherein the ligase concentration in step (d) is from about 10 to about 20 U ligase per μg DNA.

112. The. method of any one of claims 99-111, wherein the ligase is a T4 ligase.

113. The method of any one of claims 99-112, wherein no temperature increase is performed immediately after step (d).

114. A method of producing a therapeutic circular DNA vector, the method comprising:
(a) providing a solution comprising DNA molecules, wherein each DNA molecule comprises a backbone sequence and a therapeutic sequence;
(b) adding a type IIs restriction enzyme to the solution to digest the DNA molecules, thereby separating the backbone sequences from the therapeutic sequences;
(c) adding a ligase to the solution to produce a reaction in a mixture comprising:
(i) the ligase;
(ii) the type IIs restriction enzyme;
(iii) therapeutic circular DNA vectors each comprising a single therapeutic sequence, wherein the therapeutic circular DNA vectors each lack a type IIs recognition site; and
(iv) byproducts, wherein each byproduct comprises one or more type IIs restriction sites,
wherein the ratio of the therapeutic circular DNA vectors to the byproducts comprising one or more type IIs restriction sites increases as the reaction proceeds.

115. The method of claim 114, wherein some or all of the byproducts comprise one or more backbone sequences.

116. The method of claim 115, wherein some or all of the byproducts further comprise two or more therapeutic sequences.

117. The method of any one of claims 114-116, wherein some or all of the byproducts are circularized.

118. The method of any one of claims 114-117, wherein the DNA molecules of (a) are concatemers.

119. The method of any one of claims 114-118, wherein the method further comprises, prior to step (a), amplifying a template DNA vector using rolling circle amplification to generate concatemers.

120. The method of any one of claims 114-119, wherein the type IIs restriction enzyme is BsaI.

121. The method of any one of claims 114-120, wherein no restriction enzyme inactivation step precedes step (d).

122. The method of any one of claims 114-121, wherein no temperature increase is performed between steps (b) and (c).

123. The method of any one of claims 114-122, further comprising raising the temperature of the solution containing the therapeutic circular DNA vector to about 65° C.

124. The method of any one of claims 114-123, further comprising:
(e) contacting the therapeutic circular DNA vector with a topoisomerase or a helicase.

125. The method of claim 124, wherein step (e) is performed at about 37° C.

126. The method of any one of claims 114-125, further comprising:
(f) contacting linear byproducts with an exonuclease.

127. The method of claim 126, wherein step (f) is performed at about 37° C.

128. The method of any one of claims 114-123, further comprising:
(e) contacting the therapeutic circular DNA vector with a topoisomerase or a helicase; and
(f) contacting linear byproducts with an exonuclease,
wherein no enzyme inactivation step is performed between steps (e) and (f).

129. The method of claim 128, wherein step (e) occurs before step (f).

130. The method of claim 129, wherein step (f) occurs before step (e).

131. The method of any one of claims 114-130, wherein the restriction enzyme is provided at a concentration from about 0.5 U/μg to about 20 U/μg.

132. The method of claim 131, wherein the restriction enzyme is provided at a concentration from about 0.5 U/μg to about 2.5 U/μg.

133. The method of claim 132, wherein the restriction enzyme is provided at a concentration of about 2.5 U/μg.

134. The method of any one of claims 114-133, wherein step (c) comprises incubation from one to 12 hours.

135. The method of claim 134, wherein step (c) comprises incubation for about one hour.

136. The method of any one of claims 114-135, wherein the ligase is provided at a concentration no greater than 20 U ligase per μg DNA (U/μg).

137. The method of claim 136, wherein the ligase is provided at a concentration of about 10 U/μg.

138. The method of any one of claims 114-137, wherein the ligase is T4 ligase.

139. The method of any one of claims 104-138, wherein the topoisomerase is provided at a concentration no greater than 10 U topoisomerase per ag DNA (U/μg).

140. The method of any one of claims 104-139, wherein the topoisomerase is a type II topoisomerase.

141. The method of any one of claims 104-140, wherein the topoisomerase is gyrase or topoisomerase IV.

142. The method of any one of claims 104-141, wherein the exonuclease is provided at a concentration from about 0.5 U/μg to about 20 U/μg.

143. The method of any one of claims 104-142, wherein step (f) is performed two or more times.

144. The method of any one of claims 104-143, wherein step (f) comprises incubation from one hour to 12 hours.

145. The method of any one of claims 104-144, wherein the exonuclease is T5 exonuclease.

146. The method of any one of claims 104-145, further comprising:
(g) running the therapeutic circular DNA vector through a column; and/or
(h) precipitating the therapeutic circular DNA vector with isopropyl alcohol.

147. The method of any one of claims 104-146, wherein step (b) is performed using site-specific primers.

148. The method of any one of claims 104-147, wherein step (b) is performed using random primers.

149. The method of any one of claims 104-148, wherein no gel extraction step is performed before step (d).

150. The method of any one of claims 104-149, wherein the amount of the therapeutic circular DNA in the solution of step (d) is at least 2.0% of the amount of the DNA molecule in step (a) by weight.

151. The method of any one of claims 104-150, wherein the amount of the therapeutic circular DNA produced in step (d) is at least 2.0 mg.

152. The method of any one of claims 104-151, wherein the concentration of the therapeutic circular DNA in the solution after step (d) is at least 5.0 μg/mL prior to any purification or concentration being performed.

153. The method of any one of claims 104-152, wherein the volume of the solution of step (d) is at least 5.0 liters.

154. The method of any one of claims 104-153, wherein steps (b) through (d) are performed in a reaction vessel having a volume of at least 1.0 liter.

155. A method of producing a therapeutic circular DNA vector, the method comprising:
(a) providing a mixture of DNA comprising a plurality of linear therapeutic DNA fragments and a plurality of linear backbone DNA fragments, wherein each linear therapeutic DNA fragment comprises a therapeutic sequence and self-complementary ends, wherein the plurality of linear therapeutic DNA fragments and linear backbone DNA fragments are at a cumulative DNA concentration from 20 μg/mL to 160 μg/mL; and
(b) performing a ligation reaction by contacting the mixture of DNA with a ligase at a concentration from 10 to 20 U ligase per μg DNA to produce a therapeutic circular DNA vector.

156. The method of claim 155, wherein the mixture of DNA was produced by a type IIs restriction digest reaction, wherein a type IIs restriction enzyme cleaves the linear therapeutic DNA fragments from the linear backbone DNA fragments, wherein the self-complementary ends are type IIs overhangs.

157. A method of producing a therapeutic circular DNA vector, the method comprising:
(a) producing a mixture of DNA comprising a plurality of linear therapeutic DNA fragments and a plurality of linear backbone DNA fragments by a type IIs restriction digest reaction, wherein a type IIs restriction enzyme cleaves the linear therapeutic DNA fragments from the linear backbone DNA fragments, wherein each linear therapeutic DNA fragment comprises a therapeutic sequence and self-complementary type IIs overhangs, wherein the plurality of linear therapeutic DNA fragments and linear backbone DNA fragments are at a cumulative DNA concentration from 20 μg/mL to 160 μg/mL; and
(b) performing a ligation reaction by contacting the mixture of DNA with a ligase at a concentration from 10 to 20 U ligase per μg DNA to produce a therapeutic circular DNA vector.

158. The method of any one of claims 155-157, wherein the cumulative DNA concentration of step (a) is achieved by adjusting the cumulative DNA concentration immediately after the type IIs restriction digest.

159. The method of any one of claims 155-158, wherein the cumulative DNA concentration immediately after the type IIs restriction digest is diluted to achieve the cumulative DNA concentration of step (a).

160. The method of any one of claims 155-159, wherein the cumulative DNA concentration immediately after the type IIs restriction digest is from 100 μg/mL to 300 μg/mL.

161. The method of any one of claims 155-160, wherein the cumulative DNA concentration of step (a) is diluted to about 10% to about 80% of the cumulative DNA concentration immediately after the type IIs restriction digest.

162. The method of any one of claims 155-161, wherein the cumulative DNA concentration of step (a) is from about 40 μg/mL to about 80 μg/mL.

163. The method of any one of claims 155-162, wherein the type IIs restriction enzyme in the type IIs restriction digest reaction is at a concentration from about 0.5 to about 2.5 U per μg DNA.

164. The method of any one of claims 155-163, wherein the ligase is at a concentration of about 10 U/ug.

165. The method of any one of claims 155-164, wherein the ligation reaction is carried out for at least five hours.

166. The method of claim 165, wherein the ligation reaction is carried out for 18-24 hours.

167. The method of any one of claims 155-166, wherein the ligase is a T4 ligase.

168. The method of any one of claims 156-167, wherein the type IIs restriction enzyme in the type IIs restriction digest reaction is at a concentration from about 0.5 to about 2.5 U per μg DNA.

169. The method of any one of claims 156-168, wherein the type IIs restriction digest reaction is carried out for no more than two hours.

170. The method of claim 169, wherein the type IIs restriction digest reaction is carried out for 10 minutes to one hour.

171. The method of any one of claims 156-170, wherein the type IIs restriction enzyme is BsaI.

172. The method of any one of claims 156-171, wherein the type IIs overhangs each comprise four bases.

173. The method of claim 172, wherein two and only two of the four bases are A or T.

174. The method of any one of claims 156-183 wherein the type IIs overhangs comprise AAAA or AACC.

175. The method of any one of claims 155-174, further comprising:
(c) contacting the therapeutic circular DNA vector with a topoisomerase or a helicase.

176. The method of any one of claims 155-174, further comprising:
(d) contacting the linear backbone fragments with an exonuclease.

177. The method of any one of claims 155-174, further comprising:
(c) contacting the therapeutic circular DNA vector with a topoisomerase or a helicase; and
(d) contacting the linear backbone fragments with an exonuclease.

178. The method of any one of claims 175-177, wherein no enzyme inactivation step is performed between steps (c) and (d).

179. The method of claim 178, wherein step (c) occurs before step (d).

180. The method of claim 178, wherein step (d) occurs before step (c).

181. The method of any one of claims 175 or 177-180, wherein the topoisomerase is provided at a concentration no greater than 10 U topoisomerase per g DNA (U/μg).

182. The method of any one of claims 175 or 177-181, wherein the topoisomerase is a type II topoisomerase.

183. The method of any one of claims 175 or 177-181, wherein the topoisomerase is gyrase or topoisomerase IV.

184. The method of any one of claims 176-183, wherein the exonuclease is provided at a concentration from about 0.5 U/μg to about 20 U/μg.

185. The method of any one of claims 176-184, wherein step (d) is performed two or more times.

186. The method of any one of claims 176-185, wherein step (d) comprises incubation from one hour to 18 hours.

187. The method of claim 186, wherein step (d) comprises incubation from 3-18 hours.

188. The method of any one of claims 176-187, wherein the exonuclease is T5 exonuclease.

189. The method of any one of claims 155-188, further comprising:
(e) running the therapeutic circular DNA vector through a column; and/or
(f) precipitating the therapeutic circular DNA vector with isopropyl alcohol.

190. The method of any one of claims 155-189, wherein the amount of the therapeutic circular DNA produced in step (b) is at least 1.0 mg.

191. The method of any one of claims 155-190, wherein the concentration of the therapeutic circular DNA in the solution after step (b) is at least 5 μg/mL without any purification or concentration being performed.

192. The method of any one of claims 155-191, wherein the volume of the solution of step (d) is at least five liters.

193. The method of any one of claims 155-192, wherein step (b) is performed in a reaction vessel having a volume of at least one liter.

194. The method of any one of claims 155-193, wherein the mixture of DNA is a product of in vitro amplification.

195. The method of claim 194, wherein the in vitro amplification is a polymerase-mediated rolling-circle amplification.

196. The method of any one of claims 155-195, wherein the method does not comprise a gel-extraction step.

197. The method of any one of claims 155-196, wherein the DNA mixture comprises only one species of linear backbone DNA fragment.

198. A method of producing a supercoiled therapeutic circular DNA vector, the method comprising:
(a) providing a sample comprising a therapeutic circular DNA vector in relaxed circular form, wherein the therapeutic circular DNA vector comprises a therapeutic sequence;
(b) contacting the sample with a gyrase, wherein the concentration of the gyrase is about 1.5 U per mg of therapeutic circular DNA vector, thereby producing a composition of supercoiled therapeutic circular DNA vector.

199. The method of claim 198, wherein the sample of (a) further comprises linear DNA byproducts, and wherein the method further comprises, after (b), contacting the composition of supercoiled therapeutic circular DNA vector with an exonuclease under conditions suitable to digest linear DNA byproducts.

200. A method of producing a supercoiled therapeutic circular DNA vector, the method comprising:
(a) providing a sample comprising a therapeutic circular DNA vector in relaxed circular form and linear DNA byproducts, wherein the therapeutic circular DNA vector comprises a therapeutic sequence;
(b) contacting the sample with an exonuclease under conditions suitable to digest the linear DNA byproducts to form a digested sample; and
(c) contacting the digested sample with a gyrase, wherein the concentration of the gyrase is greater than 0.1 U per mg of therapeutic circular DNA vector and less than 1.5 U per mg of therapeutic circular DNA vector, thereby producing a supercoiled therapeutic circular DNA vector.

201. The method of claim 200, wherein the exonuclease is a T5 exonuclease.

202. The method of any one of claims 198-201, further comprising, before step (a), contacting a linear therapeutic fragment with a ligase to produce the therapeutic circular DNA vector.

203. The method of any one of claims 198-201, wherein the ligase is a T4 ligase.

204. The method of claim 202 or 203, further comprising, before contacting the linear therapeutic fragment with the ligase, digesting a linear concatemer comprising a therapeutic sequence with a restriction enzyme to cut a first site and a second site per unit of the linear concatemer, wherein the first and second sites flank the therapeutic sequence and form self-complementary overhangs, thereby producing the linear therapeutic fragment and the linear DNA byproducts.

205. The method of any one of claims 198-204, wherein the supercoiled therapeutic circular DNA vector is within a composition of therapeutic circular DNA vectors, wherein at least 70% of the therapeutic circular DNA vectors are supercoiled.

206. The method of any one of claims 1-205, wherein the therapeutic sequence is greater than 5 kb.

207. The method of any one of claims 1-206, wherein the therapeutic sequence comprises two or more transcription units.

208. The method of any one of claims 1-207, wherein the therapeutic sequence encodes one or more therapeutic proteins.

209. The method of claim 208, wherein the one or more therapeutic proteins is a multimeric protein.

210. The method of any one of claims 1-209, wherein the therapeutic sequence encodes a therapeutic nucleic acid.

211. The method of claim 210, wherein the therapeutic nucleic acid is an RNA molecule.

212. The method of claim 211, wherein the RNA molecule is a self-replicating RNA molecule, a short hairpin RNA, or a microRNA.

213. The method of any one of claims 1-212, wherein the therapeutic circular DNA vector is formulated as a pharmaceutical composition.

214. The method of any one of claims 1-213, further comprising formulating the therapeutic circular DNA vector in a pharmaceutically acceptable carrier to produce a pharmaceutical composition.

215. The method of claim 213 or 214, wherein the pharmaceutical composition comprises at least 1.0 mg of the therapeutic circular DNA vector in a pharmaceutically acceptable carrier.

216. The method of claim 214 or 215, wherein the therapeutic circular DNA vector in the pharmaceutical composition is at least 70% supercoiled monomer.

217. The method of any one of claims 213-216, wherein the pharmaceutical composition comprises no more than 1.0% of residual protein or backbone sequence.

218. The method of any one of claims 213-217, wherein the pharmaceutical composition comprises <1.0% protein content by mass, less than <1.0% RNA content by mass, and less than <5 EU/mg endotoxin.

219. A pharmaceutical composition produced by the method of any one of claims 213-218.

220. A method of expressing a therapeutic sequence in an individual, wherein the method comprises administering to the individual the pharmaceutical composition of claim 219.

221. A method of treating a disease or disorder in an individual in need thereof, the method comprising administering to the individual the pharmaceutical composition of claim 219.

222. The method of claim 220 or 221, wherein the method comprises in vivo electrotransfer.

223. The method of claim 222, wherein the in vivo electrotransfer induces expression of the therapeutic sequence in skin, skeletal muscle, tumor, eye, or lung of the individual.

224. A therapeutic circular DNA vector comprising a therapeutic sequence having a 3′ end and 5′ end, wherein the 3′ end of the therapeutic sequence is connected to the 5′ end of the therapeutic sequence by a four-base pair sequence comprising at least two consecutive adenines (A's)

225. The therapeutic circular DNA vector of claim 224, wherein the four-base pair sequence consists of AAAA.

226. The therapeutic circular DNA vector of claim 225, wherein the therapeutic circular DNA vector comprises a nucleic acid sequence having 85% sequence identity to SEQ ID NO: 1.

227. The therapeutic circular DNA vector of claim 226, wherein the therapeutic circular DNA vector comprises SEQ ID NO: 1.

228. The therapeutic circular DNA vector of claim 224, wherein two and only two consecutive bases of the four-base pair sequence are AA.

229. The therapeutic circular DNA vector of claim 228, wherein the four-base pair sequence consists of AACC.

230. The therapeutic circular DNA vector of claim 229, wherein the therapeutic circular DNA vector comprises a nucleic acid sequence having 85% sequence identity to SEQ ID NO: 3.

231. The therapeutic circular DNA vector of claim 230, wherein the therapeutic circular DNA vector comprises SEQ ID NO: 3.

232. A pharmaceutical composition comprising the therapeutic circular DNA vector of any one of claims 224-231.

233. The pharmaceutical composition of claim 232, wherein the pharmaceutical composition comprises at least 1.0 mg of the therapeutic circular DNA vector in a pharmaceutically acceptable carrier.

234. The pharmaceutical composition of claim 232 or 233, wherein the therapeutic circular DNA vector is at least 70% supercoiled monomer.

235. The pharmaceutical composition of any one of claims 232-234, wherein the pharmaceutical composition comprises no more than 1.0% of residual protein or backbone sequence.

236. The pharmaceutical composition of any one of claims 232-235, wherein the pharmaceutical composition comprises <1.0% protein content by mass, less than <1.0% RNA content by mass, and less than <5 EU/mg endotoxin.

237. A method of expressing a therapeutic sequence in an individual, wherein the method comprises administering to the individual the pharmaceutical composition of any one of claims 232-236.

238. A method of treating an ocular disease or disorder in an individual in need thereof, the method comprising administering to the individual the pharmaceutical composition of any one of claims 232-236.

239. The method of claim 237 or 238, wherein the method comprises delivering the therapeutic circular DNA vector to an eye of the individual by in vivo electrotransfer.

240. The method of any one of claims 220-223 or 237-239, wherein the individual is a human.
